(19) United States
(12) Patent Application Publication
(10) Pub. No.: US 2007/0048742 A1
(43) Pub. Date: Mar. 1, 2007

(54) Detection method for chromosome abnormality and microarray chip

(76) Inventors: Jason-Jongho Kang, Seoul (KR); Eun-Hee Oh, Anyang-city (KR); Hyun-Woong Kang, Seoul (KR); Ji-Hyun Lee, Uijeongbu-city (KR); Chang-Joon Bae, Seoul (KR); Jong-Ho Lee, Seoul (KR); Jeong-Sun Seo, Seoul (KR)

Correspondence Address: CHRISTIE, PARKER & HALE, LLP, PO BOX 7068, PASADENA, CA, 91109-7068, US

(21) Appl. No.: 11/211,185

(22) Filed: Aug. 23, 2005

(30) Foreign Application Priority Data
Aug. 23, 2004 (KR) 10-2004-0066384

(51) Int. Cl. C12Q 1/68

(52) U.S. Cl. 435/6

(57) ABSTRACT

The present invention relates to a method of detecting a chromosome abnormality and a microarray chip for detecting a chromosome abnormality. More specifically, the present invention is directed to a method of detection for chromosome abnormality in order to easily diagnosing the health condition and disease of the subject by detecting of the chromosome abnormality rapidly and precisely, and to providing a probe for diagnosis of disease by identifying chromosome abnormality specific to the disease.

### CROSS REFERENCE TO RELATED APPLICATION

This application claims priority to and the benefit of Korean Application No. 10-2004-0066384 filed in the Korean Patent Office on Aug. 23, 2004, the entire content of which is incorporated hereinto by reference.

### FIELD OF THE INVENTION

The present invention relates to a method of detection for chromosome abnormality. More specifically, the present invention is directed to a method of detection for chromosome abnormality in order to easily diagnose the health condition and disease of the subject by detecting a chromosome abnormality rapidly and precisely, and to provides a probe for diagnosis of a disease by identifying the chromosome abnormality specific to the disease.

### BACKGROUND OF THE INVENTION

Chromosome abnormality is associated with genetic defect and degenerative disease. The chromosome abnormality can be a deletion or duplication of a chromosome, a deletion or duplication of a part of chromosome, or a break, translocation, or inversion in the chromosome. The chromosome abnormality is a disturbance in the genetic balance and causes fetal death or serious defect in physical and mental states. For examples, Down's syndrome is a common abnormality of chromosome number caused by the presence of a third chromosome 21. Edwards syndrome (18+), Patau syndrome (13+), Turner syndrome (XO) and Klinefelter syndrome (XXY) also belong to abnormalities in chromosome number.

The chromosome abnormality can be detected by using Karyotype, and Fluorescent In Situ Hybridization (FISH). The detection methods have disadvantages in terms of time, labor and accuracy. Moreover, the Karyotype requires much time in cell culture. FISH can only be used for samples where the nucleic acid sequence and chromosomal location are known. Comparative genome hybridization (CGH) can be used to avoid problems of FISH. CGH can analyze a whole genome to detect the part where chromosome number abnormality occurs. However, the disadvantage of CGH has a low resolution compared to FISH.

In a different approach, DNA microarrays can be used for detecting the chromosome abnormality. DNA microarray systems are classified into cDNA microarrays, oligonucleotide microarrays, and genome microarrays, depending upon the bio-molecules immobilized on the microarray. Even though cDNA microarrays and oligonucleotide microarrays are easily prepared, the systems have the disadvantages of the limitation in the number of probes immobilized on the microarray, high cost of probe preparation, and difficulty in detecting a chromosome abnormality located external to the probe. In particular, for genomic DNA microarray systems, although the probe can be easily made, and can detect chromosome abnormalities in the expansive area of the chromosome, and also in intron areas of the chromosome, it is difficult to prepare a large number of DNA fragments where the chromosomal location and function are identified.

Bacterial Artificial Chromosome (BAC) clones includes BAC vectors containing a certain size fragment of the whole human genome. Because a BAC clone includes one fragment of a human DNA, the BAC clone corresponding to a specific part of a chromosome can be arrayed by analyzing the nucleic acid sequence and chromosomal location of the inserted DNA. Thus, specific DNA fragments can be easily obtained from each BAC clone.

### SUMMARY OF THE INVENTION

In one embodiment, the present invention provides a method of preparing a genomic DNA microarray for detecting chromosome abnormality.

In another embodiment, the present invention provides a method of preparing a microarray in which at least two probes are immobilized in one spot.

In yet another embodiment, the present invention provides a DNA chip for detecting chromosome abnormality in the whole human genome.

In yet another embodiment, the present invention provides a DNA chip for detecting at least one disease selected from the group consisting of Down's syndrome, Patau syndrome, Edwards syndrome, Turner syndrome, Klinefelter syndrome, ATR-16 (alpha-thalassemia retardation) syndrome, CAT1A (Charcot-Marie-Tooth neuropathy 1A), Cri-du-chat syndrome, Digeorge syndrome, HNPP (Hereditary Neuropathy with liability to pressure palsies) disease, Kallmann syndrome, Miller-Dieker syndrome, Prader-willi syndrome, Rubinstein-taybi syndrome, Smith-magenes syndrome, Steroid-sulfatase gene syndrome, Williams syndrome, Wolf-hirschhorn syndrome, and constitutional diseases checked pre- or post-natally.

In yet another embodiment, the present invention provides a method of detecting chromosome abnormality with a microarray for use in detecting chromosome abnormality.

In yet another embodiment of the present invention is provided with a diagnosis method for disease of chromosome number abnormality by using a microarray for detecting chromosome abnormality.

In yet another embodiment of the present invention is provided with a method for detecting chromosome abnormality specific to disease by using the microarray for detecting chromosome abnormality.

### BRIEF DESCRIPTION OF THE DRAWING

The patent or application file contains at least one drawing executed in color. Copies of this patent or patent application publication with color drawing(s) will be provided by the Office upon request and payment of the necessary fee.

A more complete appreciation of the invention, and many of the attendant advantages thereof, will be readily apparent as the same becomes better understood by reference to the following detailed description when considered in conjunction with the accompanying drawing, wherein:

FIG. 1 is a restriction map of the vector pECBAC1.

FIG. 2a to FIG. 2h represent the chromosomal location of the probes used for preparing the BAC chip in accordance with Example 2.

FIG. 3 shows structure of BAC chip in accordance with Example 2.

FIG. 4a to FIG. 4f show the location of specific probe in chromosome on BAC chip accordance with Example 2.

FIG. 5 represents analysis result for BAC chip of female patient with Patau syndrome.

FIG. 6 represents analysis result for BAC chip of male patient with Patau syndrome.

FIG. 7 represents analysis result for BAC chip of male patient with Edwards syndrome.

FIG. 8 represents analysis result for BAC chip of female patient with Edwards syndrome.

FIG. 9 represents analysis result for BAC chip of female patient with Down's syndrome.

FIG. 10 represents analysis result for BAC chip of female patient with duplication of chromosome 21 and X chromosome (karyotype XXY).

FIG. 11 represents analysis result for BAC chip of patient with Turner syndrome.

FIG. 12 represents analysis result for BAC chip of patient with Klinefelter syndrome.

FIG. 13 represents analysis result for BAC chip of normal woman.

FIG. 14 represents analysis result for BAC chip of normal man.

FIG. 15 represents analysis result for BAC chip of female patient with Down's syndrome.

### DETAILED DESCRIPTION

The present invention relates to a method of preparation for a genomic DNA microarray for detecting a chromosome abnormality comprising the steps of:

(a) identifying genomic DNA fragment inserted to each clone which is obtained from organism's genomic library;

(b) selecting target chromosomal region to be detected for chromosome abnormality;

(c) selecting at least a genomic DNA fragment as a probe for detecting the target chromosomal region in genomic DNA fragments identified in step (a),

wherein the probe has a single chromosomal locus, includes repetitive sequence in the amount of at most 85% of the probe nucleotide, and its nucleotide sequence constitute whole or a part of the target chromosomal region, or complementary sequence thereof; and

(d) immobilizing the probe in a microarray.

In an embodiment, the present invention relates to a method of preparing a microarray including a step of immobilizing at least one probe thereon selected from the group consisting of RNA fragment, cDNA fragment, oligomer and genomic DNA fragment. The microarray includes at least two kinds of probes immobilized in a spot, and the probes immobilized in a spot includes a nucleotide selected from the group consisting of whole chromosome, parts of chromosome, and a complementary DNA thereof showing the same phenotype in an abnormality of chromosome number, and the phenotype is a gene amplification, or a gene deletion.

In another embodiment, the present invention provides aDNA chip for detecting chromosome abnormality in the whole human genomic DNA, the DNA chip comprising a microarray containing at least one immobilized genomic DNA fragment selected from the group consisting of DNA fragment 1 to 25851 of Table 1, in appendix A hereto, as described in the detailed description of the present application, at least one oligomer including 20 to 100 bp of consecutive nucleotide sequence of the genomic DNA fragment, or at least one 100 bp to 10 kbp cDNA derived from the genomic DNA fragment.

In addition, the present invention provides a DNA chip for detecting selected from the group consisting of Down's syndrome, Patau syndrome, Edwards syndrome, Turner syndrome, Klinefelter syndrome, ATR-16 (alpha-thalassemia retardation) syndrome, CAT1A (Charcot-Marie-Tooth neuropathy 1A), Cri-du-chat syndrome, Digeorge syndrome, HNPP (Hereditary Neuropathy with liability to pressure palsies), Kallmann syndrome, Miller-Dieker syndrome, Prader-willi syndrome, Rubinstein-taybi syndrome, Smith-magenes syndrome, Steroid-sulfatase gene syndrome, Williams syndrome, Wolf-hirschhorn syndrome, and constitutional diseases checked pre- or post-natally.

The present invention provides a method of detecting chromosome abnormality comprising the steps of:

(a) preparing a microarray for detecting chromosome abnormality;

(b) reacting sample genomic DNA in a sample which is labeled by first detectable labeling material, and the reference DNA labeled by second detectable labeling material in the same mixture ratio, with the probe immobilized on the microarray, and measuring hybridization ratio of the sample genomic DNA or the reference DNA with the probe respectively; and

(c) analyzing chromosome abnormality of the sample genomic DNA on the basis of the hybridization ratio.

In an embodiment, the present invention provides a method of detecting chromosome abnormality comprising the steps of:

(a) selecting a probe including consecutive 20 to 100 bp of genomic DNA fragment, and immobilizing the probe on microarray, wherein the genomic DNA fragment is BAC DNA in Table 1 or BAC DNA in Table 2 as described in the detailed description of the present application, and the probe has a single chromosomal locus, and includes repetitive sequence in the amount of at most 85% of the probe nucleotide;

(b) reacting sample genomic DNA in a sample which is labeled by first detectable labeling material, and the reference DNA labeled by second detectable labeling material in the same mixture ratio, with the probe immobilized on the microarray, and measuring hybridization ratio of the sample genomic DNA or the reference DNA with the probe respectively; and

(c) deciding chromosome abnormality of the sample genomic DNA as follows:

No chromosome abnormality, if the hybridization ratio of the target genomic DNA is the same as that of the reference DNA,

Chromosome amplification, if the hybridization ratio of the target genomic DNA is higher than that of the reference DNA.

Chromosome deletion, if the hybridization ratio of the sample genomic DNA is lower than that of the reference DNA.

In an embodiment, the present invention provides a method of detection abnormality of chromosome number comprising steps of:

(a) immobilizing at least one probe thereon selected from the group consisting of RNA fragment, cDNA fragment, oligomer and genomic DNA fragment,

wherein the microarray includes at least two kinds of probes immobilized in a spot, and the probes immobilized in a spot includes a nucleotide selected from the group consisting of whole chromosome, parts of chromosome, and a complementary DNA thereof showing the same phenotype in an abnormality of chromosome number, and the phenotype is a gene amplification, or a gene deletion.

(b) reacting sample genomic DNA in a sample which is labeled by first detectable labeling material, and the reference DNA labeled by second detectable labeling material in the same mixture ratio, with the probe immobilized on the microarray, and measuring hybridization ratio of the sample genomic DNA or the reference DNA with the probe respectively; and

(c) deciding chromosome abnormality of the sample genomic DNA as follows:

No chromosome abnormality, if the hybridization ratio of the sample genomic DNA is the same as that of the reference DNA is decided to produce no chromosome abnormality,

Chromosome amplification, if the hybridization ratio of the sample genomic DNA is higher than that of the reference DNA.

Chromosome deletion, if the hybridization ratio of the sample genomic DNA is lower than that of the reference DNA.

In an embodiment, the present invention provides a method of detection a disease-specific chromosome abnormality comprising the steps of:

(a) identifying a genomic DNA fragment inserted to each clone which is obtained from organism's genomic library;

(b) immobilizing the entire or a part of genomic DNA fragment identified in step (a) on a microarray;

(b) reacting the sample genomic DNA of patient which is labeled by a first detectable labeling material, and a reference DNA labeled by a second detectable labeling material in the same mixture ratio, with the probe immobilized on the microarray, and measuring hybridization ratio of the sample genomic DNA or the reference DNA with the probe respectively; and

(c) detecting a region and type of a chromosome abnormality of the patient by analyzing the hybridization ratio.

The present invention will be described in more detail.

As used herein, the term, “organism” means plant, insect, animal, or human.

As used herein, the term, “genomic DNA microarray” means “a microarray which the genomic DNA fragment of the organism is immobilized thereon. The genomic DNA microarray can be a genomic DNA fragment which is obtained by separating and amplifying the each clone constituting a genomic library.

As used herein, the term, “clone” means a cell or cell line transformed by a vector including fragments of whole genome of the organism. The vector, cell and cell line are generally used for preparing genomic DNA library. For example, the vector includes BAC (Bacterial Artificial Chromosome) such as pECBAC1 and pBeloBACII, HAC (Human Artificial Chromosome), TAC (Transformation competent Artificial Chromosome), YAC (Yeast Artificial Chromosome) such as pYAC4, Pac (P1-derived Artificial chromosome) such as pCYPAC1, P1 (Phage) such as pAd10sacBII, and cosmid, but not limited thereto. A host can be suitably selected for each vector. The examples of the host include E. coli, yeast, and virus. The genomic library and clone can be prepared according to the general method in the art.

As used herein, the term, “probe” can be one which is immobilized on a microarray and can hybridize the homologous DNA in a sample. The probe can be DNA, RNA, cDNA or mRNA, or oligomer DNA. The probe includes repetitive sequence in the amount of at most 85% of the probe nucleotide, preferably at most 70%, more preferably at most 50%, most preferably at most 40%. The probe is 10 to 350 kb of a genomic DNA, 20 to 300 bp of oligomer, preferably 20 to 100 bp of oligomer, and 100 bp to 10 kbp of cDNA or RNA, but not limited thereto. Preferably, the probe has a single chromosomal locus.

Microarray

The microarray of the present invention is prepared by method comprising a step of immobilizing at least one probe thereon selected from the group consisting of RNA fragment, cDNA fragment, oligomer and genomic DNA fragment. The microarray includes at least two kinds of probes immobilized in a spot, and the probes immobilized in a spot includes a nucleotide selected from the group consisting of whole chromosome, parts of chromosome, and a complementary DNA thereof showing the same phenotype in an abnormality of chromosome number, and the phenotype is a gene amplification, or a gene deletion.

The probe is a genomic DNA fragment, or RNA fragment, cDNA fragment or oligomer derived from the genomic DNA fragment. The probe can be selected suitably for the usage of the microarray. The microarray can be prepared by the general method in the art. For example, the probe is immobilized on a substrate for microarray through physical or chemical binding. The substrate can be any one which is widely used in the art. Preferably, the substrate can be functional group for immobilizing the probe on the surface of the substrate, nitrocellulose, or material being capable of forming three dimensional structure. The examples of substrates include silicone wafer, glass, polycarbonate, membrane, polymer films such as polystyrene, or polyurethane, and porous material.

The probe can be immobilized according to the general immobilization method used in the preparation of the DNA microarray, for examples photolithography, piezoelectric printing, micropippetting method and spotting method.

The probe can be immobilized at the concentration of 2 to 100 pg on a spot of microarray. One kind of probe having a same nucleotide sequence can be immobilized in a spot. Alternatively, at least two kinds of probe having different nucleotide sequences can be immobilized in a spot together. In addition, the number of spot on microarray is at least one fold, preferably 2 to 3 fold. The shape of spot is circular with 50 to 500 um of diameter, and 10 to 500 um of interval between the spots. The size and density of spot can be adjusted suitably depending upon the resolution of the analyzing system for the microarray.

For the microarray of the present invention, micro well plate, for example 96-well plate can be used so that the sample molecule, labeling, hybridization, and washing process can be performed with the automatic machines (for examples, Biomek, and Genetix robot).

Genomic DNA Fragment

The genomic DNA fragment can be obtained from whole chromosome of the organism to produce a fragment having suitable size and nucleotide sequence, obtained from the genome library of the organism, or synthesized chemically.

The size of genomic DNA fragment can be adjusted depending upon the resolution of the analyzing system for microarray. For example, the genomic DNA fragment is in the mean size of 100 to 350 kb. In considering that Micro-deletion of the minimal mutation of chromosome abnormality in whole chromosome is 1 Mb, the BAC chip of the present invention including the genomic DNA with mean size of 100 to 350 kb provides high resolution of analyzing the chromosome abnormality.

The Table 1, and Table 2 show the name, chromosomal location, and the related genetic information of each BAC clone. By using the chromosomal location of the BAC clone, the nucleotide sequence of genomic DNA can be identified on the web site providing the database of human genome sequence for example http://www.ncbi.nlm.nih.gov/BLAST, in particular http://www.ncbi.nlm.nih.gov/BLAST/Blast.cgi?CMD=Web&LAYOUT=TwoWindows &AUTO_FORMAT=Semiauto&ALIGNMENTS=50&ALIGNMENT_VIEW=Pairwise &CLIENT=web&DATABASE=nr&DESCRIPTIONS=100&ENTREZ_QUERY=%28n one %29&EXPECT=10&FILTER=L&FORMAT_OBJECT=Alignment&FORMAT_TYPE=HTML&NCBI_GI=on&PAGE=Nucleotides&PROGRAM=blastn&SERVICE=plain&SET_DEFAULTS.x=34&SET_DEFAULTS.y:=8&SHOW_OVERVIEW=on&END_OF_HTTPGET=Yes&SHOW_LINKOUT=yes&GET_SEOUENCE=yes. In Table 1, the A-1 is referred to chromosome, and A-2 is referred to the chromosomal location.

The genomic DNA fragments obtained from the genomic library are shown in Table 1, and can be used for probes of the genomic DNA microarray. The genomic DNA microarray can includes genomic DNA fragments which are corresponding to the whole genome of the organism to be detected for chromosome abnormality, or a genomic DNA fragments which are corresponding for a gene of interest or specific chromosomal region to be detected for chromosome abnormality. When the chromosome abnormality can be detected for the whole genome, the number of genomic DNA fragment can be calculated by dividing the size of organism genome with the size of genomic DNA fragment. For example, if the genomic DNA microarray is used for detecting chromosome abnormality in whole human genome, the size of human genome 3×109 bp is divided with mean size of 100 to 350 kb of genomic DNA fragment to obtain about 30,000 to 8571 of genomic DNA fragments or clones. As the size of genomic DNA fragment increase, the number of genomic DNA fragment or clone decrease. The genomic DNA fragments of Table 1 include the whole human genome.

In addition, the genomic DNA fragments of Table 2 can be used for probe. The genomic DNA fragments of Table 2 are chromosomal regions showing characteristic abnormality with high frequency that are selected from the whole human genome. The BAC library including 1440 of BAC DNA are deposited on Aug. 6, 2005 to the Korean Collection for Type Culture in Korea Research Institute of Bioscience and Biotechnology, and thus are given as accession number of KCTC 10681 BP.

TABLE 2

| | Name | Chromosomal location | Chromosomal location | | |
|---|---|---|---|---|---|
| No | of BAC DNA | identified by FISH | start | end | size |
| 1 | BAC230_P11 | 1p36.33 | 1529227 | 1629227 | 100000 |
| 2 | BAC101_I16 | 1p36.33 | 1544102 | 1644102 | 100000 |
| 3 | BAC176_F17 | 1p36.33 | 1965497 | 2072101 | 106604 |
| 4 | BAC38_N15 | 1p36.33 | 2071926 | 2157699 | 85773 |
| 5 | BAC141_N19 | 1p36.32 | 3342971 | 3443382 | 100411 |
| 6 | BAC171_P09 | 1p36.32 | 4519322 | 4603310 | 83988 |
| 7 | BAC90_A08 | 1p36.22 | 10901796 | 11050196 | 148400 |
| 8 | BAC180_C01 | 1p36.22 | 11483863 | 11588661 | 104798 |
| 9 | BAC91_A07 | 1p36.21 | 14463027 | 14559007 | 95980 |
| 10 | BAC80_C03 | 1p36.13 | 15568474 | 15753918 | 185444 |
| 11 | BAC145_B18 | 1p36.13 | 17433404 | 17530502 | 97098 |
| 12 | BAC68_M07 | 1p36.12 | 21346731 | 21390543 | 43812 |
| 13 | 8AC133_M03 | 1p36.12 | 21445582 | 21545172 | 99590 |
| 14 | BAC13_F18 | 1p36.12 | 21849691 | 21936333 | 86642 |
| 15 | BAC236_J05 | 1p36.11 | 25799868 | 25886691 | 86823 |
| 16 | BAC37_K05 | 1p36.11-1p35.3 | 27536304 | 27732094 | 195790 |
| 17 | BAC46_E05 | 1p35.3 | 27979812 | 28065764 | 85952 |
| 18 | BAC120_F15 | 1p35.3 | 27989288 | 28065750 | 76462 |
| 19 | BAC63_I09 | 1p35.3 | 29786955 | 29876434 | 89479 |
| 20 | BAC146_G09 | 1p35.2 | 31602874 | 31685710 | 82836 |
| 21 | BAC124_B01 | 1p35.2 | 32122405 | 32230934 | 108529 |
| 22 | BAC20_I15 | 1p35.1 | 34156770 | 34286546 | 129776 |
| 23 | BAC96_E21 | 1p34.3 | 35350185 | 35456363 | 106178 |
| 24 | BAC92_N23 | 1p34.3 | 36280093 | 36388589 | 108496 |
| 25 | BAC33_L19 | 1p34.3 | 37955994 | 38065753 | 109759 |
| 26 | BAC162_J22 | 1p34.3-1p34.2 | 39595361 | 39679116 | 83755 |
| 27 | BAC215_J18 | 1p34.2 | 39775792 | 39897211 | 121419 |
| 28 | BAC108_F03 | 1p34.2 | 43182933 | 43268729 | 85796 |
| 29 | BAC108_H02 | 1p34.1 | 44746375 | 44848074 | 101699 |
| 30 | BAC96_M17 | 1p33 | 46133544 | 46237625 | 104081 |
| 31 | BAC24_E20 | 1p33 | 47088207 | 47198549 | 110342 |
| 32 | BAC141_H16 | 1p33 | 47825865 | 47921500 | 95635 |
| 33 | BAC29_J22 | 1p32.3 | 53141519 | 53247758 | 106239 |
| 34 | BAC142_G14 | 1p32.3-1p32.2 | 55324477 | 55424617 | 100140 |
| 35 | BAC193_L08 | 1p32.2 | 56307529 | 56402186 | 94657 |
| 36 | BAC22_P12 | 1p32.2 | 56900186 | 56982456 | 82270 |
| 37 | BAC71_J20 | 1p32.1 | 58918422 | 59022531 | 104109 |
| 38 | BAC81_F02 | 1p31.3 | 63750835 | 63875564 | 124729 |
| 39 | BAC15_M17 | 1p31.3 | 64332444 | 64429163 | 96719 |
| 40 | BAC104_G18 | 1p31.3 | 64641455 | 64741455 | 100000 |
| 41 | BAC77_K04 | 1p31.1 | 77299984 | 77465197 | 165213 |
| 42 | BAC189_F17 | 1p31.1 | 82701695 | 82779000 | 77305 |
| 43 | BAC78_B10 | 1p22.3 | 85084501 | 85204460 | 119959 |
| 44 | BAC74_F02 | 1p22.3 | 85383840 | 85492628 | 108788 |
| 45 | BAC118_I08 | 1p22.3 | 85917769 | 86011273 | 93504 |
| 46 | BAC59_G14 | 1p22.3 | 87204295 | 87316444 | 112149 |
| 47 | BAC58_B15 | 1p22.2 | 88628011 | 88720304 | 92293 |
| 48 | BAC53_D06 | 1p22.1 | 92355122 | 92456222 | 101100 |
| 49 | BAC153_B07 | 1p22.1 | 93003468 | 93079689 | 76221 |
| 50 | BAC65_M16 | 1p21.3 | 97378301 | 97457183 | 78882 |
| 51 | BAC20_G18 | 1p21.2 | 99893823 | 100009725 | 115902 |
| 52 | BAC114_F11 | 1p21.2 | 100940682 | 101041214 | 100532 |
| 53 | BAC22_M23 | 1p21.1 | 103514919 | 103639324 | 124405 |
| 54 | BAC51_G07 | 1p13.3 | 109427279 | 109546841 | 119562 |
| 55 | BAC127_H20 | 1p13.3 | 109757320 | 109851630 | 94310 |
| 56 | BAC91_J05 | 1p13.3 | 110271674 | 110383682 | 112008 |
| 57 | BAC132_I13 | 1p13.3 | 110690967 | 110796298 | 105331 |
| 58 | BAC157_J21 | 1p13.2 | 111161469 | 111262835 | 101366 |
| 59 | BAC227_N14 | 1p13.2 | 112331213 | 112421336 | 90123 |
| 60 | BAC79_K19 | 1p13.2 | 113757204 | 113845226 | 88022 |
| 61 | BAC59_B21 | 1p13.2 | 114031312 | 114140336 | 109024 |
| 62 | BAC113_H08 | 1p13.2 | 114377591 | 114451274 | 73683 |
| 63 | BAC60_F24 | 1p13.1 | 116586168 | 116695431 | 109263 |
| 64 | BAC29_M19 | 1p12 | 119519154 | 119604651 | 85497 |
| 65 | BAC123_I23 | 1q21.1 | 143124471 | 143219958 | 95487 |
| 66 | BAC78_H12 | 1q21.1 | 144772302 | 144872184 | 99882 |
| 67 | BAC1_O10 | 1q21.2-1q21.3 | 147975235 | 148087596 | 112361 |
| 68 | BAC85_N11 | 1q22 | 153324733 | 153420512 | 95779 |
| 69 | BAC215_N06 | 1q23.1 | 153939839 | 154026899 | 87060 |
| 70 | BAC235_K22 | 1q23.1-1q23.2 | 156223065 | 156316653 | 93588 |
| 71 | BAC183_F06 | 1q23.2 | 157165153 | 157241636 | 76483 |
| 72 | BAC249_I07 | 1q23.3 | 157852867 | 157951479 | 98612 |
| 73 | BAC96_I22 | 1q23.3 | 160045810 | 160143154 | 97344 |
| 74 | BAC157_C20 | 1q23.3 | 161881152 | 161958986 | 77834 |
| 75 | BAC177_L21 | 1q24.1 | 163760676 | 163858802 | 98126 |
| 76 | BAC84_E09 | 1q24.2 | 167891944 | 168000214 | 108270 |
| 77 | BAC72_N18 | 1q24.3 | 169998539 | 170103444 | 104905 |
| 78 | BAC155_J12 | 1q25.1 | 171121193 | 171205926 | 84733 |
| 79 | BAC81_C10 | 1q25.2 | 174263264 | 174334501 | 71237 |
| 80 | BAC165_M06 | 1q25.3 | 181278012 | 181364471 | 86459 |
| 81 | BAC109_M23 | 1q31.1 | 183567842 | 183706519 | 138677 |
| 82 | BAC82_F03 | 1q31.1 | 185022538 | 185123541 | 101003 |
| 83 | BAC25_H07 | 1q31.2 | 189675318 | 189773791 | 98473 |
| 84 | BAC59_M15 | 1q32.1 | 198546696 | 198675552 | 128856 |
| 85 | BAC80_G04 | 1q32.1 | 200225576 | 200325648 | 100072 |
| 86 | BAC118_L22 | 1q32.2 | 206836035 | 206935481 | 99446 |
| 87 | BAC84_O24 | 1q32.3 | 208823946 | 208921789 | 97843 |
| 88 | BAC170_F20 | 1q41 | 215527062 | 215620214 | 93152 |
| 89 | BAC42_F06 | 1q42.11 | 220912852 | 221021312 | 108460 |
| 90 | BAC169_C15 | 1q42.12 | 221854174 | 221940299 | 86125 |
| 91 | BAC139_G10 | 1q42.12 | 222528984 | 222625007 | 96023 |
| 92 | BAC50_H09 | 1q42.12 | 223840274 | 223945589 | 105315 |
| 93 | BAC128_O05 | 1q42.13 | 226601318 | 226716208 | 114890 |
| 94 | BAC147_E05 | 1q42.2 | 228714708 | 228801924 | 87216 |
| 95 | BAC118_H14 | 1q42.2 | 229716839 | 229873996 | 157157 |
| 96 | BAC40_F20 | 1q43 | 233676313 | 233767529 | 91216 |
| 97 | BAC97_D21 | 1q43 | 234066319 | 234144573 | 78254 |
| 98 | BAC132_D14 | 1q43 | 234777122 | 234889732 | 112610 |
| 99 | BAC6_N24 | 1q43 | 238506181 | 238657979 | 151798 |
| 100 | BAC37_P24 | 1q43 | 238718843 | 238814667 | 95824 |
| 101 | BAC168_C19 | 1q44 | 240499661 | 240601271 | 101610 |
| 102 | BAC134_B22 | 1q44 | 241086985 | 241187406 | 100421 |
| 103 | BAC92_E04 | 1q44 | 242668435 | 242826516 | 158081 |
| 104 | BAC19_D07 | 2p25.3 | 2017353 | 2153020 | 135667 |
| 105 | BAC153_L04 | 2p25.3 | 2130417 | 2206225 | 75808 |
| 106 | BAC34_P10 | 2p25.2 | 5967022 | 6058231 | 91209 |
| 107 | BAC165_O21 | 2p25.1 | 10318108 | 10420429 | 102321 |
| 108 | BAC29_C11 | 2p25.1 | 10563637 | 10661950 | 98313 |
| 109 | BAC76_D18 | 2p25.1 | 10572001 | 10663835 | 91834 |
| 110 | BAC247_B10 | 2p24.3 | 12216199 | 12291035 | 74836 |
| 111 | BAC99_G21 | 2p24.3 | 15156103 | 15242636 | 86533 |
| 112 | BAC126_O17 | 2p24.3 | 16059687 | 16152219 | 92532 |
| 113 | BAC239_O18 | 2p24.1 | 19283343 | 19416770 | 133427 |
| 114 | BAC167_M04 | 2p24.1 | 20441436 | 20533992 | 92556 |
| 115 | BAC64_M02 | 2p24.1 | 23127766 | 23259344 | 131578 |
| 116 | BAC146_F07 | 2p23.3 | 24196322 | 24312610 | 116288 |
| 117 | BAC37_E18 | 2p23.3 | 26239453 | 26346959 | 107506 |
| 118 | BAC176_M18 | 2p23.2 | 28152357 | 28237203 | 84846 |
| 119 | BAC97_N06 | 2p23.2 | 29382986 | 29494136 | 111150 |
| 120 | BAC252_B21 | 2p23.2 | 29791097 | 29940728 | 149631 |
| 121 | BAC137_N18 | 2p23.1 | 31471075 | 31559877 | 88802 |
| 122 | BAC175_J02 | 2p23.1 | 31547136 | 31639069 | 91933 |
| 123 | BAC90_P18 | 2p22.3 | 35880117 | 36001593 | 121476 |
| 124 | BAC166_F20 | 2p22.3 | 36077128 | 36161827 | 84699 |
| 125 | BAC145_H13 | 2p22.1 | 40487134 | 40589110 | 101976 |
| 126 | BAC32_A06 | 2p21 | 45100050 | 45213616 | 113566 |
| 127 | BAC143_E05 | 2p21 | 46141794 | 46251030 | 109236 |
| 128 | BAC143_E05 | 2p21 | 46141794 | 46251030 | 109236 |
| 129 | BAC52_F13 | 2p21 | 47579480 | 47691043 | 111563 |
| 130 | BAC120_P03 | 2p16.3 | 47845212 | 48002887 | 157675 |
| 131 | BAC118_O22 | 2p16.3 | 47920517 | 48020124 | 99607 |
| 132 | BAC43_G23 | 2p16.3-2p16.2 | 52875863 | 53027462 | 151599 |
| 133 | BAC65_K04 | 2p16.1 | 55274201 | 55360186 | 85985 |
| 134 | BAC142_G22 | 2p16.1 | 56656320 | 56746512 | 90192 |
| 135 | BAC122_G07 | 2p16.1 | 61056471 | 61156577 | 100106 |
| 136 | BAC209_G19 | 2p14 | 65279460 | 65413463 | 134003 |
| 137 | BAC15_M23 | 2p14 | 65872899 | 65971038 | 98139 |
| 138 | BAC12_G01 | 2p14 | 66621241 | 66738940 | 117699 |
| 139 | BAC140_N16 | 2p14 | 67904530 | 67995136 | 90606 |
| 140 | BAC10_H13 | 2p13.3 | 70532183 | 70664988 | 132805 |
| 141 | BAC145_L06 | 2p13.2 | 71897634 | 71982468 | 84834 |
| 142 | BAC83_P03 | 2p13.2 | 72809831 | 72900359 | 90528 |
| 143 | BAC165_F20 | 2p13.2 | 73335636 | 73442011 | 106375 |
| 144 | BAC92_C01 | 2p12 | 76516204 | 76659322 | 143118 |
| 145 | BAC23_H04 | 2p12 | 79284953 | 79287486 | 2533 |
| 146 | BAC144_A07 | 2p12 | 79710126 | 79791630 | 81504 |
| 147 | BAC69_J21 | 2p12 | 82679356 | 82770285 | 90929 |
| 148 | BAC125_H18 | 2p11.2 | 84236834 | 84361221 | 124387 |
| 149 | BAC185_D04 | 2p11.2 | 84925449 | 85008840 | 83391 |
| 150 | BAC120_P19 | 2q11.2 | 96746116 | 96841468 | 95352 |
| 151 | BAC103_I04 | 2q11.2 | 100047624 | 100165050 | 117426 |
| 152 | BAC96_P09 | 2q11.2 | 101955120 | 102051808 | 96688 |
| 153 | BAC24_D14 | 2q12.1 | 102878427 | 102974217 | 95790 |
| 154 | BAC65_B13 | 2q12.2 | 106171053 | 106283646 | 112593 |
| 155 | BAC82_P19 | 2q12.2-2q12.3 | 107078856 | 107194169 | 115313 |
| 156 | BAC20_N21 | 2q12.3 | 108313490 | 108418580 | 105090 |
| 157 | BAC83_K08 | 2q13 | 113976889 | 114072208 | 95319 |
| 158 | BAC167_P18 | 2q14.1 | 116238411 | 116338753 | 100342 |
| 159 | BAC92_C18 | 2q14.2 | 119580327 | 119667173 | 86846 |
| 160 | BAC169_P06 | 2q14.3 | 124001323 | 124111921 | 110598 |
| 161 | BAC63_N08 | 2q14.3 | 128189768 | 128317548 | 127780 |
| 162 | BAC219_N21 | 2q14.3 | 129045452 | 129139403 | 93951 |
| 163 | BAC50_E06 | 2q14.3 | 129276736 | 129375469 | 98733 |
| 164 | BAC64_P09 | 2q21.1 | 130798725 | 130896452 | 97727 |
| 165 | BAC232_L16 | 2q21.2 | 133396374 | 133477510 | 81136 |
| 166 | BAC39_N20 | 2q21.2 | 134321293 | 134412045 | 90752 |
| 167 | BAC106_B08 | 2q21.2 | 135049602 | 135174621 | 125019 |
| 168 | BAC203_A08 | 2q21.3 | 137136390 | 137224316 | 87926 |
| 169 | BAC143_C23 | 2q22.1 | 138466776 | 138567212 | 100436 |
| 170 | BAC80_L11 | 2q22.1 | 141407568 | 141494343 | 86775 |
| 171 | BAC27_B01 | 2q22.2 | 142890598 | 142988238 | 97640 |
| 172 | BAC1_H07 | 2q22.3 | 146431858 | 146528367 | 96509 |
| 173 | BAC61_N08 | 2q22.3 | 146889891 | 147020573 | 130682 |
| 174 | BAC183_G19 | 2q22.3 | 148571685 | 148675857 | 104172 |
| 175 | BAC2_G24 | 2q23.3 | 153582938 | 153682713 | 99775 |
| 176 | BAC80_A06 | 2q23.3 | 154624152 | 154704864 | 80712 |
| 177 | BAC143_A10 | 2q24.2 | 160586550 | 160651020 | 64470 |
| 178 | BAC16_C12 | 2q24.2 | 162362900 | 162478343 | 115443 |
| 179 | BAC84_K05 | 2q24.3 | 165757755 | 165891389 | 133634 |
| 180 | BAC13_D10 | 2q31.1 | 175410950 | 175540734 | 129784 |
| 181 | BAC12_F20 | 2q31.2 | 180083767 | 180172257 | 88490 |
| 182 | BAC68_H23 | 2q32.1 | 184616113 | 184716113 | 100000 |
| 183 | BAC157_F03 | 2q32.1 | 184947921 | 185019810 | 71889 |
| 184 | BAC108_L17 | 2q32.2 | 190109495 | 190203100 | 93605 |
| 185 | BAC96_G06 | 2q32.2 | 190537424 | 190629363 | 91939 |
| 186 | BAC69_D21 | 2q32.3 | 192223633 | 192321196 | 97563 |
| 187 | BAC131_M09 | 2q32.3 | 196103995 | 196198815 | 94820 |
| 188 | BAC80_E09 | 2q33.1 | 198318250 | 198442088 | 123838 |
| 189 | BAC73_P04 | 2q33.1 | 202416889 | 202510456 | 93567 |
| 190 | BAC169_F10 | 2q33.2 | 204163343 | 204290158 | 126815 |
| 191 | BAC11_G01 | 2q33.3 | 206629769 | 206708802 | 79033 |
| 192 | BAC106_A14 | 2q33.3 | 207531505 | 207621456 | 89951 |
| 193 | BAC84_P12 | 2q33.3 | 208528037 | 208641718 | 113681 |
| 194 | BAC51_G24 | 2q34 | 210722184 | 210815860 | 93676 |
| 195 | BAC52_M13 | 2q34 | 211225694 | 211345982 | 120288 |
| 196 | BAC53_J20 | 2q34 | 211285930 | 211370955 | 85025 |
| 197 | BAC142_B20 | 2q34 | 211478043 | 211559272 | 81229 |
| 198 | BAC189_L05 | 2q35 | 215765277 | 215881636 | 116359 |
| 199 | BAC57_M03 | 2q35 | 219901806 | 220024698 | 122892 |
| 200 | BAC232_M13 | 2q35 | 220601959 | 220735687 | 133728 |
| 201 | BAC175_D13 | 2q35 | 221616979 | 221691527 | 74548 |
| 202 | BAC116_P06 | 2q36.1 | 223887636 | 223989934 | 102298 |
| 203 | BAC31_A16 | 2q36.2 | 226016682 | 226099535 | 82853 |
| 204 | BAC26_H23 | 2q36.3 | 230365419 | 230498721 | 133302 |
| 205 | BAC92_I07 | 2q37.1 | 231353147 | 231452691 | 99544 |
| 206 | BAC123_I06 | 2q37.1 | 233442324 | 233544202 | 101878 |
| 207 | BAC75_N17 | 2q37.1 | 233670472 | 233771526 | 101054 |
| 208 | BAC168_I09 | 2q37.1 | 233930130 | 234032857 | 102727 |
| 209 | BAC72_M15 | 2q37.1-2q37.2 | 235920318 | 236006465 | 86147 |
| 210 | BAC40_D08 | 2q37.2 | 236846749 | 236940679 | 93930 |
| 211 | BAC15_M19 | 2q37.3 | 237796557 | 237875116 | 78559 |
| 212 | BAC131_H16 | 2q37.3 | 238677217 | 238785504 | 108287 |
| 213 | BAC45_F02 | 3p26.3 | 275051 | 340022 | 64971 |
| 214 | BAC179_L14 | 3p26.3 | 1388914 | 1470006 | 81092 |
| 215 | BAC83_J18 | 3p26.2 | 3277224 | 3396544 | 119320 |
| 216 | BAC51_F01 | 3p26.1 | 7716397 | 7826256 | 109859 |
| 217 | BAC154_G23 | 3p25.3 | 8485609 | 8637667 | 152058 |
| 218 | BAC169_O13 | 3p25.3 | 8913703 | 9027785 | 114082 |
| 219 | BAC241_P15 | 3p25.3 | 9548591 | 9694711 | 146120 |
| 220 | BAC165_K17 | 3p25.2 | 12356300 | 12457958 | 101658 |
| 221 | BAC142_N07 | 3p25.1 | 14368582 | 14473229 | 104647 |
| 222 | BAC156_D05 | 3p24.2 | 23728515 | 23834333 | 105818 |
| 223 | BAC12_O03 | 3p24.2 | 25406725 | 25490112 | 83387 |
| 224 | BAC195_G14 | 3p24.1 | 27907117 | 27983880 | 76763 |
| 225 | BAC60_B02 | 3p24.1 | 29606006 | 29717251 | 111245 |
| 226 | BAC144_C11 | 3p22.3 | 35624058 | 35725997 | 101939 |
| 227 | BAC175_I18 | 3p22.3 | 36827747 | 36904902 | 77155 |
| 228 | BAC85_G09 | 3p22.3 | 37495978 | 37596686 | 100708 |
| 229 | BAC202_D18 | 3p22.2 | 39426298 | 39505044 | 78746 |
| 230 | BAC1_J07 | 3p22.1 | 41026807 | 41161409 | 134602 |
| 231 | BAC57_M07 | 3p21.33 | 44026108 | 44123419 | 97311 |
| 232 | BAC230_M11 | 3p21.31 | 44939097 | 45037027 | 97930 |
| 233 | BAC97_N23 | 3p21.31 | 45157018 | 45255447 | 98429 |
| 234 | BAC206_J21 | 3p21.31 | 45592613 | 45692595 | 99982 |
| 235 | BAC218_K17 | 3p21.31 | 50259524 | 50394367 | 134843 |
| 236 | BAC232_N06 | 3p21.31 | 50259585 | 50394368 | 134783 |
| 237 | BAC80_H11 | 3p21.31 | 51212640 | 51335514 | 122874 |
| 238 | BAC233_H20 | 3p21.31 | 51409024 | 51492346 | 83322 |
| 239 | BAC201_F16 | 3p21.2 | 51903826 | 51985597 | 81771 |
| 240 | BAC241_O06 | 3p21.1 | 52358933 | 52466419 | 107486 |
| 241 | BAC220_H15 | 3p21.1 | 53826039 | 53910910 | 84871 |
| 242 | BAC82_N23 | 3p14.3 | 54569991 | 54690327 | 120336 |
| 243 | BAC209_K05 | 3p14.3 | 55417906 | 55513268 | 95362 |
| 244 | BAC117_E10 | 3p14.3 | 55786520 | 55896480 | 109960 |
| 245 | BAC167_G07 | 3p14.3 | 56829214 | 56917105 | 87891 |
| 246 | BAC237_F09 | 3p14.3 | 57274644 | 57374644 | 100000 |
| 247 | BAC24_K03 | 3p14.2 | 59534562 | 59657247 | 122685 |
| 248 | BAC107_H04 | 3p14.2 | 60488717 | 60588995 | 100278 |
| 249 | BAC8_G11 | 3p14.2 | 61820632 | 61930362 | 109730 |
| 250 | BAC73_M11 | 3p14.2 | 63255713 | 63406796 | 151083 |
| 251 | BAC109_M19 | 3p14.1 | 64283237 | 64380231 | 96994 |
| 252 | BAC162_A19 | 3p14.1 | 65847882 | 65933524 | 85642 |
| 253 | BAC26_E03 | 3p14.1 | 68575144 | 68732074 | 156930 |
| 254 | BAC37_G21 | 3p13 | 71664491 | 71809306 | 144815 |
| 255 | BAC170_J09 | 3p13 | 72773163 | 72848368 | 75205 |
| 256 | BAC40_O18 | 3p12.3 | 79049212 | 79187519 | 138307 |
| 257 | BAC30_D18 | 3p12.1 | 86349564 | 86439457 | 89893 |
| 258 | BAC189_J06 | 3p12.1 | 87022335 | 87131219 | 108884 |
| 259 | BAC131_I23 | 3q12.1 | 99552954 | 99668465 | 115511 |
| 260 | BAC150_J01 | 3q12.2 | 101727427 | 101802045 | 74618 |
| 261 | BAC71_P12 | 3q12.3 | 102367380 | 102497142 | 129762 |
| 262 | BAC71_C14 | 3q13.11 | 106673835 | 106786786 | 112951 |
| 263 | BAC152_C16 | 3q13.12 | 109047504 | 109184907 | 137403 |
| 264 | BAC22_O17 | 3q13.2 | 114175050 | 114255992 | 80942 |
| 265 | BAC77_F10 | 3q13.2 | 114448879 | 114538525 | 89646 |
| 266 | BAC13_P22 | 3q13.2 | 114674650 | 114760656 | 86006 |
| 267 | BAC89_H07 | 3q13.33 | 122192318 | 122295140 | 102822 |
| 268 | BAC106_M21 | 3q21.2 | 125180747 | 125270946 | 90199 |
| 269 | BAC125_G19 | 3q21.2 | 126119672 | 126223738 | 104066 |
| 270 | BAC82_M18 | 3q21.2 | 126158673 | 126245625 | 86952 |
| 271 | BAC247_C06 | 3q21.3 | 129863236 | 129949580 | 86344 |
| 272 | BAC199_J09 | 3q22.1 | 133298059 | 133389923 | 91864 |
| 273 | BAC156_P13 | 3q22.1 | 134740871 | 134840157 | 99286 |
| 274 | BAC26_P08 | 3q22.2 | 136124485 | 136211253 | 86768 |
| 275 | BAC58_N16 | 3q22.2 | 136181252 | 136303768 | 122516 |
| 276 | BAC128_C03 | 3q22.3 | 138678996 | 138778794 | 99798 |
| 277 | BAC177_G04 | 3q23 | 1408794261 | 40999667 | 120241 |
| 278 | BAC155_H02 | 3q23 | 142427963 | 142532188 | 104225 |
| 279 | BAC129_C01 | 3q23 | 143905174 | 144040596 | 135422 |
| 280 | BAC23_N18 | 3q24 | 146432672 | 146523249 | 90577 |
| 281 | BAC96_G23 | 3q25.1 | 151595540 | 151710306 | 114766 |
| 282 | BAC133_H24 | 3q25.1 | 152272915 | 152430445 | 157530 |
| 283 | BAC25_B02 | 3q25.2 | 155320960 | 155423256 | 102296 |
| 284 | BAC173_F07 | 3q25.2 | 156118339 | 156231600 | 113261 |
| 285 | BAC22_K22 | 3q25.31 | 158295702 | 158382561 | 86859 |
| 286 | BAC27_D22 | 3q25.33 | 161337687 | 161441234 | 103547 |
| 287 | BAC78_P09 | 3q26.2 | 169265612 | 169369696 | 104084 |
| 288 | BAC215_M07 | 3q26.2 | 170100379 | 170186644 | 86265 |
| 289 | BAC28_H10 | 3q26.2 | 170633386 | 170738207 | 104821 |
| 290 | BAC97_L20 | 3q26.2 | 170638276 | 170738208 | 99932 |
| 291 | BAC143_K08 | 3q26.2 | 171033889 | 171122369 | 88480 |
| 292 | BAC87_K21 | 3q26.2 | 171403660 | 171503660 | 100000 |
| 293 | BAC46_F17 | 3q26.31 | 174232148 | 174347613 | 115465 |
| 294 | BAC178_H09 | 3q26.33 | 180549356 | 180619418 | 70062 |
| 295 | BAC144_B04 | 3q27.1 | 185218255 | 185303855 | 85600 |
| 296 | BAC97_O04 | 3q27.3 | 188040831 | 188144881 | 104050 |
| 297 | BAC195_F06 | 3q27.3 | 188642971 | 188723000 | 80029 |
| 298 | BAC213_I18 | 3q28 | 189306333 | 189443795 | 137462 |
| 299 | BAC144_N20 | 3q28 | 189898622 | 190003887 | 105265 |
| 300 | BAC74_E06 | 3q28 | 190429027 | 190528063 | 99036 |
| 301 | BAC119_D15 | 3q28 | 191342273 | 191418794 | 76521 |
| 302 | BAC173_O07 | 3q28 | 193343108 | 193452092 | 108984 |
| 303 | BAC61_O10 | 3q29 | 195596154 | 195712149 | 115995 |
| 304 | BAC243_N21 | 3q29 | 198051616 | 198128635 | 77019 |
| 305 | BAC120_B11 | 4p16.3 | 1099898 | 1204523 | 104625 |
| 306 | BAC192_N22 | 4p16.3 | 1200621 | 1300621 | 100000 |
| 307 | BAC50_H08 | 4p16.3 | 1776527 | 1908757 | 132230 |
| 308 | BAC67_I12 | 4p16.3 | 1864852 | 1972573 | 107721 |
| 309 | BAC163_J20 | 4p16.3 | 2790797 | 2871745 | 80948 |
| 310 | BAC139_E10 | 4p16.1 | 9556380 | 9652476 | 96096 |
| 311 | BAC100_B07 | 4p15.33 | 13288541 | 13398230 | 109689 |
| 312 | BAC75_N19 | 4p15.32 | 16974124 | 17099823 | 125699 |
| 313 | BAC193_F03 | 4p15.32 | 17367199 | 17450187 | 82988 |
| 314 | BAC21_I15 | 4p15.2 | 23831167 | 23940545 | 109378 |
| 315 | BAC12_K01 | 4p15.1 | 32069422 | 32161106 | 91684 |
| 316 | BAC41_K05 | 4p14 | 36442390 | 36544031 | 101641 |
| 317 | BAC84_I22 | 4p14 | 38032971 | 38156751 | 123780 |
| 318 | BAC168_J10 | 4p12 | 45977853 | 46074537 | 96684 |
| 319 | BAC33_M04 | 4p12 | 47957511 | 48056918 | 99407 |
| 320 | BAC42_P11 | 4q12 | 54716943 | 54821019 | 104076 |
| 321 | BAC165_H02 | 4q12 | 54995186 | 55093244 | 98058 |
| 322 | BAC75_G11 | 4q12 | 55848404 | 55944290 | 95886 |
| 323 | BAC214_I01 | 4q12 | 57726223 | 57826223 | 100000 |
| 324 | BAC100_O09 | 4q13.1 | 62036340 | 62118160 | 81820 |
| 325 | BAC78_K15 | 4q13.1 | 62761481 | 62851834 | 90353 |
| 326 | BAC179_A18 | 4q13.1 | 63862374 | 63964772 | 102398 |
| 327 | BAC139_N16 | 4q13.3 | 72009059 | 72093583 | 84524 |
| 328 | BAC1_C16 | 4q13.3 | 74783613 | 74901992 | 118379 |
| 329 | BAC102_F24 | 4q21.21 | 81799446 | 81881241 | 81795 |
| 330 | BAC157_D13 | 4q21.22 | 83820369 | 83932235 | 111866 |
| 331 | BAC193_O16 | 4q21.23 | 85867755 | 85973014 | 105259 |
| 332 | BAC54_J04 | 4q22.1 | 89180491 | 89277356 | 96865 |
| 333 | BAC1_I15 | 4q22.1 | 90492506 | 90606556 | 114050 |
| 334 | BAC78_N01 | 4q22.3 | 97634697 | 97741878 | 107181 |
| 335 | BAC30_I03 | 4q23 | 100411176 | 100530689 | 119513 |
| 336 | BAC10_A09 | 4q23 | 100581693 | 100671934 | 90241 |
| 337 | BAC27_J08 | 4q23 | 100916701 | 101004480 | 87779 |
| 338 | BAC95_P20 | 4q25 | 111049673 | 111150282 | 100609 |
| 339 | BAC229_P17 | 4q25 | 111655288 | 111731605 | 76317 |
| 340 | BAC34_N19 | 4q25 | 111915393 | 112021472 | 106079 |
| 341 | BAC83_F06 | 4q26 | 114603042 | 114688213 | 85171 |
| 342 | BAC221_K14 | 4q27 | 121870773 | 121973195 | 102422 |
| 343 | BAC247_H12 | 4q27 | 123744201 | 123837522 | 93321 |
| 344 | BAC161_L08 | 4q27 | 124041826 | 124124416 | 82590 |
| 345 | BAC232_P08 | 4q27 | 124184113 | 124310627 | 126514 |
| 346 | BAC63_P04 | 4q28.1 | 125266736 | 125348478 | 81742 |
| 347 | BAC121_P19 | 4q28.1 | 129090754 | 129191093 | 100339 |
| 348 | BAC67_B03 | 4q28.2 | 131408902 | 131490991 | 82089 |
| 349 | BAC168_J03 | 4q31.1 | 140224982 | 140299705 | 74723 |
| 350 | BAC69_L20 | 4q31.1 | 141286228 | 141403891 | 117663 |
| 351 | BAC69_L20 | 4q31.1 | 141286228 | 141403891 | 117663 |
| 352 | BAC144_H01 | 4q31.22 | 147642911 | 147729656 | 86745 |
| 353 | BAC5_D24 | 4q31.23 | 149536611 | 149631657 | 95046 |
| 354 | BAC140_G19 | 4q31.3 | 155060187 | 155136574 | 76387 |
| 355 | BAC1_P14 | 4q31.3 | 155704299 | 155813148 | 108849 |
| 356 | BAC233_I20 | 4q32.1 | 156535865 | 156628759 | 92894 |
| 357 | BAC89_D11 | 4q32.2 | 163369661 | 163461565 | 91904 |
| 358 | BAC186_J15 | 4q32.2 | 163431102 | 163537164 | 106062 |
| 359 | BAC169_F02 | 4q32.3 | 167030360 | 167110077 | 79717 |
| 360 | BAC162_P10 | 4q34.1 | 172602064 | 172693157 | 91093 |
| 361 | BAC92_B05 | 4q34.1 | 172872049 | 172970925 | 98876 |
| 362 | BAC152_C21 | 4q34.2-4q34.3 | 178192116 | 178298099 | 105983 |
| 363 | BAC83_J21 | 4q34.3 | 179975486 | 180173585 | 198099 |
| 364 | BAC143_I10 | 4q35.1 | 187586554 | 187695645 | 109091 |
| 365 | BAC84_P22 | 4q35.2 | 188216178 | 188328346 | 112168 |
| 366 | BAC56_F05 | 4q35.2 | 188968683 | 189104028 | 135345 |
| 367 | BAC158_A23 | 4q35.2 | 190234901 | 190346311 | 111410 |
| 368 | BAC63_F14 | 5p15.33 | 1383248 | 1478276 | 95028 |
| 369 | BAC104_G21 | 5p15.33 | 2392625 | 2500908 | 108283 |
| 370 | BAC143_B15 | 5p15.33 | 2593631 | 2676384 | 82753 |
| 371 | BAC179_F12 | 5p15.33 | 4043129 | 4155896 | 112767 |
| 372 | BAC84_C03 | 5p15.32 | 4956086 | 5045269 | 89183 |
| 373 | BAC135_L06 | 5p15.2 | 11432189 | 11516715 | 84526 |
| 374 | BAC116_I11 | 5p15.2 | 14113570 | 14200996 | 87426 |
| 375 | BAC237_J04 | 5p15.1 | 15830104 | 15952420 | 122316 |
| 376 | BAC41_O16 | 5p14.1 | 24674261 | 24795492 | 121231 |
| 377 | BAC152_H04 | 5p14.1 | 25835648 | 25931618 | 95970 |
| 378 | BAC100_E07 | 5p13.3 | 31783566 | 31893767 | 110201 |
| 379 | BAC209_I22 | 5p13.2 | 34914492 | 35023227 | 108735 |
| 380 | BAC109_H04 | 5p13.2 | 36109618 | 36211959 | 102341 |
| 381 | BAC83_M16 | 5p13.2 | 37222924 | 37338505 | 115581 |
| 382 | BAC161_K04 | 5p13.2 | 37875677 | 37972753 | 97076 |
| 383 | BAC241_H11 | 5p13.1 | 39405266 | 39499441 | 94175 |
| 384 | BAC16_L22 | 5p13.1 | 40272172 | 40382917 | 110745 |
| 385 | BAC230_024 | 5p13.1 | 41257415 | 41335975 | 78564 |
| 386 | BAC69_A02 | 5p12 | 44348282 | 44438045 | 89763 |
| 387 | BAC185_O16 | 5q11.2 | 55730356 | 55813966 | 83610 |
| 388 | BAC171_A01 | 5q11.2 | 55902967 | 56006034 | 103067 |
| 389 | BAC135_P04 | 5q11.2 | 56365829 | 56475300 | 109471 |
| 390 | BAC144_P08 | 5q12.1 | 59915000 | 60011227 | 96227 |
| 391 | BAC140_C04 | 5q12.3 | 63995671 | 64086508 | 90837 |
| 392 | BAC8_P07 | 5q13.1 | 67945766 | 68063060 | 117294 |
| 393 | BAC166_A05 | 5q13.2 | 68713044 | 68825351 | 112307 |
| 394 | BAC154_I09 | 5q13.2 | 72447161 | 72539988 | 92827 |
| 395 | BAC119_I19 | 5q13.3 | 74537858 | 74659960 | 122102 |
| 396 | BAC96_B13 | 5q13.3 | 76731517 | 76833372 | 101855 |
| 397 | BAC12_E06 | 5q14.2 | 81639696 | 81793688 | 153992 |
| 398 | BAC12_E06 | 5q14.2 | 81639696 | 81793688 | 153992 |
| 399 | BAC25_M19 | 5q14.3 | 85450007 | 85531324 | 81317 |
| 400 | BAC12_H03 | 5q14.3 | 86606408 | 86713620 | 107212 |
| 401 | BAC237_N24 | 5q15 | 93007232 | 93115689 | 108457 |
| 402 | BAC13_G12 | 5q15 | 93698163 | 93793097 | 94934 |
| 403 | BAC147_D19 | 5q15 | 94626072 | 94724988 | 98916 |
| 404 | BAC83_I19 | 5q15 | 95778663 | 95881677 | 103014 |
| 405 | BAC104_D13 | 5q15 | 96056568 | 96153781 | 97213 |
| 406 | BAC152_J19 | 5q21.1 | 99914737 | 99990930 | 76193 |
| 407 | BAC45_L07 | 5q21.2 | 104257847 | 104376897 | 119050 |
| 408 | BAC126_A01 | 5q21.3 | 106644568 | 106753285 | 108717 |
| 409 | BAC179_F17 | 5q21.3 | 106806064 | 106904524 | 98460 |
| 410 | BAC178_H02 | 5q21.3 | 108330078 | 108399671 | 69593 |
| 411 | BAC168_M02 | 5q23.1 | 115345365 | 115450275 | 104910 |
| 412 | BAC161_C19 | 5q23.1 | 119061130 | 119158649 | 97519 |
| 413 | BAC5_I04 | 5q23.1 | 121476924 | 121593597 | 116673 |
| 414 | BAC53_A02 | 5q23.2 | 127228668 | 127338607 | 109939 |
| 415 | BAC222_P07 | 5q23.3 | 131375427 | 131467441 | 92014 |
| 416 | BAC141_I18 | 5q23.3 | 131440022 | 131516627 | 76605 |
| 417 | BAC211_D16 | 5q23.3 | 131878875 | 131961364 | 82489 |
| 418 | BAC161_A19 | 5q23.3 | 131968600 | 132079204 | 110604 |
| 419 | BAC140_H19 | 5q23.3-5q31.1 | 132229533 | 132311479 | 81946 |
| 420 | BAC89_H06 | 5q31.1 | 134529828 | 134659147 | 129319 |
| 421 | BAC59_N13 | 5q31.1 | 135439012 | 135550777 | 111765 |
| 422 | BAC116_I13 | 5q31.2 | 137577978 | 137709614 | 131636 |
| 423 | BAC167_K06 | 5q31.3 | 139541045 | 139644553 | 103508 |
| 424 | BAC175_L19 | 5q31.3 | 140567630 | 140660439 | 92809 |
| 425 | BAC143_A21 | 5q33.1 | 149516321 | 149609852 | 93531 |
| 426 | BAC200_J07 | 5q33.1 | 151126208 | 151238596 | 112388 |
| 427 | BAC83_D21 | 5q33.2 | 153819352 | 153916855 | 97503 |
| 428 | BAC123_K15 | 5q33.3 | 155629907 | 155734609 | 104702 |
| 429 | BAC176_G19 | 5q33.3 | 156351854 | 156433256 | 81402 |
| 430 | BAC57_G23 | 5q33.3 | 156594581 | 156717106 | 122525 |
| 431 | BAC196_J01 | 5q33.3 | 158543486 | 158640034 | 96548 |
| 432 | BAC111_D04 | 5q34 | 164113207 | 164239181 | 125974 |
| 433 | BAC176_E04 | 5q34 | 166941825 | 167039129 | 97304 |
| 434 | BAC89_H17 | 5q34-5q35.1 | 168376075 | 168480282 | 104207 |
| 435 | BAC131_C11 | 5q35.1 | 168693850 | 168837210 | 143360 |
| 436 | BAC88_D19 | 5q35.1 | 170011190 | 170101610 | 90420 |
| 437 | BAC153_I06 | 5q35.1 | 172693702 | 172783677 | 89975 |
| 438 | BAC68_H03 | 5q35.2 | 175146964 | 175240550 | 93586 |
| 439 | BAC76_C20 | 5q35.3 | 179664936 | 179751801 | 86865 |
| 440 | BAC53_P23 | 5q35.3 | 180104303 | 180191401 | 87098 |
| 441 | BAC178_M23 | 6p25.3 | 327940 | 435330 | 107390 |
| 442 | BAC80_J07 | 6p25.2 | 3327847 | 3445875 | 118028 |
| 443 | BAC14_E06 | 6p24.3 | 7132474 | 7235216 | 102742 |
| 444 | BAC86_N04 | 6p24.3 | 7612567 | 7755902 | 143335 |
| 445 | BAC86_G12 | 6p24.3 | 8077041 | 8163454 | 86413 |
| 446 | BAC132_J18 | 6p24.2 | 10948696 | 11032899 | 84203 |
| 447 | BAC183_J04 | 6p24.2 | 11369965 | 11464799 | 94834 |
| 448 | BAC155_N23 | 6p23 | 13577743 | 13668135 | 90392 |
| 449 | BAC2_J04 | 6p23 | 14035329 | 14130836 | 95507 |
| 450 | BAC247_M13 | 6p22.3 | 16464939 | 16549251 | 84312 |
| 451 | BAC97_H14 | 6p22.3 | 16577540 | 16703377 | 125837 |
| 452 | BAC33_F13 | 6p22.3 | 18315025 | 18450781 | 135756 |
| 453 | BAC113_K09 | 6p22.2 | 26003786 | 26113453 | 109667 |
| 454 | BAC147_A07 | 6p22.2 | 26198407 | 26293609 | 95202 |
| 455 | BAC144_D21 | 6p22.1 | 26516805 | 26584384 | 67579 |
| 456 | BAC40_N06 | 6p22.1 | 27945544 | 28035164 | 89620 |
| 457 | BAC131_E23 | 6p22.1 | 28938843 | 29029937 | 91094 |
| 458 | BAC249_P17 | 6p22.1 | 30020334 | 30084550 | 64216 |
| 459 | BAC56_M01 | 6p21.33 | 30305452 | 30413667 | 108215 |
| 459 | BAC56_M01 | 6p21.33 | 30305452 | 30413667 | 108215 |
| 460 | BAC157_G20 | 6p21.33 | 31721207 | 31817554 | 96347 |
| 461 | BAC219_C01 | 6p21.33 | 31762751 | 31834023 | 71272 |
| 462 | BAC104_F07 | 6p21.32 | 32150565 | 32250565 | 100000 |
| 463 | BAC100_M24 | 6p21.32 | 32165526 | 32261355 | 95829 |
| 464 | BAC97_I22 | 6p21.32 | 32327923 | 32449055 | 121132 |
| 465 | BAC97_H10 | 6p21.32 | 33151035 | 33287147 | 136112 |
| 466 | BAC134_F04 | 6p21.31 | 34587962 | 34672169 | 84207 |
| 467 | BAC90_M07 | 6p21.31 | 35382847 | 35485645 | 102798 |
| 468 | BAC116_P17 | 6p21.2 | 37164127 | 37247071 | 82944 |
| 469 | BAC92_I06 | 6p21.2 | 37513923 | 37632041 | 118118 |
| 470 | BAC171_B22 | 6p21.2 | 38732297 | 38840059 | 107762 |
| 471 | BAC52_H13 | 6p21.1 | 41236351 | 41368071 | 131720 |
| 472 | BAC57_A01 | 6p21.1 | 41922832 | 42024668 | 101836 |
| 473 | BAC92_M03 | 6p21.1 | 42239869 | 42341207 | 101338 |
| 474 | BAC135_A14 | 6p21.1 | 42651562 | 42747153 | 95591 |
| 475 | BAC236_A07 | 6p21.1 | 43111539 | 43197110 | 85571 |
| 476 | BAC183_P10 | 6p21.1 | 43942767 | 44025264 | 82497 |
| 477 | BAC189_K20 | 6p21.1 | 44356894 | 44478326 | 121432 |
| 478 | BAC174_C09 | 6p12.3 | 46929715 | 46993329 | 63614 |
| 479 | BAC139_M20 | 6p12.3 | 50172533 | 50292946 | 120413 |
| 480 | BAC162_J19 | 6p12.1 | 56262554 | 56380600 | 118046 |
| 481 | BAC162_J19 | 6p12.1 | 56262554 | 56380600 | 118046 |
| 482 | BAC71_N14 | 6p11.2 | 58391514 | 58542673 | 151159 |
| 483 | BAC81_H13 | 6q11.1 | 62328653 | 62414409 | 85756 |
| 484 | BAC133_M12 | 6q12 | 64239824 | 64341013 | 101189 |
| 485 | BAC120_A16 | 6q13 | 70700735 | 70810374 | 109639 |
| 486 | BAC76_A23 | 6q13 | 70860671 | 70964530 | 103859 |
| 487 | BAC162_D08 | 6q14.1 | 76478903 | 76535139 | 56236 |
| 488 | BAC81_G12 | 6q14.1 | 82561237 | 82674123 | 112886 |
| 489 | BAC86_L20 | 6q14.3 | 84976376 | 85076412 | 100036 |
| 490 | BAC115_D12 | 6q15 | 91137394 | 91240142 | 102748 |
| 491 | BAC143_P17 | 6q16.1 | 94679861 | 94784749 | 104888 |
| 492 | BAC31_M14 | 6q16.1 | 99382797 | 99485973 | 103176 |
| 493 | BAC167_F05 | 6q16.2 | 99584343 | 99678126 | 93783 |
| 494 | BAC97_H04 | 6q16.3 | 102478954 | 102612601 | 133647 |
| 495 | BAC154_I05 | 6q21 | 105656605 | 105768825 | 112220 |
| 496 | BAC246_A19 | 6q21 | 108507838 | 108597656 | 89818 |
| 497 | BAC39_M24 | 6q21 | 112905422 | 113022750 | 117328 |
| 498 | BAC103_P05 | 6q22.1 | 117787053 | 117887130 | 100077 |
| 499 | BAC80_O04 | 6q22.1 | 117792318 | 117887834 | 95516 |
| 500 | BAC22_E09 | 6q22.31 | 119574560 | 119674017 | 99457 |
| 501 | BAC228_A01 | 6q22.31 | 121679834 | 121790568 | 110734 |
| 502 | BAC83_D24 | 6q22.33 | 128687494 | 128791735 | 104241 |
| 503 | BAC1_F23 | 6q22.33 | 130146277 | 130254755 | 108478 |
| 504 | BAC129_J17 | 6q23.2 | 133504700 | 133594592 | 89892 |
| 505 | BAC90_H10 | 6q23.2 | 134078786 | 134156272 | 77486 |
| 506 | BAC236_B24 | 6q23.3 | 135464059 | 135540770 | 76711 |
| 507 | BAC113_O10 | 6q23.3 | 137065220 | 137181888 | 116668 |
| 508 | BAC68_C07 | 6q23.3 | 138061876 | 138167468 | 105592 |
| 509 | BAC39_N24 | 6q24.1 | 139922591 | 140008940 | 86349 |
| 510 | BAC160_I11 | 6q24.2 | 143427462 | 143528640 | 101178 |
| 511 | BAC129_A04 | 6q24.2 | 144198379 | 144288434 | 90055 |
| 512 | BAC14_M20 | 6q24.3 | 147711750 | 147849045 | 137295 |
| 513 | BAC92_E13 | 6q25.1 | 151184392 | 151279230 | 94838 |
| 514 | BAC53_E01 | 6q25.2 | 152322947 | 152496014 | 173067 |
| 515 | BAC52_C20 | 6q25.2 | 153022753 | 153136122 | 113369 |
| 516 | BAC152_N22 | 6q25.2 | 154170326 | 154271332 | 101006 |
| 517 | BAC200_M04 | 6q25.3 | 156943382 | 157121914 | 178532 |
| 518 | BAC147_C16 | 6q26 | 161849647 | 161957397 | 107750 |
| 519 | BAC19_O23 | 6q26 | 162893233 | 162992417 | 99184 |
| 520 | BAC121_P18 | 6q27 | 170425617 | 170539243 | 113626 |
| 521 | BAC12_F16 | 6q27 | 170669842 | 170761982 | 92140 |
| 522 | BAC39_K09 | 7p22.2 | 3630616 | 3713227 | 82611 |
| 523 | BAC161_M22 | 7p21.3 | 7009111 | 7098367 | 89256 |
| 524 | BAC100_K13 | 7p21.3 | 7571817 | 7679440 | 107623 |
| 525 | BAC139_D05 | 7p21.2 | 13661531 | 13749490 | 87959 |
| 526 | BAC127_G14 | 7p21.2 | 13833486 | 13949998 | 116512 |
| 527 | BAC65_K10 | 7p21.2 | 15377907 | 15468648 | 90741 |
| 528 | BAC62_J14 | 7p21.1 | 18412490 | 18490806 | 78316 |
| 529 | BAC62_N06 | 7p21.1 | 19166467 | 19281617 | 115150 |
| 530 | BAC112_K01 | 7p21.1 | 19670225 | 19755999 | 85774 |
| 531 | BAC199_B07 | 7p15.3 | 22444638 | 22538756 | 94118 |
| 532 | BAC131_G08 | 7p15.3 | 24609515 | 24699055 | 89540 |
| 533 | BAC31_C02 | 7p15.2 | 26222518 | 26316901 | 94383 |
| 534 | BAC218_N01 | 7p15.2 | 26910635 | 27022265 | 111630 |
| 535 | BAC139_K10 | 7p15.1 | 29289704 | 29390944 | 101240 |
| 536 | BAC100_E09 | 7p15.1 | 29732976 | 29860598 | 127622 |
| 537 | BAC161_H20 | 7p14.3 | 31530956 | 31630649 | 99693 |
| 538 | BAC161_H20 | 7p14.3 | 31530956 | 31630649 | 99693 |
| 539 | BAC75_J03 | 7p14.3-7p14.2 | 34623872 | 34720952 | 97080 |
| 540 | BAC89_K07 | 7p14.2 | 35606033 | 35688146 | 82113 |
| 541 | BAC24_A20 | 7p14.2 | 36031436 | 36140361 | 108925 |
| 542 | BAC122_F23 | 7p14.1 | 41133806 | 41233648 | 99842 |
| 543 | BAC5_A09 | 7p14.1 | 41745208 | 41846675 | 101467 |
| 544 | BAC71_J10 | 7p14.1-7p13 | 43081253 | 43180257 | 99004 |
| 545 | BAC151_A17 | 7p13 | 44176973 | 44272054 | 95081 |
| 546 | BAC34_E12 | 7p13 | 44212893 | 44341318 | 128425 |
| 547 | BAC46_P02 | 7p13 | 44852407 | 44944240 | 91833 |
| 548 | BAC221_F17 | 7p13 | 45835262 | 45922041 | 86779 |
| 549 | BAC193_I24 | 7p12.3 | 49202211 | 49287690 | 85479 |
| 550 | BAC25_N13 | 7p12.2 | 50399061 | 50512825 | 113764 |
| 551 | BAC34_L05 | 7p12.1 | 51096787 | 51202357 | 105570 |
| 552 | BAC80_I11 | 7p12.1 | 51571921 | 51692862 | 120941 |
| 553 | BAC15_B08 | 7p11.2 | 54776241 | 54876343 | 100102 |
| 554 | BAC1_I06 | 7p11.2 | 55017732 | 55133757 | 116025 |
| 555 | BAC85_D22 | 7p11.2 | 55817180 | 55910610 | 93430 |
| 556 | BAC61_A07 | 7q11.21 | 65335669 | 65430762 | 95093 |
| 557 | BAC217_M14 | 7q11.21 | 65339164 | 65430744 | 91580 |
| 558 | BAC57_L05 | 7q11.21 | 66072861 | 66163679 | 90818 |
| 559 | BAC145_L15 | 7q11.22 | 67764969 | 67850122 | 85153 |
| 560 | BAC100_L06 | 7q11.22 | 70663949 | 70763379 | 99430 |
| 561 | BAC78_I13 | 7q11.23 | 75147619 | 75245296 | 97677 |
| 562 | BAC226_J16 | 7q11.23 | 76164831 | 76265712 | 100881 |
| 563 | BAC1_P18 | 7q21.11 | 82207500 | 82304693 | 97193 |
| 564 | BAC186_B10 | 7q21.12 | 86244082 | 86350193 | 106111 |
| 565 | BAC29_K23 | 7q21.13 | 89834600 | 89920809 | 86209 |
| 566 | BAC105_F06 | 7q21.2 | 91349596 | 91437614 | 88018 |
| 567 | BAC3_J18 | 7q21.3 | 92458298 | 92558298 | 100000 |
| 568 | BAC169_J07 | 7q21.3 | 93565930 | 93653925 | 87995 |
| 569 | BAC185_E15 | 7q21.3 | 95188997 | 95278650 | 89653 |
| 570 | BAC26_C02 | 7q21.3 | 95681898 | 95835665 | 153767 |
| 571 | BAC42_D19 | 7q22.1 | 98119555 | 98208373 | 88818 |
| 572 | BAC144_G04 | 7q22.1 | 98612019 | 98687537 | 75518 |
| 573 | BAC109_M06 | 7q22.1 | 100331404 | 100477299 | 145895 |
| 574 | BAC96_K03 | 7q22.1 | 103235136 | 103351127 | 115991 |
| 575 | BAC76_I16 | 7q22.2 | 103705826 | 103810688 | 104862 |
| 576 | BAC144_C20 | 7q22.3 | 106054979 | 106164861 | 109882 |
| 577 | BAC100_N11 | 7q31.1 | 107120128 | 107270903 | 150775 |
| 578 | BAC232_P13 | 7q31.1 | 107832386 | 107924546 | 92160 |
| 579 | BAC1_B12 | 7q31.1 | 113798958 | 113963458 | 164500 |
| 580 | BAC202_P14 | 7q31.2 | 115932803 | 116032803 | 100000 |
| 581 | BAC83_N01 | 7q31.2 | 116333333 | 116446111 | 112778 |
| 582 | BAC147_C10 | 7q31.31 | 117299453 | 117390823 | 91370 |
| 583 | BAC85_H06 | 7q31.32 | 121082433 | 121162248 | 79815 |
| 584 | BAC127_C14 | 7q31.33 | 125873679 | 125995631 | 121952 |
| 585 | BAC237_O10 | 7q32.1 | 127101569 | 127188701 | 87132 |
| 586 | BAC195_F16 | 7q32.1 | 128053726 | 128147876 | 94150 |
| 587 | BAC30_F19 | 7q32.2 | 129116260 | 129183867 | 67607 |
| 588 | BAC67_N20 | 7q32.3 | 131767431 | 131864286 | 96855 |
| 589 | BAC142_H10 | 7q32.3 | 131782819 | 131907168 | 124349 |
| 590 | BAC238_G23 | 7q33 | 132901781 | 132989341 | 87560 |
| 591 | BAC32_O17 | 7q33 | 133468648 | 133576345 | 107697 |
| 592 | BAC79_G17 | 7q33 | 134506668 | 134606668 | 100000 |
| 593 | BAC125_H09 | 7q33-7q34 | 137598759 | 137627395 | 28636 |
| 594 | BAC221_K10 | 7q34 | 141596616 | 141723924 | 127308 |
| 595 | BAC33_O07 | 7q35 | 142967468 | 143074534 | 107066 |
| 596 | BAC120_E18 | 7q35 | 143318120 | 143346776 | 28656 |
| 597 | BAC175_L18 | 7q35 | 146272399 | 146361708 | 89309 |
| 598 | BAC80_H22 | 7q36.1 | 148285316 | 148423335 | 138019 |
| 599 | BAC124_O22 | 7q36.1 | 151697370 | 151850031 | 152661 |
| 600 | BAC140_I02 | 7q36.2 | 153967519 | 154041364 | 73845 |
| 601 | BAC15_O23 | 7q36.3 | 154718525 | 154803893 | 85368 |
| 602 | BAC171_D10 | 7q36.3 | 154958401 | 155048450 | 90049 |
| 603 | BAC96_C15 | 7q36.3 | 155799091 | 155892125 | 93034 |
| 604 | BAC82_L19 | 7q36.3 | 156991499 | 157116280 | 124781 |
| 605 | BAC218_F21 | 8p23.3 | 250545 | 352706 | 102161 |
| 606 | BAC78_A19 | 8p23.3 | 318079 | 432847 | 114768 |
| 607 | BAC46_E24 | 8p23.3 | 1289206 | 1436249 | 147043 |
| 608 | BAC1_J24 | 8p23.2 | 5245270 | 5385392 | 140122 |
| 609 | BAC90_M06 | 8p23.1 | 8587065 | 8699899 | 112834 |
| 610 | BAC145_N24 | 8p23.1 | 11652681 | 11739056 | 86375 |
| 611 | BAC144_F08 | 8p22 | 16650748 | 16765353 | 114605 |
| 612 | BAC27_O21 | 8p22 | 17469300 | 17552371 | 83071 |
| 613 | BAC31_D08 | 8p21.3 | 19608435 | 19708854 | 100419 |
| 614 | BAC176_L07 | 8p21.3 | 21744025 | 21837170 | 93145 |
| 615 | BAC51_G11 | 8p21.2 | 25246073 | 25345850 | 99777 |
| 616 | BAC97_F20 | 8p21.1 | 28503253 | 28617640 | 114387 |
| 617 | BAC117_I18 | 8p21.1 | 28594064 | 28676186 | 82122 |
| 618 | BAC74_F23 | 8p12 | 30007346 | 30107346 | 100000 |
| 619 | BAC11_C18 | 8p12 | 31671705 | 31784398 | 112693 |
| 620 | BAC15_F07 | 8p12 | 38270045 | 38427465 | 157420 |
| 621 | BAC76_E16 | 8p11.21 | 40082921 | 40214595 | 131674 |
| 622 | BAC168_N13 | 8p11.21 | 41906856 | 42024511 | 117655 |
| 623 | BAC247_M21 | 8p11.21 | 42108045 | 42180707 | 72662 |
| 624 | BAC155_D08 | 8p11.21 | 42247378 | 42333227 | 85849 |
| 625 | BAC19_P21 | 8p11.1 | 43205552 | 43297867 | 92315 |
| 626 | BAC145_H17 | 8q11.21 | 49370589 | 49456490 | 85901 |
| 627 | BAC78_O16 | 8q12.1 | 56309657 | 56410659 | 101002 |
| 628 | BAC106_L05 | 8q12.1 | 56843940 | 56938557 | 94617 |
| 629 | BAC152_G16 | 8q12.1 | 58417894 | 58513751 | 95857 |
| 630 | BAC50_F01 | 8q13.2-8q13.3 | 69343304 | 69453731 | 110427 |
| 631 | BAC36_H08 | 8q13.3 | 70414306 | 70488717 | 74411 |
| 632 | BAC177_N09 | 8q21.11 | 74385120 | 74467931 | 82811 |
| 633 | BAC217_O23 | 8q21.13 | 79730906 | 79806826 | 75920 |
| 634 | BAC178_P02 | 8q21.13 | 81338263 | 81446926 | 108663 |
| 635 | BAC57_O15 | 8q21.13 | 82359567 | 82463826 | 104259 |
| 636 | BAC37_H09 | 8q21.3 | 88864626 | 89001470 | 136844 |
| 637 | BAC90_P15 | 8q21.3 | 90921974 | 91013418 | 91444 |
| 638 | BAC92_K10 | 8q21.3 | 91300794 | 91427428 | 126634 |
| 639 | BAC55_M03 | 8q22.1 | 96258015 | 96396873 | 138858 |
| 640 | BAC157_F12 | 8q22.3 | 102400715 | 102507065 | 106350 |
| 641 | BAC169_B05 | 8q23.1 | 107953597 | 108043167 | 89570 |
| 642 | BAC20_K18 | 8q23.1 | 108814200 | 108922667 | 108467 |
| 643 | BAC176_H16 | 8q23.3 | 116825035 | 116915090 | 90055 |
| 644 | BAC199_P22 | 8q24.11-8q24.12 | 119018437 | 119106743 | 88306 |
| 645 | BAC200_E17 | 8q24.12 | 119283899 | 119352610 | 68711 |
| 646 | BAC248_F14 | 8q24.12 | 120377587 | 120491239 | 113652 |
| 647 | BAC53_L17 | 8q24.21 | 127997591 | 128081281 | 83690 |
| 648 | BAC192_N08 | 8q24.21 | 128687398 | 128764964 | 77566 |
| 649 | BAC195_N23 | 8q24.21 | 131222488 | 131303457 | 80969 |
| 650 | BAC100_A11 | 8q24.22 | 133903220 | 133999544 | 96324 |
| 651 | BAC113_P08 | 8q24.22 | 134131816 | 134232254 | 100438 |
| 652 | BAC8_N14 | 8q24.22 | 134220116 | 134334477 | 114361 |
| 653 | BAC139_M03 | 8q24.22 | 134433350 | 134526100 | 92750 |
| 654 | BAC90_C04 | 8q24.22 | 136160999 | 136280543 | 119544 |
| 655 | BAC76_N02 | 8q24.23-8q24.3 | 139845147 | 139967047 | 121900 |
| 656 | BAC145_J12 | 8q24.3 | 144691670 | 144777802 | 86132 |
| 657 | BAC193_J11 | 8q24.3 | 145682925 | 145793387 | 110462 |
| 658 | BAC22_E06 | 9p24.3 | 650298 | 795587 | 145289 |
| 659 | BAC44_B22 | 9p24.3 | 981631 | 1081631 | 100000 |
| 660 | BAC75_D17 | 9p24.2 | 2524765 | 2622926 | 98161 |
| 661 | BAC133_K20 | 9p24.1 | 5015679 | 5132647 | 116968 |
| 662 | BAC71_J11 | 9p23 | 12416070 | 12531583 | 115513 |
| 663 | BAC139_H09 | 9p22.3 | 14420099 | 14498736 | 78637 |
| 664 | BAC59_H01 | 9p22.2 | 17575042 | 17683618 | 108576 |
| 665 | BAC49_B18 | 9p21.3 | 19971123 | 20106319 | 135196 |
| 666 | BAC160_C10 | 9p21.3 | 20495209 | 20587875 | 92666 |
| 667 | BAC88_P21 | 9p21.3 | 23737636 | 23838679 | 101043 |
| 668 | BAC27_C20 | 9p21.2 | 26225506 | 26314730 | 89224 |
| 669 | BAC86_G04 | 9p21.2 | 27191198 | 27277270 | 86072 |
| 670 | BAC20_M12 | 9p21.1 | 29181837 | 29310288 | 128451 |
| 671 | BAC131_N18 | 9p13.3 | 33790121 | 33879062 | 88941 |
| 672 | BAC125_M17 | 9p13.3 | 35102040 | 35198836 | 96796 |
| 673 | BAC135_G04 | 9p13.3 | 36105466 | 36181669 | 76203 |
| 674 | BAC185_C04 | 9p13.2 | 37655323 | 37723149 | 67826 |
| 675 | BAC33_J02 | 9q12 | 62581444 | 62677949 | 96505 |
| 676 | BAC70_O18 | 9q21.11 | 67348747 | 67427440 | 78693 |
| 677 | BAC84_K23 | 9q21.11 | 69062864 | 69150054 | 87190 |
| 678 | BAC104_M09 | 9q21.12 | 71009439 | 71109439 | 100000 |
| 679 | BAC40_J09 | 9q21.13 | 72744111 | 72847261 | 103150 |
| 680 | BAC84_F11 | 9q21.13 | 73098742 | 73207323 | 108581 |
| 681 | BAC42_K01 | 9q21.2 | 74830860 | 74918475 | 87615 |
| 682 | BAC91_J21 | 9q21.31 | 77580758 | 77668728 | 87970 |
| 683 | BAC6_B03 | 9q21.32 | 80500889 | 80628266 | 127377 |
| 684 | BAC84_E19 | 9q21.32 | 82047788 | 82128275 | 80487 |
| 685 | BAC165_B09 | 9q21.33 | 84270378 | 84369622 | 99244 |
| 686 | BAC90_K21 | 9q22.31 | 893424248 | 9491580 | 149156 |
| 687 | BAC37_F17 | 9q22.31 | 90722945 | 90830015 | 107070 |
| 688 | BAC26_C01 | 9q22.31 | 90892891 | 90996423 | 103532 |
| 689 | BAC10_P08 | 9q22.32 | 93140524 | 93256784 | 116260 |
| 690 | 8AC57_M21 | 9q22.32 | 93249016 | 93351194 | 102178 |
| 691 | BAC221_I19 | 9q22.32 | 93471627 | 93586111 | 114484 |
| 692 | BAC100_N15 | 9q22.33 | 96054306 | 96140321 | 86015 |
| 693 | BAC97_K18 | 9q31.1 | 99512741 | 99599878 | 87137 |
| 694 | BAC165_P10 | 9q31.1-9q31.2 | 103755835 | 103834517 | 78682 |
| 695 | BAC90_A22 | 9q31.3 | 107024180 | 107106560 | 82380 |
| 696 | BAC106_J20 | 9q31.3 | 108852499 | 108988543 | 136044 |
| 697 | BAC20_C01 | 9q33.1 | 113648519 | 113746943 | 98424 |
| 698 | BAC96_N01 | 9q33.1 | 116793491 | 116880320 | 86829 |
| 699 | BAC157_D19 | 9q33.2 | 118784003 | 118862485 | 78482 |
| 700 | BAC130_H19 | 9q33.2 | 120338913 | 120423574 | 84661 |
| 701 | BAC63_L05 | 9q33.3 | 123799754 | 123888484 | 88730 |
| 702 | BAC193_F12 | 9q33.3 | 124581774 | 124688531 | 106757 |
| 703 | BAC65_E14 | 9q33.3 | 125187291 | 125291185 | 103894 |
| 704 | BAC118_A06 | 9q34.11 | 126906055 | 127004351 | 98296 |
| 705 | BAC142_B18 | 9q34.11 | 128553065 | 128650979 | 97914 |
| 706 | BAC125_M06 | 9q34.13 | 129109267 | 129246545 | 137278 |
| 707 | BAC108_J10 | 9q34.13-9q34.2 | 131129138 | 131282389 | 153251 |
| 708 | BAC142_E13 | 9q34.2 | 131458712 | 131520298 | 61586 |
| 709 | BAC233_P12 | 9q34.2 | 131862396 | 131977760 | 115364 |
| 710 | BAC110 G13 | 9q34.3 | 133510576 | 133654238 | 143662 |
| 711 | BAC4_J24 | 9q34.3 | 133797918 | 133900983 | 103065 |
| 712 | BAC176_M06 | 9q34.3 | 135135024 | 135224007 | 88983 |
| 713 | BAC224_C15 | 9q34.3 | 135335088 | 135409363 | 74275 |
| 714 | BAC70_M18 | 10p15.3 | 507301 | 600902 | 93601 |
| 715 | BAC176_J17 | 10p15.3 | 572068 | 694941 | 122873 |
| 716 | BAC143_B19 | 10p15.3 | 597659 | 721334 | 123675 |
| 717 | BAC143_G07 | 10p15.1 | 5963827 | 6086336 | 122509 |
| 718 | BAC198_H13 | 10p14 | 8006753 | 8114479 | 107726 |
| 719 | BAC56_M02 | 10p14-10p13 | 12140024 | 12243506 | 103482 |
| 720 | BAC116_L03 | 10p13 | 16525061 | 16624279 | 99218 |
| 721 | BAC243_L13 | 10p13 | 17178306 | 17266514 | 88208 |
| 722 | BAC145_N12 | 10p13-10p12.33 | 17205131 | 17321613 | 116482 |
| 723 | BAC82_K06 | 10p12.33 | 17558154 | 17645168 | 87014 |
| 724 | BAC145_P22 | 10p12.31 | 21361713 | 21453075 | 91362 |
| 725 | BAC250_J11 | 10p12.31 | 21811214 | 21911214 | 100000 |
| 726 | BAC25_H08 | 10p12.31-10p12.2 | 22556133 | 22638966 | 82833 |
| 727 | BAC15_O16 | 10p12.1 | 27221761 | 27336873 | 115112 |
| 728 | BAC142_K07 | 10p11.22 | 32621307 | 32707719 | 86412 |
| 729 | BAC142_J05 | 10p11.22 | 33084633 | 33212564 | 127931 |
| 730 | BAC144_N08 | 10p11.22 | 33467719 | 33552030 | 84311 |
| 731 | BAC175_I06 | 10p11.21 | 36015749 | 36099117 | 83368 |
| 732 | BAC21_L23 | 10q11.21 | 42874803 | 42955854 | 81051 |
| 733 | BAC90_D13 | 10q11.21 | 44684197 | 44775710 | 91513 |
| 734 | BAC155_H03 | 10q11.23 | 50040690 | 50110260 | 69570 |
| 735 | BAC81_M04 | 10q11.23 | 51601728 | 51741054 | 139326 |
| 736 | BAC51_G06 | 10q11.23-10q21.1 | 52128261 | 52238123 | 109862 |
| 737 | BAC140_E20 | 10q21.1 | 57705451 | 57796037 | 90586 |
| 738 | BAC193_I07 | 10q21.2 | 60947995 | 61025763 | 77768 |
| 739 | BAC193_A18 | 10q21.3 | 63844348 | 63925172 | 80824 |
| 740 | BAC4_P01 | 10q21.3 | 63947074 | 64016579 | 69505 |
| 741 | BAC169_F05 | 10q21.3 | 69498253 | 69604439 | 106186 |
| 742 | BAC83_F04 | 10q22.1 | 71188135 | 71338083 | 149948 |
| 743 | BAC144_M14 | 10q22.1 | 72889622 | 72989168 | 99546 |
| 744 | BAC24_E16 | 10q22.2 | 74925460 | 75061766 | 136306 |
| 745 | BAC178_D22 | 10q22.2 | 76282392 | 76389602 | 107210 |
| 746 | BAC90_G04 | 10q22.3 | 81698075 | 81789502 | 91427 |
| 747 | BAC117_P03 | 10q22.3 | 81797342 | 81912853 | 115511 |
| 748 | BAC64_K18 | 10q23.2 | 86958211 | 87047797 | 89586 |
| 749 | BAC110_F03 | 10q23.31 | 89283957 | 89369550 | 85593 |
| 750 | BAC145_F01 | 10q23.31 | 90182931 | 90262986 | 80055 |
| 751 | BAC142_J19 | 10q23.32 | 93594192 | 93662580 | 68388 |
| 752 | BAC84_A21 | 10q24.1 | 98397975 | 98501150 | 103175 |
| 753 | BAC106_B16 | 10q24.1 | 98779960 | 98877800 | 97840 |
| 754 | BAC41_E05 | 10q24.2-10q24.31 | 101596178 | 101686004 | 89826 |
| 755 | BAC56_I15 | 10q24.31 | 101685920 | 101772111 | 86191 |
| 756 | BAC86_I06 | 10q24.31 | 102240676 | 102336242 | 95566 |
| 757 | BAC15_P02 | 10q24.31 | 102416960 | 102575827 | 158867 |
| 758 | BAC222_E18 | 10q24.32 | 103120322 | 103220322 | 100000 |
| 759 | BAC244_H22 | 10q24.32 | 104239748 | 104335274 | 95526 |
| 760 | BAC152_K02 | 10q24.33 | 105110509 | 105206316 | 95807 |
| 761 | BAC155_M23 | 10q25.1 | 111246702 | 111331118 | 84416 |
| 762 | BAC62_C09 | 10q25.2 | 111651840 | 111771364 | 119524 |
| 763 | BAC82_E10 | 10q25.2 | 113123669 | 113210309 | 86640 |
| 764 | BAC81_O02 | 10q25.2 | 113737299 | 113855386 | 118087 |
| 765 | BAC186_N19 | 10q25.3 | 115385210 | 115457685 | 72475 |
| 766 | BAC165_N05 | 10q25.3 | 117363541 | 117432812 | 69271 |
| 767 | BAC252_B16 | 10q25.3 | 117507118 | 117615785 | 108667 |
| 768 | BAC81_O19 | 10q26.11 | 120923633 | 121017704 | 94071 |
| 769 | BAC218_E11 | 10q26.13 | 122868888 | 122973190 | 104302 |
| 770 | BAC154_E03 | 10q26.13 | 122993729 | 123096429 | 102700 |
| 771 | BAC4_G24 | 10q26.13 | 123239656 | 123346756 | 107100 |
| 772 | BAC82_K12 | 10q26.13 | 123582762 | 123661024 | 78262 |
| 773 | BAC245_F15 | 10q26.13 | 123738959 | 123838959 | 100000 |
| 774 | BAC223_O05 | 10q26.13 | 123921313 | 124021313 | 100000 |
| 775 | BAC48_I12 | 10q26.13 | 126272420 | 126368622 | 96202 |
| 776 | BAC100_I08 | 10q26.2 | 128443084 | 128531120 | 88036 |
| 777 | BAC97_D05 | 10q26.2 | 129258546 | 129386780 | 128234 |
| 778 | BAC167_G16 | 10q26.2 | 129266346 | 129344774 | 78428 |
| 779 | BAC9_B16 | 10q26.2 | 129960395 | 130068173 | 107778 |
| 780 | BAC92_D03 | 10q26.2 | 130018442 | 130120882 | 102440 |
| 781 | BAC42_H14 | 10q26.2-10q26.3 | 130163822 | 130239698 | 75876 |
| 782 | BAC165_D01 | 11p15.5 | 202636 | 299652 | 97016 |
| 783 | BAC121_J14 | 11p15.5 | 1280556 | 1387145 | 106589 |
| 784 | BAC60_J23 | 11p15.4 | 2818881 | 2906307 | 87426 |
| 785 | BAC252_K02 | 11p15.4 | 2854019 | 2981251 | 127232 |
| 786 | BAC193_P15 | 11p15.4 | 3772345 | 3882944 | 110599 |
| 787 | BAC121_J11 | 11p15.4 | 6306709 | 6432048 | 125339 |
| 788 | BAC91_J13 | 11p15.4 | 6429065 | 6516435 | 87370 |
| 789 | BAC219_F22 | 11p15.4 | 6512284 | 6635767 | 123483 |
| 790 | BAC96_J11 | 11p15.4 | 8053861 | 8153863 | 100002 |
| 791 | BAC195_A16 | 11p15.4 | 8325549 | 8417830 | 92281 |
| 792 | BAC32_K13 | 11p15.4 | 8674397 | 8794353 | 119956 |
| 793 | BAC185_G10 | 11p15.4 | 8916322 | 9023171 | 106849 |
| 794 | BAC252_G23 | 11p15.4 | 10553762 | 10672376 | 118614 |
| 795 | BAC88_B15 | 11p15.3 | 11739865 | 11899945 | 160080 |
| 796 | BAC82_C10 | 11p15.3-11p15.2 | 13730691 | 13837197 | 106506 |
| 797 | BAC82_C10 | 11p15.3-11p15.2 | 13730691 | 13837197 | 106506 |
| 798 | BAC35_I01 | 11p15.2 | 14803295 | 14910127 | 106832 |
| 799 | BAC90_G07 | 11p15.1 | 17607390 | 17704731 | 97341 |
| 800 | BAC117_D15 | 11p15.1 | 18372788 | 18497301 | 124513 |
| 801 | BAC1_K02 | 11p15.1 | 19825540 | 19990184 | 164644 |
| 802 | BAC163_D07 | 11p14.2 | 26981648 | 27075621 | 93973 |
| 803 | BAC65_I22 | 11p14.1 | 29017623 | 29122987 | 105364 |
| 804 | BAC112_A18 | 11p13 | 32313285 | 32413285 | 100000 |
| 805 | BAC12_F24 | 11p13 | 33749488 | 33902148 | 152660 |
| 806 | BAC161_E20 | 11p13 | 34092319 | 34196786 | 104467 |
| 807 | BAC222_A06 | 11p13 | 34484400 | 34551351 | 66951 |
| 808 | BAC19_L09 | 11p13 | 34552227 | 34638295 | 86068 |
| 809 | BAC97_I05 | 11p13 | 34603277 | 34696088 | 92811 |
| 810 | BAC248_P18 | 11p12 | 40193589 | 40278708 | 85119 |
| 811 | BAC12_H05 | 11p11.2 | 44161904 | 44309630 | 147726 |
| 812 | BAC167_H05 | 11p11.2 | 44485857 | 44618197 | 132340 |
| 813 | BAC33_M02 | 11p11.2 | 47188450 | 47274767 | 86317 |
| 814 | BAC59_H09 | 11p11.12 | 49793929 | 49886457 | 92528 |
| 815 | BAC50_K14 | 11q12.1 | 58999265 | 59139348 | 140083 |
| 816 | BAC101_B24 | 11q12.2 | 60469478 | 60579900 | 110422 |
| 817 | BAC197_D09 | 11q12.2 | 60688490 | 60788201 | 99711 |
| 818 | BAC195_K21 | 11q12.3 | 62057816 | 62124368 | 66552 |
| 819 | BAC228_L09 | 11q13.1 | 63850581 | 63932674 | 82093 |
| 820 | BAC185_L04 | 11q13.1 | 64334520 | 64476963 | 142443 |
| 821 | BAC122_I18 | 11q13.1 | 64588074 | 64679498 | 91424 |
| 822 | BAC192_C22 | 11q13.1 | 64898539 | 64988035 | 89496 |
| 823 | BAC128_J04 | 11q13.1 | 65534728 | 65648775 | 114047 |
| 824 | BAC149_E07 | 11q13.2 | 65714738 | 65823126 | 108388 |
| 825 | BAC241_H14 | 11q13.3 | 68319721 | 68425839 | 106118 |
| 826 | BAC39_F05 | 11q13.3 | 69043658 | 69121709 | 78051 |
| 827 | BAC220_G02 | 11q13.3 | 69182334 | 69259376 | 77042 |
| 828 | BAC75_E17 | 11q13.3 | 69936997 | 70049899 | 112902 |
| 829 | BAC241_C13 | 11q13.4 | 73012763 | 73102284 | 89521 |
| 830 | BAC96_G07 | 11q13.4 | 74057111 | 74142572 | 85461 |
| 831 | BAC116_D07 | 11q13.5 | 75278508 | 75379403 | 100895 |
| 832 | BAC222_F09 | 11q13.5 | 75969454 | 76054927 | 85473 |
| 833 | BAC144_K18 | 11q13.5 | 76047779 | 76169016 | 121237 |
| 834 | BAC38_N11 | 11q14.2 | 86519435 | 86668488 | 149053 |
| 835 | BAC184_B04 | 11q14.3 | 91603538 | 91696080 | 92542 |
| 836 | BAC153_K19 | 11q21 | 94550362 | 94622935 | 72573 |
| 837 | BAC100_H09 | 11q22.1 | 99947567 | 100047832 | 100265 |
| 838 | BAC12_L24 | 11q22.1 | 100578409 | 100626985 | 48576 |
| 839 | BAC44_C18 | 11q22.3 | 104566935 | 104664678 | 97743 |
| 840 | BAC120_O18 | 11q22.3 | 107588166 | 107708395 | 120229 |
| 841 | BAC33_D18 | 11q22.3 | 109234138 | 109351664 | 117526 |
| 842 | BAC75_I11 | 11q23.1 | 111169265 | 111269905 | 100640 |
| 843 | BAC185_E02 | 11q23.1 | 111234728 | 111320236 | 85508 |
| 844 | BAC183_F14 | 11q23.1-11q23.2 | 112724164 | 112827988 | 103824 |
| 845 | BAC145_H08 | 11q23.3 | 115598814 | 115691695 | 92881 |
| 846 | BAC64_I15 | 11q23.3 | 117811398 | 117913740 | 102342 |
| 847 | BAC141_J03 | 11q23.3 | 118668010 | 118770489 | 102479 |
| 848 | BAC158_L17 | 11q23.3 | 118749885 | 118832906 | 83021 |
| 849 | BAC72_F10 | 11q24.1 | 123038674 | 123124317 | 85643 |
| 850 | BAC250_C10 | 11q24.2 | 124815841 | 124908810 | 92969 |
| 851 | BAC121_O09 | 11q24.2 | 126933008 | 127039688 | 106680 |
| 852 | BAC21_O18 | 11q24.3 | 129002983 | 129098874 | 95891 |
| 853 | BAC173_G12 | 11q24.3 | 129783884 | 129885684 | 101800 |
| 854 | BAC91_K14 | 11q25 | 133215093 | 133319321 | 104228 |
| 855 | BAC155_L08 | 12p13.33 | 457779 | 579581 | 121802 |
| 856 | BAC252_M17 | 12p13.33 | 842480 | 1009672 | 167192 |
| 857 | BAC73_O21 | 12p13.33 | 2099018 | 2224808 | 125790 |
| 858 | BAC36_D21 | 12p13.32 | 4279271 | 4365063 | 85792 |
| 859 | BAC115_B18 | 12p13.32 | 4541734 | 4618455 | 76721 |
| 860 | BAC13_A13 | 12p13.32 | 4807113 | 4904890 | 97777 |
| 861 | BAC90_L22 | 12p13.31 | 5580513 | 5683936 | 103423 |
| 862 | BAC188_D23 | 12p13.31 | 6174973 | 6283885 | 108912 |
| 863 | BAC34_J07 | 12p13.31 | 6612012 | 6703618 | 91606 |
| 864 | BAC157_O02 | 12p13.2 | 11685957 | 11787018 | 101061 |
| 865 | BAC25_K04 | 12p13.2 | 12258963 | 12364784 | 105821 |
| 866 | BAC174_C05 | 12p13.1 | 13408446 | 13498007 | 89561 |
| 867 | BAC88_B12 | 12p12.3 | 15790531 | 15865371 | 74840 |
| 868 | BAC140_E24 | 12p12.3 | 16317159 | 16406706 | 89547 |
| 869 | BAC107_B02 | 12p12.1 | 22813677 | 22905673 | 91996 |
| 870 | BAC168_P16 | 12p12.1 | 22959249 | 23048411 | 89162 |
| 871 | BAC69_O13 | 12p12.1 | 24808990 | 24917833 | 108843 |
| 872 | BAC121_J23 | 12p12.1 | 26190999 | 26316525 | 125526 |
| 873 | BAC247_C10 | 12p11.22 | 27906426 | 27986661 | 80235 |
| 874 | BAC42_L03 | 12p11.21 | 30981388 | 31093757 | 112369 |
| 875 | BAC143_H17 | 12p11.21 | 31017454 | 31100025 | 82571 |
| 876 | BAC76_B24 | 12p11.21 | 31436672 | 31560085 | 123413 |
| 877 | BAC1_B14 | 12q12 | 39296553 | 39397884 | 101331 |
| 878 | BAC54_H10 | 12q13.11 | 46633675 | 46743355 | 109680 |
| 879 | BAC217_M07 | 12q13.12 | 47563540 | 47681466 | 117926 |
| 880 | BAC127_P09 | 12q13.13 | 50991842 | 51084867 | 93025 |
| 881 | BAC41_I18 | 12q13.2 | 53220032 | 53326648 | 106616 |
| 882 | BAC115_I13 | 12q13.2 | 54222467 | 54381658 | 159191 |
| 883 | BAC196_D16 | 12q13.3 | 55679177 | 55822021 | 142844 |
| 884 | BAC209_C17 | 12q14.1 | 56466353 | 56573783 | 107430 |
| 885 | BAC108_G22 | 12q14.1 | 60560744 | 60667273 | 106529 |
| 886 | BAC46_N17 | 12q14.2-12q14.3 | 63267705 | 63350142 | 82437 |
| 887 | BAC10_M07 | 12q14.3 | 64498983 | 64596611 | 97628 |
| 888 | BAC234_L21 | 12q15 | 67299354 | 67386403 | 87049 |
| 889 | BAC65_E02 | 12q15 | 67329351 | 67467208 | 137857 |
| 890 | BAC232_J22 | 12q15 | 67483818 | 67571143 | 87325 |
| 891 | BAC154_D12 | 12q15 | 69015053 | 69103442 | 88389 |
| 892 | BAC97_B21 | 12q21.1 | 70221428 | 70314926 | 93498 |
| 893 | BAC59_C14 | 12q21.31 | 83676538 | 83761433 | 84895 |
| 894 | BAC234_E19 | 12q21.32 | 87395480 | 87464496 | 69016 |
| 895 | BAC221_N20 | 12q21.33 | 90992471 | 91092471 | 100000 |
| 896 | BAC84_C20 | 12q21.33-12q22 | 91164576 | 91256290 | 91714 |
| 897 | BAC13_H06 | 12q23.1 | 94840160 | 94915619 | 75459 |
| 898 | BAC1_M18 | 12q23.1 | 97623317 | 97726732 | 103415 |
| 899 | BAC164_J21 | 12q23.2 | 101306542 | 101405928 | 99386 |
| 900 | BAC140_P13 | 12q23.3 | 103578626 | 103654929 | 76303 |
| 901 | BAC22_C06 | 12q23.3 | 105831773 | 105927649 | 95876 |
| 902 | BAC176_A04 | 12q24.12 | 110314011 | 110391539 | 77528 |
| 903 | BAC28_J11 | 12q24.13 | 112416415 | 112451780 | 35365 |
| 904 | BAC82_A13 | 12q24.21 | 114660134 | 114769434 | 109300 |
| 905 | BAC168_A15 | 12q24.22 | 115714208 | 115781756 | 67548 |
| 906 | BAC8_N02 | 12q24.23 | 116557552 | 116658134 | 100582 |
| 907 | BAC162_L24 | 12q24.31 | 120707246 | 120787926 | 80680 |
| 908 | BAC142_E09 | 12q24.31 | 123170158 | 123251181 | 81023 |
| 909 | BAC71_K16 | 12q24.32 | 126642245 | 126758124 | 115879 |
| 910 | BAC22_N22 | 12q24.32 | 127649467 | 127753489 | 104022 |
| 911 | BAC136_E22 | 12q24.33 | 129557401 | 129637183 | 79782 |
| 912 | BAC199_L02 | 13q12.11 | 18433040 | 18557718 | 124678 |
| 913 | BAC77_M02 | 13q12.11 | 20501827 | 20604163 | 102336 |
| 914 | BAC171_B19 | 13q12.12 | 22441596 | 22517054 | 75458 |
| 915 | BAC91_O18 | 13q12.13 | 24894344 | 25001216 | 106872 |
| 916 | BAC158_N18 | 13q12.2 | 26509344 | 26611583 | 102239 |
| 917 | BAC142_B19 | 13q12.3 | 26903129 | 26975050 | 71921 |
| 918 | BAC157_J18 | 13q13.1 | 30498508 | 30608121 | 109613 |
| 919 | BAC43_L23 | 13q13.1 | 30654883 | 30772823 | 117940 |
| 920 | BAC29_D09 | 13q13.2 | 32350284 | 32450376 | 100092 |
| 921 | BAC88_K22 | 13q13.3-13q14.11 | 37873884 | 37963755 | 89871 |
| 922 | BAC147_I15 | 13q14.11 | 42693523 | 42796905 | 103382 |
| 923 | BAC80_F19 | 13q14.12 | 43108705 | 43282650 | 173945 |
| 924 | BAC101_E02 | 13q14.13 | 43890098 | 43988227 | 98129 |
| 925 | BAC122_P16 | 13q14.2 | 45081284 | 45171613 | 90329 |
| 926 | BAC181_L23 | 13q14.2 | 46642007 | 46777050 | 135043 |
| 927 | BAC235_D20 | 13q14.2 | 48290056 | 48404451 | 114395 |
| 928 | BAC66_B10 | 13q14.2 | 48406430 | 48528622 | 122192 |
| 929 | BAC92_J05 | 13q14.3 | 48798348 | 48916233 | 117885 |
| 930 | BAC27_O11 | 13q14.3 | 48961232 | 49073011 | 111779 |
| 931 | BAC29_C16 | 13q14.3 | 51114561 | 51221600 | 107039 |
| 932 | BAC175_N19 | 13q14.3 | 52838057 | 52940262 | 102205 |
| 933 | BAC63_I22 | 13q21.2 | 57707871 | 57835668 | 127797 |
| 934 | BAC51_L06 | 13q21.32 | 63680942 | 63786166 | 105224 |
| 935 | BAC168_M20 | 13q21.32 | 64995345 | 65079169 | 83824 |
| 936 | BAC117_L08 | 13q21.33 | 69982812 | 70071910 | 89098 |
| 937 | BAC96_N17 | 13q21.33 | 70389090 | 70480502 | 91412 |
| 938 | BAC154_E21 | 13q22.1 | 72656512 | 72726261 | 69749 |
| 939 | BAC60_F12 | 13q22.2 | 73657363 | 73803146 | 145783 |
| 940 | BAC145_C09 | 13q22.3 | 76266771 | 76342289 | 75518 |
| 941 | BAC97_A19 | 13q31.1 | 77564263 | 77668779 | 104516 |
| 942 | BAC29_K13 | 13q31.2 | 85975636 | 86083353 | 107717 |
| 943 | BAC166_A03 | 13q32.1 | 92965119 | 93055136 | 90017 |
| 944 | BAC84_E11 | 13q32.1 | 94659834 | 94770009 | 110175 |
| 945 | BAC143_B05 | 13q32.2 | 96859138 | 96937794 | 78656 |
| 946 | BAC73_O17 | 13q32.3 | 99162365 | 99247785 | 85420 |
| 947 | BAC155_B10 | 13q33.1 | 101157413 | 101252806 | 95393 |
| 948 | BAC233_L22 | 13q33.1 | 101437725 | 101517569 | 79844 |
| 949 | BAC193_H21 | 13q33.2-13q33.3 | 104755638 | 104837582 | 81944 |
| 950 | BAC41_E02 | 13q34 | 108139728 | 108221657 | 81929 |
| 951 | BAC123_J22 | 13q34 | 108999812 | 109091630 | 91818 |
| 952 | BAC232_G07 | 13q34 | 111934948 | 112008920 | 73972 |
| 953 | BAC96_A02 | 13q34 | 112109608 | 112214195 | 104587 |
| 954 | BAC12_N17 | 14q11.2 | 20035291 | 20150935 | 115644 |
| 955 | BAC239_I12 | 14q11.2 | 20282829 | 20367983 | 85154 |
| 956 | BAC72_L13 | 14q11.2 | 21088968 | 21193828 | 104860 |
| 957 | BAC59_O07 | 14q11.2 | 21481543 | 21600761 | 119218 |
| 958 | BAC1_O24 | 14q11.2 | 22482663 | 22558749 | 76086 |
| 959 | BAC222_N17 | 14q12 | 24873567 | 24972673 | 99106 |
| 960 | BAC157_P13 | 14q12 | 29805641 | 29916230 | 110589 |
| 961 | BAC162_N24 | 14q13.1 | 31864138 | 31963407 | 99269 |
| 962 | BAC96_D15 | 14q13.3 | 35007093 | 35115839 | 108746 |
| 963 | BAC90_M15 | 14q21.2 | 44752439 | 44845981 | 93542 |
| 964 | BAC143_A23 | 14q21.3 | 48544048 | 48632002 | 87954 |
| 965 | BAC237_C10 | 14q21.3 | 48544120 | 48632001 | 87881 |
| 966 | BAC72_P15 | 14q22.1 | 50337759 | 50409455 | 71696 |
| 967 | BAC25_M16 | 14q22.2 | 53008282 | 53185744 | 177462 |
| 968 | BAC80_J19 | 14q22.3 | 54708525 | 54815562 | 107037 |
| 969 | BAC96_P02 | 14q23.1 | 59083638 | 59189617 | 105979 |
| 970 | BAC58_J08 | 14q23.3 | 63556203 | 63683632 | 127429 |
| 971 | BAC100_O07 | 14q24.2 | 68679164 | 68772800 | 93636 |
| 972 | BAC33_I21 | 14q24.3 | 73887705 | 74077420 | 189715 |
| 973 | BAC119_F08 | 14q24.3 | 73892236 | 73982504 | 90268 |
| 974 | BAC57_M11 | 14q24.3 | 74400348 | 74516407 | 116059 |
| 975 | BAC97_I21 | 14q24.3 | 75209065 | 75310728 | 101663 |
| 976 | BAC102_L12 | 14q24.3 | 75724560 | 75840076 | 115516 |
| 977 | BAC65_N03 | 14q24.3 | 75883770 | 76078979 | 195209 |
| 978 | BAC145_C03 | 14q31.1 | 79022350 | 79117023 | 94673 |
| 979 | BAC248_M08 | 14q31.1 | 79483085 | 79564860 | 81775 |
| 980 | BAC233_P09 | 14q31.1 | 79888231 | 80024386 | 136155 |
| 981 | BAC175_D05 | 14q31.2 | 81605769 | 81731060 | 125291 |
| 982 | BAC11_N10 | 14q31.3 | 84038159 | 84139903 | 101744 |
| 983 | BAC22_P14 | 14q31.3 | 86626547 | 86734974 | 108427 |
| 984 | BAC81_E17 | 14q32.11 | 88182118 | 88284822 | 102704 |
| 985 | BAC100_B22 | 14q32.12 | 91569769 | 91738410 | 168641 |
| 986 | BAC215_G20 | 14q32.13 | 94090075 | 94172652 | 82577 |
| 987 | BAC97_N03 | 14q32.13-14q32.2 | 94275090 | 94378036 | 102946 |
| 988 | BAC36_J22 | 14q32.2 | 97860860 | 98005633 | 144773 |
| 989 | BAC115_N23 | 14q32.2 | 98697552 | 98823198 | 125646 |
| 990 | BAC88_C21 | 14q32.31 | 99829211 | 99957537 | 128326 |
| 991 | BAC26_I17 | 14q32.32 | 101194408 | 101354107 | 159699 |
| 992 | BAC166_E01 | 14q32.33 | 102174928 | 102273866 | 98938 |
| 993 | BAC142_N17 | 14q32.33 | 102189891 | 102198272 | 8381 |
| 994 | BAC69_K17 | 14q32.33 | 104675881 | 104792706 | 116825 |
| 995 | BAC13_B06 | 14q32.33 | 104687477 | 104823462 | 135985 |
| 996 | BAC232_G16 | 14q32.33 | 104721924 | 104808158 | 86234 |
| 997 | BAC27_E18 | 14q32.33 | 104976687 | 105066558 | 89871 |
| 998 | BAC220_P13 | 15q11.2 | 22518053 | 22598139 | 80086 |
| 999 | BAC88_I16 | 15q11.2 | 22852377 | 22953047 | 100670 |
| 1000 | BAC91_O02 | 15q12 | 25543075 | 25674894 | 131819 |
| 1001 | BAC182_D23 | 15q13.1 | 27526984 | 27607080 | 80096 |
| 1002 | BAC242_O02 | 15q13.3 | 29011346 | 29098099 | 86753 |
| 1003 | BAC19_L10 | 15q13.3 | 29030253 | 29160435 | 130182 |
| 1004 | BAC59_F17 | 15q13.3 | 30061880 | 30165725 | 103845 |
| 1005 | BAC127_N19 | 15q14 | 31788290 | 31898829 | 110539 |
| 1006 | BAC170_H09 | 15q14 | 32768081 | 32871486 | 103405 |
| 1007 | BAC197_N21 | 15q14 | 33750051 | 33870988 | 120937 |
| 1008 | BAC182_L10 | 15q14 | 37457384 | 37540818 | 83434 |
| 1009 | BAC83_E21 | 15q15.1 | 37825287 | 37928025 | 102738 |
| 1010 | BAC23_H19 | 15q15.1 | 38225441 | 38329716 | 104275 |
| 1011 | BAC49_P16 | 15q15.1 | 39384833 | 39487228 | 102395 |
| 1012 | BAC161_M10 | 15q15.1 | 39673333 | 39757633 | 84300 |
| 1013 | BAC91_O04 | 15q15.1 | 40301292 | 40407875 | 106583 |
| 1014 | BAC100_H20 | 15q21.1 | 45682643 | 45772966 | 90323 |
| 1015 | BAC66_J01 | 15q21.1 | 46292257 | 46429916 | 137659 |
| 1016 | BAC40_C05 | 15q21.1-15q21.2 | 47330628 | 47446498 | 115870 |
| 1017 | BAC68_C12 | 15q21.2 | 48245378 | 48370691 | 125313 |
| 1018 | BAC187_A04 | 15q21.2 | 49137168 | 49264115 | 126947 |
| 1019 | BAC222_P21 | 15q21.2 | 49150129 | 49262147 | 112018 |
| 1020 | BAC236_E23 | 15q21.2 | 49297143 | 49400567 | 103424 |
| 1021 | BAC142_K19 | 15q21.3 | 55919806 | 56002050 | 82244 |
| 1022 | BAC57_A19 | 15q22.2 | 57142789 | 57243284 | 100495 |
| 1023 | BAC176_G02 | 15q22.2 | 61203838 | 61317446 | 113608 |
| 1024 | BAC37_B13 | 15q22.31 | 62353316 | 62438404 | 85088 |
| 1025 | BAC84_I01 | 15q23 | 67585063 | 67680523 | 95460 |
| 1026 | BAC106_B15 | 15q24.1 | 70538353 | 70619749 | 81396 |
| 1027 | BAC197_J09 | 15q24.1 | 72665709 | 72778385 | 112676 |
| 1028 | BAC147_N01 | 15q24.1 | 72665762 | 72780247 | 114485 |
| 1029 | BAC138_F17 | 15q24.1 | 72860554 | 72960554 | 100000 |
| 1030 | BAC81_K22 | 15q24.3 | 75491303 | 75665329 | 174026 |
| 1031 | BAC10_E08 | 15q25.1 | 78812634 | 78922848 | 110214 |
| 1032 | BAC128_O24 | 15q25.2 | 80963443 | 81073037 | 109594 |
| 1033 | BAC102_M10 | 15q25.3 | 86140215 | 86272137 | 131922 |
| 1034 | BAC231_E18 | 15q26.1 | 88037414 | 88137414 | 100000 |
| 1035 | BAC11_J18 | 15q26.1 | 88594162 | 88725488 | 131326 |
| 1036 | BAC120_J06 | 15q26.2 | 93555790 | 93685902 | 130112 |
| 1037 | BAC8_I18 | 15q26.3 | 98667917 | 98780539 | 112622 |
| 1038 | BAC184_K14 | 16p13.3 | 74386 | 167160 | 92774 |
| 1039 | BAC142_G12 | 16p13.3 | 516853 | 617469 | 100616 |
| 1040 | BAC252_I22 | 16p13.3 | 2066703 | 2157935 | 91232 |
| 1041 | BAC59_B08 | 16p13.3 | 3119948 | 3203303 | 83355 |
| 1042 | BAC60_M03 | 16p13.3 | 3283122 | 3376833 | 93711 |
| 1043 | BAC49_J01 | 16p13.3 | 3291540 | 3426981 | 135441 |
| 1044 | BAC100_H02 | 16p13.2 | 6631478 | 6759323 | 127845 |
| 1045 | BAC20_K17 | 16p13.13 | 11199508 | 11336242 | 136734 |
| 1046 | BAC68_B16 | 16p13.13 | 12457720 | 12554899 | 97179 |
| 1047 | BAC97_C21 | 16p13.12 | 13428306 | 13524235 | 95929 |
| 1048 | BAC152_G18 | 16p13.11 | 14932120 | 15017722 | 85602 |
| 1049 | BAC31_G15 | 16p12.3 | 17278739 | 17375100 | 96361 |
| 1050 | BAC51_A24 | 16p12.3 | 19409805 | 19495668 | 85863 |
| 1051 | BAC191_F16 | 16p12.2 | 23386957 | 23486670 | 99713 |
| 1052 | BAC62_C07 | 16p12.2 | 24027759 | 24116057 | 88298 |
| 1053 | BAC171_P05 | 16p12.1 | 27757955 | 27862558 | 104603 |
| 1054 | BAC59_L05 | 16p12.1 | 28278385 | 28377886 | 99501 |
| 1055 | BAC246_C01 | 16p11.2 | 31097476 | 31234190 | 136714 |
| 1056 | BAC153_M14 | 16q12.1 | 50001053 | 50080041 | 78988 |
| 1057 | BAC36_L03 | 16q12.2 | 56073230 | 56171585 | 98355 |
| 1058 | BAC145_A03 | 16q12.2 | 56135828 | 56229224 | 93396 |
| 1059 | BAC153_E19 | 16q13 | 57377357 | 57487277 | 109920 |
| 1060 | BAC96_G02 | 16q21-16q22.1 | 64983819 | 65114723 | 130904 |
| 1061 | BAC137_J20 | 16q22.1 | 66272391 | 66382811 | 110420 |
| 1062 | BAC61_H22 | 16q22.1 | 67703953 | 67824302 | 120349 |
| 1063 | BAC144_N12 | 16q22.1 | 69177980 | 69273587 | 95607 |
| 1064 | BAC53_H20 | 16q22.1 | 69428547 | 69529481 | 100934 |
| 1065 | BAC120_I17 | 16q22.1 | 70392282 | 70521354 | 129072 |
| 1066 | BAC61_N20 | 16q22.3 | 72757131 | 72853305 | 96174 |
| 1067 | BAC84_E07 | 16q23.1 | 78664463 | 78779551 | 115088 |
| 1068 | BAC200_K17 | 16q23.2 | 80619782 | 80726340 | 106558 |
| 1069 | BAC170_D16 | 16q24.1 | 84690347 | 84790381 | 100034 |
| 1070 | BAC54_O09 | 16q24.2 | 88328837 | 88430965 | 102128 |
| 1071 | BAC96_I21 | 16q24.3 | 89112516 | 89248303 | 135787 |
| 1072 | BAC72_M12 | 16q24.3 | 89373875 | 89454739 | 80864 |
| 1073 | BAC101_G17 | 16q24.3 | 89551931 | 89633927 | 81996 |
| 1074 | BAC49_N19 | 17p13.3 | 448041 | 582881 | 134840 |
| 1075 | BAC132_D18 | 17p13.3 | 706378 | 816464 | 110086 |
| 1076 | BAC122_B18 | 17p13.3 | 2720563 | 2820563 | 100000 |
| 1077 | BAC73_C22 | 17p13.2 | 3554395 | 3640306 | 85911 |
| 1078 | BAC155_O16 | 17p13.2 | 5929934 | 6048208 | 118274 |
| 1079 | BAC106_L03 | 17p13.1 | 6958834 | 7058622 | 99788 |
| 1080 | BAC195_B24 | 17p13.1 | 7265310 | 7430003 | 164693 |
| 1081 | BAC88_C08 | 17p13.1 | 7695752 | 7791177 | 95425 |
| 1082 | BAC1_L24 | 17p13.1 | 8512342 | 8625373 | 113031 |
| 1083 | BAC24_E11 | 17p13.1 | 9934734 | 10074883 | 140149 |
| 1084 | BAC59_B19 | 17p13.1 | 10715377 | 10845411 | 130034 |
| 1085 | BAC140_N23 | 17p12 | 11555629 | 11635751 | 80122 |
| 1086 | BAC36_P09 | 17p12 | 12335482 | 12466672 | 131190 |
| 1087 | BAC90_H16 | 17p11.2 | 17283727 | 17383307 | 99580 |
| 1088 | BAC132_L20 | 17p11.2 | 18811539 | 18902037 | 90498 |
| 1089 | BAC132_O07 | 17p11.2 | 19427051 | 19515261 | 88210 |
| 1090 | BAC10_I20 | 17p11.2 | 19739497 | 19816424 | 76927 |
| 1091 | BAC244_N11 | 17q11.2 | 27192282 | 27305126 | 112844 |
| 1092 | BAC97_C05 | 17q11.2 | 27551162 | 27642561 | 91399 |
| 1093 | BAC57_D07 | 17q11.2 | 28990730 | 29103499 | 112769 |
| 1094 | BAC221_C05 | 17q11.2 | 29697594 | 29784748 | 87154 |
| 1095 | BAC137_H11 | 17q11.2 | 29779899 | 29879900 | 100001 |
| 1096 | BAC107_E15 | 17q11.2 | 30367709 | 30530816 | 163107 |
| 1097 | BAC57_O05 | 17q11.2 | 31413038 | 31530855 | 117817 |
| 1098 | BAC8_J15 | 17q11.2 | 32828946 | 32917154 | 88208 |
| 1099 | BAC57_B10 | 17q11.2 | 33260909 | 33359668 | 98759 |
| 1100 | BAC91_E18 | 17q12 | 33995596 | 34131953 | 136357 |
| 1101 | BAC157_E20 | 17q12 | 34327316 | 34410851 | 83535 |
| 1102 | BAC106_G05 | 17q12 | 37148612 | 37328388 | 179776 |
| 1103 | BAC68_N12 | 17q12 | 38108081 | 38202819 | 94738 |
| 1104 | BAC217_J01 | 17q12 | 38110864 | 38182903 | 72039 |
| 1105 | BAC84_G20 | 17q21.1 | 38376852 | 38526220 | 149368 |
| 1106 | BAC195_G10 | 17q21.2 | 38845470 | 38949564 | 104094 |
| 1107 | BAC65_F13 | 17q21.2 | 39110224 | 39207293 | 97069 |
| 1108 | BAC128_M18 | 17q21.2 | 40563559 | 40663559 | 100000 |
| 1109 | BAC130_L05 | 17q21.2 | 40754608 | 40842257 | 87649 |
| 1110 | BAC211_I10 | 17q21.2 | 41140783 | 41280649 | 139866 |
| 1111 | BAC163_I15 | 17q21.2 | 41200452 | 41299660 | 99208 |
| 1112 | BAC193_O02 | 17q21.31 | 41559995 | 41637965 | 77970 |
| 1113 | BAC84_D23 | 17q21.31 | 42393262 | 42504510 | 111248 |
| 1115 | BAC81_K18 | 17q21.33 | 48182023 | 48260693 | 78670 |
| 1116 | BAC40_M12 | 17q22 | 53407641 | 53537896 | 130255 |
| 1117 | BAC42_E17 | 17q22 | 53783127 | 53908716 | 125589 |
| 1118 | BAC131_P23 | 17q22 | 53885405 | 53989110 | 103705 |
| 1119 | BAC48_F01 | 17q23.2 | 55661859 | 55760140 | 98281 |
| 1120 | BAC133_G08 | 17q23.2 | 56781474 | 56878242 | 96768 |
| 1121 | BAC66_I09 | 17q23.2 | 59940950 | 60023235 | 82285 |
| 1122 | BAC88_D15 | 17q23.3 | 62186901 | 62270623 | 83722 |
| 1123 | BAC153_M16 | 17q24.2 | 65122531 | 65241573 | 119042 |
| 1124 | BAC56_P20 | 17q24.2 | 65679239 | 65843567 | 164328 |
| 1125 | BAC38_O07 | 17q24.2 | 67110244 | 67221898 | 111654 |
| 1126 | BAC60_H19 | 17q24.3 | 70380143 | 70464143 | 84000 |
| 1127 | BAC48_H06 | 17q24.3 | 70620306 | 70727359 | 107053 |
| 1128 | BAC59_E12 | 17q25.1 | 75192996 | 75307205 | 114209 |
| 1129 | BAC171_D05 | 17q25.3 | 76192302 | 76267901 | 75599 |
| 1130 | BAC36_L05 | 17q25.3 | 77054437 | 77153882 | 99445 |
| 1131 | BAC82_B17 | 17q25.3 | 80506579 | 80616674 | 110095 |
| 1132 | BAC43_M02 | 18p11.32 | 1480552 | 1589598 | 109046 |
| 1133 | BAC72_K17 | 18p11.32 | 1832320 | 1937191 | 104871 |
| 1134 | BAC191_N20 | 18p11.31 | 4325614 | 4399606 | 73992 |
| 1135 | BAC106_H08 | 18p11.31 | 6097510 | 6188241 | 90731 |
| 1136 | BAC34_E05 | 18p11.23 | 7496917 | 7595313 | 98396 |
| 1137 | BAC30_E07 | 18p11.22 | 9880416 | 9984699 | 104283 |
| 1138 | BAC167_F11 | 18p11.22-18p11.21 | 10702382 | 10800044 | 97662 |
| 1139 | BAC86_A18 | 18p11.21 | 12912296 | 13026904 | 114608 |
| 1140 | BAC210_D05 | 18p11.21 | 13026802 | 13116696 | 89894 |
| 1141 | BAC127_L04 | 18q11.2 | 18833401 | 18962393 | 128992 |
| 1142 | BAC131_F03 | 18q11.2 | 19909813 | 19990240 | 80427 |
| 1143 | BAC86_N24 | 18q12.1 | 23800943 | 23902381 | 101438 |
| 1144 | BAC19_B03 | 18q12.1 | 28253813 | 28342253 | 88440 |
| 1145 | BAC171_I13 | 18q12.2 | 31336856 | 31441278 | 104422 |
| 1146 | BAC241_H18 | 18q21.1 | 45850630 | 45929489 | 78859 |
| 1147 | BAC111_I02 | 18q21.1 | 45974589 | 46073220 | 98631 |
| 1148 | BAC1_E24 | 18q21.1 | 46382064 | 46501454 | 119390 |
| 1149 | BAC144_H12 | 18q21.1 | 46766348 | 46864168 | 97820 |
| 1150 | BAC120_C22 | 18q21.1 | 48099636 | 48208222 | 108586 |
| 1151 | BAC251_G19 | 18q21.2 | 49958497 | 50062023 | 103526 |
| 1152 | BAC248_E22 | 18q21.2 | 50005255 | 50089139 | 83884 |
| 1153 | BAC71_L11 | 18q21.2-18q21.31 | 52421762 | 52566954 | 145192 |
| 1154 | BAC1_O22 | 18q21.31 | 53894403 | 53991228 | 96825 |
| 1155 | BAC78_M19 | 18q21.32 | 54734920 | 54862666 | 127746 |
| 1156 | BAC190_E09 | 18q21.33 | 59046186 | 59143498 | 97312 |
| 1157 | BAC93_C13 | 18q21.33 | 59143422 | 59226399 | 82977 |
| 1158 | BAC63_E12 | 18q21.33 | 59409262 | 59496927 | 87665 |
| 1159 | BAC39_C18 | 18q21.33-18q22.1 | 59717050 | 59828190 | 111140 |
| 1160 | BAC96_D20 | 18q22.1 | 64375723 | 64491394 | 115671 |
| 1161 | BAC196_P15 | 18q22.3 | 67164106 | 67246946 | 82840 |
| 1162 | BAC97_B02 | 18q22.3 | 70059067 | 70164618 | 105551 |
| 1163 | BAC51_E18 | 18q23 | 71505225 | 71607424 | 102199 |
| 1164 | BAC92_P18 | 18q23 | 72756269 | 72854279 | 98010 |
| 1165 | BAC96_L08 | 19p13.3 | 241127 | 322089 | 80962 |
| 1166 | BAC75_B14 | 19p13.3 | 3128150 | 3239601 | 111451 |
| 1167 | BAC33_L13 | 19p13.3 | 3362990 | 3515482 | 152492 |
| 1168 | BAC65_N13 | 19p13.3 | 3672362 | 3784579 | 112217 |
| 1169 | BAC37_C21 | 19p13.3 | 4138670 | 4319043 | 180373 |
| 1170 | BAC57_D10 | 19p13.3 | 5668164 | 5790697 | 122533 |
| 1171 | BAC110_G12 | 19p13.3 | 5754441 | 5881582 | 127141 |
| 1172 | BAC15_C01 | 19p13.3 | 5809184 | 5915297 | 106113 |
| 1173 | BAC78_A24 | 19p13.3 | 6783304 | 6872943 | 89639 |
| 1174 | BAC247_P10 | 19p13.2 | 7723334 | 7822406 | 99072 |
| 1175 | BAC242_P14 | 19p13.2 | 10175575 | 10260895 | 85320 |
| 1176 | BAC193_N03 | 19p13.2 | 11296365 | 11423757 | 127392 |
| 1177 | BAC50_B05 | 19p13.2 | 12799308 | 12904737 | 105429 |
| 1178 | BAC131_D05 | 19p13.2 | 13333297 | 13405823 | 72526 |
| 1179 | BAC63_L12 | 19p13.12 | 15173143 | 15211462 | 38319 |
| 1180 | BAC232_P18 | 19p13.12 | 16504868 | 16606286 | 101418 |
| 1181 | BAC246_J06 | 19p13.11 | 18074719 | 18177597 | 102878 |
| 1182 | BAC192_L06 | 19p13.11 | 18500870 | 18570701 | 69831 |
| 1183 | BAC195_K16 | 19p13.11 | 19864394 | 19973711 | 109317 |
| 1184 | BAC144_L05 | 19q12 | 36700414 | 36843108 | 142694 |
| 1185 | BAC90_H02 | 19q13.12 | 40345497 | 40468196 | 122699 |
| 1186 | BAC162_E14 | 19q13.13 | 42500153 | 42612049 | 111896 |
| 1187 | BAC162_K04 | 19q13.2 | 44526162 | 44654219 | 128057 |
| 1188 | BAC146_F01 | 19q13.2 | 45387806 | 45488625 | 100819 |
| 1189 | BAC162_J04 | 19q13.2 | 45952164 | 46028945 | 76781 |
| 1190 | BAC249_F13 | 19q13.2 | 46383337 | 46477055 | 93718 |
| 1191 | BAC239_N12 | 19q13.31 | 48749455 | 48848106 | 98651 |
| 1192 | BAC183_D06 | 19q13.31 | 48847871 | 48996197 | 148326 |
| 1193 | BAC78_D10 | 19q13.31 | 48847942 | 48958616 | 110674 |
| 1194 | BAC233_C05 | 19q13.32 | 50661471 | 50753092 | 91621 |
| 1195 | BAC30_K23 | 19q13.32 | 51219159 | 51308806 | 89647 |
| 1196 | BAC168_H24 | 19q13.33 | 54081400 | 54161818 | 80418 |
| 1197 | BAC130_J22 | 19q13.41 | 55996991 | 56086613 | 89622 |
| 1198 | BAC165_O12 | 19q13.41 | 57215236 | 57306157 | 90921 |
| 1199 | BAC53_O15 | 19q13.42 | 58885334 | 59049697 | 164363 |
| 1200 | BAC168_E17 | 19q13.42 | 60716170 | 60824234 | 108064 |
| 1201 | BAC223_A15 | 19q13.43 | 61937176 | 62037176 | 100000 |
| 1202 | BAC48_A12 | 19q13.43 | 62753996 | 62869459 | 115463 |
| 1203 | BAC40_E05 | 20p13 | 950567 | 1060691 | 110124 |
| 1204 | BAC109_E03 | 20p13 | 3557532 | 3654929 | 97397 |
| 1205 | BAC127_K22 | 20p12.3 | 5079550 | 5170764 | 91214 |
| 1206 | BAC85_D15 | 20p12.3 | 5603512 | 5703512 | 100000 |
| 1207 | BAC155_K05 | 20p12.3 | 8754431 | 8844829 | 90398 |
| 1208 | BAC143_E21 | 20p12.2 | 9239562 | 9338364 | 98802 |
| 1209 | BAC86_O12 | 20p12.2 | 10579633 | 10716155 | 136522 |
| 1210 | BAC247_K09 | 20p12.2 | 10612988 | 10710034 | 97046 |
| 1211 | BAC165_M10 | 20p12.1 | 12571678 | 12660146 | 88468 |
| 1212 | BAC86_O11 | 20p12.1 | 14772916 | 14926315 | 153399 |
| 1213 | BAC81_M07 | 20p12.1 | 15322403 | 15449323 | 126920 |
| 1214 | BAC81_K08 | 20p12.1 | 16169962 | 16258799 | 88837 |
| 1215 | BAC29_A11 | 20p12.1 | 16650508 | 16782248 | 131740 |
| 1216 | BAC132_A05 | 20p12.1 | 17009215 | 17102828 | 93613 |
| 1217 | BAC96_K10 | 20p12.1 | 17408006 | 17520565 | 112559 |
| 1218 | BAC44_G02 | 20p11.23 | 18648077 | 18758594 | 110517 |
| 1219 | BAC75_H16 | 20p11.23 | 20351941 | 20460637 | 108696 |
| 1220 | BAC63_I12 | 20p11.22 | 21412498 | 21517095 | 104597 |
| 1221 | BAC110_E03 | 20p11.21 | 24994915 | 25108843 | 113928 |
| 1222 | BAC36_M13 | 20p11.21 | 25200289 | 25373995 | 173706 |
| 1223 | BAC204_A05 | 20q11.21 | 32067010 | 32156330 | 89320 |
| 1224 | BAC183_P06 | 20q11.21 | 32067013 | 32148815 | 81802 |
| 1225 | BAC140_D09 | 20q11.23 | 38075528 | 38200551 | 125023 |
| 1226 | BAC43_F22 | 20q12 | 38383033 | 38554426 | 171393 |
| 1227 | BAC239_E02 | 20q12 | 39551098 | 39642464 | 91366 |
| 1228 | BAC97_K09 | 20q12 | 39964163 | 40041129 | 76966 |
| 1229 | BAC21_G16 | 20q12 | 40401091 | 40510999 | 109908 |
| 1230 | BAC52_H17 | 20q12 | 41478073 | 41592599 | 114526 |
| 1231 | BAC177_F04 | 20q12 | 41559912 | 41663035 | 103123 |
| 1232 | BAC75_I24 | 20q13.12 | 43269482 | 43355740 | 86258 |
| 1233 | BAC12_K18 | 20q13.13 | 47716616 | 47825988 | 109372 |
| 1234 | BAC193_H13 | 20q13.13 | 49715263 | 49817640 | 102377 |
| 1235 | BAC72_N10 | 20q13.2 | 53356476 | 53451975 | 95499 |
| 1236 | BAC51_F06 | 20q13.2 | 53617860 | 53841898 | 224038 |
| 1237 | BAC97_O05 | 20q13.2 | 53929620 | 54032536 | 102916 |
| 1238 | BAC63_G09 | 20q13.32 | 56793070 | 56914693 | 121623 |
| 1239 | BAC142_K22 | 20q13.32 | 58382628 | 58464614 | 81986 |
| 1240 | BAC42_C03 | 20q13.33 | 60237784 | 60340737 | 102953 |
| 1241 | BAC90_N02 | 20q13.33 | 63316793 | 63410107 | 93314 |
| 1242 | BAC29_L21 | 21q11.2 | 14659343 | 14766232 | 106889 |
| 1243 | BAC190_J11 | 21q11.2 | 14989525 | 15073544 | 84019 |
| 1244 | BAC183_C22 | 21q21.1 | 20059719 | 20183055 | 123336 |
| 1245 | BAC100_H22 | 21q21.2 | 23812356 | 23913099 | 100743 |
| 1246 | BAC65_O07 | 21q21.3 | 26143952 | 26259891 | 115939 |
| 1247 | BAC249_C23 | 21q21.3 | 27101711 | 27203592 | 101881 |
| 1248 | BAC36_D16 | 21q21.3 | 29290000 | 29388816 | 98816 |
| 1249 | BAC84_O06 | 21q22.11 | 31350858 | 31449318 | 98460 |
| 1250 | BAC11_L10 | 21q22.11 | 31828260 | 31921565 | 93305 |
| 1251 | BAC63_J12 | 21q22.11 | 31921400 | 32041535 | 120135 |
| 1252 | BAC155_L05 | 21q22.11 | 32707790 | 32832682 | 124892 |
| 1253 | BAC203_B21 | 21q22.11 | 33491072 | 33591479 | 100407 |
| 1254 | BAC182_I05 | 21q22.11 | 33761592 | 33830417 | 68825 |
| 1255 | BAC56_C18 | 21q22.11 | 34147688 | 34327992 | 180304 |
| 1256 | BAC82_F19 | 21q22.11-21q22.12 | 34571538 | 34748392 | 176854 |
| 1257 | BAC65_N02 | 21q22.12 | 34803249 | 34906451 | 103202 |
| 1258 | BAC117_H13 | 21q22.12 | 34833743 | 34963940 | 130197 |
| 1259 | BAC166_I08 | 21q22.12 | 34868737 | 34960579 | 91842 |
| 1260 | BAC203_M13 | 21q22.12 | 35058114 | 35132553 | 74439 |
| 1261 | BAC165_I10 | 21q22.12 | 36053804 | 36155088 | 101284 |
| 1262 | BAC8_G15 | 21q22.13 | 37269976 | 37359485 | 89509 |
| 1263 | BAC213_C09 | 21q22.13 | 38108436 | 38207891 | 99455 |
| 1264 | BAC141_F01 | 21q22.2 | 38644900 | 38741144 | 96244 |
| 1265 | BAC42_O16 | 21q22.2 | 39024524 | 39171652 | 147128 |
| 1266 | BAC88_N19 | 21q22.2 | 40233536 | 40332751 | 99215 |
| 1267 | BAC88_N19 | 21q22.2 | 40233536 | 40332751 | 99215 |
| 1268 | BAC109_E20 | 21q22.3 | 41447533 | 41570422 | 122889 |
| 1269 | BAC37_M17 | 21q22.3 | 42144949 | 42259302 | 114353 |
| 1270 | BAC145_K01 | 21q22.3 | 42289089 | 42380366 | 91277 |
| 1271 | BAC46_A15 | 21q22.3 | 42795447 | 42880952 | 85505 |
| 1272 | BAC8_N04 | 21q22.3 | 43416843 | 43506866 | 90023 |
| 1273 | BAC96_C04 | 21q22.3 | 43877962 | 43987382 | 109420 |
| 1274 | BAC1_C02 | 21q22.3 | 43963093 | 44050254 | 87161 |
| 1275 | BAC155_O07 | 21q22.3 | 44553929 | 44644142 | 90213 |
| 1276 | BAC80_E04 | 21q22.3 | 45015415 | 45125892 | 110477 |
| 1277 | BAC136_D13 | 21q22.3 | 45031012 | 45127430 | 96418 |
| 1278 | BAC209_K24 | 21q22.3 | 45127713 | 45222279 | 94566 |
| 1279 | BAC207_B17 | 21q22.3 | 45503537 | 45611138 | 107601 |
| 1280 | BAC88_N11 | 21q22.3 | 45782179 | 45934096 | 151917 |
| 1281 | BAC147_A13 | 21q22.3 | 46763053 | 46873853 | 110800 |
| 1282 | BAC185_A10 | 22q11.21 | 16585502 | 16703290 | 117788 |
| 1283 | BAC173_K08 | 22q11.21 | 17035175 | 17154309 | 119134 |
| 1284 | BAC82_N03 | 22q11.21 | 17981523 | 18115068 | 133545 |
| 1285 | BAC73_G13 | 22q11.21 | 18054142 | 18133800 | 79658 |
| 1286 | BAC45_L11 | 22q11.21 | 18114912 | 18236616 | 121704 |
| 1287 | BAC135_D20 | 22q11.21 | 18250892 | 18337061 | 86169 |
| 1288 | BAC235_E11 | 22q11.21 | 19372279 | 19502515 | 130236 |
| 1289 | BAC122_J17 | 22q11.21 | 19501143 | 19658729 | 157586 |
| 1290 | BAC170_F13 | 22q11.22-22q11.23 | 21764932 | 21893837 | 128905 |
| 1291 | BAC209_P14 | 22q11.23 | 21768683 | 21855396 | 86713 |
| 1292 | BAC193_N18 | 22q11.23 | 22663632 | 22778729 | 115097 |
| 1293 | BAC96_M16 | 22q11.23 | 23426152 | 23569727 | 143575 |
| 1294 | BAC199_I14 | 22q11.23 | 24009545 | 24110169 | 100624 |
| 1295 | BAC40_G01 | 22q12.1 | 25065786 | 25203548 | 137762 |
| 1296 | BAC12_F19 | 22q12.1 | 26160169 | 26288390 | 128221 |
| 1297 | BAC161_N14 | 22q12.1 | 26715479 | 26846451 | 130972 |
| 1298 | BAC143_A19 | 22q12.1 | 27639126 | 27747271 | 108145 |
| 1299 | BAC229_K08 | 22q12.2 | 27941326 | 28020526 | 79200 |
| 1300 | BAC1_K13 | 22q12.2 | 28020430 | 28154454 | 134024 |
| 1301 | BAC191_G18 | 22q12.2 | 28157561 | 28257068 | 99507 |
| 1302 | BAC37_J03 | 22q12.2 | 28378252 | 28491078 | 112826 |
| 1303 | BAC117_O16 | 22q12.2 | 28401441 | 28491126 | 89685 |
| 1304 | BAC219_K02 | 22q12.2 | 30000418 | 30121260 | 120842 |
| 1305 | BAC103_G23 | 22q12.2 | 30053433 | 30178199 | 124766 |
| 1306 | BAC140_B15 | 22q12.3 | 30652122 | 30741131 | 89009 |
| 1307 | BAC196_J04 | 22q12.3 | 31510114 | 31604206 | 94092 |
| 1308 | BAC90_G19 | 22q12.3 | 32218076 | 32325756 | 107680 |
| 1309 | BAC19_A04 | 22q12.3 | 34450431 | 34562284 | 111853 |
| 1310 | BAC19_A04 | 22q12.3 | 34450431 | 34562284 | 111853 |
| 1311 | BAC131_H09 | 22q12.3 | 34942382 | 35069724 | 127342 |
| 1312 | BAC240_E16 | 22q12.3 | 34978590 | 35078590 | 100000 |
| 1313 | BAC25_B14 | 22q12.3 | 35220469 | 35316635 | 96166 |
| 1314 | BAC15_I17 | 22q12.3-22q13.1 | 35727018 | 35920037 | 193019 |
| 1315 | BAC190_O21 | 22q13.1 | 37244771 | 37336583 | 91812 |
| 1316 | BAC161_F18 | 22q13.1 | 37343267 | 37470851 | 127584 |
| 1317 | BAC200_M11 | 22q13.1 | 37470767 | 37565068 | 94301 |
| 1318 | BAC33_J12 | 22q13.1 | 37665513 | 37766108 | 100595 |
| 1319 | BAC180_G12 | 22q13.1 | 37777327 | 37882548 | 105221 |
| 1320 | BAC154_K23 | 22q13.1 | 37781962 | 37930897 | 148935 |
| 1321 | BAC143_E09 | 22q13.1 | 38562566 | 38648365 | 85799 |
| 1322 | BAC59_J18 | 22q13.2 | 39272711 | 39360769 | 88058 |
| 1323 | BAC145_D16 | 22q13.2 | 39357110 | 39501404 | 144294 |
| 1324 | BAC238_J21 | 22q13.2 | 41310641 | 41426781 | 116140 |
| 1325 | BAC122_O04 | 22q13.2 | 41549637 | 41728984 | 179347 |
| 1326 | BAC189_F08 | 22q13.31 | 42701860 | 42817073 | 115213 |
| 1327 | BAC78_L06 | 22q13.31 | 44894709 | 45052574 | 157865 |
| 1328 | BAC131_L06 | 22q13.31 | 45208045 | 45330257 | 122212 |
| 1329 | BAC73_C03 | 22q13.31 | 46149882 | 46235766 | 85884 |
| 1330 | BAC33_J04 | 22q13.33 | 47939426 | 48070084 | 130658 |
| 1331 | BAC248_E20 | 22q13.33 | 49066764 | 49168930 | 102166 |
| 1332 | BAC32_O13 | Xp22.33 | 2367225 | 2472415 | 105190 |
| 1333 | BAC153_J20 | Xp22.22 | 9231921 | 9331921 | 100000 |
| 1334 | BAC143_P20 | Xp22.22 | 9657664 | 9752433 | 94769 |
| 1335 | BAC166_K19 | Xp22.22 | 10663707 | 10748286 | 84579 |
| 1336 | BAC72_G18 | Xp22.22 | 12319091 | 12401604 | 82513 |
| 1337 | BAC161_F20 | Xp22.22 | 12914583 | 13020166 | 105583 |
| 1338 | BAC122_M06 | Xp22.22 | 15041048 | 15127934 | 86886 |
| 1339 | BAC16_E18 | Xp22.13 | 17523395 | 17618824 | 95429 |
| 1340 | BAC180_G08 | Xp22.13 | 17595852 | 17697987 | 102135 |
| 1341 | BAC43_L14 | Xp22.13 | 18014360 | 18109910 | 95550 |
| 1342 | BAC230_I10 | Xp22.13 | 18418346 | 18519739 | 101393 |
| 1343 | BAC120_N07 | Xp22.11 | 22568240 | 22649249 | 81009 |
| 1344 | BAC96_B05 | Xp22.11 | 23470896 | 23562686 | 91790 |
| 1345 | BAC89_J08 | Xp22.11 | 23884296 | 23972217 | 87921 |
| 1346 | BAC62_H18 | Xp22.11 | 24047294 | 24118361 | 71067 |
| 1347 | BAC46_K11 | Xp22.11-Xp21.3 | 24330893 | 24427534 | 96641 |
| 1348 | BAC63_K16 | Xp21.1 | 31188097 | 31274907 | 86810 |
| 1349 | BAC118_L05 | Xp21.1 | 31952484 | 32061404 | 108920 |
| 1350 | BAC63_O15 | Xp21.1 | 32228238 | 32315594 | 87356 |
| 1351 | BAC46_M08 | Xp21.1 | 36573217 | 36660687 | 87470 |
| 1352 | BAC117_O07 | Xp11.4 | 37239448 | 37316488 | 77040 |
| 1353 | BAC131_J03 | Xp11.4 | 37277870 | 37355452 | 77582 |
| 1354 | BAC139_J06 | Xp11.4 | 40516388 | 40597919 | 81531 |
| 1355 | BAC26_B20 | Xp11.4 | 40895658 | 41036563 | 140905 |
| 1356 | BAC7_O11 | Xp11.3 | 42812523 | 42911660 | 99137 |
| 1357 | BAC176_A16 | Xp11.3-Xp11.23 | 46479187 | 46613095 | 133908 |
| 1358 | BAC128_N06 | Xp11.23 | 47119509 | 47209251 | 89742 |
| 1359 | BAC122_O17 | Xp11.23 | 47809280 | 47961298 | 152018 |
| 1360 | BAC128_N09 | Xp11.23 | 48157296 | 48285768 | 128472 |
| 1361 | BAC39_M13 | Xp11.23-Xp11.22 | 48754981 | 48862262 | 107281 |
| 1362 | BAC31_P15 | Xp11.22 | 52139259 | 52258150 | 118891 |
| 1363 | BAC39_H16 | Xp11.21 | 53736905 | 53871606 | 134701 |
| 1364 | BAC247_M15 | Xp11.21 | 54018343 | 54093869 | 75526 |
| 1365 | BAC24_I13 | Xq11.2 | 61594055 | 61731370 | 137315 |
| 1366 | BAC141_P21 | Xq12 | 64074633 | 64164626 | 89993 |
| 1367 | BAC59_K08 | Xq12 | 65770669 | 65879205 | 108536 |
| 1368 | BAC23_O07 | Xq13.1 | 66854379 | 66980784 | 126405 |
| 1369 | BAC178_B15 | Xq13.1 | 67201571 | 67283943 | 82372 |
| 1370 | BAC142_O01 | Xq13.1 | 69624480 | 69723593 | 99113 |
| 1371 | BAC182_P03 | Xq13.1 | 70330068 | 70442678 | 112610 |
| 1372 | BAC110_I17 | Xq13.2 | 72536471 | 72672674 | 136203 |
| 1373 | BAC170_L05 | Xq21.1 | 75898755 | 75986911 | 88156 |
| 1374 | BAC179_M02 | Xq21.1 | 76059930 | 76145025 | 85095 |
| 1375 | BAC20_K10 | Xq21.1 | 81102663 | 81225786 | 123123 |
| 1376 | BAC76_H11 | Xq21.32 | 91454180 | 91583204 | 129024 |
| 1377 | BAC211_I16 | Xq21.32 | 91626890 | 91723415 | 96525 |
| 1378 | BAC6_B17 | Xq21.33 | 94907491 | 95018810 | 111319 |
| 1379 | BAC57_E05 | Xq22.1 | 99271404 | 99396307 | 124903 |
| 1380 | BAC209_I17 | Xq22.1 | 99773224 | 99870655 | 97431 |
| 1381 | BAC43_D09 | Xq22.2 | 101760102 | 101854994 | 94892 |
| 1382 | BAC141_P03 | Xq22.2 | 102384569 | 102488663 | 104094 |
| 1383 | BAC56_M03 | Xq22.3 | 105782457 | 105878590 | 96133 |
| 1384 | BAC200_I13 | Xq22.3 | 106430413 | 106532237 | 101824 |
| 1385 | BAC55_G15 | Xq23 | 108440983 | 108527568 | 86585 |
| 1386 | BAC58_E08 | Xq23 | 109151622 | 109281412 | 129790 |
| 1387 | BAC41_C23 | Xq23 | 110655724 | 110747997 | 92273 |
| 1388 | BAC62_N08 | Xq23 | 113650688 | 113789455 | 138767 |
| 1389 | BAC103_A22 | Xq13.1 | 113730419 | 113790321 | 59902 |
| 1390 | BAC165_P14 | Xq23 | 114260198 | 114320905 | 60707 |
| 1391 | BAC51_H24 | Xq24 | 118202334 | 118300323 | 97989 |
| 1392 | BAC85_A21 | Xq25 | 121792442 | 121879410 | 86968 |
| 1393 | BAC176_E20 | Xq25 | 122759251 | 122835121 | 75870 |
| 1394 | BAC247_N09 | Xq25 | 126960660 | 127038666 | 78006 |
| 1395 | BAC31_O21 | Xq26.2 | 130517423 | 130622376 | 104953 |
| 1396 | BAC69_N21 | Xq26.2 | 131539749 | 131676083 | 136334 |
| 1397 | BAC41_L18 | Xq26.2 | 132210618 | 132366873 | 156255 |
| 1398 | BAC36_B11 | Xq26.3 | 132584762 | 132668298 | 83536 |
| 1399 | BAC247_B11 | Xq26.3 | 133966096 | 134066393 | 100297 |
| 1400 | BAC247_B11 | Xq26.3 | 133966096 | 134066393 | 100297 |
| 1401 | BAC21_J11 | Xq26.3 | 134290254 | 134389273 | 99019 |
| 1402 | BAC122_G10 | Xq26.3 | 134635371 | 134716821 | 81450 |
| 1403 | BAC170_L14 | Xq26.3 | 135259977 | 135369152 | 109175 |
| 1404 | BAC214_M20 | Xq27.1 | 136942761 | 137030129 | 87368 |
| 1405 | BAC14_M14 | Xq27.1 | 138763382 | 138844208 | 80826 |
| 1406 | BAC233_N08 | Xq27.2 | 139007118 | 139089725 | 82607 |
| 1407 | BAC77_O23 | Xq27.2 | 139194173 | 139297048 | 102875 |
| 1408 | BAC29_D20 | Xq27.2 | 139914549 | 140061750 | 147201 |
| 1409 | BAC61_I07 | Xq27.3 | 142324669 | 142420377 | 95708 |
| 1410 | BAC166_J20 | Xq27.3 | 142911155 | 142996763 | 85608 |
| 1411 | BAC55_F19 | Xq28 | 148732366 | 148825233 | 92867 |
| 1412 | BAC139_D06 | Xq28 | 149378105 | 149484441 | 106336 |
| 1413 | BAC139_P13 | Xq28 | 150490992 | 150588703 | 97711 |
| 1414 | BAC139_P13 | Xq28 | 150490992 | 150588703 | 97711 |
| 1415 | BAC43_D10 | Xq28 | 150786623 | 150954986 | 168363 |
| 1416 | BAC27_B24 | Xq28 | 151283476 | 151429154 | 145678 |
| 1417 | BAC123_P03 | Xq28 | 151447703 | 151577035 | 129332 |
| 1418 | BAC170_P07 | Xq28 | 151858476 | 151941545 | 83069 |
| 1419 | BAC171_F11 | Xq28 | 152144739 | 152273778 | 129039 |
| 1420 | BAC31_I13 | Xq28 | 152329278 | 152475058 | 145780 |
| 1421 | BAC90_L23 | Xq28 | 152581837 | 152669189 | 87352 |
| 1422 | BAC154_L24 | Xq28 | 153124985 | 153224985 | 100000 |
| 1423 | BAC202_C09 | Yp11.31 | 2491867 | 2571557 | 79690 |
| 1424 | BAC158_N08 | Yp11.2 | 6836586 | 6898386 | 61800 |
| 1425 | BAC52_L18 | Yp11.2 | 8869376 | 8974494 | 105118 |
| 1426 | BAC10_I08 | Yp11.2 | 9166337 | 9295674 | 129337 |
| 1427 | BAC58_H14 | Yq11.221 | 15973469 | 16055735 | 82266 |
| 1428 | BAC94_F15 | Yq11.221 | 16086505 | 16186505 | 100000 |
| 1429 | BAC36_J10 | Yq11.221 | 17732213 | 17821055 | 88842 |
| 1430 | BAC126_D02 | Yq11.221 | 19141327 | 19298090 | 156763 |
| 1431 | BAC220_P01 | Yq11.222 | 19768053 | 19854639 | 86586 |
| 1432 | BAC80_J09 | Yq11.222 | 21539003 | 21673125 | 134122 |
| 1433 | BAC60_N12 | Yq11.223 | 21949617 | 22050893 | 101276 |
| 1434 | BAC37_G04 | Yq11.223 | 22257679 | 22340650 | 82971 |
| 1435 | BAC147_N13 | Yq11.223 | 22338341 | 22486942 | 148601 |
| 1436 | BAC132_A12 | Yq11.223 | 22866346 | 22914575 | 48229 |
| 1437 | BAC170_J12 | Yq11.223 | 24637082 | 24720962 | 83880 |
| 1438 | BAC41_I24 | Yq11.23 | 25990166 | 26129583 | 139417 |
| 1439 | BAC194_K10 | Yq11.23 | 26527037 | 26658969 | 131932 |
| 1440 | BAC154_P04 | Yq12 | 50112233 | 50203629 | 91396 |

Genome Library

The genomic library can be prepared according to the conventional method. For example, human BAC DNA can be obtained by performing the process including preparation of BAC library, sequence analysis of a part of BAC DNA inserted into BAC clone, analysis of chromosomal location of BAC DNA by using the homology analysis the analyzed nucleotide sequence with identified human genome sequence, analysis of whole nucleotide sequence, reconfirmation of chromosomal location of BAC DNA by using FISH ((Fluorescent In Situ Hybridization), and the recovery of BAC DNA from the BAC clone. The process of the obtaining the BAC DNA will be explained in more detail.

(1) Preparation of Genomic Library

The human genome is separated, and cleavaged with treatment of restriction enzyme (for examples HindIII, and BamHI) and/or sonication, and performed by Pulse Field Gel Electrophoresis to produce DNA fragment having the mean size of 100 to 350 Kb. The DNA fragment is introduced into the BAC, PAC, P1, YAC or cosmid vector which is treated with the same restriction enzyme, and then transformed to host cell, for example E. coli. The cell line including the human genomic DNA fragment is referred to as a clone. The present inventor made 96,768 clones in total according to the above method.

(2) Identification of Location and Whole Nucleotide Sequence of the Genomic DNA Fragment Inserted into the Clone

The genomic DNA fragment in each clone is performed by homology analysis with the known human genome sequence. The databases used for the homology analysis are NCBI, UC Santa Cruz, and Sanger Institute.

For homology analysis, the end-sequencing of both ends (for example, 100 to 700 bp nucleotides) of the inserted genomic DNA fragment is performed by sequencing with primers specific to the vector. The end-sequencing can be easily performed, because the nucleotide sequence and restriction map of vector are identified. The homology analysis is performed by inputting the nucleotide sequence of both ends into the database of the human genome sequence, and the consistent region is confirmed to chromosomal location and entire nucleotide sequencing of the genomic DNA fragment are confirmed.

(3) Reconfirmation of Chromosomal Location of Genomic DNA Fragment by Using FISH

The reconfirmation step can be omitted, but performed for confirming the location on genome of the genomic DNA fragment.

The genomic DNA fragment is used as a probe, labeled by a fluorescent material, and then reacted with human chromosome in the metaphase of cell division. The chromosomal location of the genomic DNA fragment is reconfirmed by detecting the fluorescent signal.

(4) Recovery of the Genomic DNA Fragment from the Clone

The genomic DNA fragment is recovered from each clone according to the conventional method, or by using the commercial recovery kit

According to the above steps, the genomic DNA fragment inserted into the each clone is identified. The characteristics of genomic DNA fragment such as chromosomal location, nucleotide sequence, function, related gene or disease information are collected, and the genomic DNA fragment can be selected for the suitable use of DNA chip. In particular, when a DNA chip for diagnosing a specific disease, each genomic DNA fragment corresponding to chromosome abnormality related to the occurrence and the prognosis of disease can be easily recovered from each clone.

In an embodiment of the present invention, genomic DNA fragments of the BAC library of Korean people are shown in Table 1.

Preparation of Genomic DNA Microarray for Detecting Chromosome Abnormality

The present invention relates to a method of preparing DNA microarray for detecting chromosome abnormality contains the steps of:

(a) identifying genomic DNA fragment inserted to each clone which is obtained from organism's genomic library;

(b) selecting target chromosomal region to be detected for chromosome abnormality;

(c) selecting from genomic DNA fragments identified in step (a) at least a genomic DNA fragment as a probe for detecting the sample chromosomal region, and

(d) immobilizing the probe in a microarray.

In step (a), the genomic DNA fragment can be at least one selected from the group consisting of genomic DNA fragments in Table 1, and preferably can be selected depending on the chromosomal region to be detected for chromosome abnormality, and diseases.

In step (b), the chromosome abnormality is selected from the group consisting of mutation of nucleotide sequence, amplification in the number of the chromosome, deletion in the number of chromosome, chromosome inversion, and chromosome translocation, preferably the abnormality of chromosome number. The examples of the diseases associated with the abnormality of chromosome number are Down's syndrome, Patau syndrome, Edwards syndrome, Turner syndrome, Klinefelter syndrome, ATR-16 (alpha-thalassemia retardation-16), CMT1A (Charcot-Marie-Tooth neuropathy 1A), Cri-du-chat syndrome, Digeorge syndrome, HNPP disease (Hereditary Neuropathy with Liability to Pressure palsies), Kallmann syndrome Miller-Dieker syndrome, Prader-willi syndrome, Rubinstein-taybi syndrome, Smith-magenes syndrome, Steroid-sulfatase gene syndrome, Williams syndrome and Wolf-hirschhorn syndrome.

In step (b), the target chromosome region is a region showing disease-specific mutation, for examples, a chromosome region showing the amplification or deletion of chromosome number, which can be easily selected by a person of ordinary skill in the art those who the present invention pertains to.

In step (c), the genomic DNA fragments for detecting the chromosome region are selected. The genomic DNA fragments are suitably selected by analyzing the chromosome location, nucleotide sequence, association with disease, and function. Preferably, the genomic DNA fragment contains repetitive sequence at the amount of at most 85% of nucleotide sequence, and has single locus on chromosome. The probe can be the whole or parts of target chromosome, or homologous sequences thereof.

In step (d), the selected probe can be immobilized on a substrate for microarray according to the conventional method. For example, all the DNA of genomic DNA library vector [symbol] DNA can be used for probe, and the DNA is partially cleaved with sonication and then immobilized on the substrate. The process is general method for preparing the genomic DNA microarray, and thus can be easily performed by a person of ordinary skill in the art which the present invention pertains to.

As examples, the present invention provides a microarray or DNA chip for detecting each disease.

(1) Down's Syndrome

Depending on the karyotype, Down's syndrome is classified into trisomy21, mosaicism, and translocation, which occur with frequencies of 93%, 2˜3%, and 3˜5%, respectively. The present invention relates to diagnosing the karyotype of Trisomy 21.

DNA chip for detecting Down's syndrome includes a microarray containing at least one immobilized genomic DNA fragment selected from the group consisting of genomic DNA fragment 1 to 34 of Table 3, at least one oligomer of 20 to 100 bp consecutive nucleotide within the fragment, or at least one cDNA of 100 bp to 10 kbp derived from the fragment. When at least two kinds of probes are mixed and used, the probes can be mixed in equal proportions and immobilized in a single spot.

TABLE 3

| | | | Chromosomal location | | |
|---|---|---|---|---|---|
| | | Chromosomal location | in Human genome | | |
| BAC DNA | No. | identified by FISH | Start point | End point | size |
| BAC8_G15 | 1 | 21 | 37269976 | 37359485 | 89509 |
| BAC11_L10 | 2 | 21q22.11 | 31828260 | 31921565 | 93305 |
| BAC29_L21 | 3 | 21q11.2 | 14659343 | 14766232 | 106889 |
| BAC36_D16 | 4 | 21q21.3 | 29290000 | 29388816 | 98816 |
| BAC37_M17 | 5 | 21q22.3 | 42144949 | 42259302 | 114353 |
| BAC42_O16 | 6 | 21q22.2 | 39024524 | 39171652 | 147128 |
| BAC56_C18 | 7 | 21q22.11 | 34147688 | 34327992 | 180304 |
| BAC65_N02 | 8 | 21q22.12 | 34803249 | 34906451 | 103202 |
| BAC82_F19 | 9 | 21q22.11 | 34571538 | 34748392 | 176854 |
| BAC88_N11 | 10 | 21q22.3 | 45782179 | 45934096 | 151917 |
| BAC88_N19 | 11 | 21q22.2 | 40233536 | 40332751 | 99215 |
| BAC96_C04 | 12 | 21q22.3 | 43877962 | 43987382 | 109420 |
| BAC109_E20 | 13 | 21q22.3 | 41447534 | 41570422 | 122888 |
| BAC117_H13 | 14 | 21q22.12 | 34833747 | 34963940 | 130193 |
| BAC141_F01 | 15 | 21q22.2 | 38644900 | 38741144 | 96244 |
| BAC145_K01 | 16 | 21q22.3 | 42289089 | 42380366 | 91277 |
| BAC147_A13 | 17 | 21q22.3 | 46763053 | 46873853 | 110800 |
| BAC182_I05 | 18 | 21q22.11 | 33761721 | 33830417 | 68696 |
| BAC190_J11 | 19 | 21q11.2 | 14989525 | 15073483 | 83958 |
| BAC203_B21 | 20 | 21q22.11 | 33491072 | 33591479 | 100407 |
| BAC203_M13 | 21 | 21q22.12 | 35058114 | 35132553 | 74439 |
| BAC207_B17 | 22 | 21q22.3 | 45503537 | 45611138 | 107601 |
| BAC213_C09 | 23 | 21q22.13 | 38108433 | 38207914 | 99481 |
| BAC1_C02 | 24 | 21q22.3 | 43963093 | 44050254 | 87161 |
| BAC84_O06 | 25 | 21q22.11 | 31350858 | 31449318 | 98460 |
| BAC88_N19 | 26 | 21q22.2 | 40233536 | 40332751 | 99215 |
| BAC100_H22 | 27 | 21q21.2 | 23812356 | 23913099 | 100743 |
| BAC155_L05 | 28 | 21q22.11 | 32707790 | 32832682 | 124892 |
| BAC165_I10 | 29 | 21q22.12 | 36053804 | 36155088 | 101284 |
| BAC166_I08 | 30 | 21q22.12 | 34868737 | 34960579 | 91842 |
| BAC183_C22 | 31 | 21q21.1 | 20059719 | 20183055 | 123336 |
| BAC46_A15 | 32 | 21q22.3 | 42795447 | 42880952 | 85505 |
| BAC65_O07 | 33 | 21q21.3 | 26143952 | 26259891 | 115939 |
| BAC136_D13 | 34 | 21q22.3 | 45031012 | 45127373 | 96361 |

(2) Patau Syndrome

Patau syndrome can be diagnosed for Karyotype trisomy 13. A DNA chip for detecting Patau syndrome includes a microarray containing at least one immobilized genomic DNA fragment selected from the group consisting of genomic DNA fragment 1 to 34 of Table 4, at least one oligomer of 20 to 100 bp consecutive nucleotide within the fragment, or at least one cDNA of 100 bp to 10 kbp derived from the fragment. When at least two kinds of probes are mixed and used, the probes can be mixed in equal proportions and immobilized in a single spot.

TABLE 4

| | |
|---|---|
| | Chromosomal location |
| Chromosomal location | in Human genome |

| BAC DNA | No. | identified by FISH | Start point | End point | size |
|---|---|---|---|---|---|
| BAC27_O11 | 1 | 13q14.3 | 48961232 | 49073011 | 111779 |
| BAC123_J22 | 2 | 13q34 | 108999812 | 109091630 | 91818 |
| BAC145_C09 | 3 | 13q22.3 | 76266771 | 76342289 | 75518 |
| BAC155_B10 | 4 | 13q33.1 | 101157413 | 101252806 | 95393 |
| BAC157_J18 | 5 | 13q13.1 | 30498508 | 30608121 | 109613 |
| BAC199_L02 | 6 | 13q12.11 | 18433040 | 18557718 | 124678 |
| BAC232_G07 | 7 | 13q34 | 111934948 | 112008920 | 73972 |
| BAC235_D20 | 8 | 13q14.2 | 48290038 | 48404451 | 114413 |
| BAC29_D09 | 9 | 13q13.2 | 32350284 | 32450376 | 100092 |
| BAC29_K13 | 10 | 13q31.2 | 85975636 | 86083353 | 107717 |
| BAC60_F12 | 11 | 13q22.2 | 73657363 | 73803146 | 145783 |
| BAC63_I22 | 12 | 13q21.2 | 57707871 | 57835668 | 127797 |
| BAC73_O17 | 13 | 13q32.3 | 99162365 | 99247785 | 85420 |
| BAC77_M02 | 14 | 13q12.11 | 20501827 | 20604163 | 102336 |
| BAC80_F19 | 15 | 13q14.12 | 43108705 | 43282650 | 173945 |
| BAC84_E11 | 16 | 13q32.1 | 94659834 | 94770009 | 110175 |
| BAC88_K22 | 17 | 13q13.3 | 37873884 | 37963755 | 89871 |
| BAC91_O18 | 18 | 13q12.13 | 24894344 | 25001216 | 106872 |
| BAC92_J05 | 19 | 13q14.3 | 48798348 | 48916233 | 117885 |
| BAC96_A02 | 20 | 13q34 | 112109608 | 112214195 | 104587 |
| BAC96_N17 | 21 | 13q21.33 | 70389090 | 70480502 | 91412 |
| BAC97_A19 | 22 | 13q31.1 | 77564263 | 77668779 | 104516 |
| BAC143_B05 | 23 | 13q32.2 | 96859138 | 96937794 | 78656 |
| BAC147_I15 | 24 | 13q14.11 | 42693523 | 42796905 | 103382 |
| BAC154_E21 | 25 | 13q22.1 | 72656512 | 72726261 | 69749 |
| BAC166_A03 | 26 | 13q32.1 | 92965119 | 93055136 | 90017 |
| BAC168_M20 | 27 | 13q21.32 | 64995345 | 65079169 | 83824 |
| BAC171_B19 | 28 | 13q12.12 | 22441596 | 22517054 | 75458 |
| BAC175_N19 | 29 | 13q14.3 | 52838057 | 52940262 | 102205 |
| BAC193_H21 | 30 | 13q33.3 | 104755638 | 104837582 | 81944 |
| BAC233_L22 | 31 | 13q33.1 | 101437725 | 101517500 | 79775 |
| BAC29_C16 | 32 | 13q14.3 | 51114561 | 51221600 | 107039 |
| BAC66_B10 | 33 | 13q14.2 | 48406430 | 48528622 | 122192 |
| BAC122_P16 | 34 | 13q14.2 | 45081284 | 45171613 | 90329 |

(3) Edwards Syndrome

Edwards syndrome can be diagnosed for Karyotype trisomy 18. A DNA chip for detecting Edwards syndrome includes a microarray containing at least one immobilized genomic DNA fragment selected from the group consisting of genomic DNA fragment 1 to 28 of Table 5, at least one oligomer of 20 to 100 bp consecutive nucleotide within the fragment, or at least one cDNA of 100 bp to 10 kbp derived from the fragment. When at least two kinds of probes are mixed and used, the probes can be mixed in equal proportions and immobilized in a single spot.

TABLE 5

| | | | Chromosomal location | | |
|---|---|---|---|---|---|
| | | Chromosomal location | in Human genome | | |
| BAC DNA | No. | identified by FISH | Start point | End point | size |
| BAC19_B03 | 1 | 18q12.1 | 28253813 | 28342253 | 88440 |
| BAC43_M02 | 2 | 18p11.32 | 1480552 | 1589598 | 109046 |
| BAC51_E18 | 3 | 18q23 | 71505225 | 71607424 | 102199 |
| BAC63_E12 | 4 | 18q21.33 | 59409262 | 59496927 | 87665 |
| BAC93_C13 | 5 | 18q21.33 | 59143422 | 59226399 | 82977 |
| BAC106_H08 | 6 | 18p11.31 | 6097510 | 6188241 | 90731 |
| BAC144_H12 | 7 | 18q21.1 | 46766348 | 46864168 | 97820 |
| BAC167_F11 | 8 | 18p11.22 | 10702382 | 10800044 | 97662 |
| BAC190_E09 | 9 | 18q21.33 | 59046171 | 59143498 | 97327 |
| BAC210_D05 | 10 | 18p11.21 | 13026802 | 13116696 | 89894 |
| BAC241_H18 | 11 | 18q21.1 | 45850630 | 45929489 | 78859 |
| BAC251_G19 | 12 | 18q21.2 | 49958497 | 50062023 | 103526 |
| BAC1_E24 | 13 | 18q21.1 | 46382064 | 46501454 | 119390 |
| BAC1_O22 | 14 | 18q21.31 | 53894403 | 53991228 | 96825 |
| BAC30_E07 | 15 | 18p11.22 | 9880416 | 9984699 | 104283 |
| BAC34_E05 | 16 | 18p11.23 | 7496917 | 7595313 | 98396 |
| BAC39_C18 | 17 | 18q21.33 | 59717050 | 59828190 | 111140 |
| BAC71_L11 | 18 | 18q21.2 | 52421762 | 52566954 | 145192 |
| BAC72_K17 | 19 | 18p11.32 | 1832320 | 1937191 | 104871 |
| BAC78_M19 | 20 | 18q21.32 | 54734920 | 54862666 | 127746 |
| BAC86_A18 | 21 | 18p11.21 | 12912296 | 13026904 | 114608 |
| BAC86_N24 | 22 | 18q12.1 | 23800943 | 23902381 | 101438 |
| BAC96_D20 | 23 | 18q22.1 | 64375723 | 64491394 | 115671 |
| BAC131_F03 | 24 | 18q11.2 | 19909813 | 19990240 | 80427 |
| BAC171_I13 | 25 | 18q12.2 | 31336856 | 31441278 | 104422 |
| BAC191_N20 | 26 | 18p11.31 | 4325614 | 4399606 | 73992 |
| BAC196_P15 | 27 | 18q22.3 | 67164106 | 67246946 | 82840 |
| BAC97_B02 | 28 | 18q22.3 | 70059067 | 70164618 | 105551 |

(4) Turner Syndrome

Turner syndrome can be diagnosed for Karyotype 45, XO. A DNA chip for detecting Turner syndrome includes a microarray containing at least one immobilized genomic DNA fragment selected from the group consisting of genomic DNA fragment 1 to 75 of Table 6, at least one oligomer of 20 to 100 bp consecutive nucleotide within the fragment, or at least one cDNA of 100 bp to 10 kbp derived from the fragment. When at least two kinds of probes are mixed and used, the probes can be mixed in equal proportions and immobilized in a single spot.

TABLE 6

| | | | Chromosomal location | | |
|---|---|---|---|---|---|
| | | Chromosomal location | in Human genome | | |
| BAC DNA | No. | identified by FISH | Start point | End point | size |
| BAC14_M14 | 1 | Xq27.1 | 138763382 | 138844208 | 80826 |
| BAC23_O07 | 2 | Xq13.1 | 66854379 | 66980791 | 126412 |
| BAC27_B24 | 3 | Xq28 | 151283476 | 151429154 | 145678 |
| BAC29_D20 | 4 | Xq27.2 | 139914549 | 140061750 | 147201 |
| BAC31_I13 | 5 | Xq28 | 152329278 | 152475058 | 145780 |
| BAC32_O13 | 6 | Xp22.33 | 2367225 | 2472415 | 105190 |
| BAC39_H16 | 7 | Xp11.21 | 53736905 | 53871606 | 134701 |
| BAC39_M13 | 8 | Xp11.23 | 48754981 | 48862262 | 107281 |
| BAC41_L18 | 9 | Xq26.2 | 132210618 | 132366873 | 156255 |
| BAC43_L14 | 10 | Xp22.13 | 18014360 | 18109910 | 95550 |
| BAC51_H24 | 11 | Xq24 | 118202334 | 118300323 | 97989 |
| BAC55_F19 | 12 | Xq28 | 148732366 | 148825233 | 92867 |
| BAC57_E05 | 13 | Xq22.1 | 99271404 | 99396307 | 124903 |
| BAC58_E08 | 14 | Xq23 | 109151622 | 109281412 | 129790 |
| BAC59_K08 | 15 | Xq12 | 65770669 | 65879205 | 108536 |
| BAC62_N08 | 16 | Xq23 | 113650688 | 113789455 | 138767 |
| BAC72_G18 | 17 | Xp22.22 | 12319091 | 12401604 | 82513 |
| BAC85_A21 | 18 | Xq25 | 121792442 | 121879410 | 86968 |
| BAC89_J08 | 19 | Xp22.11 | 23884296 | 23972217 | 87921 |
| BAC90_L23 | 20 | Xq28 | 152581837 | 152669189 | 87352 |
| BAC96_B05 | 21 | Xp22.11 | 23470896 | 23562686 | 91790 |
| BAC103_A22 | 22 | Xq13.1 | 113730419 | 113790321 | 59902 |
| BAC117_O07 | 23 | Xp11.4 | 37239463 | 37316488 | 77025 |
| BAC118_L05 | 24 | Xp21.1 | 31952492 | 32061404 | 108912 |
| BAC122_M06 | 25 | Xp22.22 | 15041048 | 15127865 | 86817 |
| BAC123_P03 | 26 | Xq28 | 151447703 | 151577032 | 129329 |
| BAC247_B11 | 27 | Xq26.3 | 133966096 | 134066393 | 100297 |
| BAC154_P04 | 28 | Xq28 | 153518069 | 153609465 | 91396 |
| BAC166_K19 | 29 | Xp22.22 | 10663707 | 10748286 | 84579 |
| BAC170_L05 | 30 | Xq21.1 | 75898755 | 75986911 | 88156 |
| BAC170_L14 | 31 | Xq26.3 | 135259977 | 135369152 | 109175 |
| BAC170_P07 | 32 | Xq28 | 151858476 | 151941545 | 83069 |
| BAC176_A16 | 33 | Xp11.23 | 46479187 | 46613095 | 133908 |
| BAC182_P03 | 34 | Xq13.1 | 70330182 | 70442678 | 112496 |
| BAC211_I16 | 35 | Xq21.32 | 91626890 | 91723415 | 96525 |
| BAC230_I10 | 36 | Xp22.13 | 18418398 | 18519739 | 101341 |
| BAC233_N08 | 37 | Xq27.2 | 139007118 | 139089715 | 82597 |
| BAC247_B11 | 38 | Xq26.3 | 133966096 | 134066393 | 100297 |
| 8AC247_M15 | 39 | Xp11.21 | 54018343 | 54093883 | 75540 |
| BAC6_B17 | 40 | Xq21.33 | 94907491 | 95018810 | 111319 |
| BAC20_K10 | 41 | Xq21.1 | 81102663 | 81225786 | 123123 |
| BAC24_I13 | 42 | Xq11.2 | 61594055 | 61731370 | 137315 |
| BAC31_O21 | 43 | Xq26.2 | 130517423 | 130622376 | 104953 |
| BAC31_P15 | 44 | Xp11.22 | 52139259 | 52258150 | 118891 |
| BAC36_B11 | 45 | Xq26.3 | 132584762 | 132668298 | 83536 |
| BAC41_C23 | 46 | Xq23 | 110655724 | 110747997 | 92273 |
| BAC46_K11 | 47 | Xp22.11 | 24330893 | 24427534 | 96641 |
| BAC56_M03 | 48 | Xq22.3 | 105782457 | 105878590 | 96133 |
| BAC61_I07 | 49 | Xq27.3 | 142324669 | 142420377 | 95708 |
| BAC62_H18 | 50 | Xp22.11 | 24047294 | 24118361 | 71067 |
| BAC63_K16 | 51 | Xp21.1 | 31188097 | 31274907 | 86810 |
| BAC63_O15 | 52 | Xp21.1 | 32228237 | 32315594 | 87357 |
| BAC69_N21 | 53 | Xq26.2 | 131539749 | 131676083 | 136334 |
| BAC110_I17 | 54 | Xq13.2 | 72536471 | 72672678 | 136207 |
| BAC120_N07 | 56 | Xp22.11 | 22568306 | 22649249 | 80943 |
| BAC122_G10 | 57 | Xq26.3 | 134635394 | 134716821 | 81427 |
| BAC128_N09 | 58 | Xp11.23 | 48157296 | 48285785 | 128489 |
| BAC131_J03 | 59 | Xp11.4 | 37277870 | 37355452 | 77582 |
| BAC139_D0 | 60 | Xq28 | 149378105 | 149484441 | 106336 |
| BAC142_O01 | 61 | Xq13.1 | 69624480 | 69723593 | 99113 |
| BAC143_P20 | 62 | Xp22.22 | 9657664 | 9752433 | 94769 |
| BAC161_F20 | 63 | Xp22.22 | 12914583 | 13020166 | 105583 |
| BAC165_P14 | 64 | Xq23 | 114260198 | 114320905 | 60707 |
| BAC166_J20 | 65 | Xq27.3 | 142911155 | 142996763 | 85608 |
| BAC176_E20 | 66 | Xq25 | 122759251 | 122835121 | 75870 |
| BAC178_B15 | 67 | Xq13.1 | 67201571 | 67283943 | 82372 |
| BAC179_M02 | 68 | Xq21.1 | 76059930 | 76145025 | 85095 |
| BAC180_G08 | 69 | Xp22.13 | 17595852 | 17697987 | 102135 |
| BAC200_I13 | 70 | Xq22.3 | 106430413 | 106532237 | 101824 |
| BAC247_N09 | 71 | Xq25 | 126960592 | 127038666 | 78074 |
| BAC122_O17 | 72 | Xp11.23 | 47809333 | 47961298 | 151965 |
| BAC139_J06 | 73 | Xp11.4 | 40516388 | 40597919 | 81531 |
| BAC141_P21 | 74 | Xq12 | 64074633 | 64164626 | 89993 |
| BAC128_N06 | 75 | Xp11.23 | 47119510 | 47209251 | 89741 |

(5) Klinefelter Syndrome

Klinefelter syndrome can be diagnosed for Karyotype XXY. A DNA chip for detecting Klinefelter syndrome includes a microarray containing at least one immobilized genomic DNA fragment selected from the group consisting of genomic DNA fragment 1 to 10 of Table 714, at least one oligomer of 20 to 100 bp consecutive nucleotide within the fragment, or at least one cDNA of 100 bp to 10 kbp derived from the fragment. When at least two kinds of probes are mixed and used, the probes can be mixed in equal proportions and immobilized in a single spot.

TABLE 7

| | | | Chromosomal location | | |
|---|---|---|---|---|---|
| | | Chromosomal location | in Human genome | | |
| BAC DNA | No. | identified by FISH | Start point | End point | size |
| BAC10_I08 | 1 | Yp11.2 | 9166337 | 9295674 | 129337 |
| BAC52_L18 | 2 | Yp11.2 | 8869376 | 8974494 | 105118 |
| BAC126_D02 | 3 | Yq11.221 | 19141327 | 19298090 | 156763 |
| BAC147_N13 | 4 | Yq11.223 | 22338341 | 22486942 | 148601 |
| BAC170_J12 | 5 | Yq11.223 | 24637082 | 24720962 | 83880 |
| BAC194_K10 | 6 | Yq11.23 | 26527037 | 26658969 | 131932 |
| BAC37_G04 | 7 | Yq11.223 | 22257679 | 22340650 | 82971 |
| BAC41_I24 | 8 | Yq11.23 | 25990166 | 26129583 | 139417 |
| BAC202_C09 | 9 | Yp11.31 | 2491867 | 2571557 | 79690 |
| BAC220_P01 | 10 | Yq11.222 | 19768073 | 19854639 | 86566 |

(6) ATR-16 (Alpha-Thalassemia Retardation-16) Syndrome

ATR-16 syndrome can be diagnosed for Karyotype deletion 16p13.3. A DNA chip for detecting ATR-16 syndrome includes a microarray containing at least one immobilized genomic DNA fragment selected from the group consisting of genomic DNA fragment 1 to 141 of Table 8, at least one oligomer of 20 to 100 bp consecutive nucleotide within the fragment, or at least one cDNA of 100 bp to 10 kbp derived from the fragment. When at least two kinds of probes are mixed and used, the probes can be mixed in equal proportions and immobilized in a single spot.

TABLE 8

| | | | Chromosomal location | | |
|---|---|---|---|---|---|
| | | Chromosomal location | in Human genome | | |
| BAC DNA | No. | identified by FISH | Start point | End point | size |
| BAC142_G12 | 1 | 16p13.3 | 516853 | 617469 | 100616 |
| BAC142_H12 | 2 | 16p13.3 | 516853 | 617469 | 100616 |
| BAC250_L07 | 3 | 16p13.3 | 605415 | 704070 | 98655 |
| BAC208_A15 | 4 | 16p13.3 | 720641 | 804644 | 84003 |
| BAC249_C21 | 5 | 16p13.3 | 734807 | 812203 | 77396 |
| BAC230_J12 | 6 | 16p13.3 | 734842 | 845667 | 110825 |
| BAC239_F18 | 7 | 16p13.3 | 834107 | 906859 | 72752 |
| BAC208_D07 | 8 | 16p13.3 | 863029 | 954010 | 90981 |
| BAC61_H17 | 9 | 16p13.3 | 979429 | 1055483 | 76054 |
| BAC35_O10 | 10 | 16p13.3 | 1137200 | 1256188 | 118988 |
| BAC78_M22 | 11 | 16p13.3 | 1256110 | 1355806 | 99696 |
| BAC68_D12 | 12 | 16p13.3 | 1409355 | 1501907 | 92552 |
| BAC109_L07 | 13 | 16p13.3 | 1424231 | 1540754 | 116523 |
| BAC8_E11 | 14 | 16p13.3 | 1424249 | 1525919 | 101670 |
| BAC248_B09 | 15 | 16p13.3 | 1540615 | 1646306 | 105691 |
| BAC175_F10 | 16 | 16p13.3 | 1561044 | 1690272 | 129228 |
| BAC247_K17 | 17 | 16p13.3 | 1561065 | 1679951 | 118886 |
| BAC205_E14 | 18 | 16p13.3 | 1561358 | 1660710 | 99352 |
| BAC247_B19 | 19 | 16p13.3 | 1561392 | 1673861 | 112469 |
| BAC30_J11 | 20 | 16p13.3 | 1563688 | 1646307 | 82619 |
| BAC204_J01 | 21 | 16p13.3 | 1563745 | 1693582 | 129837 |
| BAC160_N03 | 22 | 16p13.3 | 1563753 | 1680019 | 116266 |
| BAC43_H21 | 23 | 16p13.3 | 1594978 | 1673856 | 78878 |
| BAC60_D04 | 24 | 16p13.3 | 1646223 | 1750238 | 104015 |
| BAC101_E17 | 25 | 16p13.3 | 1646223 | 1801163 | 154940 |
| BAC26_C21 | 26 | 16p13.3 | 1660633 | 1801085 | 140452 |
| BAC161_M07 | 27 | 16p13.3 | 1693519 | 1820649 | 127130 |
| BAC208_N11 | 28 | 16p13.3 | 1750132 | 1831984 | 81852 |
| BAC32_K07 | 29 | 16p13.3 | 1840125 | 1953739 | 113614 |
| BAC133_O06 | 30 | 16p13.3 | 1848661 | 1928752 | 80091 |
| BAC124_D13 | 31 | 16p13.3 | 1883046 | 1970703 | 87657 |
| BAC252_I22 | 32 | 16p13.3 | 2066703 | 2157935 | 91232 |
| BAC40_G22 | 33 | 16p13.3 | 2066719 | 2152652 | 85933 |
| BAC53_J01 | 34 | 16p13.3 | 2076740 | 2196453 | 119713 |
| BAC21_B08 | 35 | 16p13.3 | 2076741 | 2156740 | 79999 |
| BAC116_P01 | 36 | 16p13.3 | 2156572 | 2269699 | 113127 |
| BAC184_B18 | 37 | 16p13.3 | 2196281 | 2311075 | 114794 |
| BAC221_H21 | 38 | 16p13.3 | 2260244 | 2352925 | 92681 |
| BAC144_E21 | 39 | 16p13.3 | 2269617 | 2357113 | 87496 |
| BAC124_A07 | 40 | 16p13.3 | 2270740 | 2352937 | 82197 |
| BAC153_B21 | 41 | 16p13.3 | 2328215 | 2410350 | 82135 |
| BAC71_B14 | 42 | 16p13.3 | 2374582 | 2496938 | 122356 |
| BAC236_I05 | 43 | 16p13.3 | 2400543 | 2491319 | 90776 |
| BAC153_A19 | 44 | 16p13.3 | 2405886 | 2513847 | 107961 |
| BAC140_F05 | 45 | 16p13.3 | 2496850 | 2685986 | 189136 |
| BAC150_A04 | 46 | 16p13.3 | 2858237 | 2947506 | 89269 |
| BAC40_D16 | 47 | 16p13.3 | 2931257 | 3032329 | 101072 |
| BAC67_A23 | 48 | 16p13.3 | 2936919 | 3022730 | 85811 |
| BAC109_H14 | 49 | 16p13.3 | 2972416 | 2975331 | 2915 |
| BAC188_G04 | 50 | 16p13.3 | 3022629 | 3105478 | 82849 |
| BAC155_O09 | 51 | 16p13.3 | 3023051 | 3182151 | 159100 |
| BAC144_H13 | 52 | 16p13.3 | 3032234 | 3148709 | 116475 |
| BAC158_C07 | 53 | 16p13.3 | 3032240 | 3105415 | 73175 |
| BAC59_B08 | 54 | 16p13.3 | 3119948 | 3203303 | 83355 |
| BAC84_C17 | 55 | 16p13.3 | 3178597 | 3261330 | 82733 |
| BAC177_J02 | 56 | 16p13.3 | 3179971 | 3281431 | 101460 |
| BAC193_J09 | 57 | 16p13.3 | 3196566 | 3283228 | 86662 |
| BAC136_D19 | 58 | 16p13.3 | 3202961 | 3301749 | 98788 |
| BAC60_M03 | 59 | 16p13.3 | 3283122 | 3376833 | 93711 |
| BAC49_J01 | 60 | 16p13.3 | 3291540 | 3426981 | 135441 |
| BAC23_K05 | 61 | 16p13.3 | 3348066 | 3454552 | 106486 |
| BAC102_D21 | 62 | 16p13.3 | 3496130 | 3589157 | 93027 |
| BAC24_F07 | 63 | 16p13.3 | 3533952 | 3635083 | 101131 |
| BAC178_J13 | 64 | 16p13.3 | 3546307 | 3652872 | 106565 |
| BAC65_A23 | 65 | 16p13.3 | 3558897 | 3676918 | 118021 |
| BAC171_C18 | 66 | 16p13.3 | 3588928 | 3676900 | 87972 |
| BAC19_P16 | 67 | 16p13.3 | 3617142 | 3721871 | 104729 |
| BAC8_E02 | 69 | 16p13.3 | 3676801 | 3808206 | 131405 |
| BAC176_N19 | 70 | 16p13.3 | 3676829 | 3777606 | 100777 |
| BAC175_E01 | 71 | 16p13.3 | 3721790 | 3808222 | 86432 |
| BAC243_O03 | 72 | 16p13.3 | 3764111 | 3900077 | 135966 |
| BAC77_O20 | 73 | 16p13.3 | 3764182 | 3869131 | 104949 |
| BAC33_C23 | 74 | 16p13.3 | 3799426 | 3925947 | 126521 |
| BAC33_F18 | 75 | 16p13.3 | 3802918 | 3900073 | 97155 |
| BAC19_O18 | 76 | 16p13.3 | 3966246 | 4073662 | 107416 |
| BAC65_O23 | 77 | 16p13.3 | 3970031 | 4109399 | 139368 |
| BAC229_B09 | 78 | 16p13.3 | 4003896 | 4170949 | 167053 |
| BAC151_N12 | 79 | 16p13.3 | 4218427 | 4321997 | 103570 |
| BAC153_I13 | 80 | 16p13.3 | 4218497 | 4321935 | 103438 |
| BAC72_C06 | 81 | 16p13.3 | 4220542 | 4330838 | 110296 |
| BAC45_N08 | 82 | 16p13.3 | 4266234 | 4362182 | 95948 |
| BAC168_G05 | 83 | 16p13.3 | 4362069 | 4480722 | 118653 |
| BAC42_O01 | 84 | 16p13.3 | 4396737 | 4495151 | 98414 |
| BAC156_J23 | 85 | 16p13.3 | 4425040 | 4567524 | 142484 |
| BAC234_H14 | 86 | 16p13.3 | 4445317 | 4533510 | 88193 |
| BAC235_L13 | 87 | 16p13.3 | 4480585 | 4567481 | 86896 |
| BAC10_M16 | 88 | 16p13.3 | 4508020 | 4607666 | 99646 |
| BAC211_B22 | 89 | 16p13.3 | 4508293 | 4627920 | 119627 |
| BAC210_A20 | 90 | 16p13.3 | 4533344 | 4621828 | 88484 |
| BAC196_B08 | 91 | 16p13.3 | 4533365 | 4627937 | 94572 |
| BAC224_L21 | 92 | 16p13.3 | 4586681 | 4730252 | 143571 |
| BAC134_C23 | 93 | 16p13.3 | 4607574 | 4752288 | 144714 |
| BAC119_O20 | 94 | 16p13.3 | 4621969 | 4714235 | 92266 |
| BAC140_K07 | 95 | 16p13.3 | 4621997 | 4733318 | 111321 |
| BAC142_C21 | 96 | 16p13.3 | 4621999 | 4733320 | 111321 |
| BAC167_N23 | 97 | 16p13.3 | 4663676 | 4805399 | 141723 |
| BAC55_A12 | 98 | 16p13.3 | 4677472 | 4752292 | 74820 |
| BAC44_A02 | 99 | 16p13.3 | 4714152 | 4839582 | 125430 |
| BAC101_M09 | 100 | 16p13.3 | 4775516 | 4872170 | 96654 |
| BAC198_G05 | 101 | 16p13.3 | 4819843 | 4916448 | 96605 |
| BAC109_N12 | 102 | 16p13.3 | 4907696 | 5003470 | 95774 |
| BAC170_K10 | 103 | 16p13.3 | 4911373 | 5005974 | 94601 |
| BAC226_C04 | 104 | 16p13.3 | 4951023 | 5036890 | 85867 |
| BAC2_E10 | 105 | 16p13.3 | 4951031 | 5036957 | 85926 |
| BAC120_L14 | 106 | 16p13.3 | 4962603 | 5051450 | 88847 |
| BAC218_E19 | 107 | 16p13.3 | 4980243 | 5082645 | 102402 |
| BAC51_I09 | 108 | 16p13.3 | 5012543 | 5120158 | 107615 |
| BAC2_E13 | 109 | 16p13.3 | 5044334 | 5192459 | 148125 |
| BAC190_H14 | 110 | 16p13.3 | 5068428 | 5209986 | 141558 |
| BAC171_K23 | 111 | 16p13.3 | 5090122 | 5207172 | 117050 |
| BAC244_M04 | 112 | 16p13.3 | 5101386 | 5193865 | 92479 |
| BAC36_C15 | 113 | 16p13.3 | 5196878 | 5329816 | 132938 |
| BAC24_D20 | 114 | 16p13.3 | 5391862 | 5556472 | 164610 |
| BAC19_E20 | 115 | 16p13.3 | 5465141 | 5543370 | 78229 |
| BAC14_H19 | 116 | 16p13.3 | 5555371 | 5699321 | 143950 |
| BAC114_K10 | 117 | 16p13.3 | 5556391 | 5631850 | 75459 |
| BAC184_G18 | 118 | 16p13.3 | 5566359 | 5668195 | 101836 |
| BAC64_C21 | 119 | 16p13.3 | 5613773 | 5714264 | 100491 |
| BAC234_M19 | 120 | 16p13.3 | 5631770 | 5714201 | 82431 |
| BAC96_I19 | 121 | 16p13.3 | 5699219 | 5790183 | 90964 |
| BAC90_K16 | 122 | 16p13.3 | 5753259 | 5875363 | 122104 |
| BAC58_J20 | 123 | 16p13.3 | 5768424 | 5894466 | 126042 |
| BAC31_C20 | 124 | 16p13.3 | 5769964 | 5864724 | 94760 |
| BAC120_O17 | 125 | 16p13.3 | 5928274 | 6026041 | 97767 |
| BAC208_M03 | 126 | 16p13.3 | 5971951 | 6086197 | 114246 |
| BAC144_L17 | 127 | 16p13.3 | 6002063 | 6086183 | 84120 |
| BAC59_H17 | 128 | 16p13.3 | 6061952 | 6182471 | 120519 |
| BAC177_H05 | 129 | 16p13.3 | 6151410 | 6254645 | 103235 |
| BAC145_J04 | 130 | 16p13.3 | 6174552 | 6272687 | 98135 |
| BAC58_M10 | 131 | 16p13.3-16p13.2 | 6298637 | 6409056 | 110419 |
| BAC136_F13 | 132 | 16p13.3-16p13.2 | 6318127 | 6409104 | 90977 |
| BAC97_A23 | 133 | 16p13.3-16p13.2 | 6388512 | 6464944 | 76432 |
| BAC147_C23 | 134 | 16p13.2 | 6458270 | 6533019 | 74749 |
| BAC9_J02 | 135 | 16p13.2 | 6560276 | 6737905 | 177629 |
| BAC100_H02 | 136 | 16p13.2 | 6631478 | 6759323 | 127845 |
| BAC81_M09 | 137 | 16p13.2 | 6833738 | 6961166 | 127428 |
| BAC18_P05 | 138 | 16p13.2 | 6854156 | 6965698 | 111542 |
| BAC31_G11 | 139 | 16p13.2 | 6856529 | 6869084 | 12555 |
| BAC105_C09 | 140 | 16p13.2 | 6868931 | 6977422 | 108491 |
| BAC20_H18 | 141 | 16p13.2 | 6890490 | 6970337 | 79847 |

(7) CMT1A (Charcot-Marie-Tooth Neuropathy 1A)

CMT1A syndrome can be diagnosed for Karyotype duplication 17p12 or segmental trisomy. A DNA chip for detecting CMT1A syndrome includes a microarray containing at least one immobilized genomic DNA fragment selected from the group consisting of genomic DNA fragment 1 to 235 of Table 9, at least one oligomer of 20 to 100 bp consecutive nucleotide within the fragment, or at least one cDNA of 100 bp to 10 kbp derived from the fragment. When at least two kinds of probes are mixed and used, the probes can be mixed in equal proportions and immobilized in a single spot.

TABLE 9

| | | | Chromosomal location | | |
|---|---|---|---|---|---|
| | | Chromosomal location | in Human genome | | |
| BAC DNA | No. | identified by FISH | Start point | End point | size |
| BAC88_C04 | 1 | 17p12 | 11612545 | 11736590 | 124045 |
| BAC102_K14 | 2 | 17p12 | 11660452 | 11769301 | 108849 |
| BAC14_K07 | 3 | 17p12 | 11736490 | 11856270 | 119780 |
| BAC140_D17 | 4 | 17p12 | 11822406 | 11948686 | 126280 |
| BAC83_N02 | 5 | 17p12 | 11825171 | 11937915 | 112744 |
| BAC242_M01 | 6 | 17p12 | 11841526 | 11926614 | 85088 |
| BAC22_A20 | 7 | 17p12 | 11856171 | 11948680 | 92509 |
| BAC32_E15 | 8 | 17p12 | 11919713 | 12005808 | 86095 |
| BAC80_D06 | 9 | 17p12 | 12005708 | 12096400 | 90692 |
| BAC244_J05 | 10 | 17p12 | 12006228 | 12111185 | 104957 |
| BAC70_M12 | 11 | 17p12 | 12082452 | 12186139 | 103687 |
| BAC148_O20 | 12 | 17p12 | 12113892 | 12209981 | 96089 |
| BAC51_B20 | 13 | 17p12 | 12127085 | 12218074 | 90989 |
| BAC233_L11 | 14 | 17p12 | 12167376 | 12241723 | 74347 |
| BAC195_G15 | 15 | 17p12 | 12204374 | 12284440 | 80066 |
| BAC154_C22 | 16 | 17p12 | 12238722 | 12314874 | 76152 |
| BAC195_F23 | 17 | 17p12 | 12262726 | 12364643 | 101917 |
| BAC116_C19 | 18 | 17p12 | 12269714 | 12361238 | 91524 |
| BAC26_H06 | 19 | 17p12 | 12277688 | 12374424 | 96736 |
| BAC168_E04 | 20 | 17p12 | 12291909 | 12410866 | 118957 |
| BAC200_M22 | 21 | 17p12 | 12295157 | 12374406 | 79249 |
| BAC36_P09 | 22 | 17p12 | 12335482 | 12466672 | 131190 |
| BAC76_M23 | 23 | 17p12 | 12376887 | 12475905 | 99018 |
| BAC52_M10 | 24 | 17p12 | 12378033 | 12466745 | 88712 |
| BAC218_N09 | 25 | 17p12 | 12378061 | 12470272 | 92211 |
| BAC150_P01 | 26 | 17p12 | 12677770 | 12786896 | 109126 |
| BAC79_B09 | 27 | 17p12 | 12678501 | 12784001 | 105500 |
| BAC22_K11 | 28 | 17p12 | 12783918 | 12889769 | 105851 |
| BAC176_C19 | 29 | 17p12 | 12820958 | 12920520 | 99562 |
| BAC15_A10 | 30 | 17p12 | 12867910 | 12963846 | 95936 |
| BAC106_P16 | 31 | 17p12 | 12880618 | 12959994 | 79376 |
| BAC249_K10 | 32 | 17p12 | 12918381 | 13029611 | 111230 |
| BAC184_L01 | 33 | 17p12 | 13016097 | 13100250 | 84153 |
| BAC189_J07 | 34 | 17p12 | 13029466 | 13127270 | 97804 |
| BAC61_D08 | 35 | 17p12 | 13060432 | 13215127 | 154695 |
| BAC244_L09 | 36 | 17p12 | 13194210 | 13287251 | 93041 |
| BAC109_M13 | 37 | 17p12 | 13264510 | 13388356 | 123846 |
| BAC114_G12 | 38 | 17p12 | 13269452 | 13373578 | 104126 |
| BAC95_C03 | 39 | 17p12 | 13287109 | 13379964 | 92855 |
| BAC197_M12 | 40 | 17p12 | 13325685 | 13424799 | 99114 |
| BAC52_F06 | 41 | 17p12 | 13329586 | 13477712 | 148126 |
| BAC194_H24 | 42 | 17p12 | 13448677 | 13537070 | 88393 |
| BAC50_A03 | 43 | 17p12 | 13501040 | 13630149 | 129109 |
| BAC70_B18 | 44 | 17p12 | 13534777 | 13622165 | 87388 |
| BAC226_F13 | 45 | 17p12 | 13552445 | 13656979 | 104534 |
| BAC50_K13 | 46 | 17p12 | 13565013 | 13654926 | 89913 |
| BAC30_K11 | 47 | 17p12 | 13725159 | 13812828 | 87669 |
| BAC175_C21 | 48 | 17p12 | 13744679 | 13842394 | 97715 |
| BAC77_I01 | 49 | 17p12 | 13776654 | 13858386 | 81732 |
| BAC39_N11 | 50 | 17p12 | 13776676 | 13891236 | 114560 |
| BAC189_P23 | 51 | 17p12 | 13784869 | 13860050 | 75181 |
| BAC88_G20 | 52 | 17p12 | 13891681 | 14022617 | 130936 |
| BAC108_M02 | 53 | 17p12 | 13954262 | 14068912 | 114650 |
| BAC159_G08 | 54 | 17p12 | 13988264 | 14070150 | 81886 |
| BAC171_P01 | 55 | 17p12 | 14070322 | 14146599 | 76277 |
| BAC153_J07 | 56 | 17p12 | 14120716 | 14200791 | 80075 |
| BAC89_P08 | 57 | 17p12 | 14174783 | 14283750 | 108967 |
| BAC102_C20 | 58 | 17p12 | 14195965 | 14348778 | 152813 |
| BAC127_A05 | 59 | 17p12 | 14196573 | 14296928 | 100355 |
| BAC42_K14 | 60 | 17p12 | 14258761 | 14446116 | 187355 |
| BAC235_I13 | 61 | 17p12 | 14336905 | 14421756 | 84851 |
| BAC38_I19 | 62 | 17p12 | 14336912 | 14455025 | 118113 |
| BAC118_C17 | 63 | 17p12 | 14392821 | 14476828 | 84007 |
| BAC132_H11 | 64 | 17p12 | 14454928 | 14546494 | 91566 |
| BAC38_O21 | 65 | 17p12 | 14514381 | 14604050 | 89669 |
| BAC156_I11 | 66 | 17p12 | 14531677 | 14613398 | 81721 |
| BAC168_M23 | 67 | 17p12 | 14537106 | 14616548 | 79442 |
| BAC170_M14 | 68 | 17p12 | 14711135 | 14808289 | 97154 |
| BAC33_L21 | 69 | 17p12 | 14804500 | 14913795 | 109295 |
| BAC185_D08 | 70 | 17p12 | 14884357 | 14981705 | 97348 |
| BAC65_C19 | 71 | 17p12 | 14960572 | 15058918 | 98346 |
| BAC219_K23 | 72 | 17p12 | 14976175 | 15056221 | 80046 |
| BAC157_G01 | 73 | 17p12 | 15093558 | 15208862 | 115304 |
| BAC193_J12 | 74 | 17p12 | 15110864 | 15227229 | 116365 |
| BAC140_P21 | 75 | 17p12 | 15117703 | 15211297 | 93594 |
| BAC69_D20 | 76 | 17p12 | 15132146 | 15234625 | 102479 |
| BAC163_B05 | 77 | 17p12 | 15147290 | 15251664 | 104374 |
| BAC127_A13 | 78 | 17p12 | 15267749 | 15397538 | 129789 |
| BAC59_O13 | 79 | 17p12 | 15291315 | 15400674 | 109359 |
| BAC171_I03 | 80 | 17p12 | 15293504 | 15387860 | 94356 |
| BAC103_H17 | 81 | 17p12 | 15293548 | 15384732 | 91184 |
| BAC240_L04 | 82 | 17p12 | 15305705 | 15364092 | 58387 |
| BAC180_E05 | 83 | 17p12 | 15314834 | 15404065 | 89231 |
| BAC113_L01 | 84 | 17p12 | 15320794 | 15426185 | 105391 |
| BAC193_F10 | 85 | 17p12 | 15344377 | 15420149 | 75772 |
| BAC80_J24 | 86 | 17p12 | 15387712 | 15472607 | 84895 |
| BAC227_C12 | 87 | 17p12 | 15406530 | 15494411 | 87881 |
| BAC232_P15 | 88 | 17p12 | 15433058 | 15552649 | 119591 |
| BAC154_G20 | 89 | 17p12 | 15467403 | 15549983 | 82580 |
| BAC64_B16 | 90 | 17p12 | 15620998 | 15755383 | 134385 |
| BAC198_H06 | 91 | 17p12 | 15657045 | 15755442 | 98397 |
| BAC120_H23 | 92 | 17p12 | 15657050 | 15782155 | 125105 |
| BAC182_G04 | 93 | 17p12 | 15739595 | 15819054 | 79459 |
| BAC97_H12 | 94 | 17p12 | 15784582 | 15941885 | 157303 |
| BAC102_C09 | 95 | 17p12-17p11.2 | 15818957 | 20693695 | 4874738 |
| BAC41_E16 | 96 | 17p12 | 15847215 | 15926899 | 79684 |
| BAC150_L13 | 97 | 17p12 | 15854845 | 15963018 | 108173 |
| BAC124_J04 | 98 | 17p12 | 15897252 | 15992971 | 95719 |
| BAC96_G24 | 99 | 17p12 | 15938321 | 16028412 | 90091 |
| BAC66_G13 | 100 | 17p12 | 15941785 | 16044299 | 102514 |
| BAC184_B05 | 101 | 17p12 | 15962872 | 16044301 | 81429 |
| BAC60_L08 | 102 | 17p12 | 15962873 | 16060283 | 97410 |
| BAC189_J17 | 103 | 17p12-17p11.2 | 16143400 | 16227881 | 84481 |
| BAC88_H17 | 104 | 17p12-17p11.2 | 16143415 | 16236657 | 93242 |
| BAC42_K11 | 105 | 17p11.2 | 16289956 | 16394927 | 104971 |
| BAC240_E14 | 106 | 17p11.2 | 16394845 | 16489073 | 94228 |
| BAC148_O18 | 107 | 17p11.2 | 16420254 | 16530913 | 110659 |
| BAC125_N20 | 108 | 17p11.2 | 16557887 | 16711927 | 154040 |
| BAC175_H10 | 109 | 17p11.2 | 16557890 | 16636429 | 78539 |
| BAC146_D01 | 110 | 17p11.2 | 16557904 | 16636434 | 78530 |
| BAC62_D24 | 111 | 17p11.2 | 16654529 | 16751004 | 96475 |
| BAC186_K22 | 112 | 17p11.2 | 16724226 | 16796771 | 72545 |
| BAC33_P23 | 113 | 17p11.2 | 16779978 | 16866784 | 86806 |
| BAC14_G14 | 114 | 17p11.2 | 16796638 | 16913606 | 116968 |
| BAC72_P14 | 115 | 17p11.2 | 16866681 | 16988595 | 121914 |
| BAC131_K12 | 116 | 17p11.2 | 16909687 | 16990173 | 80486 |
| BAC197_O11 | 117 | 17p11.2 | 17025266 | 17165284 | 140018 |
| BAC26_I07 | 118 | 17p11.2 | 17025339 | 17091291 | 65952 |
| BAC36_F12 | 119 | 17p11.2 | 17074074 | 17165053 | 90979 |
| BAC129_D23 | 120 | 17p11.2 | 17158746 | 17245353 | 86607 |
| BAC101_K17 | 121 | 17p11.2 | 17245267 | 17333785 | 88518 |
| BAC123_D23 | 122 | 17p11.2 | 17245267 | 17357212 | 111945 |
| BAC89_C18 | 123 | 17p11.2 | 17276220 | 17391839 | 115619 |
| BAC90_H16 | 124 | 17p11.2 | 17283727 | 17383307 | 99580 |
| BAC198_N13 | 125 | 17p11.2 | 17283752 | 17390460 | 106708 |
| BAC154_C08 | 126 | 17p11.2 | 17295654 | 17419340 | 123686 |
| BAC179_F21 | 127 | 17p11.2 | 17324665 | 17419329 | 94664 |
| BAC189_N01 | 128 | 17p11.2 | 17390295 | 17465331 | 75036 |
| BAC20_P07 | 129 | 17p11.2 | 17419240 | 17530721 | 111481 |
| BAC167_A18 | 130 | 17p11.2 | 17419255 | 17572653 | 153398 |
| BAC95_I12 | 131 | 17p11.2 | 17482800 | 17572594 | 89794 |
| BAC155_B06 | 132 | 17p11.2 | 17484086 | 17572609 | 88523 |
| BAC56_J17 | 133 | 17p11.2 | 17551815 | 17665243 | 113428 |
| BAC36_C18 | 134 | 17p11.2 | 17579594 | 17676682 | 97088 |
| BAC170_M10 | 135 | 17p11.2 | 17588731 | 17678229 | 89498 |
| BAC236_B14 | 136 | 17p11.2 | 17698446 | 17776240 | 77794 |
| BAC76_J10 | 137 | 17p11.2 | 17709665 | 17806257 | 96592 |
| BAC165_E09 | 138 | 17p11.2 | 17723644 | 17806266 | 82622 |
| BAC249_G12 | 139 | 17p11.2 | 17776414 | 17890030 | 113616 |
| BAC41_D18 | 140 | 17p11.2 | 17889883 | 18025228 | 135345 |
| BAC131_M11 | 141 | 17p11.2 | 17916386 | 17992329 | 75943 |
| BAC103_D11 | 142 | 17p11.2 | 17935441 | 18032930 | 97489 |
| BAC57_N06 | 143 | 17p11.2 | 18030897 | 18165192 | 134295 |
| BAC197_E01 | 145 | 17p11.2 | 18044616 | 18128976 | 84360 |
| BAC20_D12 | 146 | 17p11.2 | 18128850 | 18219224 | 90374 |
| BAC238_N13 | 147 | 17p11.2 | 18165108 | 18259978 | 94870 |
| BAC57_C22 | 148 | 17p11.2 | 18202515 | 18354860 | 152345 |
| BAC184_A04 | 149 | 17p11.2 | 18206784 | 18295425 | 88641 |
| BAC70_D14 | 150 | 17p11.2 | 18206800 | 18295476 | 88676 |
| BAC186_E14 | 151 | 17p11.2 | 18332672 | 18452458 | 119786 |
| BAC97_I20 | 152 | 17p11.2 | 18393829 | 18513020 | 119191 |
| BAC199_D08 | 153 | 17p11.2 | 18403056 | 18513020 | 109964 |
| BAC154_P06 | 154 | 17p11.2 | 18421956 | 18508299 | 86343 |
| BAC39_C07 | 155 | 17p11.2 | 18507215 | 18613862 | 106647 |
| BAC228_C10 | 156 | 17p11.2 | 18594104 | 18712014 | 117910 |
| BAC81_H03 | 157 | 17p11.2 | 18594109 | 18726670 | 132561 |
| BAC58_P21 | 158 | 17p11.2 | 18635594 | 18755396 | 119802 |
| BAC233_A12 | 159 | 17p11.2 | 18737655 | 18849936 | 112281 |
| BAC153_J19 | 160 | 17p11.2 | 18746164 | 18831879 | 85715 |
| BAC189_M02 | 161 | 17p11.2 | 18773717 | 18895449 | 121732 |
| BAC132_A14 | 162 | 17p11.2 | 18805385 | 18918916 | 113531 |
| BAC95_F14 | 163 | 17p11.2 | 18807265 | 18895443 | 88178 |
| BAC191_B21 | 164 | 17p11.2 | 18808618 | 18884963 | 76345 |
| BAC132_L20 | 165 | 17p11.2 | 18811539 | 18902037 | 90498 |
| BAC232_D17 | 166 | 17p11.2 | 18812507 | 18903860 | 91353 |
| BAC77_C07 | 167 | 17p11.2 | 18831796 | 18927011 | 95215 |
| BAC61_M05 | 168 | 17p11.2 | 18845354 | 19050538 | 205184 |
| BAC57_D11 | 169 | 17p11.2 | 18897566 | 19000464 | 102898 |
| BAC77_B01 | 170 | 17p11.2 | 18952608 | 19072845 | 120237 |
| BAC84_B04 | 171 | 17p11.2 | 19079201 | 19218785 | 139584 |
| BAC160_J02 | 172 | 17p11.2 | 19225463 | 19266078 | 40615 |
| BAC109_F04 | 173 | 17p11.2 | 19244444 | 19304469 | 60025 |
| BAC31_F09 | 174 | 17p11.2 | 19244499 | 19310543 | 66044 |
| BAC54_L12 | 175 | 17p11.2 | 19244500 | 19313051 | 68551 |
| BAC183_B05 | 176 | 17p11.2 | 19265193 | 19337891 | 72698 |
| BAC97_K11 | 177 | 17p11.2 | 19313826 | 19387684 | 73858 |
| BAC148_B12 | 178 | 17p11.2 | 19402824 | 19492063 | 89239 |
| BAC26_B11 | 179 | 17p11.2 | 19526736 | 19640708 | 113972 |
| BAC12_C18 | 180 | 17p11.2 | 19534378 | 19640758 | 106380 |
| BAC92_D22 | 181 | 17p11.2 | 19534392 | 19651908 | 117516 |
| BAC69_L23 | 182 | 17p11.2 | 19545442 | 19651910 | 106468 |
| BAC175_P18 | 183 | 17p11.2 | 19563692 | 19648388 | 84696 |
| BAC146_B24 | 184 | 17p11.2 | 19650991 | 19746822 | 95831 |
| BAC27_K06 | 185 | 17p11.2 | 19651809 | 19749152 | 97343 |
| BAC70_N14 | 186 | 17p11.2 | 19665863 | 19786182 | 120319 |
| BAC171_D20 | 187 | 17p11.2 | 19680409 | 19788794 | 108385 |
| BAC116_O21 | 189 | 17p11.2 | 19718981 | 19786183 | 67202 |
| BAC10_I20 | 190 | 17p11.2 | 19739497 | 19816424 | 76927 |
| BAC96_I07 | 191 | 17p11.2 | 19816311 | 19903394 | 87083 |
| BAC249_G09 | 192 | 17p11.2 | 19867726 | 19989856 | 122130 |
| BAC44_J06 | 193 | 17p11.2 | 19891394 | 19971811 | 80417 |
| BAC193_P10 | 194 | 17p11.2 | 19926766 | 20039699 | 112933 |
| BAC156_C17 | 195 | 17p11.2 | 20083455 | 20215023 | 131568 |
| BAC36_G17 | 196 | 17p11.2 | 20083487 | 20218743 | 135256 |
| BAC11_J06 | 197 | 17p11.2 | 20193464 | 20281761 | 88297 |
| BAC124_E01 | 198 | 17p11.2 | 20228503 | 20332288 | 103785 |
| BAC117_D16 | 199 | 17p11.2 | 20270202 | 20378561 | 108359 |
| BAC188_P16 | 200 | 17p11.2 | 20340291 | 20436954 | 96663 |
| BAC233_L09 | 201 | 17p11.2 | 20457471 | 20549617 | 92146 |
| BAC20_M05 | 202 | 17p11.2 | 20457497 | 20526040 | 68543 |
| BAC69_F02 | 203 | 17p11.2 | 20477768 | 20580390 | 102622 |
| BAC237_C22 | 204 | 17p11.2 | 20499729 | 20600363 | 100634 |
| BAC60_H06 | 205 | 17p11.2 | 20503572 | 20599301 | 95729 |
| BAC141_A08 | 206 | 17p11.2 | 20511752 | 20596573 | 84821 |
| BAC40_H06 | 207 | 17p11.2 | 20525954 | 20630471 | 104517 |
| BAC142_P01 | 208 | 17p11.2 | 20596491 | 20693726 | 97235 |
| BAC48_M08 | 209 | 17p11.2 | 20706155 | 20795955 | 89800 |
| BAC223_H04 | 210 | 17p11.2 | 20734592 | 20851649 | 117057 |
| BAC195_E10 | 211 | 17p11.2 | 20769887 | 20847051 | 77164 |
| BAC200_J14 | 212 | 17p11.2 | 20811622 | 20895723 | 84101 |
| BAC223_I12 | 213 | 17p11.2 | 20811641 | 20905322 | 93681 |
| BAC28_P01 | 214 | 17p11.2 | 20851520 | 20989326 | 137806 |
| BAC119_A16 | 215 | 17p11.2 | 20955668 | 21104977 | 149309 |
| BAC4_G22 | 216 | 17p11.2 | 21004734 | 21099992 | 95258 |
| BAC118_H17 | 217 | 17p11.2 | 21017214 | 21121333 | 104119 |
| BAC40_J07 | 218 | 17p11.2 | 21028143 | 21121354 | 93211 |
| BAC239_L05 | 219 | 17p11.2 | 21036358 | 21175468 | 139110 |
| BAC32_C06 | 220 | 17p11.2 | 21134931 | 21313722 | 178791 |
| BAC248_A23 | 221 | 17p11.2 | 21214133 | 21296062 | 81929 |
| BAC142_A03 | 223 | 17p11.2 | 21214197 | 21315277 | 101080 |
| BAC61_I13 | 224 | 17p11.2 | 21277948 | 21385816 | 107868 |
| BAC236_B19 | 225 | 17p11.2 | 21315182 | 21425247 | 110065 |
| BAC100_J04 | 226 | 17p11.2 | 21393550 | 21547843 | 154293 |
| BAC139_B06 | 227 | 17p11.2-17p11.1 | 21411493 | 25423821 | 4012328 |
| BAC130_E22 | 228 | 17p11.2 | 21425175 | 21493695 | 68520 |
| BAC68_G08 | 229 | 17p11.2 | 21458460 | 21552363 | 93903 |
| BAC26_L13 | 230 | 17p11.2 | 21843173 | 21970661 | 127488 |
| BAC118_E14 | 231 | 17p11.2 | 21953893 | 22050818 | 96925 |
| BAC236_B22 | 232 | 17p11.2 | 22031416 | 22050802 | 19386 |
| BAC81_F21 | 233 | 17p11.2 | 22050715 | 22087819 | 37104 |
| BAC168_D15 | 234 | 17p11.2 | 22050731 | 22088509 | 37778 |
| BAC199_H24 | 235 | 17p11.2 | 22090493 | 22173463 | 82970 |

(8) Cri-du-chat Syndrome

Cri-du-chat syndrome can be diagnosed for Karyotype deletion 5p15.1-3. A DNA chip for detecting Cri-du-chat syndrome includes a microarray containing at least one immobilized genomic DNA fragment selected from the group consisting of genomic DNA fragment 1 to 299 of Table 10, at least one oligomer of 20 to 100 bp consecutive nucleotide within the fragment, or at least one cDNA of 100 bp to 10 kbp derived from the fragment. When at least two kinds of probes are mixed and used, the probes can be mixed in equal proportions and immobilized in a single spot.

TABLE 10

| | | | Chromosomal location | | |
|---|---|---|---|---|---|
| | | Chromosomal location | in Human genome | | |
| BAC DNA | No. | identified by FISH | Start point | End point | size |
| BAC252_L21 | 1 | 5q15.33 | 128831 | 243188 | 114357 |
| BAC86_M05 | 2 | 5p15.33 | 388345 | 566699 | 178354 |
| BAC28_G03 | 3 | 5p15.33 | 388362 | 470975 | 82613 |
| BAC175_M08 | 4 | 5p15.33 | 556957 | 688609 | 131652 |
| BAC117_B21 | 5 | 5p15.33 | 709724 | 796074 | 86350 |
| BAC249_I16 | 6 | 5p15.33 | 726563 | 784281 | 57718 |
| BAC243_K22 | 7 | 5p15.33 | 815908 | 825718 | 9810 |
| BAC191_O13 | 8 | 5p15.33 | 847979 | 960171 | 112192 |
| BAC169_O14 | 9 | 5p15.33 | 867344 | 883983 | 16639 |
| BAC68_E06 | 10 | 5p15.33 | 886552 | 969978 | 83426 |
| BAC6_B15 | 11 | 5p15.33 | 924729 | 1017218 | 92489 |
| BAC247_H03 | 12 | 5p15.33 | 969882 | 1048818 | 78936 |
| BAC61_H11 | 13 | 5p15.33 | 1102296 | 1194500 | 92204 |
| BAC90_N23 | 14 | 5p15.33 | 1106547 | 1241252 | 134705 |
| BAC135_O15 | 15 | 5p15.33 | 1169635 | 1257820 | 88185 |
| BAC18_O05 | 16 | 5p15.33 | 1273885 | 1292497 | 18612 |
| BAC122_J11 | 17 | 5p15.33 | 1370397 | 1445571 | 75174 |
| BAC211_M10 | 18 | 5p15.33 | 1370460 | 1489245 | 118785 |
| BAC131_N10 | 19 | 5p15.33 | 1370720 | 1470782 | 100062 |
| BAC63_F14 | 20 | 5p15.33 | 1383248 | 1478276 | 95028 |
| BAC237_H02 | 21 | 5p15.33 | 1415292 | 1510531 | 95239 |
| BAC34_B10 | 22 | 5p15.33 | 1470700 | 1584463 | 113763 |
| BAC34_B09 | 23 | 5p15.33 | 1470720 | 1584462 | 113742 |
| BAC29_F02 | 24 | 5p15.33 | 1505944 | 1611560 | 105616 |
| BAC213_E13 | 25 | 5p15.33 | 1621532 | 1721634 | 100102 |
| BAC217_D13 | 26 | 5p15.33 | 1650124 | 1737212 | 87088 |
| BAC84_A09 | 27 | 5p15.33 | 1738470 | 1740415 | 1945 |
| BAC175_E04 | 28 | 5p15.33 | 1794107 | 1915566 | 121459 |
| BAC151_C12 | 29 | 5p15.33 | 1817251 | 1903460 | 86209 |
| BAC247_N16 | 30 | 5p15.33 | 1817260 | 1915551 | 98291 |
| BAC179_N23 | 31 | 5p15.33 | 1823775 | 1915550 | 91775 |
| BAC69_N04 | 32 | 5p15.33 | 1907352 | 1999043 | 91691 |
| BAC158_L04 | 33 | 5p15.33 | 1907353 | 2037573 | 130220 |
| BAC244_C11 | 34 | 5p15.33 | 1928449 | 2040646 | 112197 |
| BAC125_G18 | 35 | 5p15.33 | 2033309 | 2152985 | 119676 |
| BAC83_J08 | 36 | 5p15.33 | 2037456 | 2122146 | 84690 |
| BAC197_J20 | 37 | 5p15.33 | 2037469 | 2120445 | 82976 |
| BAC105_B18 | 38 | 5p15.33 | 2045977 | 2165957 | 119980 |
| BAC5_H06 | 39 | 5p15.33 | 2052649 | 2153058 | 100409 |
| BAC66_C02 | 40 | 5p15.33 | 2062344 | 2165598 | 103254 |
| BAC193_G12 | 41 | 5p15.33 | 2062491 | 2165598 | 103107 |
| BAC36_K24 | 42 | 5p15.33 | 2311466 | 2447317 | 135851 |
| BAC107_O02 | 43 | 5p15.33 | 2366344 | 2447305 | 80961 |
| BAC104_G21 | 44 | 5p15.33 | 2392625 | 2500908 | 108283 |
| BAC32_I12 | 45 | 5p15.33 | 2483226 | 2612064 | 128838 |
| BAC183_B03 | 46 | 5p15.33 | 2514663 | 2612031 | 97368 |
| BAC143_B15 | 47 | 5p15.33 | 2593631 | 2676384 | 82753 |
| BAC61_O23 | 48 | 5p15.33 | 2648303 | 2732500 | 84197 |
| BAC72_D04 | 49 | 5p15.33 | 2653960 | 2774274 | 120314 |
| BAC199_F16 | 50 | 5p15.33 | 2734487 | 2817350 | 82863 |
| BAC109_N14 | 51 | 5p15.33 | 2928605 | 3058308 | 129703 |
| BAC33_L02 | 52 | 5p15.33 | 2942822 | 3033354 | 90532 |
| BAC181_E12 | 53 | 5p15.33 | 2999465 | 3098692 | 99227 |
| BAC50_C18 | 54 | 5p15.33 | 3018427 | 3136089 | 117662 |
| BAC108_J05 | 55 | 5p15.33 | 3058222 | 3137214 | 78992 |
| BAC77_E02 | 56 | 5p15.33 | 3088284 | 3242722 | 154438 |
| BAC65_N10 | 57 | 5p15.33 | 3112302 | 3233567 | 121265 |
| BAC110_N16 | 58 | 5p15.33 | 3112302 | 3227037 | 114735 |
| BAC163_J10 | 59 | 5p15.33 | 3217190 | 3347078 | 129888 |
| BAC71_C17 | 60 | 5p15.33 | 3253808 | 3363041 | 109233 |
| BAC141_O08 | 61 | 5p15.33 | 3276587 | 3357763 | 81176 |
| BAC250_D10 | 62 | 5p15.33 | 3412389 | 3490837 | 78448 |
| BAC15_D09 | 63 | 5p15.33 | 3412578 | 3514389 | 101811 |
| BAC176_H02 | 64 | 5p15.33 | 3452446 | 3531939 | 79493 |
| BAC67_N07 | 65 | 5p15.33 | 3452451 | 3585059 | 132608 |
| BAC120_L17 | 66 | 5p15.33 | 3476647 | 3562003 | 85356 |
| BAC76_L20 | 67 | 5p15.33 | 3514301 | 3662180 | 147879 |
| BAC87_O17 | 68 | 5p15.33 | 3602731 | 3701408 | 98677 |
| BAC233_H08 | 69 | 5p15.33 | 3633160 | 3728291 | 95131 |
| BAC237_E02 | 70 | 5p15.33 | 3743316 | 3828612 | 85296 |
| BAC34_E06 | 71 | 5p15.33 | 3792211 | 3881749 | 89538 |
| BAC44_F08 | 72 | 5p15.33 | 3813985 | 3892313 | 78328 |
| BAC124_N21 | 73 | 5p15.33 | 3828483 | 3915914 | 87431 |
| BAC40_D07 | 74 | 5p15.33 | 3925500 | 4049114 | 123614 |
| BAC224_A08 | 75 | 5p15.33 | 3956314 | 4034034 | 77720 |
| BAC187_O05 | 76 | 5p15.33 | 3959084 | 4029775 | 70691 |
| BAC179_F12 | 77 | 5p15.33 | 4043129 | 4155896 | 112767 |
| BAC106_P07 | 78 | 5p15.33 | 4141817 | 4241225 | 99408 |
| BAC244_A11 | 79 | 5p15.33 | 4141817 | 4221304 | 79487 |
| BAC35_C02 | 80 | 5p15.33 | 4241136 | 4359629 | 118493 |
| BAC177_O18 | 81 | 5p15.33 | 4267069 | 4364669 | 97600 |
| BAC72_L14 | 82 | 5p15.33 | 4323690 | 4437198 | 113508 |
| BAC53_I03 | 83 | 5p15.33 | 4329299 | 4432009 | 102710 |
| BAC59_H16 | 84 | 5p15.33 | 4364587 | 4454823 | 90236 |
| BAC97_J19 | 85 | 5p15.33 | 4364592 | 4463852 | 99260 |
| BAC124_O14 | 86 | 5p15.33 | 4387784 | 4467693 | 79909 |
| BAC250_F13 | 87 | 5p15.33-5p15.32 | 4454740 | 4545759 | 91019 |
| BAC120_J18 | 88 | 5p15.33-5p15.32 | 4467502 | 4547938 | 80436 |
| BAC240_E17 | 89 | 5p15.32 | 4502902 | 4587167 | 84265 |
| BAC36_E17 | 90 | 5p15.32 | 4577336 | 4666070 | 88734 |
| BAC141_O06 | 91 | 5p15.32 | 4579061 | 4655990 | 76929 |
| BAC2_N07 | 92 | 5p15.32 | 4593594 | 4688789 | 95195 |
| BAC83_D14 | 93 | 5p15.32 | 4800338 | 4884578 | 84240 |
| BAC57_H16 | 94 | 5p15.32 | 4886171 | 4976171 | 90000 |
| BAC37_D06 | 95 | 5p15.32 | 4924559 | 5061562 | 137003 |
| BAC84_C03 | 96 | 5p15.32 | 4956086 | 5045269 | 89183 |
| BAC1_J16 | 97 | 5p15.32 | 4993956 | 5102984 | 109028 |
| BAC97_J08 | 98 | 5p15.32 | 5013176 | 5102912 | 89736 |
| BAC165_I11 | 99 | 5p15.32 | 5031490 | 5121401 | 89911 |
| BAC97_E15 | 100 | 5p15.32 | 5118171 | 5235263 | 117092 |
| BAC10_D10 | 101 | 5p15.32 | 5135932 | 5220090 | 84158 |
| BAC227_O20 | 102 | 5p15.32 | 5219991 | 5230429 | 10438 |
| BAC154_L08 | 103 | 5p15.32 | 5335391 | 5416971 | 81580 |
| BAC26_I23 | 104 | 5p15.32 | 5380009 | 5470289 | 90280 |
| BAC62_E03 | 105 | 5p15.32 | 5481841 | 5566009 | 84168 |
| BAC89_L15 | 106 | 5p15.32 | 5520243 | 5604993 | 84750 |
| BAC56_N04 | 107 | 5p15.32 | 5536158 | 5633949 | 97791 |
| BAC24_M22 | 108 | 5p15.32 | 5562090 | 5699062 | 136972 |
| BAC42_P07 | 109 | 5p15.32 | 5597451 | 5781858 | 184407 |
| BAC188_F16 | 110 | 5p15.32 | 5604912 | 5699391 | 94479 |
| BAC163_H05 | 111 | 5p15.32 | 5657047 | 5741353 | 84306 |
| BAC229_M02 | 112 | 5p15.32 | 5701489 | 5772649 | 71160 |
| BAC29_P17 | 113 | 5p15.32 | 5719879 | 5818933 | 99054 |
| BAC33_N01 | 114 | 5p15.32 | 5719892 | 5822943 | 103051 |
| BAC39_I10 | 115 | 5p15.32 | 5735602 | 5818929 | 83327 |
| BAC36_I19 | 116 | 5p15.32 | 5778220 | 5910617 | 132397 |
| BAC106_I10 | 117 | 5p15.32 | 5782424 | 5871910 | 89486 |
| BAC86_I08 | 118 | 5p15.32 | 5782482 | 5886208 | 103726 |
| BAC33_J10 | 119 | 5p15.32 | 5782486 | 5880522 | 98036 |
| BAC61_P05 | 120 | 5p15.32 | 5874152 | 6004581 | 130429 |
| BAC166_N22 | 121 | 5p15.32 | 5935417 | 6023493 | 88076 |
| BAC53_N21 | 122 | 5p15.32 | 6081058 | 6172404 | 91346 |
| BAC1l0_I11 | 123 | 5p15.32 | 6120889 | 6262800 | 141911 |
| BAC247_N06 | 124 | 5p15.32 | 6120949 | 6224149 | 103200 |
| BAC119_O22 | 125 | 5p15.32 | 6275540 | 6369479 | 93939 |
| BAC234_M21 | 126 | 5p15.32 | 6285814 | 6396449 | 110635 |
| BAC241_K11 | 127 | 5p15.32 | 6304331 | 6396468 | 92137 |
| BAC130_E06 | 128 | 5p15.32-5p15.31 | 6317183 | 6400159 | 82976 |
| BAC46_G07 | 129 | 5p15.31 | 6410311 | 6505117 | 94806 |
| BAC32_F22 | 130 | 5p15.31 | 6511597 | 6619797 | 108200 |
| BAC69_I08 | 131 | 5p15.31 | 6553803 | 6693232 | 139429 |
| BAC50_B21 | 132 | 5p15.31 | 6555883 | 6693288 | 137405 |
| BAC149_O19 | 133 | 5p15.31 | 6560580 | 6693307 | 132727 |
| BAC33_N12 | 134 | 5p15.31 | 6560581 | 6666502 | 105921 |
| BAC147_H05 | 135 | 5p15.31 | 6570634 | 6669877 | 99243 |
| BAC142_E02 | 136 | 5p15.31 | 6583924 | 6672248 | 88324 |
| BAC155_M05 | 137 | 5p15.31 | 6652308 | 6732951 | 80643 |
| BAC149_B17 | 138 | 5p15.31 | 6693091 | 6792131 | 99040 |
| BAC6_N11 | 139 | 5p15.31 | 6693130 | 6866700 | 173570 |
| BAC37_H22 | 140 | 5p15.31 | 6721112 | 6888557 | 167445 |
| BAC157_E23 | 141 | 5p15.31 | 6722523 | 6798606 | 76083 |
| BAC118_O10 | 142 | 5p15.31 | 6762018 | 6850028 | 88010 |
| BAC170_N24 | 143 | 5p15.31 | 6771312 | 6847747 | 76435 |
| BAC175_I10 | 144 | 5p15.31 | 6778744 | 6904953 | 126209 |
| BAC18_N03 | 145 | 5p15.31 | 6809305 | 6892938 | 83633 |
| BAC83_B11 | 146 | 5p15.31 | 6840472 | 6946171 | 105699 |
| BAC49_E10 | 147 | 5p15.31 | 6867799 | 7003195 | 135396 |
| BAC103_K13 | 148 | 5p15.31 | 6956146 | 7042218 | 86072 |
| BAC197_N10 | 149 | 5p15.31 | 6958987 | 7053242 | 94255 |
| BAC220_D03 | 150 | 5p15.31 | 7053123 | 7145737 | 92614 |
| BAC176_D13 | 151 | 5p15.31 | 7103549 | 7205706 | 102157 |
| BAC73_M02 | 152 | 5p15.31 | 7103553 | 7215572 | 112019 |
| BAC56_D02 | 153 | 5p15.31 | 7239544 | 7314081 | 74537 |
| BAC81_P13 | 154 | 5p15.31 | 7375569 | 7479791 | 104222 |
| BAC175_H06 | 155 | 5p15.31 | 7404269 | 7527619 | 123350 |
| BAC221_L09 | 156 | 5p15.31 | 7436025 | 7530461 | 94436 |
| BAC222_D22 | 157 | 5p15.31 | 7484041 | 7626224 | 142183 |
| BAC175_D24 | 158 | 5p15.31 | 7580061 | 7652998 | 72937 |
| BAC231_L23 | 159 | 5p15.31 | 7583058 | 7678239 | 95181 |
| BAC90_M21 | 160 | 5p15.31 | 7602187 | 7688914 | 86727 |
| BAC9_B08 | 161 | 5p15.31 | 7641355 | 7735395 | 94040 |
| BAC238_I05 | 162 | 5p15.31 | 7745038 | 7757404 | 12366 |
| BAC21_E20 | 163 | 5p15.31 | 7757333 | 7849579 | 92246 |
| BAC84_N11 | 164 | 5p15.31 | 7887418 | 8010671 | 123253 |
| BAC34_K02 | 165 | 5p15.31 | 7910703 | 8021552 | 110849 |
| BAC160_I22 | 166 | 5p15.31 | 7912970 | 8007042 | 94072 |
| BAC33_B11 | 167 | 5p15.31 | 7920413 | 8033099 | 112686 |
| BAC96_F16 | 168 | 5p15.31 | 7925092 | 8021535 | 96443 |
| BAC30_K13 | 169 | 5p15.31 | 7962366 | 8115000 | 152634 |
| BAC124_H05 | 170 | 5p15.31 | 8081113 | 8205507 | 124394 |
| BAC57_O10 | 171 | 5p15.31 | 8114311 | 8206811 | 92500 |
| BAC235_G05 | 172 | 5p15.31 | 8114320 | 8224909 | 110589 |
| BAC161_P17 | 173 | 5p15.31 | 8114912 | 8232707 | 117795 |
| BAC243_D13 | 174 | 5p15.31 | 8127624 | 8224903 | 97279 |
| BAC110_N11 | 175 | 5p15.31 | 8198007 | 8290343 | 92336 |
| BAC18_P07 | 176 | 5p15.31 | 8287106 | 8359001 | 71895 |
| BAC51_P11 | 177 | 5p15.31 | 8358916 | 8456371 | 97455 |
| BAC124_J05 | 178 | 5p15.31 | 8358919 | 8456626 | 97707 |
| BAC230_G14 | 179 | 5p15.31 | 8408700 | 8508314 | 99614 |
| BAC44_G01 | 180 | 5p15.31 | 8421405 | 8535069 | 113664 |
| BAC168_H12 | 181 | 5p15.31 | 8491499 | 8601289 | 109790 |
| BAC56_B13 | 182 | 5p15.31 | 8536307 | 8656833 | 120526 |
| BAC199_F09 | 183 | 5p15.31 | 8543888 | 8669041 | 125153 |
| BAC46_J07 | 184 | 5p15.31 | 8728105 | 8821340 | 93235 |
| BAC105_N05 | 185 | 5p15.31 | 8921235 | 9012278 | 91043 |
| BAC119_M02 | 186 | 5p15.31 | 9116078 | 9207071 | 90993 |
| BAC81_E06 | 187 | 5p15.31 | 9116796 | 9220688 | 103892 |
| BAC234_K14 | 189 | 5p15.31 | 9116796 | 9195293 | 78497 |
| BAC30_M01 | 190 | 5p15.31 | 9238496 | 9333916 | 95420 |
| BAC160_M10 | 191 | 5p15.31 | 9260537 | 9349611 | 89074 |
| BAC170_I24 | 192 | 5p15.31 | 9300484 | 9396870 | 96386 |
| BAC69_A21 | 193 | 5p15.31 | 9305100 | 9392660 | 87560 |
| BAC67_I08 | 194 | 5p15.31 | 9309085 | 9394182 | 85097 |
| BAC186_E12 | 195 | 5p15.31 | 9309085 | 9403929 | 94844 |
| BAC143_N22 | 196 | 5p15.31 | 9511195 | 9591791 | 80596 |
| BAC252_N08 | 197 | 5p15.31 | 9710106 | 9842173 | 132067 |
| BAC64_P22 | 198 | 5p15.31 | 9737672 | 9835771 | 98099 |
| BAC240_K06 | 199 | 5p15.31 | 9775378 | 9856192 | 80814 |
| BAC192_E03 | 200 | 5p15.2 | 9983867 | 10077298 | 93431 |
| BAC176_A01 | 201 | 5p15.2 | 10010645 | 10092340 | 81695 |
| BAC170_E02 | 202 | 5p15.2 | 10010648 | 10092339 | 81691 |
| BAC24_O17 | 203 | 5p15.2 | 10099859 | 10205628 | 105769 |
| BAC196_H04 | 204 | 5p15.2 | 10221116 | 10306923 | 85807 |
| BAC235_E04 | 205 | 5p15.2 | 10221119 | 10310483 | 89364 |
| BAC66_F06 | 206 | 5p15.2 | 10383275 | 10491963 | 108688 |
| BAC22_P08 | 207 | 5p15.2 | 10441541 | 10451636 | 10095 |
| BAC108_J16 | 208 | 5p15.2 | 10487587 | 10635782 | 148195 |
| BAC236_B20 | 209 | 5p15.2 | 10514853 | 10595988 | 81135 |
| BAC8_J11 | 210 | 5p15.2 | 10534437 | 10643735 | 109298 |
| BAC221_P01 | 211 | 5p15.2 | 10543333 | 10643749 | 100416 |
| BAC115_A21 | 212 | 5p15.2 | 10551145 | 10656181 | 105036 |
| BAC141_L07 | 213 | 5p15.2 | 10621042 | 10705776 | 84734 |
| BAC132_I24 | 214 | 5p15.2 | 10903009 | 10976903 | 73894 |
| BAC106_I04 | 215 | 5p15.2 | 10903034 | 11012288 | 109254 |
| BAC175_F12 | 216 | 5p15.2 | 10921671 | 11011856 | 90185 |
| BAC220_L14 | 217 | 5p15.2 | 10955940 | 11036262 | 80322 |
| BAC75_F09 | 218 | 5p15.2 | 11007356 | 11103833 | 96477 |
| BAC169_M11 | 219 | 5p15.2 | 11017396 | 11106608 | 89212 |
| BAC172_K04 | 220 | 5p15.2 | 11017447 | 11104158 | 86711 |
| BAC180_C09 | 221 | 5p15.2 | 11030916 | 11127830 | 96914 |
| BAC248_F02 | 223 | 5p15.2 | 11055973 | 11145667 | 89694 |
| BAC97_A16 | 224 | 5p15.2 | 11067372 | 11167650 | 100278 |
| BAC119_K22 | 225 | 5p15.2 | 11073012 | 11170040 | 97028 |
| BAC151_L09 | 226 | 5p15.2 | 11153127 | 11258317 | 105190 |
| BAC242_E15 | 227 | 5p15.2 | 11163141 | 11277292 | 114151 |
| BAC43_F04 | 228 | 5p15.2 | 11167549 | 11277305 | 109756 |
| BAC230_D02 | 229 | 5p15.2 | 11173922 | 11263682 | 89760 |
| BAC21_B09 | 230 | 5p15.2 | 11183558 | 11277294 | 93736 |
| BAC151_L10 | 231 | 5p15.2 | 11202701 | 11296775 | 94074 |
| BAC82_A09 | 232 | 5p15.2 | 11208897 | 11342678 | 133781 |
| BAC187_A21 | 233 | 5p15.2 | 11334358 | 11425391 | 91033 |
| BAC135_L06 | 234 | 5p15.2 | 11432189 | 11516715 | 84526 |
| BAC173_O13 | 235 | 5p15.2 | 11868196 | 11962786 | 94590 |
| BAC77_N16 | 236 | 5p15.2 | 11890831 | 12037300 | 146469 |
| BAC183_D16 | 237 | 5p15.2 | 11947432 | 11948541 | 1109 |
| BAC153_M02 | 238 | 5p15.2 | 11985373 | 12078591 | 93218 |
| BAC55_L20 | 239 | 5p15.2 | 12007189 | 12130976 | 123787 |
| BAC169_I12 | 240 | 5p15.2 | 12037141 | 12163680 | 126539 |
| BAC223_P22 | 241 | 5p15.2 | 12037200 | 12114532 | 77332 |
| BAC123_G09 | 242 | 5p15.2 | 12069850 | 12152486 | 82636 |
| BAC128_E04 | 243 | 5p15.2 | 12069858 | 12217234 | 147376 |
| BAC23_J19 | 245 | 5p15.2 | 12333920 | 12462134 | 128214 |
| BAC155_G24 | 246 | 5p15.2 | 12493865 | 12577999 | 84134 |
| BAC55_N03 | 247 | 5p15.2 | 12549463 | 12659027 | 109564 |
| BAC182_I10 | 248 | 5p15.2 | 12789041 | 12879371 | 90330 |
| BAC175_O23 | 249 | 5p15.2 | 12823902 | 12927401 | 103499 |
| BAC131_F09 | 250 | 5p15.2 | 12823908 | 12918460 | 94552 |
| BAC58_I20 | 251 | 5p15.2 | 13145730 | 13282407 | 136677 |
| BAC4_P05 | 252 | 5p15.2 | 13218831 | 13314311 | 95480 |
| BAC237_H15 | 253 | 5p15.2 | 13218887 | 13345272 | 126385 |
| BAC178_K12 | 254 | 5p15.2 | 13290125 | 13362317 | 72192 |
| BAC151_H24 | 255 | 5p15.2 | 13312339 | 13456042 | 143703 |
| BAC50_P10 | 256 | 5p15.2 | 13405771 | 13556376 | 150605 |
| BAC96_K16 | 257 | 5p15.2 | 13663563 | 13761343 | 97780 |
| BAC230_P16 | 258 | 5p15.2 | 13679676 | 13787634 | 107958 |
| BAC132_B03 | 259 | 5p15.2 | 13694033 | 13787634 | 93601 |
| BAC186_M06 | 260 | 5p15.2 | 13702438 | 13787606 | 85168 |
| BAC43_K23 | 261 | 5p15.2 | 13787542 | 13890083 | 102541 |
| BAC68_C18 | 262 | 5p15.2 | 13828755 | 13966810 | 138055 |
| BAC37_K08 | 263 | 5p15.2 | 13895801 | 14048071 | 152270 |
| BAC37_B18 | 264 | 5p15.2 | 13929802 | 14031020 | 101218 |
| BAC221_L15 | 265 | 5p15.2 | 13929803 | 14014005 | 84202 |
| BAC145_J23 | 266 | 5p15.2 | 14030939 | 14113682 | 82743 |
| BAC116_I11 | 267 | 5p15.2 | 14113570 | 14200996 | 87426 |
| BAC31_M08 | 268 | 5p15.2 | 14113580 | 14279930 | 166350 |
| BAC54_J02 | 269 | 5p15.2 | 14166160 | 14263204 | 97044 |
| BAC210_L05 | 270 | 5p15.2 | 14187227 | 14287219 | 99992 |
| BAC19_F07 | 271 | 5p15.2 | 14187228 | 14284154 | 96926 |
| BAC160_B20 | 272 | 5p15.2 | 14264088 | 14340226 | 76138 |
| BAC194_K22 | 273 | 5p15.2 | 14303315 | 14381518 | 78203 |
| BAC139_F07 | 274 | 5p15.2 | 14307969 | 14418261 | 110292 |
| BAC86_B20 | 275 | 5p15.2 | 14308956 | 14409273 | 100317 |
| BAC188_F22 | 276 | 5p15.2 | 14330839 | 14415180 | 84341 |
| BAC77_O18 | 278 | 5p15.2 | 14390377 | 14491264 | 100887 |
| BAC107_D13 | 279 | 5p15.2 | 14392683 | 14541546 | 148863 |
| BAC33_C10 | 280 | 5p15.2 | 14464342 | 14562801 | 98459 |
| BAC184_J06 | 281 | 5p15.2 | 14475733 | 14576694 | 100961 |
| BAC134_D14 | 282 | 5p15.2 | 14491127 | 14578216 | 87089 |
| BAC92_A22 | 283 | 5p15.2 | 14525113 | 14627138 | 102025 |
| BAC209_E18 | 284 | 5p15.2 | 14537284 | 14621205 | 83921 |
| BAC57_F19 | 285 | 5p15.2 | 14597896 | 14703597 | 105701 |
| BAC120_G13 | 286 | 5p15.2 | 14621106 | 14710419 | 89313 |
| BAC91_H15 | 287 | 5p15.2 | 14626996 | 14727920 | 100924 |
| BAC46_M22 | 288 | 5p15.2 | 14705239 | 14793277 | 88038 |
| BAC123_B17 | 289 | 5p15.2 | 14734183 | 14814350 | 80167 |
| BAC53_I05 | 290 | 5p15.2 | 14737265 | 14831516 | 94251 |
| BAC148_B24 | 291 | 5p15.2 | 14835587 | 14939381 | 103794 |
| BAC58_H19 | 292 | 5p15.2 | 14840914 | 14950347 | 109433 |
| BAC41_B17 | 293 | 5p15.2 | 14886624 | 14888802 | 2178 |
| BAC46_L15 | 294 | 5p15.2 | 14925975 | 15023813 | 97838 |
| BAC90_N09 | 295 | 5p15.1 | 15127322 | 15215506 | 88184 |
| BAC186_N20 | 296 | 5p15.1 | 15163246 | 15258844 | 95598 |
| BAC176_B19 | 297 | 5p15.1 | 15199196 | 15258839 | 59643 |
| BAC34_K19 | 298 | 5p15.1 | 15352613 | 15459965 | 107352 |
| BAC154_K02 | 299 | 5p15.1 | 15352629 | 15479384 | 126755 |

(9) Digeorge Syndrome

Digeorge syndrome can be diagnosed for Karyotype deletion 22q11.1-2. A DNA chip for detecting Digeorge syndrome includes a microarray containing at least one immobilized genomic DNA fragment selected from the group consisting of genomic DNA fragment 1 to 231 of Table 11, at least one oligomer of 20 to 100 bp consecutive nucleotide within the fragment, or at least one cDNA of 100 bp to 10 kbp derived from the fragment. When at least two kinds of probes are mixed and used, the probes can be mixed in equal proportions and immobilized in a single spot.

TABLE 11

| | | | Chromosomal location | | |
|---|---|---|---|---|---|
| | | Chromosomal location | in Human genome | | |
| BAC DNA | No. | identified by FISH | Start point | End point | size |
| BAC250_H12 | 1 | 22q11.1 | 14440002 | 14531618 | 91616 |
| BAC100_D18 | 2 | 22q11.1 | 14446040 | 14550110 | 104070 |
| BAC53_N19 | 3 | 22q11.1 | 14458203 | 14564968 | 106765 |
| BAC76_P10 | 4 | 22q11.1 | 14461693 | 14573397 | 111704 |
| BAC66_L06 | 5 | 22q11.1 | 14493146 | 14622833 | 129687 |
| BAC147_I21 | 6 | 22q11.1 | 14512518 | 14620326 | 107808 |
| BAC31_C17 | 7 | 22q11.1 | 14527993 | 14620341 | 92348 |
| BAC244_D17 | 8 | 22q11.1 | 14531459 | 14620306 | 88847 |
| BAC73_K15 | 9 | 22q11.1 | 14536252 | 14623463 | 87211 |
| BAC40_F02 | 10 | 22q11.1 | 14540246 | 14626410 | 86164 |
| BAC102_D06 | 11 | 22q11.1 | 14550028 | 14653950 | 103922 |
| BAC162_H14 | 12 | 22q11.1 | 14579035 | 14671429 | 92394 |
| BAC188_N15 | 13 | 22q11.1 | 14585344 | 14681110 | 95766 |
| BAC113_O16 | 14 | 22q11.1 | 14585375 | 14671412 | 86037 |
| BAC37_N12 | 15 | 22q11.1 | 14627643 | 14720248 | 92605 |
| BAC149_L12 | 16 | 22q11.1 | 14790152 | 14871100 | 80948 |
| BAC21_C10 | 17 | 22q11.1 | 14796596 | 14907673 | 111077 |
| BAC139_G13 | 18 | 22q11.1 | 14907574 | 14992678 | 85104 |
| BAC58_H04 | 19 | 22q11.1 | 15510822 | 15690031 | 179209 |
| BAC55_L03 | 20 | 22q11.1 | 15569914 | 15681450 | 111536 |
| BAC44_H07 | 21 | 22q11.1 | 15571388 | 15699027 | 127639 |
| BAC19_C05 | 22 | 22q11.1 | 15685657 | 15797579 | 111922 |
| BAC29_M04 | 23 | 22q11.1 | 15723976 | 15827284 | 103308 |
| BAC169_I04 | 24 | 22q11.1 | 15847567 | 15934193 | 86626 |
| BAC173_K06 | 25 | 22q11.1 | 15875991 | 15971846 | 95855 |
| BAC162_K19 | 26 | 22q11.1 | 15902446 | 15971783 | 69337 |
| BAC237_N05 | 27 | 22q11.1 | 15902926 | 15989830 | 86904 |
| BAC228_A03 | 28 | 22q11.1 | 15950440 | 16070110 | 119670 |
| BAC53_L11 | 29 | 22q11.1 | 15961011 | 16091553 | 130542 |
| BAC222_F01 | 30 | 22q11.1 | 15998269 | 16014027 | 15758 |
| BAC193_J18 | 31 | 22q11.1 | 16008296 | 16103110 | 94814 |
| BAC240_C17 | 32 | 22q11.1 | 16013856 | 16097804 | 83948 |
| BAC76_E24 | 33 | 22q11.1 | 16128049 | 16239481 | 111432 |
| BAC167_L20 | 34 | 22q11.1 | 16128093 | 16222881 | 94788 |
| BAC186_A22 | 35 | 22q11.1-22q11.21 | 16239729 | 16320835 | 81106 |
| BAC123_N08 | 36 | 22q11.1-22q11.21 | 16248820 | 16340395 | 91575 |
| BAC124_P04 | 37 | 22q11.1-22q11.21 | 16261651 | 16364570 | 102919 |
| BAC39_O22 | 38 | 22q11.1-22q11.21 | 16264299 | 16384261 | 119962 |
| BAC42_J03 | 39 | 22q11.1-22q11.21 | 16290522 | 16378093 | 87571 |
| BAC148_D17 | 40 | 22q11.1-22q11.21 | 16292074 | 16384262 | 92188 |
| BAC108_K18 | 41 | 22q11.21 | 16311382 | 16407939 | 96557 |
| BAC158_K10 | 42 | 22q11.21 | 16332003 | 16437272 | 105269 |
| BAC158_H10 | 43 | 22q11.21 | 16332012 | 16437272 | 105260 |
| BAC86_G14 | 44 | 22q11.21 | 16352247 | 16506400 | 154153 |
| BAC200_F18 | 45 | 22q11.21 | 16352250 | 16442890 | 90640 |
| BAC217_L17 | 46 | 22q11.21 | 16364460 | 16478369 | 113909 |
| BAC147_M10 | 47 | 22q11.21 | 16381988 | 16491342 | 109354 |
| BAC53_H10 | 48 | 22q11.21 | 16454669 | 16535942 | 81273 |
| BAC153_I21 | 49 | 22q11.21 | 16454671 | 16579897 | 125226 |
| BAC133_O24 | 50 | 22q11.21 | 16478219 | 16579900 | 101681 |
| BAC186_I17 | 51 | 22q11.21 | 16478283 | 16564378 | 86095 |
| BAC80_O05 | 52 | 22q11.21 | 16584219 | 16591901 | 7682 |
| BAC185_A10 | 53 | 22q11.21 | 16585502 | 16703290 | 117788 |
| BAC196_C16 | 54 | 22q11.21 | 16599272 | 16679947 | 80675 |
| BAC114_F12 | 55 | 22q11.21 | 16660186 | 16742612 | 82426 |
| BAC41_M07 | 56 | 22q11.21 | 16718938 | 16892330 | 173392 |
| BAC67_K22 | 57 | 22q11.21 | 16718996 | 16840296 | 121300 |
| BAC167_B18 | 58 | 22q11.21 | 16788052 | 16883295 | 95243 |
| BAC168_C21 | 59 | 22q11.21 | 16794117 | 16901758 | 107641 |
| BAC62_N23 | 60 | 22q11.21 | 16799783 | 16893727 | 93944 |
| BAC197_M06 | 61 | 22q11.21 | 16808948 | 16883274 | 74326 |
| BAC218_F18 | 62 | 22q11.21 | 16809022 | 16893819 | 84797 |
| BAC103_E14 | 63 | 22q11.21 | 16813623 | 16901754 | 88131 |
| BAC214_I04 | 64 | 22q11.21 | 16892175 | 16969954 | 77779 |
| BAC55_I17 | 65 | 22q11.21 | 16899786 | 16989320 | 89534 |
| BAC52_P06 | 66 | 22q11.21 | 17035984 | 17383792 | 347808 |
| BAC181_O15 | 67 | 22q11.21 | 17248725 | 17362247 | 113522 |
| BAC32_N17 | 68 | 22q11.21 | 17248730 | 17340545 | 91815 |
| BAC161_L23 | 69 | 22q11.21 | 17416386 | 17496392 | 80006 |
| BAC204_B09 | 70 | 22q11.21 | 17440670 | 17536372 | 95702 |
| BAC196_A22 | 71 | 22q11.21 | 17440685 | 17537334 | 96649 |
| BAC88_I06 | 72 | 22q11.21 | 17520575 | 17642310 | 121735 |
| BAC169_K21 | 73 | 22q11.21 | 17684994 | 17767999 | 83005 |
| BAC141_K20 | 74 | 22q11.21 | 17685059 | 17767958 | 82899 |
| BAC145_P12 | 75 | 22q11.21 | 17721046 | 17883837 | 162791 |
| BAC224_F10 | 76 | 22q11.21 | 17754423 | 17865240 | 110817 |
| BAC193_E02 | 77 | 22q11.21 | 17827433 | 17911496 | 84063 |
| BAC127_G04 | 78 | 22q11.21 | 17888656 | 17992847 | 104191 |
| BAC176_N08 | 79 | 22q11.21 | 17888677 | 17952256 | 63579 |
| BAC103_G19 | 80 | 22q11.21 | 17966944 | 18084491 | 117547 |
| BAC82_N03 | 81 | 22q11.21 | 17981523 | 18115068 | 133545 |
| BAC195_M18 | 82 | 22q11.21 | 17992737 | 18084436 | 91699 |
| BAC73_G13 | 83 | 22q11.21 | 18054142 | 18133800 | 79658 |
| BAC140_J13 | 84 | 22q11.21 | 18054155 | 18192798 | 138643 |
| BAC45_L11 | 85 | 22q11.21 | 18114912 | 18236616 | 121704 |
| BAC60_K03 | 86 | 22q11.21 | 18141717 | 18295173 | 153456 |
| BAC154_P05 | 87 | 22q11.21 | 18193984 | 18282769 | 88785 |
| BAC126_N11 | 88 | 22q11.21 | 18194724 | 18293115 | 98391 |
| BAC193_H09 | 89 | 22q11.21 | 18194732 | 18293104 | 98372 |
| BAC135_D20 | 90 | 22q11.21 | 18250892 | 18337061 | 86169 |
| BAC101_M14 | 91 | 22q11.21 | 18337634 | 18458154 | 120520 |
| BAC179_L07 | 92 | 22q11.21 | 18337654 | 18422012 | 84358 |
| BAC198_G21 | 93 | 22q11.21 | 18507946 | 18624974 | 117028 |
| BAC177_K05 | 94 | 22q11.21 | 18536694 | 18620613 | 83919 |
| BAC186_B16 | 95 | 22q11.21 | 18536732 | 18620602 | 83870 |
| BAC107_J10 | 96 | 22q11.21 | 19020077 | 20180677 | 1160600 |
| BAC120_J19 | 97 | 22q11.21 | 19027528 | 19993993 | 966465 |
| BAC210_F18 | 98 | 22q11.21 | 19054027 | 19145632 | 91605 |
| BAC249_B23 | 99 | 22q11.21 | 19145479 | 19256475 | 110996 |
| BAC32_E13 | 100 | 22q11.21 | 19182855 | 19339950 | 157095 |
| BAC132_K21 | 101 | 22q11.21 | 19339819 | 19497300 | 157481 |
| BAC201_I03 | 102 | 22q11.21 | 19341954 | 19453109 | 111155 |
| BAC235_E11 | 103 | 22q11.21 | 19372279 | 19502515 | 130236 |
| BAC144_P14 | 104 | 22q11.21 | 19372296 | 19502516 | 130220 |
| BAC25_K01 | 105 | 22q11.21 | 19384280 | 19502531 | 118251 |
| BAC26_P01 | 106 | 22q11.21 | 19384282 | 19534559 | 150277 |
| BAC128_P06 | 107 | 22q11.21 | 19395280 | 19477701 | 82421 |
| BAC35_O12 | 108 | 22q11.21 | 19397402 | 19496086 | 98684 |
| BAC90_N16 | 109 | 22q11.21 | 19408575 | 19501234 | 92659 |
| BAC33_G05 | 110 | 22q11.21 | 19443074 | 19568360 | 125286 |
| BAC122_J17 | 111 | 22q11.21 | 19501143 | 19658729 | 157586 |
| BAC130_B10 | 112 | 22q11.21 | 19555616 | 19641776 | 86160 |
| BAC224_D06 | 113 | 22q11.21 | 19564867 | 19641786 | 76919 |
| BAC142_L17 | 114 | 22q11.21 | 19664731 | 19803112 | 138381 |
| BAC146_P20 | 115 | 22q11.21 | 19678462 | 19803117 | 124655 |
| BAC18_C02 | 116 | 22q11.21 | 19678523 | 19777877 | 99354 |
| BAC222_A17 | 117 | 22q11.21 | 19678545 | 19777881 | 99336 |
| BAC198_E11 | 118 | 22q11.21 | 19716578 | 19815275 | 98697 |
| BAC226_I20 | 119 | 22q11.21 | 19777760 | 19849125 | 71365 |
| BAC192_K12 | 120 | 22q11.21 | 19893709 | 19976514 | 82805 |
| BAC23_G16 | 121 | 22q11.21 | 20029109 | 20101281 | 72172 |
| BAC51_N20 | 122 | 22q11.21 | 20180535 | 20388075 | 207540 |
| BAC175_B17 | 123 | 22q11.21 | 20286359 | 20388061 | 101702 |
| BAC178_G14 | 124 | 22q11.21 | 20286359 | 20364442 | 78083 |
| BAC194_H12 | 125 | 22q11.21 | 20332560 | 20439626 | 107066 |
| BAC235_A17 | 126 | 22q11.21 | 20404870 | 20488319 | 83449 |
| BAC196_E04 | 127 | 22q11.22 | 20606478 | 20710316 | 103838 |
| BAC247_B13 | 128 | 22q11.22 | 20617859 | 20708571 | 90712 |
| BAC154_H02 | 129 | 22q11.22 | 20627415 | 20710314 | 82899 |
| BAC108_G12 | 130 | 22q11.22 | 20670620 | 20762524 | 91904 |
| BAC100_M23 | 131 | 22q11.22 | 20705924 | 20809656 | 103732 |
| BAC120_H20 | 132 | 22q11.22 | 20710227 | 20809640 | 99413 |
| BAC53_C19 | 133 | 22q11.22 | 20742121 | 20829845 | 87724 |
| BAC52_E21 | 134 | 22q11.22 | 20881109 | 20995571 | 114462 |
| BAC90_H14 | 135 | 22q11.22 | 20881135 | 20993568 | 112433 |
| BAC191_I21 | 136 | 22q11.22 | 20892828 | 20993581 | 100753 |
| BAC226_N02 | 137 | 22q11.22 | 20892871 | 20995580 | 102709 |
| BAC123_E03 | 138 | 22q11.22 | 20892879 | 20997817 | 104938 |
| BAC249_A07 | 139 | 22q11.22 | 20912733 | 20993584 | 80851 |
| BAC166_G20 | 140 | 22q11.22 | 20924662 | 21039259 | 114597 |
| BAC91_D24 | 141 | 22q11.22 | 20997706 | 21097832 | 100126 |
| BAC182_N11 | 142 | 22q11.22 | 21020717 | 21099894 | 79177 |
| BAC150_I15 | 143 | 22q11.22 | 21096868 | 21202228 | 105360 |
| BAC71_L18 | 144 | 22q11.22 | 21142547 | 21215379 | 72832 |
| BAC232_H01 | 145 | 22q11.22 | 21191557 | 21248454 | 56897 |
| BAC125_M16 | 146 | 22q11.22 | 21241766 | 21327536 | 85770 |
| BAC190_K15 | 147 | 22q11.22 | 21286489 | 21375817 | 89328 |
| BAC146_J19 | 148 | 22q11.22 | 21315371 | 21393736 | 78365 |
| BAC17_E13 | 149 | 22q11.22 | 21327420 | 21439293 | 111873 |
| BAC219_F19 | 150 | 22q11.22 | 21393660 | 21509256 | 115596 |
| BAC102_D24 | 151 | 22q11.22 | 21393663 | 21509338 | 115675 |
| BAC204_K06 | 152 | 22q11.22 | 21534791 | 21625441 | 90650 |
| BAC188_H10 | 153 | 22q11.22 | 21686971 | 21780809 | 93838 |
| BAC235_G04 | 154 | 22q11.22 | 21687009 | 21771769 | 84760 |
| BAC170_F13 | 155 | 22q11.22-22q11.23 | 21764932 | 21893837 | 128905 |
| BAC169_G07 | 156 | 22q11.22-22q11.23 | 21780173 | 21893276 | 113103 |
| BAC92_P17 | 157 | 22q11.23 | 21830862 | 21962549 | 131687 |
| BAC187_I13 | 158 | 22q11.23 | 21866189 | 21962607 | 96418 |
| BAC157_I03 | 159 | 22q11.23 | 21956169 | 22105616 | 149447 |
| BAC100_N23 | 160 | 22q11.23 | 21962455 | 22054299 | 91844 |
| BAC61_M11 | 161 | 22q11.23 | 21990926 | 22085595 | 94669 |
| BAC237_I19 | 162 | 22q11.23 | 22013628 | 22096825 | 83197 |
| BAC123_B21 | 163 | 22q11.23 | 22259401 | 22346943 | 87542 |
| BAC170_B02 | 164 | 22q11.23 | 22369460 | 22494813 | 125353 |
| BAC157_N19 | 165 | 22q11.23 | 22429737 | 22547624 | 117887 |
| BAC132_J01 | 166 | 22q11.23 | 22430289 | 22575272 | 144983 |
| BAC193_N18 | 167 | 22q11.23 | 22663632 | 22778729 | 115097 |
| BAC171_J23 | 168 | 22q11.23 | 22705907 | 22782265 | 76358 |
| BAC221_D14 | 169 | 22q11.23 | 22757104 | 22853964 | 96860 |
| BAC89_L04 | 170 | 22q11.23 | 22778616 | 22866947 | 88331 |
| BAC156_O21 | 171 | 22q11.23 | 22779895 | 22853951 | 74056 |
| BAC233_D21 | 172 | 22q11.23 | 22865300 | 22951470 | 86170 |
| BAC117_I21 | 173 | 22q11.23 | 22886592 | 22927463 | 40871 |
| BAC28_L13 | 174 | 22q11.23 | 22922829 | 23034685 | 111856 |
| BAC49_H20 | 175 | 22q11.23 | 22935020 | 23025268 | 90248 |
| BAC67_E13 | 176 | 22q11.23 | 22946015 | 23034703 | 88688 |
| BAC40_E10 | 177 | 22q11.23 | 22951401 | 23035946 | 84545 |
| BAC247_C09 | 178 | 22q11.23 | 22951401 | 23059880 | 108479 |
| BAC228_H17 | 179 | 22q11.23 | 23006886 | 23135328 | 128442 |
| BAC154_L05 | 180 | 22q11.23 | 23034602 | 23119122 | 84520 |
| BAC193_G11 | 181 | 22q11.23 | 23127527 | 23272351 | 144824 |
| BAC170_F04 | 182 | 22q11.23 | 23135206 | 23216592 | 81386 |
| BAC60_H22 | 183 | 22q11.23 | 23137736 | 23228258 | 90522 |
| BAC158_B02 | 184 | 22q11.23 | 23181023 | 23272427 | 91404 |
| BAC199_J01 | 185 | 22q11.23 | 23235170 | 23350054 | 114884 |
| BAC80_K23 | 186 | 22q11.23 | 23276735 | 23453590 | 176855 |
| BAC96_M16 | 187 | 22q11.23 | 23426152 | 23569727 | 143575 |
| BAC190_C04 | 189 | 22q11.23 | 23428857 | 23523195 | 94338 |
| BAC240_A03 | 190 | 22q11.23 | 23453434 | 23534019 | 80585 |
| BAC238_H12 | 191 | 22q11.23 | 23523024 | 23633387 | 110363 |
| BAC65_K03 | 192 | 22q11.23 | 23565930 | 23681963 | 116033 |
| BAC234_L03 | 193 | 22q11.23 | 23565941 | 23657335 | 91394 |
| BAC136_C03 | 194 | 22q11.23 | 23569636 | 23663437 | 93801 |
| BAC220_K20 | 195 | 22q11.23 | 23666075 | 23771023 | 104948 |
| BAC76_P14 | 196 | 22q11.23 | 23671008 | 23793198 | 122190 |
| BAC231_H07 | 197 | 22q11.23 | 23676101 | 23771011 | 94910 |
| BAC118_H08 | 198 | 22q11.23 | 23691316 | 23837599 | 146283 |
| BAC183_F16 | 199 | 22q11.23 | 23764146 | 23829650 | 65504 |
| BAC151_N20 | 200 | 22q11.23 | 23764185 | 23880311 | 116126 |
| BAC62_A01 | 201 | 22q11.23 | 23797003 | 23907552 | 110549 |
| BAC220_E12 | 202 | 22q11.23 | 23831447 | 23990525 | 159078 |
| BAC184_B17 | 203 | 22q11.23 | 23842007 | 23933583 | 91576 |
| BAC193_E06 | 204 | 22q11.23 | 24009516 | 24089848 | 80332 |
| BAC199_I14 | 205 | 22q11.23 | 24009545 | 24110169 | 100624 |
| BAC226_C08 | 206 | 22q11.23 | 24031544 | 24120179 | 88635 |
| BAC175_P10 | 207 | 22q11.23 | 24050012 | 24136148 | 86136 |
| BAC12_C06 | 208 | 22q11.23 | 24067463 | 24164283 | 96820 |
| BAC159_K10 | 209 | 22q11.23 | 24067472 | 24190731 | 123259 |
| BAC38_J11 | 210 | 22q11.23 | 24095308 | 24184121 | 88813 |
| BAC234_P08 | 211 | 22q11.23 | 24145639 | 24243535 | 97896 |
| BAC156_L12 | 212 | 22q11.23 | 24145705 | 24238644 | 92939 |
| BAC62_L24 | 213 | 22q11.23 | 24145709 | 24261370 | 115661 |
| BAC151_E16 | 214 | 22q11.23 | 24190588 | 24281526 | 90938 |
| BAC150_E24 | 215 | 22q11.23 | 24207364 | 24297110 | 89746 |
| BAC151_N05 | 216 | 22q11.23-22q12.1 | 24243456 | 24335182 | 91726 |
| BAC26_F06 | 217 | 22q11.23-22q12.1 | 24267318 | 24367097 | 99779 |
| BAC228_A12 | 218 | 22q12.1 | 24419161 | 24558215 | 139054 |
| BAC248_B05 | 219 | 22q12.1 | 24420814 | 24504520 | 83706 |
| BAC112_F05 | 220 | 22q12.1 | 24452320 | 24555375 | 103055 |
| BAC59_M12 | 221 | 22q12.1 | 24452365 | 24555299 | 102934 |
| BAC146_D03 | 223 | 22q12.1 | 24492191 | 24582859 | 90668 |
| BAC43_L05 | 224 | 22q12.1 | 24548598 | 24644369 | 95771 |
| BAC153_I19 | 225 | 22q12.1 | 24582788 | 24684485 | 101697 |
| BAC32_D20 | 226 | 22q12.1 | 24595242 | 24791688 | 196446 |
| BAC31_J04 | 227 | 22q12.1 | 24595244 | 24686321 | 91077 |
| BAC55_C24 | 228 | 22q12.1 | 24601225 | 24689439 | 88214 |
| BAC230_G24 | 229 | 22q12.1 | 24656694 | 24748468 | 91774 |
| BAC173_A12 | 230 | 22q12.1 | 24684348 | 24776338 | 91990 |
| BAC179_N04 | 231 | 22q12.1 | 24689358 | 24791695 | 102337 |

10) HNPP (Hereditary Neuropathy with Liability to Pressure Palsies) Disease

HNPP can be diagnosed for Karyotype deletion 17p11-12. A DNA chip for detecting HNPP includes a microarray containing at least one immobilized genomic DNA fragment selected from the group consisting of genomic DNA fragment 1 to 165 of Table 12, at least one oligomer of 20 to 100 bp consecutive nucleotide within the fragment, or at least one cDNA of 100 bp to 10 kbp derived from the fragment. When at least two kinds of probes are mixed and used, the probes can be mixed in equal proportions and immobilized in a single spot.

TABLE 12

| | | | Chromosomal location | | |
|---|---|---|---|---|---|
| | | Chromosomal location | in Human genome | | |
| BAC DNA | No. | identified by FISH | Start point | End point | size |
| BAC170_M14 | 1 | 17p12 | 14711135 | 14808289 | 97154 |
| BAC33_L21 | 2 | 17p12 | 14804500 | 14913795 | 109295 |
| BAC185_D08 | 3 | 17p12 | 14884357 | 14981705 | 97348 |
| BAC65_C19 | 4 | 17p12 | 14960572 | 15058918 | 98346 |
| BAC219_K23 | 5 | 17p12 | 14976175 | 15056221 | 80046 |
| BAC157_G01 | 6 | 17p12 | 15093558 | 15208862 | 115304 |
| BAC193_J12 | 7 | 17p12 | 15110864 | 15227229 | 116365 |
| BAC140_P21 | 8 | 17p12 | 15117703 | 15211297 | 93594 |
| BAC69_D20 | 9 | 17p12 | 15132146 | 15234625 | 102479 |
| BAC163_B05 | 10 | 17p12 | 15147290 | 15251664 | 104374 |
| BAC127_A13 | 11 | 17p12 | 15267749 | 15397538 | 129789 |
| BAC59_O13 | 12 | 17p12 | 15291315 | 15400674 | 109359 |
| BAC171_I03 | 13 | 17p12 | 15293504 | 15387860 | 94356 |
| BAC103_H17 | 14 | 17p12 | 15293548 | 15384732 | 91184 |
| BAC240_L04 | 15 | 17p12 | 15305705 | 15364092 | 58387 |
| BAC180_E05 | 16 | 17p12 | 15314834 | 15404065 | 89231 |
| BAC113_L01 | 17 | 17p12 | 15320794 | 15426185 | 105391 |
| BAC193_F10 | 18 | 17p12 | 15344377 | 15420149 | 75772 |
| BAC80_J24 | 19 | 17p12 | 15387712 | 15472607 | 84895 |
| BAC227_C12 | 20 | 17p12 | 15406530 | 15494411 | 87881 |
| BAC232_P15 | 21 | 17p12 | 15433058 | 15552649 | 119591 |
| BAC154_G20 | 22 | 17p12 | 15467403 | 15549983 | 82580 |
| BAC64_B16 | 23 | 17p12 | 15620998 | 15755383 | 134385 |
| BAC198_H06 | 24 | 17p12 | 15657045 | 15755442 | 98397 |
| BAC120_H23 | 25 | 17p12 | 15657050 | 15782155 | 125105 |
| BAC182_G04 | 26 | 17p12 | 15739595 | 15819054 | 79459 |
| BAC97_H12 | 27 | 17p12 | 15784582 | 15941885 | 157303 |
| BAC102_C09 | 28 | 17p12-17p11.2 | 15818957 | 20693695 | 4874738 |
| BAC41_E16 | 29 | 17p12 | 15847215 | 15926899 | 79684 |
| BAC150_L13 | 30 | 17p12 | 15854845 | 15963018 | 108173 |
| BAC124_J04 | 31 | 17p12 | 15897252 | 15992971 | 95719 |
| BAC96_G24 | 32 | 17p12 | 15938321 | 16028412 | 90091 |
| BAC66_G13 | 33 | 17p12 | 15941785 | 16044299 | 102514 |
| BAC184_B05 | 34 | 17p12 | 15962872 | 16044301 | 81429 |
| BAC60_L08 | 35 | 17p12 | 15962873 | 16060283 | 97410 |
| BAC189_J17 | 36 | 17p12-17p11.2 | 16143400 | 16227881 | 84481 |
| BAC88_H17 | 37 | 17p12-17p11.2 | 16143415 | 16236657 | 93242 |
| BAC42_K11 | 38 | 17p11.2 | 16289956 | 16394927 | 104971 |
| BAC240_E14 | 39 | 17p11.2 | 16394845 | 16489073 | 94228 |
| BAC148_O18 | 40 | 17p11.2 | 16420254 | 16530913 | 110659 |
| BAC125_N20 | 41 | 17p11.2 | 16557887 | 16711927 | 154040 |
| BAC175_H10 | 42 | 17p11.2 | 16557890 | 16636429 | 78539 |
| BAC146_D01 | 43 | 17p11.2 | 16557904 | 16636434 | 78530 |
| BAC62_D24 | 44 | 17p11.2 | 16654529 | 16751004 | 96475 |
| BAC186_K22 | 45 | 17p11.2 | 16724226 | 16796771 | 72545 |
| BAC33_P23 | 46 | 17p11.2 | 16779978 | 16866784 | 86806 |
| BAC14_G14 | 47 | 17p11.2 | 16796638 | 16913606 | 116968 |
| BAC72_P14 | 48 | 17p11.2 | 16866681 | 16988595 | 121914 |
| BAC131_K12 | 49 | 17p11.2 | 16909687 | 16990173 | 80486 |
| BAC197_O11 | 50 | 17p11.2 | 17025266 | 17165284 | 140018 |
| BAC26_I07 | 51 | 17p11.2 | 17025339 | 17091291 | 65952 |
| BAC36_F12 | 52 | 17p11.2 | 17074074 | 17165053 | 90979 |
| BAC129_D23 | 53 | 17p11.2 | 17158746 | 17245353 | 86607 |
| BAC101_K17 | 54 | 17p11.2 | 17245267 | 17333785 | 88518 |
| BAC123_D23 | 55 | 17p11.2 | 17245267 | 17357212 | 111945 |
| BAC89_C18 | 56 | 17p11.2 | 17276220 | 17391839 | 115619 |
| BAC90_H16 | 57 | 17p11.2 | 17283727 | 17383307 | 99580 |
| BAC198_N13 | 58 | 17p11.2 | 17283752 | 17390460 | 106708 |
| BAC154_C08 | 59 | 17p11.2 | 17295654 | 17419340 | 123686 |
| BAC179_F21 | 60 | 17p11.2 | 17324665 | 17419329 | 94664 |
| BAC189_N01 | 61 | 17p11.2 | 17390295 | 17465331 | 75036 |
| BAC20_P07 | 62 | 17p11.2 | 17419240 | 17530721 | 111481 |
| BAC167_A18 | 63 | 17p11.2 | 17419255 | 17572653 | 153398 |
| BAC95_I12 | 64 | 17p11.2 | 17482800 | 17572594 | 89794 |
| BAC155_B06 | 65 | 17p11.2 | 17484086 | 17572609 | 88523 |
| BAC56_J17 | 66 | 17p11.2 | 17551815 | 17665243 | 113428 |
| BAC36_C18 | 67 | 17p11.2 | 17579594 | 17676682 | 97088 |
| BAC170_M10 | 68 | 17p11.2 | 17588731 | 17678229 | 89498 |
| BAC236_B14 | 69 | 17p11.2 | 17698446 | 17776240 | 77794 |
| BAC76_J10 | 70 | 17p11.2 | 17709665 | 17806257 | 96592 |
| BAC165_E09 | 71 | 17p11.2 | 17723644 | 17806266 | 82622 |
| BAC249_G12 | 72 | 17p11.2 | 17776414 | 17890030 | 113616 |
| BAC41_D18 | 73 | 17p11.2 | 17889883 | 18025228 | 135345 |
| BAC131_M11 | 74 | 17p11.2 | 17916386 | 17992329 | 75943 |
| BAC103_D11 | 75 | 17p11.2 | 17935441 | 18032930 | 97489 |
| BAC57_N06 | 76 | 17p11.2 | 18030897 | 18165192 | 134295 |
| BAC197_E01 | 77 | 17p11.2 | 18044616 | 18128976 | 84360 |
| BAC20_D12 | 78 | 17p11.2 | 18128850 | 18219224 | 90374 |
| BAC238_N13 | 79 | 17p11.2 | 18165108 | 18259978 | 94870 |
| BAC57_C22 | 80 | 17p11.2 | 18202515 | 18354860 | 152345 |
| BAC184_A04 | 81 | 17p11.2 | 18206784 | 18295425 | 88641 |
| BAC70_D14 | 82 | 17p11.2 | 18206800 | 18295476 | 88676 |
| BAC186_E14 | 83 | 17p11.2 | 18332672 | 18452458 | 119786 |
| BAC97_I20 | 84 | 17p11.2 | 18393829 | 18513020 | 119191 |
| BAC199_D08 | 85 | 17p11.2 | 18403056 | 18513020 | 109964 |
| BAC154_P06 | 86 | 17p11.2 | 18421956 | 18508299 | 86343 |
| BAC39_C07 | 87 | 17p11.2 | 18507215 | 18613862 | 106647 |
| BAC228_C10 | 88 | 17p11.2 | 18594104 | 18712014 | 117910 |
| BAC81_H03 | 89 | 17p11.2 | 18594109 | 18726670 | 132561 |
| BAC58_P21 | 90 | 17p11.2 | 18635594 | 18755396 | 119802 |
| BAC233_A12 | 91 | 17p11.2 | 18737655 | 18849936 | 112281 |
| BAC153_J19 | 92 | 17p11.2 | 18746164 | 18831879 | 85715 |
| BAC189_M02 | 93 | 17p11.2 | 18773717 | 18895449 | 121732 |
| BAC132_A14 | 94 | 17p11.2 | 18805385 | 18918916 | 113531 |
| BAC95_F14 | 95 | 17p11.2 | 18807265 | 18895443 | 88178 |
| BAC191_B21 | 96 | 17p11.2 | 18808618 | 18884963 | 76345 |
| BAC132_L20 | 97 | 17p11.2 | 18811539 | 18902037 | 90498 |
| BAC232_D17 | 98 | 17p11.2 | 18812507 | 18903860 | 91353 |
| BAC77_C07 | 99 | 17p11.2 | 18831796 | 18927011 | 95215 |
| BAC61_M05 | 100 | 17p11.2 | 18845354 | 19050538 | 205184 |
| BAC57_D11 | 101 | 17p11.2 | 18897566 | 19000464 | 102898 |
| BAC77_B01 | 102 | 17p11.2 | 18952608 | 19072845 | 120237 |
| BAC84_B04 | 103 | 17p11.2 | 19079201 | 19218785 | 139584 |
| BAC160_J02 | 104 | 17p11.2 | 19225463 | 19266078 | 40615 |
| BAC109_F04 | 105 | 17p11.2 | 19244444 | 19304469 | 60025 |
| BAC31_F09 | 106 | 17p11.2 | 19244499 | 19310543 | 66044 |
| BAC54_L12 | 107 | 17p11.2 | 19244500 | 19313051 | 68551 |
| BAC183_B05 | 108 | 17p11.2 | 19265193 | 19337891 | 72698 |
| BAC97_K11 | 109 | 17p11.2 | 19313826 | 19387684 | 73858 |
| BAC148_B12 | 110 | 17p11.2 | 19402824 | 19492063 | 89239 |
| BAC26_B11 | 111 | 17p11.2 | 19526736 | 19640708 | 113972 |
| BAC12_C18 | 112 | 17p11.2 | 19534378 | 19640758 | 106380 |
| BAC92_D22 | 113 | 17p11.2 | 19534392 | 19651908 | 117516 |
| BAC69_L23 | 114 | 17p11.2 | 19545442 | 19651910 | 106468 |
| BAC175_P18 | 115 | 17p11.2 | 19563692 | 19648388 | 84696 |
| BAC146_B24 | 116 | 17p11.2 | 19650991 | 19746822 | 95831 |
| BAC27_K06 | 117 | 17p11.2 | 19651809 | 19749152 | 97343 |
| BAC70_N14 | 118 | 17p11.2 | 19665863 | 19786182 | 120319 |
| BAC171_D20 | 119 | 17p11.2 | 19680409 | 19788794 | 108385 |
| BAC116_O21 | 120 | 17p11.2 | 19718981 | 19786183 | 67202 |
| BAC10_I20 | 121 | 17p11.2 | 19739497 | 19816424 | 76927 |
| BAC96_I07 | 122 | 17p11.2 | 19816311 | 19903394 | 87083 |
| BAC249_G09 | 123 | 17p11.2 | 19867726 | 19989856 | 122130 |
| BAC44_J06 | 124 | 17p11.2 | 19891394 | 19971811 | 80417 |
| BAC193_P10 | 125 | 17p11.2 | 19926766 | 20039699 | 112933 |
| BAC156_C17 | 126 | 17p11.2 | 20083455 | 20215023 | 131568 |
| BAC36_G17 | 127 | 17p11.2 | 20083487 | 20218743 | 135256 |
| BAC11_J06 | 128 | 17p11.2 | 20193464 | 20281761 | 88297 |
| BAC124_E01 | 129 | 17p11.2 | 20228503 | 20332288 | 103785 |
| BAC117_D16 | 130 | 17p11.2 | 20270202 | 20378561 | 108359 |
| BAC188_P16 | 131 | 17p11.2 | 20340291 | 20436954 | 96663 |
| BAC233_L09 | 132 | 17p11.2 | 20457471 | 20549617 | 92146 |
| BAC20_M05 | 133 | 17p11.2 | 20457497 | 20526040 | 68543 |
| BAC69_F02 | 134 | 17p11.2 | 20477768 | 20580390 | 102622 |
| BAC237_C22 | 135 | 17p11.2 | 20499729 | 20600363 | 100634 |
| BAC60_H06 | 136 | 17p11.2 | 20503572 | 20599301 | 95729 |
| BAC141_A08 | 137 | 17p11.2 | 20511752 | 20596573 | 84821 |
| BAC40_H06 | 138 | 17p11.2 | 20525954 | 20630471 | 104517 |
| BAC142_P01 | 139 | 17p11.2 | 20596491 | 20693726 | 97235 |
| BAC48_M08 | 140 | 17p11.2 | 20706155 | 20795955 | 89800 |
| BAC223_H04 | 141 | 17p11.2 | 20734592 | 20851649 | 117057 |
| BAC195_E10 | 142 | 17p11.2 | 20769887 | 20847051 | 77164 |
| BAC200_J14 | 143 | 17p11.2 | 20811622 | 20895723 | 84101 |
| BAC223_I12 | 144 | 17p11.2 | 20811641 | 20905322 | 93681 |
| BAC28_P01 | 145 | 17p11.2 | 20851520 | 20989326 | 137806 |
| BAC119_A16 | 146 | 17p11.2 | 20955668 | 21104977 | 149309 |
| BAC4_G22 | 147 | 17p11.2 | 21004734 | 21099992 | 95258 |
| BAC118_H17 | 148 | 17p11.2 | 21017214 | 21121333 | 104119 |
| BAC40_J07 | 149 | 17p11.2 | 21028143 | 21121354 | 93211 |
| BAC239_L05 | 150 | 17p11.2 | 21036358 | 21175468 | 139110 |
| BAC32_C06 | 151 | 17p11.2 | 21134931 | 21313722 | 178791 |
| BAC248_A23 | 152 | 17p11.2 | 21214133 | 21296062 | 81929 |
| BAC142_A03 | 153 | 17p11.2 | 21214197 | 21315277 | 101080 |
| BAC61_I13 | 154 | 17p11.2 | 21277948 | 21385816 | 107868 |
| BAC236_B19 | 155 | 17p11.2 | 21315182 | 21425247 | 110065 |
| BAC100_J04 | 156 | 17p11.2 | 21393550 | 21547843 | 154293 |
| BAC139_B06 | 157 | 17p11.2-17q11.1 | 21411493 | 25423821 | 4012328 |
| BAC130_E22 | 158 | 17p11.2 | 21425175 | 21493695 | 68520 |
| BAC68_G08 | 159 | 17p11.2 | 21458460 | 21552363 | 93903 |
| BAC26_L13 | 160 | 17p11.2 | 21843173 | 21970661 | 127488 |
| BAC118_E14 | 161 | 17p11.2 | 21953893 | 22050818 | 96925 |
| BAC236_B22 | 162 | 17p11.2 | 22031416 | 22050802 | 19386 |
| BAC81_F21 | 163 | 17p11.2 | 22050715 | 22087819 | 37104 |
| BAC168_D15 | 164 | 17p11.2 | 22050731 | 22088509 | 37778 |
| BAC199_H24 | 165 | 17p11.2 | 22090493 | 22173463 | 82970 |

(11) Miller-Dieker Syndrome

Miller-Dieker syndrome can be diagnosed for Karyotype deletion 17p13.2-3. A DNA chip for detecting Miller-Dieker syndrome includes a microarray containing at least one immobilized genomic DNA fragment selected from the group consisting of genomic DNA fragment 1 to 137 of Table 13, at least one oligomer of 20 to 100 bp consecutive nucleotide within the fragment, or at least one cDNA of 100 bp to 10 kbp derived from the fragment. When at least two kinds of probes are mixed and used, the probes can be mixed in equal proportions and immobilized in a single spot.

TABLE 13

| | | Chromo- | | | |
|---|---|---|---|---|---|
| | | somal | | | |
| | | location | Chromosomal location | | |
| | | identified | in Human genome | | |
| BAC DNA | No. | by FISH | Start point | End point | size |
| BAC95_J10 | 1 | 17p13.3 | 64800 | 139216 | 74416 |
| BAC156_A11 | 2 | 17p13.3 | 64812 | 169940 | 105128 |
| BAC25_B05 | 3 | 17p13.3 | 139132 | 302271 | 163139 |
| BAC197_A20 | 4 | 17p13.3 | 154688 | 255949 | 101261 |
| BAC66_O11 | 5 | 17p13.3 | 155580 | 263887 | 108307 |
| BAC219_G17 | 6 | 17p13.3 | 169850 | 242505 | 72655 |
| BAC110_O13 | 7 | 17p13.3 | 195809 | 331942 | 136133 |
| BAC199_F13 | 8 | 17p13.3 | 195813 | 271824 | 76011 |
| BAC226_F20 | 9 | 17p13.3 | 440934 | 541847 | 100913 |
| BAC60_O14 | 10 | 17p13.3 | 440974 | 444602 | 3628 |
| BAC84_C19 | 11 | 17p13.3 | 448037 | 555316 | 107279 |
| BAC49_N19 | 12 | 17p13.3 | 448041 | 582881 | 134840 |
| BAC220_M24 | 13 | 17p13.3 | 555159 | 641658 | 86499 |
| BAC34_H06 | 14 | 17p13.3 | 555211 | 641604 | 86393 |
| BAC119_A07 | 15 | 17p13.3 | 588546 | 685736 | 97190 |
| BAC141_P05 | 16 | 17p13.3 | 713179 | 787793 | 74614 |
| BAC32_G05 | 17 | 17p13.3 | 816435 | 944490 | 128055 |
| BAC160_I09 | 18 | 17p13.3 | 852455 | 945044 | 92589 |
| BAC71_H10 | 19 | 17p13.3 | 916465 | 1060463 | 143998 |
| BAC55_J01 | 20 | 17p13.3 | 944966 | 1079378 | 134412 |
| BAC31_I23 | 21 | 17p13.3 | 944982 | 1029701 | 84719 |
| BAC104_O02 | 22 | 17p13.3 | 944989 | 1060455 | 115466 |
| BAC159_N11 | 23 | 17p13.3 | 965320 | 1090899 | 125579 |
| BAC184_G24 | 24 | 17p13.3 | 1007662 | 1106304 | 98642 |
| BAC38_B05 | 25 | 17p13.3 | 1035912 | 1370806 | 334894 |
| BAC142_M22 | 26 | 17p13.3 | 1045102 | 1110071 | 64969 |
| BAC217_L13 | 27 | 17p13.3 | 1079264 | 1380224 | 300960 |
| BAC184_G09 | 28 | 17p13.3 | 1408955 | 1507994 | 99039 |
| BAC163_F14 | 29 | 17p13.3 | 1409004 | 1521254 | 112250 |
| BAC171_F06 | 30 | 17p13.3 | 1409004 | 1488367 | 79363 |
| BAC80_A10 | 31 | 17p13.3 | 1437236 | 1549108 | 111872 |
| BAC180_C17 | 32 | 17p13.3 | 1481243 | 1632824 | 151581 |
| BAC78_F20 | 33 | 17p13.3 | 1602258 | 1771115 | 168857 |
| BAC217_H14 | 34 | 17p13.3 | 1619175 | 1728028 | 108853 |
| BAC163_D12 | 35 | 17p13.3 | 1619265 | 1742452 | 123187 |
| BAC65_K23 | 36 | 17p13.3 | 1662255 | 1828663 | 166408 |
| BAC101_E22 | 37 | 17p13.3 | 1718637 | 1873340 | 154703 |
| BAC150_P22 | 38 | 17p13.3 | 1718682 | 1828663 | 109981 |
| BAC12_I17 | 39 | 17p13.3 | 1760111 | 1873022 | 112911 |
| BAC40_H23 | 40 | 17p13.3 | 1769413 | 1864391 | 94978 |
| BAC95_G19 | 41 | 17p13.3 | 1872849 | 1975935 | 103086 |
| BAC106_M10 | 42 | 17p13.3 | 1876442 | 1956231 | 79789 |
| BAC69_C07 | 43 | 17p13.3 | 1876485 | 1970979 | 94494 |
| BAC112_N05 | 44 | 17p13.3 | 1891790 | 1995088 | 103298 |
| BAC237_F04 | 45 | 17p13.3 | 1891850 | 1975942 | 84092 |
| BAC209_C22 | 46 | 17p13.3 | 1937629 | 2050730 | 113101 |
| BAC80_M08 | 47 | 17p13.3 | 1990152 | 2084363 | 94211 |
| BAC59_L21 | 48 | 17p13.3 | 1994050 | 2150546 | 156496 |
| BAC39_H21 | 49 | 17p13.3 | 2019865 | 2171995 | 152130 |
| BAC162_C13 | 50 | 17p13.3 | 2029196 | 2090384 | 61188 |
| BAC8_D13 | 51 | 17p13.3 | 2047271 | 2178437 | 131166 |
| BAC34_O15 | 52 | 17p13.3 | 2052014 | 2142638 | 90624 |
| BAC160_N09 | 53 | 17p13.3 | 2079384 | 2171985 | 92601 |
| BAC91_M16 | 54 | 17p13.3 | 2079445 | 2178421 | 98976 |
| BAC50_H10 | 55 | 17p13.3 | 2142538 | 2237448 | 94910 |
| BAC126_D23 | 56 | 17p13.3 | 2150478 | 2231023 | 80545 |
| BAC153_G02 | 57 | 17p13.3 | 2150484 | 2236800 | 86316 |
| BAC109_H09 | 58 | 17p13.3 | 2182705 | 2372182 | 189477 |
| BAC122_J08 | 59 | 17p13.3 | 2313296 | 2407569 | 94273 |
| BAC40_D11 | 60 | 17p13.3 | 2320597 | 2456825 | 136228 |
| BAC176_L20 | 61 | 17p13.3 | 2374911 | 2468142 | 93231 |
| BAC188_G13 | 62 | 17p13.3 | 2393571 | 2468132 | 74561 |
| BAC183_G10 | 63 | 17p13.3 | 2443970 | 2545838 | 101868 |
| BAC82_M13 | 63 | 17p13.3 | 2468056 | 2594074 | 126018 |
| BAC125_I23 | 64 | 17p13.3 | 2482554 | 2576999 | 94445 |
| BAC175_K10 | 65 | 17p13.3 | 2486322 | 2577034 | 90712 |
| BAC226_F05 | 66 | 17p13.3 | 2486323 | 2577038 | 90715 |
| BAC89_M06 | 67 | 17p13.3 | 2493663 | 2588416 | 94753 |
| BAC46_G14 | 68 | 17p13.3 | 2516649 | 2594061 | 77412 |
| BAC157_B02 | 69 | 17p13.3 | 2580013 | 2690941 | 110928 |
| BAC234_G19 | 70 | 17p13.3 | 2588322 | 2702157 | 113835 |
| BAC237_P03 | 71 | 17p13.3 | 2600587 | 2690939 | 90352 |
| BAC10_A05 | 72 | 17p13.3 | 2634245 | 2734793 | 100548 |
| BAC190_F10 | 73 | 17p13.3 | 2648213 | 2748214 | 100001 |
| BAC186_M15 | 74 | 17p13.3 | 2720577 | 2893585 | 173008 |
| BAC183_M06 | 75 | 17p13.13 | 2749030 | 2834151 | 85121 |
| BAC195_P05 | 76 | 17p13.3 | 2752826 | 2834206 | 81380 |
| BAC148_F06 | 77 | 17p13.3 | 2777609 | 2863478 | 85869 |
| BAC31_H03 | 78 | 17p13.3 | 2777616 | 2863483 | 85867 |
| BAC40_E12 | 79 | 17p13.3 | 2977133 | 3080635 | 103502 |
| BAC194_G14 | 80 | 17p13.3 | 3001363 | 3090339 | 88976 |
| BAC26_P17 | 81 | 17p13.3 | 3025028 | 3125343 | 100315 |
| BAC247_D10 | 82 | 17p13.3 | 3072003 | 3151666 | 79663 |
| BAC36_N06 | 83 | 17p13.3 | 3072523 | 3180602 | 108079 |
| BAC122_O10 | 84 | 17p13.3- | 3213606 | 3374638 | 161032 |
| | | 17p13.2 | | | |
| BAC106_K05 | 85 | 17p13.3- | 3268233 | 3455857 | 187624 |
| | | 17p13.2 | | | |
| BAC154_K10 | 86 | 17p13.2 | 3323959 | 3418503 | 94544 |
| BAC232_E03 | 87 | 17p13.2 | 3378469 | 3455863 | 77394 |
| BAC35_K08 | 88 | 17p13.2 | 3511754 | 3631321 | 119567 |
| BAC73_C22 | 89 | 17p13.2 | 3554395 | 3640306 | 85911 |
| BAC200_I19 | 90 | 17p13.2 | 3610262 | 3694595 | 84333 |
| BAC217_A05 | 91 | 17p13.2 | 3674472 | 3755413 | 80941 |
| BAC186_H22 | 92 | 17p13.2 | 3692964 | 3813073 | 120109 |
| BAC33_O16 | 93 | 17p13.2 | 3770500 | 3877821 | 107321 |
| BAC192_G13 | 94 | 17p13.2 | 3777237 | 3870667 | 93430 |
| BAC151_J24 | 95 | 17p13.2 | 3972239 | 4067990 | 95751 |
| BAC246_O19 | 96 | 17p13.2 | 3972254 | 4049275 | 77021 |
| BAC145_N20 | 97 | 17p13.2 | 3972256 | 4071522 | 99266 |
| BAC237_H14 | 98 | 17p13.2 | 4078134 | 4167032 | 88898 |
| BAC118_M12 | 99 | 17p13.2 | 4182139 | 4293897 | 111758 |
| BAC8_J04 | 100 | 17p13.2 | 4182145 | 4359656 | 177511 |
| BAC177_A22 | 101 | 17p13.2 | 4182145 | 4276005 | 93860 |
| BAC73_A18 | 102 | 17p13.2 | 4188355 | 4267605 | 79250 |
| BAC144_E16 | 103 | 17p13.2 | 4215724 | 4323128 | 107404 |
| BAC6_J05 | 104 | 17p13.2 | 4236264 | 4377783 | 141519 |
| BAC142_D02 | 105 | 17p13.2 | 4275959 | 4370246 | 94287 |
| BAC52_H24 | 106 | 17p13.2 | 4293737 | 4386785 | 93048 |
| BAC157_G11 | 107 | 17p13.2 | 4374088 | 4377780 | 3692 |
| BAC236_B12 | 108 | 17p13.2 | 4433954 | 4530900 | 96946 |
| BAC51_I06 | 109 | 17p13.2 | 4511904 | 4608096 | 96192 |
| BAC165_K05 | 110 | 17p13.2 | 4534032 | 4620241 | 86209 |
| BAC39_E15 | 111 | 17p13.2 | 4669470 | 4832933 | 163463 |
| BAC1_A10 | 112 | 17p13.2 | 4732580 | 4831098 | 98518 |
| BAC219_K06 | 113 | 17p13.2 | 4756087 | 4835323 | 79236 |
| BAC27_B12 | 114 | 17p13.2 | 5060051 | 5198718 | 138667 |
| BAC189_A24 | 115 | 17p13.2 | 5142270 | 5155722 | 13452 |
| BAC163_I08 | 116 | 17p13.2 | 5181622 | 5323862 | 142240 |
| BAC19_L13 | 117 | 17p13.2 | 5252313 | 5364075 | 111762 |
| BAC58_D19 | 118 | 17p13.2 | 5258395 | 5359384 | 100989 |
| BAC140_E06 | 119 | 17p13.2 | 5258401 | 5332448 | 74047 |
| BAC50_B02 | 120 | 17p13.2 | 5264812 | 5358778 | 93966 |
| BAC134_O19 | 121 | 17p13.2 | 5306934 | 5391890 | 84956 |
| BAC38_J10 | 122 | 17p13.2 | 5358690 | 5476075 | 117385 |
| BAC61_E13 | 123 | 17p13.2 | 5391794 | 5515041 | 123247 |
| BAC124_A19 | 124 | 17p13.2 | 5391807 | 5543683 | 151876 |
| BAC233_J22 | 125 | 17p13.2 | 5394259 | 5478311 | 84052 |
| BAC11_M16 | 126 | 17p13.2 | 5404009 | 5510827 | 106818 |
| BAC152_P06 | 127 | 17p13.2 | 5406393 | 5494515 | 88122 |
| BAC112_B03 | 128 | 17p13.2 | 5406394 | 5515061 | 108667 |
| BAC76_P20 | 129 | 17p13.2 | 5476282 | 5571700 | 95418 |
| BAC86_E11 | 130 | 17p13.2 | 5515910 | 5616965 | 101055 |
| BAC93_O20 | 131 | 17p13.2 | 5562182 | 5662324 | 100142 |
| BAC22_O01 | 132 | 17p13.2 | 5594574 | 5699114 | 104540 |
| BAC221_K09 | 133 | 17p13.2 | 5624175 | 5699124 | 74949 |
| BAC196_C21 | 134 | 17p13.2 | 5624178 | 5716841 | 92663 |
| BAC59_H08 | 135 | 17p13.2 | 5644318 | 5759034 | 114716 |
| BAC238_L13 | 136 | 17p13.2 | 5716742 | 5790604 | 73862 |
| BAC166_I21 | 137 | 17p13.2 | 5739464 | 5747430 | 7966 |

(12) Prader-Willi Syndrome

Prader-willi syndrome can be diagnosed for Karyotype deletion 15q11-13. A DNA chip for detecting Prader-willi syndrome includes a microarray containing at least one immobilized genomic DNA fragment selected from the group consisting of genomic DNA fragment 1 to 321 of Table 14, at least one oligomer of 20 to 100 bp consecutive nucleotide within the fragment, or at least one cDNA of 100 bp to 10 kbp derived from the fragment. When at least two kinds of probes are mixed and used, the probes can be mixed in equal proportions and immobilized in a single spot.

TABLE 14

| | | Chromo- | | | |
|---|---|---|---|---|---|
| | | somal | Chromosomal | | |
| | | location | location in | | |
| | | identified | Human genome | | |
| BAC DNA | No. | by FISH | Start point | End point | size |
| BAC141_C03 | 1 | 15q11.2 | 18382076 | 19517632 | 1135556 |
| BAC246_K11 | 2 | 15q11.2 | 18455502 | 18533923 | 78421 |
| BAC91_F06 | 3 | 15q11.2 | 18581333 | 19519542 | 938209 |
| BAC186_E15 | 4 | 15q11.2 | 18625687 | 19546605 | 920918 |
| BAC65_A01 | 5 | 15q11.2 | 18712675 | 18813560 | 100885 |
| BAC18_M24 | 6 | 15q11.2 | 18717043 | 18813535 | 96492 |
| BAC55_G12 | 7 | 15q11.2 | 18744714 | 18844909 | 100195 |
| BAC109_M07 | 8 | 15q11.2 | 18753171 | 18844836 | 91665 |
| BAC167_P01 | 9 | 15q11.2 | 18769076 | 18844900 | 75824 |
| BAC73_M10 | 10 | 15q11.2 | 19023304 | 19119348 | 96044 |
| BAC108_J23 | 11 | 15q11.2 | 19047746 | 19150666 | 102920 |
| BAC83_C02 | 12 | 15q11.2 | 19104635 | 19232649 | 128014 |
| BAC118_L12 | 13 | 15q11.2 | 19113566 | 20974303 | 1860737 |
| BAC243_B24 | 14 | 15q11.2 | 19119293 | 20991978 | 1872685 |
| BAC40_O04 | 15 | 15q11.2 | 19129034 | 20974336 | 1845302 |
| BAC234_H10 | 16 | 15q11.2 | 19163585 | 19529225 | 365640 |
| BAC120_M08 | 17 | 15q11.2 | 19164356 | 19263488 | 99132 |
| BAC144_A10 | 18 | 15q11.2 | 19165513 | 19258620 | 93107 |
| BAC182_O07 | 19 | 15q11.2 | 19165520 | 19250988 | 85468 |
| BAC123_E02 | 20 | 15q11.2 | 19168659 | 19263492 | 94833 |
| BAC107_E19 | 21 | 15q11.2 | 19199475 | 21043782 | 1844307 |
| BAC80_A21 | 22 | 15q11.2 | 19200232 | 19319023 | 118791 |
| BAC132_D08 | 23 | 15q11.2 | 19230676 | 19355302 | 124626 |
| BAC73_E23 | 24 | 15q11.2 | 19258530 | 19367078 | 108548 |
| BAC232_J20 | 25 | 15q11.2 | 19279907 | 19360994 | 81087 |
| BAC100_H13 | 26 | 15q11.2 | 19317895 | 19399647 | 81752 |
| BAC243_J13 | 27 | 15q11.2 | 19340540 | 19425482 | 84942 |
| BAC217_O02 | 28 | 15q11.2 | 19356386 | 19391744 | 35358 |
| BAC146_C18 | 29 | 15q11.2 | 19366904 | 19793707 | 426803 |
| BAC140_H23 | 30 | 15q11.2 | 19452153 | 19868598 | 416445 |
| BAC176_G05 | 31 | 15q11.2 | 19465162 | 19545771 | 80609 |
| BAC107_A12 | 32 | 15q11.2 | 19776376 | 19878033 | 101657 |
| BAC80_L19 | 33 | 15q11.2 | 19937913 | 20069228 | 131315 |
| BAC248_A08 | 34 | 15q11.2 | 19939320 | 20062559 | 123239 |
| BAC71_N13 | 35 | 15q11.2 | 19954318 | 20050201 | 95883 |
| BAC146_H15 | 36 | 15q11.2 | 19954332 | 20077780 | 123448 |
| BAC247_N02 | 37 | 15q11.2 | 19979490 | 20062562 | 83072 |
| BAC185_A21 | 38 | 15q11.2 | 19991570 | 20083195 | 91625 |
| BAC66_M12 | 39 | 15q11.2 | 20222006 | 21156734 | 934728 |
| BAC92_A20 | 40 | 15q11.2 | 20328519 | 20421785 | 93266 |
| BAC42_J12 | 41 | 15q11.2 | 20411014 | 20565256 | 154242 |
| BAC82_A01 | 42 | 15q11.2 | 20411014 | 20515666 | 104652 |
| BAC97_H23 | 43 | 15q11.2 | 20421683 | 20506419 | 84736 |
| BAC244_M09 | 44 | 15q11.2 | 20482088 | 20570409 | 88321 |
| BAC96_G19 | 45 | 15q11.2 | 20482094 | 20607463 | 125369 |
| BAC123_I24 | 46 | 15q11.2 | 20548835 | 20665672 | 116837 |
| BAC89_J16 | 47 | 15q11.2 | 20630355 | 20733202 | 102847 |
| BAC136_C10 | 48 | 15q11.2 | 20686256 | 20771168 | 84912 |
| BAC93_A23 | 49 | 15q11.2 | 20786661 | 20886850 | 100189 |
| BAC151_I05 | 50 | 15q11.2 | 20791596 | 20875017 | 83421 |
| BAC141_K24 | 51 | 15q11.2 | 20877586 | 20983283 | 105697 |
| BAC238_B09 | 52 | 15q11.2 | 20936780 | 21022644 | 85864 |
| BAC84_J11 | 53 | 15q11.2 | 21138610 | 21245928 | 107318 |
| BAC234_N19 | 54 | 15q11.2 | 21138624 | 21205921 | 67297 |
| BAC10_E02 | 55 | 15q11.2- | 21173573 | 26526965 | 5353392 |
| | | 15q13.1 | | | |
| BAC39_B20 | 56 | 15q11.2 | 21202186 | 21225329 | 23143 |
| BAC61_D19 | 57 | 15q11.2 | 21205765 | 21306526 | 100761 |
| BAC28_D16 | 58 | 15q11.2 | 21208525 | 21226898 | 18373 |
| BAC38_K16 | 59 | 15q11.2 | 21296360 | 21441554 | 145194 |
| BAC73_A20 | 60 | 15q11.2 | 21338040 | 21431639 | 93599 |
| BAC148_L16 | 61 | 15q11.2 | 21338057 | 21431587 | 93530 |
| BAC218_E03 | 62 | 15q11.2 | 21351496 | 21431581 | 80085 |
| BAC31_M12 | 63 | 15q11.2 | 21351509 | 21453785 | 102276 |
| BAC111_B02 | 64 | 15q11.2 | 21414362 | 21495921 | 81559 |
| BAC1_L18 | 65 | 15q11.2 | 21442266 | 21544680 | 102414 |
| BAC56_G11 | 66 | 15q11.2 | 21454146 | 21597704 | 143558 |
| BAC84_I09 | 67 | 15q11.2 | 21495803 | 21613165 | 117362 |
| BAC34_E16 | 68 | 15q11.2 | 21495819 | 21588947 | 93128 |
| BAC223_H07 | 69 | 15q11.2 | 21514731 | 21603329 | 88598 |
| BAC188_N24 | 70 | 15q11.2 | 21603236 | 21716191 | 112955 |
| BAC46_D01 | 71 | 15q11.2 | 21608170 | 21643440 | 35270 |
| BAC237_N23 | 72 | 15q11.2 | 21683432 | 21782588 | 99156 |
| BAC185_J12 | 73 | 15q11.2 | 21689484 | 21798347 | 108863 |
| BAC11_E11 | 74 | 15q11.2 | 21753209 | 21868563 | 115354 |
| BAC187_K14 | 75 | 15q11.2 | 21755224 | 21827899 | 72675 |
| BAC155_L14 | 76 | 15q11.2 | 21849348 | 21941705 | 92357 |
| BAC247_M05 | 77 | 15q11.2 | 21873919 | 21970298 | 96379 |
| BAC173_O14 | 78 | 15q11.2 | 22109666 | 22196392 | 86726 |
| BAC189_O02 | 79 | 15q11.2 | 22483627 | 22596331 | 112704 |
| BAC183_O14 | 80 | 15q11.2 | 22522909 | 22609205 | 86296 |
| BAC219_F10 | 81 | 15q11.2 | 22535395 | 22598097 | 62702 |
| BAC180_F11 | 82 | 15q11.2 | 22652177 | 22727943 | 75766 |
| BAC223_H02 | 83 | 15q11.2 | 22690669 | 22776215 | 85546 |
| BAC217_F02 | 84 | 15q11.2 | 22764099 | 22852461 | 88362 |
| BAC230_G04 | 85 | 15q11.2 | 22810604 | 22905812 | 95208 |
| BAC155_J02 | 86 | 15q11.2 | 22819513 | 22906616 | 87103 |
| BAC133_K17 | 87 | 15q11.2 | 22826721 | 22953046 | 126325 |
| BAC88_I16 | 88 | 15q11.2 | 22852377 | 22953047 | 100670 |
| BAC80_D21 | 89 | 15q11.2- | 22952963 | 23059366 | 106403 |
| | | 15q12 | | | |
| BAC147_A15 | 90 | 15q12 | 23002283 | 23103304 | 101021 |
| BAC71_A18 | 91 | 15q12 | 23097994 | 23184636 | 86642 |
| BAC36_N10 | 92 | 15q12 | 23247880 | 23330804 | 82924 |
| BAC163_D17 | 93 | 15q12 | 23266062 | 23362050 | 95988 |
| BAC108_C16 | 94 | 15q12 | 23291956 | 23386063 | 94107 |
| BAC110_P13 | 95 | 15q12 | 23324937 | 23451308 | 126371 |
| BAC55_A23 | 96 | 15q12 | 23330199 | 23411751 | 81552 |
| BAC106_F15 | 97 | 15q12 | 23335336 | 23411749 | 76413 |
| BAC21_C02 | 98 | 15q12 | 23364792 | 23482854 | 118062 |
| BAC226_K20 | 99 | 15q12 | 23393743 | 23481009 | 87266 |
| BAC135_F07 | 100 | 15q12 | 23411631 | 23495077 | 83446 |
| BAC162_N18 | 101 | 15q12 | 23427584 | 23520410 | 92826 |
| BAC68_N10 | 102 | 15q12 | 23543464 | 23658889 | 115425 |
| BAC12_A24 | 103 | 15q12 | 23543470 | 23686313 | 142843 |
| BAC192_P17 | 104 | 15q12 | 23548156 | 23662752 | 114596 |
| BAC92_A21 | 105 | 15q12 | 23579789 | 23674188 | 94399 |
| BAC142_G09 | 106 | 15q12 | 23632192 | 23725644 | 93452 |
| BAC148_G03 | 107 | 15q12 | 23632351 | 23735160 | 102809 |
| BAC46_M10 | 108 | 15q12 | 23634033 | 23725651 | 91618 |
| BAC160_H06 | 109 | 15q12 | 23662610 | 23776255 | 113645 |
| BAC249_C08 | 110 | 15q12 | 23662669 | 23754493 | 91824 |
| BAC79_C22 | 111 | 15q12 | 23897093 | 24050231 | 153138 |
| BAC178_K14 | 112 | 15q12 | 23911330 | 23986611 | 75281 |
| BAC187_J01 | 113 | 15q12 | 24003385 | 24088420 | 85035 |
| BAC56_J08 | 114 | 15q12 | 24021035 | 24104986 | 83951 |
| BAC106_F07 | 115 | 15q12 | 24115546 | 24214583 | 99037 |
| BAC247_G10 | 116 | 15q12 | 24165098 | 24252655 | 87557 |
| BAC50_P08 | 117 | 15q12 | 24169248 | 24254530 | 85282 |
| BAC123_N23 | 118 | 15q12 | 24171747 | 24263411 | 91664 |
| BAC248_C13 | 119 | 15q12 | 24277267 | 24380252 | 102985 |
| BAC78_F07 | 120 | 15q12 | 24365663 | 24481932 | 116269 |
| BAC180_J22 | 121 | 15q12 | 24394174 | 24501973 | 107799 |
| BAC199_J15 | 122 | 15q12 | 24397328 | 24477098 | 79770 |
| BAC21_O06 | 123 | 15q12 | 24425649 | 24550138 | 124489 |
| BAC105_L07 | 124 | 15q12 | 24452906 | 24574011 | 121105 |
| BAC144_F12 | 125 | 15q12 | 24715131 | 24838753 | 123622 |
| BAC162_H22 | 126 | 15q12 | 24744989 | 24867883 | 122894 |
| BAC181_E03 | 127 | 15q12 | 24776471 | 24863452 | 86981 |
| BAC62_B05 | 128 | 15q12 | 24887538 | 24992463 | 104925 |
| BAC23_C04 | 129 | 15q12 | 24894441 | 25016437 | 121996 |
| BAC218_C05 | 130 | 15q12 | 25023664 | 25047824 | 24160 |
| BAC8_E18 | 131 | 15q12 | 25256341 | 25363093 | 106752 |
| BAC106_O13 | 132 | 15q12 | 25280377 | 25402891 | 122514 |
| BAC187_I13 | 133 | 15q12 | 25361444 | 25492578 | 131134 |
| BAC236_A03 | 134 | 15q12 | 25383696 | 25485582 | 101886 |
| BAC187_N03 | 135 | 15q12 | 25426775 | 25525741 | 98966 |
| BAC82_P01 | 136 | 15q12 | 25500610 | 25597528 | 96918 |
| BAC91_O02 | 137 | 15q12 | 25543075 | 25674894 | 131819 |
| BAC137_H09 | 138 | 15q12 | 25568151 | 25656759 | 88608 |
| BAC25_G19 | 139 | 15q12- | 25664673 | 25781065 | 116392 |
| | | 15q13.1 | | | |
| BAC33_E07 | 140 | 15q13.1 | 25752869 | 25869230 | 116361 |
| BAC84_O23 | 141 | 15q13.1 | 25802932 | 25912747 | 109815 |
| BAC111_H22 | 142 | 15q13.1 | 26070373 | 26168324 | 97951 |
| BAC136_O23 | 143 | 15q13.1 | 26079558 | 26168323 | 88765 |
| BAC161_D04 | 144 | 15q13.1 | 26422387 | 26505540 | 83153 |
| BAC78_I18 | 145 | 15q13.1 | 26551519 | 26677039 | 125520 |
| BAC33_G10 | 146 | 15q13.1 | 27026852 | 27200408 | 173556 |
| BAC29_F11 | 147 | 15q13.1 | 27042253 | 27170055 | 127802 |
| BAC196_I03 | 148 | 15q13.1 | 27044188 | 27144095 | 99907 |
| BAC218_L02 | 149 | 15q13.1 | 27126444 | 27292546 | 166102 |
| BAC73_H09 | 150 | 15q13.1 | 27144853 | 27232235 | 87382 |
| BAC66_D02 | 151 | 15q13.1 | 27165793 | 27258882 | 93089 |
| BAC43_H20 | 152 | 15q13.1 | 27258784 | 27388032 | 129248 |
| BAC46_I18 | 153 | 15q13.1 | 27303238 | 27439388 | 136150 |
| BAC64_N21 | 154 | 15q13.1 | 27445464 | 27539696 | 94232 |
| BAC140_F21 | 155 | 15q13.1 | 27509801 | 27620258 | 110457 |
| BAC182_D23 | 156 | 15q13.1 | 27526984 | 27607080 | 80096 |
| BAC84_A10 | 157 | 15q13.1 | 27541714 | 27643198 | 101484 |
| BAC238_I20 | 158 | 15q13.1 | 27624710 | 27728308 | 103598 |
| BAC53_B04 | 159 | 15q13.1 | 27648611 | 27787077 | 138466 |
| BAC24_E18 | 160 | 15q13.1 | 27662198 | 27764933 | 102735 |
| BAC242_C09 | 161 | 15q13.1 | 27706865 | 27798175 | 91310 |
| BAC43_E23 | 162 | 15q13.1 | 27745406 | 27836171 | 90765 |
| BAC240_P07 | 163 | 15q13.1 | 27764790 | 27884447 | 119657 |
| BAC46_D04 | 164 | 15q13.1- | 27968136 | 28067878 | 99742 |
| | | 15q13.2 | | | |
| BAC142_D15 | 165 | 15q13.1- | 27993604 | 28098643 | 105039 |
| | | 15q13.2 | | | |
| BAC143_H23 | 166 | 15q13.1- | 27993605 | 28098587 | 104982 |
| | | 15q13.2 | | | |
| BAC243_O04 | 167 | 15q13.2 | 28015293 | 28589243 | 573950 |
| BAC38_L21 | 168 | 15q13.2 | 28051211 | 28187361 | 136150 |
| BAC37_L06 | 169 | 15q13.2 | 28268014 | 28383142 | 115128 |
| BAC135_A04 | 170 | 15q13.2 | 28415534 | 28512548 | 97014 |
| BAC143_E18 | 171 | 15q13.2 | 28567143 | 28645618 | 78475 |
| BAC228_K02 | 172 | 15q13.2 | 28661319 | 28757422 | 96103 |
| BAC64_E04 | 173 | 15q13.2 | 28700479 | 28774619 | 74140 |
| BAC69_C17 | 174 | 15q13.2 | 28778775 | 28897821 | 119046 |
| BAC173_E06 | 175 | 15q13.2- | 28898602 | 28977101 | 78499 |
| | | 15q13.3 | | | |
| BAC66_J05 | 176 | 15q13.3 | 28939243 | 29044780 | 105537 |
| BAC242_O02 | 177 | 15q13.3 | 29011346 | 29098099 | 86753 |
| BAC91_I01 | 178 | 15q13.3 | 29022504 | 29117112 | 94608 |
| BAC19_L10 | 179 | 15q13.3 | 29030253 | 29160435 | 130182 |
| BAC100_C18 | 180 | 15q13.3 | 29203129 | 29327976 | 124847 |
| BAC173_P11 | 181 | 15q13.3 | 29295729 | 29390354 | 94625 |
| BAC83_O23 | 182 | 15q13.3 | 29340892 | 29427397 | 86505 |
| BAC233_L15 | 183 | 15q13.3 | 29344204 | 29436814 | 92610 |
| BAC36_N21 | 184 | 15q13.3 | 29403609 | 29544643 | 141034 |
| BAC167_N16 | 185 | 15q13.3 | 29403670 | 29502412 | 98742 |
| BAC171_B11 | 186 | 15q13.3 | 29417702 | 29485175 | 67473 |
| BAC171_K22 | 187 | 15q13.3 | 29427164 | 29516013 | 88849 |
| BAC26_K14 | 189 | 15q13.3 | 29509350 | 29596631 | 87281 |
| BAC171_K10 | 190 | 15q13.3 | 29645335 | 29728311 | 82976 |
| BAC50_A04 | 191 | 15q13.3 | 29655064 | 29746173 | 91109 |
| BAC52_C08 | 192 | 15q13.3 | 29655064 | 29727747 | 72683 |
| BAC228_H19 | 193 | 15q13.3 | 29668767 | 29783114 | 114347 |
| BAC147_A09 | 194 | 15q13.3 | 29692789 | 29784549 | 91760 |
| BAC89_K01 | 195 | 15q13.3 | 29698451 | 29790248 | 91797 |
| BAC247_I03 | 196 | 15q13.3 | 29721645 | 29796232 | 74587 |
| BAC175_A22 | 197 | 15q13.3 | 29863613 | 29946990 | 83377 |
| BAC230_B04 | 198 | 15q13.3 | 29961600 | 30061995 | 100395 |
| BAC177_J21 | 199 | 15q13.3 | 29991510 | 30080055 | 88545 |
| BAC59_F17 | 200 | 15q13.3 | 30061880 | 30165725 | 103845 |
| BAC229_A20 | 201 | 15q13.3 | 30077989 | 30158261 | 80272 |
| BAC151_L17 | 202 | 15q13.3 | 30173807 | 30295887 | 122080 |
| BAC238_I20 | 203 | 15q13.3 | 30205959 | 30293724 | 87765 |
| BAC129_F03 | 204 | 15q13.3 | 30205960 | 30308564 | 102604 |
| BAC41_A16 | 205 | 15q13.3 | 30237975 | 30348401 | 110426 |
| BAC202_J02 | 206 | 15q13.3 | 30404184 | 30508524 | 104340 |
| BAC101_D07 | 207 | 15q13.3 | 30488347 | 30638580 | 150233 |
| BAC158_C16 | 208 | 15q13.3 | 30511423 | 30599678 | 88255 |
| BAC191_N03 | 209 | 15q13.3 | 30511708 | 30602191 | 90483 |
| BAC232_D19 | 210 | 15q13.3 | 30550149 | 30665944 | 115795 |
| BAC248_H20 | 211 | 15q13.3 | 30635449 | 30730699 | 95250 |
| BAC114_L13 | 212 | 15q13.3 | 30712399 | 30815409 | 103010 |
| BAC27_K03 | 213 | 15q13.3 | 30730554 | 30827550 | 96996 |
| BAC132_K14 | 214 | 15q13.3 | 30737151 | 30815413 | 78262 |
| BAC120_D15 | 215 | 15q13.3 | 30948216 | 31038392 | 90176 |
| BAC19_K22 | 216 | 15q13.3 | 30965893 | 31069044 | 103151 |
| BAC122_N21 | 217 | 15q13.3 | 31135877 | 31222302 | 86425 |
| BAC218_O13 | 218 | 15q13.3 | 31200034 | 31283966 | 83932 |
| BAC111_F04 | 219 | 15q13.3 | 31205054 | 31302458 | 97404 |
| BAC53_P12 | 220 | 15q13.3 | 31222982 | 31308004 | 85022 |
| BAC19_B07 | 221 | 15q13.3 | 31226011 | 31344060 | 118049 |
| BAC81_N23 | 223 | 15q13.3 | 31257484 | 31390456 | 132972 |
| BAC82_D20 | 224 | 15q13.3- | 31315084 | 31436460 | 121376 |
| | | 15q14 | | | |
| BAC80_L05 | 225 | 15q13.3- | 31321681 | 31432069 | 110388 |
| | | 15q14 | | | |
| BAC248_J22 | 226 | 15q13.3- | 31343978 | 31436545 | 92567 |
| | | 15q14 | | | |
| BAC243_M11 | 227 | 15q14 | 31412580 | 31557020 | 144440 |
| BAC41_C03 | 228 | 15q14 | 31433811 | 31537139 | 103328 |
| BAC8_F11 | 229 | 15q14 | 31485307 | 31641345 | 156038 |
| BAC121_B19 | 230 | 15q14 | 31518311 | 31628605 | 110294 |
| BAC200_M14 | 231 | 15q14 | 31521493 | 31609110 | 87617 |
| BAC122_L19 | 232 | 15q14 | 31535224 | 31623920 | 88696 |
| BAC25_K09 | 233 | 15q14 | 31537793 | 31641337 | 103544 |
| BAC88_E07 | 234 | 15q14 | 31547025 | 31628579 | 81554 |
| BAC92_E24 | 235 | 15q14 | 31643325 | 31754603 | 111278 |
| BAC241_P21 | 236 | 15q14 | 31703121 | 31783313 | 80192 |
| BAC63_O23 | 237 | 15q14 | 31767146 | 31868250 | 101104 |
| BAC155_C12 | 238 | 15q14 | 31783240 | 31858316 | 75076 |
| BAC180_E02 | 239 | 15q14 | 31785499 | 31871450 | 85951 |
| BAC171_M10 | 240 | 15q14 | 31788229 | 31898892 | 110663 |
| BAC127_N19 | 241 | 15q14 | 31788290 | 31898829 | 110539 |
| BAC191_H15 | 242 | 15q14 | 31788306 | 31906494 | 118188 |
| BAC219_B07 | 243 | 15q14 | 31788306 | 31871450 | 83144 |
| BAC29_C06 | 245 | 15q14 | 31848958 | 31963408 | 114450 |
| BAC176_K23 | 246 | 15q14 | 31883016 | 31977550 | 94534 |
| BAC159_O02 | 247 | 15q14 | 31898700 | 32002677 | 103977 |
| BAC10_H19 | 248 | 15q14 | 31931369 | 32058653 | 127284 |
| BAC131_J16 | 249 | 15q14 | 31966330 | 31967483 | 1153 |
| BAC77_F03 | 250 | 15q14 | 32037899 | 32138533 | 100634 |
| BAC199_D21 | 251 | 15q14 | 32184973 | 32343104 | 158131 |
| BAC6_P15 | 252 | 15q14 | 32215774 | 32312591 | 96817 |
| BAC169_B03 | 253 | 15q14 | 32342893 | 32570241 | 227348 |
| BAC120_B13 | 254 | 15q14 | 32419599 | 32519598 | 99999 |
| BAC146_F05 | 255 | 15q14 | 32486870 | 32570171 | 83301 |
| BAC149_D20 | 256 | 15q14 | 32509069 | 32561571 | 52502 |
| BAC120_A10 | 257 | 15q14 | 32580020 | 32663436 | 83416 |
| BAC182_K03 | 258 | 15q14 | 32673895 | 32775455 | 101560 |
| BAC231_G05 | 259 | 15q14 | 32733528 | 32822895 | 89367 |
| BAC134_I22 | 260 | 15q14 | 32733723 | 32839348 | 105625 |
| BAC170_H09 | 261 | 15q14 | 32768081 | 32871486 | 103405 |
| BAC175_N11 | 262 | 15q14 | 32796541 | 32881878 | 85337 |
| BAC27_H01 | 263 | 15q14 | 32848753 | 32948522 | 99769 |
| BAC55_F01 | 264 | 15q14 | 32982887 | 33114343 | 131456 |
| BAC90_H23 | 265 | 15q14 | 32990625 | 33077539 | 86914 |
| BAC31_M04 | 266 | 15q14 | 33006609 | 33114342 | 107733 |
| BAC60_K11 | 267 | 15q14 | 33184047 | 33279272 | 95225 |
| BAC250_A10 | 268 | 15q14 | 33200731 | 33306934 | 106203 |
| BAC45_E18 | 269 | 15q14 | 33217881 | 33308444 | 90563 |
| BAC163_P19 | 270 | 15q14 | 33266803 | 33353740 | 86937 |
| BAC142_H23 | 271 | 15q14 | 33266826 | 33338983 | 72157 |
| BAC44_P10 | 272 | 15q14 | 33279169 | 33392053 | 112884 |
| BAC218_A23 | 273 | 15q14 | 33306785 | 33392053 | 85268 |
| BAC161_E23 | 274 | 15q14 | 33414002 | 33494070 | 80068 |
| BAC160_P12 | 275 | 15q14 | 33574426 | 33707079 | 132653 |
| BAC153_G01 | 276 | 15q14 | 33667443 | 33757713 | 90270 |
| BAC197_N21 | 278 | 15q14 | 33750051 | 33870988 | 120937 |
| BAC115_H22 | 279 | 15q14 | 33820741 | 33918720 | 97979 |
| BAC249_H05 | 280 | 15q14 | 33821244 | 33903301 | 82057 |
| BAC222_L11 | 281 | 15q14 | 33835171 | 33918692 | 83521 |
| BAC55_E13 | 282 | 15q14 | 33956760 | 33962431 | 5671 |
| BAC20_N09 | 283 | 15q14 | 34214543 | 34335503 | 120960 |
| BAC79_H24 | 284 | 15q14 | 34441601 | 34538530 | 96929 |
| BAC22_A19 | 285 | 15q14 | 34466660 | 34555808 | 89148 |
| BAC171_G19 | 286 | 15q14 | 34566082 | 34658669 | 92587 |
| BAC223_P15 | 287 | 15q14 | 34682676 | 34801907 | 119231 |
| BAC24_N04 | 288 | 15q14 | 34697013 | 34801917 | 104904 |
| BAC17_L11 | 289 | 15q14 | 34739458 | 34852919 | 113461 |
| BAC66_K21 | 290 | 15q14 | 34820291 | 34921791 | 101500 |
| BAC65_N23 | 291 | 15q14 | 34827464 | 35021252 | 193788 |
| BAC63_B21 | 292 | 15q14 | 34848521 | 34926999 | 78478 |
| BAC156_O16 | 293 | 15q14 | 34885392 | 35045333 | 159941 |
| BAC122_G14 | 294 | 15q14 | 34896335 | 34979136 | 82801 |
| BAC217_K06 | 295 | 15q14 | 34926842 | 35021258 | 94416 |
| BAC160_O17 | 296 | 15q14 | 34963703 | 35055599 | 91896 |
| BAC141_B17 | 297 | 15q14 | 35057616 | 35147076 | 89460 |
| BAC25_G07 | 298 | 15q14 | 35094074 | 35230543 | 136469 |
| BAC90_E02 | 299 | 15q14 | 35117069 | 35230557 | 113488 |
| BAC69_D07 | 300 | 15q14 | 35122961 | 35204471 | 81510 |
| BAC102_B15 | 301 | 15q14 | 35526554 | 35616960 | 90406 |
| BAC43_M07 | 302 | 15q14 | 35569726 | 35738635 | 168909 |
| BAC5_P23 | 303 | 15q14 | 35606122 | 35719573 | 113451 |
| BAC209_C08 | 304 | 15q14 | 35806454 | 35911436 | 104982 |
| BAC144_O16 | 305 | 15q14 | 35826271 | 35903109 | 76838 |
| BAC185_I12 | 306 | 15q14 | 35848662 | 35938309 | 89647 |
| BAC110_L08 | 307 | 15q14 | 36023853 | 36118467 | 94614 |
| BAC195_K03 | 308 | 15q14 | 36120105 | 36204585 | 84480 |
| BAC18_B05 | 309 | 15q14 | 36161393 | 36253344 | 91951 |
| BAC178_H21 | 310 | 15q14 | 36251437 | 36360038 | 108601 |
| BAC20_D10 | 311 | 15q14 | 36253281 | 36374645 | 121364 |
| BAC78_B12 | 312 | 15q14 | 36268977 | 36382680 | 113703 |
| BAC198_F21 | 313 | 15q14 | 36478468 | 36572168 | 93700 |
| BAC118_G22 | 314 | 15q14 | 36480885 | 36607004 | 126119 |
| BAC182_H19 | 315 | 15q14 | 36595183 | 36679416 | 84233 |
| BAC169_P23 | 316 | 15q14 | 36605683 | 36692826 | 87143 |
| BAC107_C14 | 317 | 15q14 | 36632563 | 36723093 | 90530 |
| BAC115_B12 | 318 | 15q14 | 36649432 | 36765705 | 116273 |
| BAC153_O07 | 319 | 15q14 | 36660865 | 36756316 | 95451 |
| BAC139_K19 | 320 | 15q14 | 36761310 | 36843001 | 81691 |
| BAC179_G22 | 321 | 15q14 | 36776220 | 36884319 | 108099 |

(13) Rubinstein-Taybi Syndrome

Rubinstein-taybi syndrome can be diagnosed for Karyotype deletion 16p13.3. A DNA chip for detecting Rubinstein-taybi syndrome includes a microarray containing at least one immobilized genomic DNA fragment selected from the group consisting of genomic DNA fragment 1 to 141 of Table 15, at least one oligomer of 20 to 100 bp consecutive nucleotide within the fragment, or at least one cDNA of 100 bp to 10 kbp derived from the fragment. When at least two kinds of probes are mixed and used, the probes can be mixed in equal proportions and immobilized in a single spot.

TABLE 15

| | | Chromosomal | Chromosomal | | |
|---|---|---|---|---|---|
| | | location | location in | | |
| | | identified | Human genome | | |
| BAC DNA | No. | by FISH | Start point | End point | size |
| BAC142_G12 | 1 | 16p13.3 | 516853 | 617469 | 100616 |
| BAC142_H12 | 2 | 16p13.3 | 516853 | 617469 | 100616 |
| BAC250_L07 | 3 | 16p13.3 | 605415 | 704070 | 98655 |
| BAC208_A15 | 4 | 16p13.3 | 720641 | 804644 | 84003 |
| BAC249_C21 | 5 | 16p13.3 | 734807 | 812203 | 77396 |
| BAC230_J12 | 6 | 16p13.3 | 734842 | 845667 | 110825 |
| BAC239_F18 | 7 | 16p13.3 | 834107 | 906859 | 72752 |
| BAC208_D07 | 8 | 16p13.3 | 863029 | 954010 | 90981 |
| BAC61_H17 | 9 | 16p13.3 | 979429 | 1055483 | 76054 |
| BAC35_O10 | 10 | 16p13.3 | 1137200 | 1256188 | 118988 |
| BAC78_M22 | 11 | 16p13.3 | 1256110 | 1355806 | 99696 |
| BAC68_D12 | 12 | 16p13.3 | 1409355 | 1501907 | 92552 |
| BAC109_L07 | 13 | 16p13.3 | 1424231 | 1540754 | 116523 |
| BAC8_E11 | 14 | 16p13.3 | 1424249 | 1525919 | 101670 |
| BAC248_B09 | 15 | 16p13.3 | 1540615 | 1646306 | 105691 |
| BAC175_F10 | 16 | 16p13.3 | 1561044 | 1690272 | 129228 |
| BAC247_K17 | 17 | 16p13.3 | 1561065 | 1679951 | 118886 |
| BAC205_E14 | 18 | 16p13.3 | 1561358 | 1660710 | 99352 |
| BAC247_B19 | 19 | 16p13.3 | 1561392 | 1673861 | 112469 |
| BAC30_J11 | 20 | 16p13.3 | 1563688 | 1646307 | 82619 |
| BAC204_J01 | 21 | 16p13.3 | 1563745 | 1693582 | 129837 |
| BAC160_N03 | 22 | 16p13.3 | 1563753 | 1680019 | 116266 |
| BAC43_H21 | 23 | 16p13.3 | 1594978 | 1673856 | 78878 |
| BAC60_D04 | 24 | 16p13.3 | 1646223 | 1750238 | 104015 |
| BAC101_E17 | 25 | 16p13.3 | 1646223 | 1801163 | 154940 |
| BAC26_C21 | 26 | 16p13.3 | 1660633 | 1801085 | 140452 |
| BAC161_M07 | 27 | 16p13.3 | 1693519 | 1820649 | 127130 |
| BAC208_N11 | 28 | 16p13.3 | 1750132 | 1831984 | 81852 |
| BAC32_K07 | 29 | 16p13.3 | 1840125 | 1953739 | 113614 |
| BAC133_O06 | 30 | 16p13.3 | 1848661 | 1928752 | 80091 |
| BAC124_D13 | 31 | 16p13.3 | 1883046 | 1970703 | 87657 |
| BAC252_I22 | 32 | 16p13.3 | 2066703 | 2157935 | 91232 |
| BAC40_G22 | 33 | 16p13.3 | 2066719 | 2152652 | 85933 |
| BAC53_J01 | 34 | 16p13.3 | 2076740 | 2196453 | 119713 |
| BAC21_B08 | 35 | 16p13.3 | 2076741 | 2156740 | 79999 |
| BAC116_P01 | 36 | 16p13.3 | 2156572 | 2269699 | 113127 |
| BAC184_B18 | 37 | 16p13.3 | 2196281 | 2311075 | 114794 |
| BAC221_H21 | 38 | 16p13.3 | 2260244 | 2352925 | 92681 |
| BAC144_E21 | 39 | 16p13.3 | 2269617 | 2357113 | 87496 |
| BAC124_A07 | 40 | 16p13.3 | 2270740 | 2352937 | 82197 |
| BAC153_B21 | 41 | 16p13.3 | 2328215 | 2410350 | 82135 |
| BAC71_B14 | 42 | 16p13.3 | 2374582 | 2496938 | 122356 |
| BAC236_I05 | 43 | 16p13.3 | 2400543 | 2491319 | 90776 |
| BAC153_A19 | 44 | 16p13.3 | 2405886 | 2513847 | 107961 |
| BAC140_F05 | 45 | 16p13.3 | 2496850 | 2685986 | 189136 |
| BAC150_A04 | 46 | 16p13.3 | 2858237 | 2947506 | 89269 |
| BAC40_D16 | 47 | 16p13.3 | 2931257 | 3032329 | 101072 |
| BAC67_A23 | 48 | 16p13.3 | 2936919 | 3022730 | 85811 |
| BAC109_H14 | 49 | 16p13.3 | 2972416 | 2975331 | 2915 |
| BAC188_G04 | 50 | 16p13.3 | 3022629 | 3105478 | 82849 |
| BAC155_O09 | 51 | 16p13.3 | 3023051 | 3182151 | 159100 |
| BAC144_H13 | 52 | 16p13.3 | 3032234 | 3148709 | 116475 |
| BAC158_C07 | 53 | 16p13.3 | 3032240 | 3105415 | 73175 |
| BAC59_B08 | 54 | 16p13.3 | 3119948 | 3203303 | 83355 |
| BAC84_C17 | 55 | 16p13.3 | 3178597 | 3261330 | 82733 |
| BAC177_J02 | 56 | 16p13.3 | 3179971 | 3281431 | 101460 |
| BAC193_J09 | 57 | 16p13.3 | 3196566 | 3283228 | 86662 |
| BAC136_D19 | 58 | 16p13.3 | 3202961 | 3301749 | 98788 |
| BAC60_M03 | 59 | 16p13.3 | 3283122 | 3376833 | 93711 |
| BAC49_J01 | 60 | 16p13.3 | 3291540 | 3426981 | 135441 |
| BAC23_K05 | 61 | 16p13.3 | 3348066 | 3454552 | 106486 |
| BAC102_D21 | 62 | 16p13.3 | 3496130 | 3589157 | 93027 |
| BAC24_F07 | 63 | 16p13.3 | 3533952 | 3635083 | 101131 |
| BAC178_J13 | 64 | 16p13.3 | 3546307 | 3652872 | 106565 |
| BAC65_A23 | 65 | 16p13.3 | 3558897 | 3676918 | 118021 |
| BAC171_C18 | 66 | 16p13.3 | 3588928 | 3676900 | 87972 |
| BAC19_P16 | 67 | 16p13.3 | 3617142 | 3721871 | 104729 |
| BAC200_A05 | 68 | 16p13.3 | 3662240 | 3764284 | 102044 |
| BAC8_E02 | 69 | 16p13.3 | 3676801 | 3808206 | 131405 |
| BAC176_N19 | 70 | 16p13.3 | 3676829 | 3777606 | 100777 |
| BAC175_E01 | 71 | 16p13.3 | 3721790 | 3808222 | 86432 |
| BAC243_O03 | 72 | 16p13.3 | 3764111 | 3900077 | 135966 |
| BAC77_O20 | 73 | 16p13.3 | 3764182 | 3869131 | 104949 |
| BAC33_C23 | 74 | 16p13.3 | 3799426 | 3925947 | 126521 |
| BAC33_F18 | 75 | 16p13.3 | 3802918 | 3900073 | 97155 |
| BAC19_O18 | 76 | 16p13.3 | 3966246 | 4073662 | 107416 |
| BAC65_O23 | 77 | 16p13.3 | 3970031 | 4109399 | 139368 |
| BAC229_B09 | 78 | 16p13.3 | 4003896 | 4170949 | 167053 |
| BAC151_N12 | 79 | 16p13.3 | 4218427 | 4321997 | 103570 |
| BAC153_I13 | 80 | 16p13.3 | 4218497 | 4321935 | 103438 |
| BAC72_C06 | 81 | 16p13.3 | 4220542 | 4330838 | 110296 |
| BAC45_N08 | 82 | 16p13.3 | 4266234 | 4362182 | 95948 |
| BAC168_G05 | 83 | 16p13.3 | 4362069 | 4480722 | 118653 |
| BAC42_O01 | 84 | 16p13.3 | 4396737 | 4495151 | 98414 |
| BAC156_J23 | 85 | 16p13.3 | 4425040 | 4567524 | 142484 |
| BAC234_H14 | 86 | 16p13.3 | 4445317 | 4533510 | 88193 |
| BAC235_L13 | 87 | 16p13.3 | 4480585 | 4567481 | 86896 |
| BAC10_M16 | 88 | 16p13.3 | 4508020 | 4607666 | 99646 |
| BAC211_B22 | 89 | 16p13.3 | 4508293 | 4627920 | 119627 |
| BAC210_A20 | 90 | 16p13.3 | 4533344 | 4621828 | 88484 |
| BAC196_B08 | 91 | 16p13.3 | 4533365 | 4627937 | 94572 |
| BAC224_L21 | 92 | 16p13.3 | 4586681 | 4730252 | 143571 |
| BAC134_C23 | 93 | 16p13.3 | 4607574 | 4752288 | 144714 |
| BAC119_O20 | 94 | 16p13.3 | 4621969 | 4714235 | 92266 |
| BAC140_K07 | 95 | 16p13.3 | 4621997 | 4733318 | 111321 |
| BAC142_C21 | 96 | 16p13.3 | 4621999 | 4733320 | 111321 |
| BAC167_N23 | 97 | 16p13.3 | 4663676 | 4805399 | 141723 |
| BAC55_A12 | 98 | 16p13.3 | 4677472 | 4752292 | 74820 |
| BAC44_A02 | 99 | 16p13.3 | 4714152 | 4839582 | 125430 |
| BAC101_M09 | 100 | 16p13.3 | 4775516 | 4872170 | 96654 |
| BAC198_G05 | 101 | 16p13.3 | 4819843 | 4916448 | 96605 |
| BAC109_N12 | 102 | 16p13.3 | 4907696 | 5003470 | 95774 |
| BAC170_K10 | 103 | 16p13.3 | 4911373 | 5005974 | 94601 |
| BAC226_C04 | 104 | 16p13.3 | 4951023 | 5036890 | 85867 |
| BAC2_E10 | 105 | 16p13.3 | 4951031 | 5036957 | 85926 |
| BAC120_L14 | 106 | 16p13.3 | 4962603 | 5051450 | 88847 |
| BAC218_E19 | 107 | 16p13.3 | 4980243 | 5082645 | 102402 |
| BAC51_I09 | 108 | 16p13.3 | 5012543 | 5120158 | 107615 |
| BAC2_E13 | 109 | 16p13.3 | 5044334 | 5192459 | 148125 |
| BAC190_H14 | 110 | 16p13.3 | 5068428 | 5209986 | 141558 |
| BAC171_K23 | 111 | 16p13.3 | 5090122 | 5207172 | 117050 |
| BAC244_M04 | 112 | 16p13.3 | 5101386 | 5193865 | 92479 |
| BAC36_C15 | 113 | 16p13.3 | 5196878 | 5329816 | 132938 |
| BAC24_D20 | 114 | 16p13.3 | 5391862 | 5556472 | 164610 |
| BAC19_E20 | 115 | 16p13.3 | 5465141 | 5543370 | 78229 |
| BAC14_H19 | 116 | 16p13.3 | 5555371 | 5699321 | 143950 |
| BAC114_K10 | 117 | 16p13.3 | 5556391 | 5631850 | 75459 |
| BAC184_G18 | 118 | 16p13.3 | 5566359 | 5668195 | 101836 |
| BAC64_C21 | 119 | 16p13.3 | 5613773 | 5714264 | 100491 |
| BAC234_M19 | 120 | 16p13.3 | 5631770 | 5714201 | 82431 |
| BAC96_I19 | 121 | 16p13.3 | 5699219 | 5790183 | 90964 |
| BAC90_K16 | 122 | 16p13.3 | 5753259 | 5875363 | 122104 |
| BAC58_J20 | 123 | 16p13.3 | 5768424 | 5894466 | 126042 |
| BAC31_C20 | 124 | 16p13.3 | 5769964 | 5864724 | 94760 |
| BAC120_O17 | 125 | 16p13.3 | 5928274 | 6026041 | 97767 |
| BAC208_M03 | 126 | 16p13.3 | 5971951 | 6086197 | 114246 |
| BAC144_L17 | 127 | 16p13.3 | 6002063 | 6086183 | 84120 |
| BAC59_H17 | 128 | 16p13.3 | 6061952 | 6182471 | 120519 |
| BAC177_H05 | 129 | 16p13.3 | 6151410 | 6254645 | 103235 |
| BAC145_J04 | 130 | 16p13.3 | 6174552 | 6272687 | 98135 |
| BAC58_M10 | 131 | 16p13.3- | 6298637 | 6409056 | 110419 |
| | | 16p13.2 | | | |
| BAC136_F13 | 132 | 16p13.3- | 6318127 | 6409104 | 90977 |
| | | 16p13.2 | | | |
| BAC97_A23 | 133 | 16p13.3- | 6388512 | 6464944 | 76432 |
| | | 16p13.2 | | | |
| BAC147_C23 | 134 | 16p13.2 | 6458270 | 6533019 | 74749 |
| BAC9_J02 | 135 | 16p13.2 | 6560276 | 6737905 | 177629 |
| BAC100_H02 | 136 | 16p13.2 | 6631478 | 6759323 | 127845 |
| BAC81_M09 | 137 | 16p13.2 | 6833738 | 6961166 | 127428 |
| BAC18_P05 | 138 | 16p13.2 | 6854156 | 6965698 | 111542 |
| BAC31_G11 | 139 | 16p13.2 | 6856529 | 6869084 | 12555 |
| BAC105_C09 | 140 | 16p13.2 | 6868931 | 6977422 | 108491 |
| BAC20_H18 | 141 | 16p13.2 | 6890490 | 6970337 | 79847 |

(14) Smith-Magenes Syndrome

Smith-magenes syndrome can be diagnosed for Karyotype deletion 17p11.2. A DNA chip for detecting Smith-magenes syndrome includes a microarray containing at least one immobilized genomic DNA fragment selected from the group consisting of genomic DNA fragment 1 to 165 of Table 16, at least one oligomer of 20 to 100 bp consecutive nucleotide within the fragment, or at least one cDNA of 100 bp to 10 kbp derived from the fragment. When at least two kinds of probes are mixed and used, the probes can be mixed in equal proportions and immobilized in a single spot.

TABLE 16

| | | Chromo- | | | |
|---|---|---|---|---|---|
| | | somal | Chromosomal | | |
| | | location | location in | | |
| | | identified | Human genome | | |
| BAC DNA | No. | by FISH | Start point | End point | size |
| BAC170_M14 | 1 | 17p12 | 14711135 | 14808289 | 97154 |
| BAC33_L21 | 2 | 17p12 | 14804500 | 14913795 | 109295 |
| BAC185_D08 | 3 | 17p12 | 14884357 | 14981705 | 97348 |
| BAC65_C19 | 4 | 17p12 | 14960572 | 15058918 | 98346 |
| BAC219_K23 | 5 | 17p12 | 14976175 | 15056221 | 80046 |
| BAC157_G01 | 6 | 17p12 | 15093558 | 15208862 | 115304 |
| BAC193_J12 | 7 | 17p12 | 15110864 | 15227229 | 116365 |
| BAC140_P21 | 8 | 17p12 | 15117703 | 15211297 | 93594 |
| BAC69_D20 | 9 | 17p12 | 15132146 | 15234625 | 102479 |
| BAC163_B05 | 10 | 17p12 | 15147290 | 15251664 | 104374 |
| BAC127_A13 | 11 | 17p12 | 15267749 | 15397538 | 129789 |
| BAC59_O13 | 12 | 17p12 | 15291315 | 15400674 | 109359 |
| BAC171_I03 | 13 | 17p12 | 15293504 | 15387860 | 94356 |
| BAC103_H17 | 14 | 17p12 | 15293548 | 15384732 | 91184 |
| BAC240_L04 | 15 | 17p12 | 15305705 | 15364092 | 58387 |
| BAC180_E05 | 16 | 17p12 | 15314834 | 15404065 | 89231 |
| BAC113_L01 | 17 | 17p12 | 15320794 | 15426185 | 105391 |
| BAC193_F10 | 18 | 17p12 | 15344377 | 15420149 | 75772 |
| BAC80_J24 | 19 | 17p12 | 15387712 | 15472607 | 84895 |
| BAC227_C12 | 20 | 17p12 | 15406530 | 15494411 | 87881 |
| BAC232_P15 | 21 | 17p12 | 15433058 | 15552649 | 119591 |
| BAC154_G20 | 22 | 17p12 | 15467403 | 15549983 | 82580 |
| BAC64_B16 | 23 | 17p12 | 15620998 | 15755383 | 134385 |
| BAC198_H06 | 24 | 17p12 | 15657045 | 15755442 | 98397 |
| BAC120_H23 | 25 | 17p12 | 15657050 | 15782155 | 125105 |
| BAC182_G04 | 26 | 17p12 | 15739595 | 15819054 | 79459 |
| BAC97_H12 | 27 | 17p12 | 15784582 | 15941885 | 157303 |
| BAC102_C09 | 28 | 17p12- | 15818957 | 20693695 | 4874738 |
| | | 17p11.2 | | | |
| BAC41_E16 | 29 | 17p12 | 15847215 | 15926899 | 79684 |
| BAC150_L13 | 30 | 17p12 | 15854845 | 15963018 | 108173 |
| BAC124_J04 | 31 | 17p12 | 15897252 | 15992971 | 95719 |
| BAC96_G24 | 32 | 17p12 | 15938321 | 16028412 | 90091 |
| BAC66_G13 | 33 | 17p12 | 15941785 | 16044299 | 102514 |
| BAC184_B05 | 34 | 17p12 | 15962872 | 16044301 | 81429 |
| BAC60_L08 | 35 | 17p12 | 15962873 | 16060283 | 97410 |
| BAC189_J17 | 36 | 17p12- | 16143400 | 16227881 | 84481 |
| | | 17p11.2 | | | |
| BAC88_H17 | 37 | 17p12- | 16143415 | 16236657 | 93242 |
| | | 17p11.2 | | | |
| BAC42_K11 | 38 | 17p11.2 | 16289956 | 16394927 | 104971 |
| BAC240_E14 | 39 | 17p11.2 | 16394845 | 16489073 | 94228 |
| BAC148_O18 | 40 | 17p11.2 | 16420254 | 16530913 | 110659 |
| BAC125_N20 | 41 | 17p11.2 | 16557887 | 16711927 | 154040 |
| BAC175_H10 | 42 | 17p11.2 | 16557890 | 16636429 | 78539 |
| BAC146_D01 | 43 | 17p11.2 | 16557904 | 16636434 | 78530 |
| BAC62_D24 | 44 | 17p11.2 | 16654529 | 16751004 | 96475 |
| BAC186_K22 | 45 | 17p11.2 | 16724226 | 16796771 | 72545 |
| BAC33_P23 | 46 | 17p11.2 | 16779978 | 16866784 | 86806 |
| BAC14_G14 | 47 | 17p11.2 | 16796638 | 16913606 | 116968 |
| BAC72_P14 | 48 | 17p11.2 | 16866681 | 16988595 | 121914 |
| BAC131_K12 | 49 | 17p11.2 | 16909687 | 16990173 | 80486 |
| BAC197_O11 | 50 | 17p11.2 | 17025266 | 17165284 | 140018 |
| BAC26_I07 | 51 | 17p11.2 | 17025339 | 17091291 | 65952 |
| BAC36_F12 | 52 | 17p11.2 | 17074074 | 17165053 | 90979 |
| BAC129_D23 | 53 | 17p11.2 | 17158746 | 17245353 | 86607 |
| BAC101_K17 | 54 | 17p11.2 | 17245267 | 17333785 | 88518 |
| BAC123_D23 | 55 | 17p11.2 | 17245267 | 17357212 | 111945 |
| BAC89_C18 | 56 | 17p11.2 | 17276220 | 17391839 | 115619 |
| BAC90_H16 | 57 | 17p11.2 | 17283727 | 17383307 | 99580 |
| BAC198_N13 | 58 | 17p11.2 | 17283752 | 17390460 | 106708 |
| BAC154_C08 | 59 | 17p11.2 | 17295654 | 17419340 | 123686 |
| BAC179_F21 | 60 | 17p11.2 | 17324665 | 17419329 | 94664 |
| BAC189_N01 | 61 | 17p11.2 | 17390295 | 17465331 | 75036 |
| BAC20_P07 | 62 | 17p11.2 | 17419240 | 17530721 | 111481 |
| BAC167_A18 | 63 | 17p11.2 | 17419255 | 17572653 | 153398 |
| BAC95_I12 | 64 | 17p11.2 | 17482800 | 17572594 | 89794 |
| BAC155_B06 | 65 | 17p11.2 | 17484086 | 17572609 | 88523 |
| BAC56_J17 | 66 | 17p11.2 | 17551815 | 17665243 | 113428 |
| BAC36_C18 | 67 | 17p11.2 | 17579594 | 17676682 | 97088 |
| BAC170_M10 | 68 | 17p11.2 | 17588731 | 17678229 | 89498 |
| BAC236_B14 | 69 | 17p11.2 | 17698446 | 17776240 | 77794 |
| BAC76_J10 | 70 | 17p11.2 | 17709665 | 17806257 | 96592 |
| BAC165_E09 | 71 | 17p11.2 | 17723644 | 17806266 | 82622 |
| BAC249_G12 | 72 | 17p11.2 | 17776414 | 17890030 | 113616 |
| BAC41_D18 | 73 | 17p11.2 | 17889883 | 18025228 | 135345 |
| BAC131_M11 | 74 | 17p11.2 | 17916386 | 17992329 | 75943 |
| BAC103_D11 | 75 | 17p11.2 | 17935441 | 18032930 | 97489 |
| BAC57_N06 | 76 | 17p11.2 | 18030897 | 18165192 | 134295 |
| BAC197_E01 | 77 | 17p11.2 | 18044616 | 18128976 | 84360 |
| BAC20_D12 | 78 | 17p11.2 | 18128850 | 18219224 | 90374 |
| BAC238_N13 | 79 | 17p11.2 | 18165108 | 18259978 | 94870 |
| BAC57_C22 | 80 | 17p11.2 | 18202515 | 18354860 | 152345 |
| BAC184_A04 | 81 | 17p11.2 | 18206784 | 18295425 | 88641 |
| BAC70_D14 | 82 | 17p11.2 | 18206800 | 18295476 | 88676 |
| BAC186_E14 | 83 | 17p11.2 | 18332672 | 18452458 | 119786 |
| BAC97_I20 | 84 | 17p11.2 | 18393829 | 18513020 | 119191 |
| BAC199_D08 | 85 | 17p11.2 | 18403056 | 18513020 | 109964 |
| BAC154_P06 | 86 | 17p11.2 | 18421956 | 18508299 | 86343 |
| BAC39_C07 | 87 | 17p11.2 | 18507215 | 18613862 | 106647 |
| BAC228_C10 | 88 | 17p11.2 | 18594104 | 18712014 | 117910 |
| BAC81_H03 | 89 | 17p11.2 | 18594109 | 18726670 | 132561 |
| BAC58_P21 | 90 | 17p11.2 | 18635594 | 18755396 | 119802 |
| BAC233_A12 | 91 | 17p11.2 | 18737655 | 18849936 | 112281 |
| BAC153_J19 | 92 | 17p11.2 | 18746164 | 18831879 | 85715 |
| BAC189_M02 | 93 | 17p11.2 | 18773717 | 18895449 | 121732 |
| BAC132_A14 | 94 | 17p11.2 | 18805385 | 18918916 | 113531 |
| BAC95_F14 | 95 | 17p11.2 | 18807265 | 18895443 | 88178 |
| BAC191_B21 | 96 | 17p11.2 | 18808618 | 18884963 | 76345 |
| BAC132_L20 | 97 | 17p11.2 | 18811539 | 18902037 | 90498 |
| BAC232_D17 | 98 | 17p11.2 | 18812507 | 18903860 | 91353 |
| BAC77_C07 | 99 | 17p11.2 | 18831796 | 18927011 | 95215 |
| BAC61_M05 | 100 | 17p11.2 | 18845354 | 19050538 | 205184 |
| BAC57_D11 | 101 | 17p11.2 | 18897566 | 19000464 | 102898 |
| BAC77_B01 | 102 | 17p11.2 | 18952608 | 19072845 | 120237 |
| BAC84_B04 | 103 | 17p11.2 | 19079201 | 19218785 | 139584 |
| BAC160_J02 | 104 | 17p11.2 | 19225463 | 19266078 | 40615 |
| BAC109_F04 | 105 | 17p11.2 | 19244444 | 19304469 | 60025 |
| BAC31_F09 | 106 | 17p11.2 | 19244499 | 19310543 | 66044 |
| BAC54_L12 | 107 | 17p11.2 | 19244500 | 19313051 | 68551 |
| BAC183_B05 | 108 | 17p11.2 | 19265193 | 19337891 | 72698 |
| BAC97_K11 | 109 | 17p11.2 | 19313826 | 19387684 | 73858 |
| BAC148_B12 | 110 | 17p11.2 | 19402824 | 19492063 | 89239 |
| BAC26_B11 | 111 | 17p11.2 | 19526736 | 19640708 | 113972 |
| BAC12_C18 | 112 | 17p11.2 | 19534378 | 19640758 | 106380 |
| BAC92_D22 | 113 | 17p11.2 | 19534392 | 19651908 | 117516 |
| BAC69_L23 | 114 | 17p11.2 | 19545442 | 19651910 | 106468 |
| BAC175_P18 | 115 | 17p11.2 | 19563692 | 19648388 | 84696 |
| BAC146_B24 | 116 | 17p11.2 | 19650991 | 19746822 | 95831 |
| BAC27_K06 | 117 | 17p11.2 | 19651809 | 19749152 | 97343 |
| BAC70_N14 | 118 | 17p11.2 | 19665863 | 19786182 | 120319 |
| BAC171_D20 | 119 | 17p11.2 | 19680409 | 19788794 | 108385 |
| BAC116_O21 | 120 | 17p11.2 | 19718981 | 19786183 | 67202 |
| BAC10_I20 | 121 | 17p11.2 | 19739497 | 19816424 | 76927 |
| BAC96_I07 | 122 | 17p11.2 | 19816311 | 19903394 | 87083 |
| BAC249_G09 | 123 | 17p11.2 | 19867726 | 19989856 | 122130 |
| BAC44_J06 | 124 | 17p11.2 | 19891394 | 19971811 | 80417 |
| BAC193_P10 | 125 | 17p11.2 | 19926766 | 20039699 | 112933 |
| BAC156_C17 | 126 | 17p11.2 | 20083455 | 20215023 | 131568 |
| BAC36_G17 | 127 | 17p11.2 | 20083487 | 20218743 | 135256 |
| BAC11_J06 | 128 | 17p11.2 | 20193464 | 2028176 | 88297 |
| BAC124_E01 | 129 | 17p11.2 | 20228503 | 20332288 | 103785 |
| BAC117_D16 | 130 | 17p11.2 | 20270202 | 20378561 | 108359 |
| BAC188_P16 | 131 | 17p11.2 | 20340291 | 20436954 | 96663 |
| BAC233_L09 | 132 | 17p11.2 | 20457471 | 20549617 | 92146 |
| BAC20_M05 | 133 | 17p11.2 | 20457497 | 20526040 | 68543 |
| BAC69_F02 | 134 | 17p11.2 | 20477768 | 20580390 | 102622 |
| BAC237_C22 | 135 | 17p11.2 | 20499729 | 20600363 | 100634 |
| BAC60_H06 | 136 | 17p11.2 | 20503572 | 20599301 | 95729 |
| BAC141_A08 | 137 | 17p11.2 | 20511752 | 20596573 | 84821 |
| BAC40_H06 | 138 | 17p11.2 | 20525954 | 20630471 | 104517 |
| BAC142_P01 | 139 | 17p11.2 | 20596491 | 20693726 | 97235 |
| BAC48_M08 | 140 | 17p11.2 | 20706155 | 20795955 | 89800 |
| BAC223_H04 | 141 | 17p11.2 | 20734592 | 20851649 | 117057 |
| BAC195_E10 | 142 | 17p11.2 | 20769887 | 20847051 | 77164 |
| BAC200_J14 | 143 | 17p11.2 | 20811622 | 20895723 | 84101 |
| BAC223_I12 | 144 | 17p11.2 | 20811641 | 20905322 | 93681 |
| BAC28_P01 | 145 | 17p11.2 | 20851520 | 20989326 | 137806 |
| BAC119_A16 | 146 | 17p11.2 | 20955668 | 21104977 | 149309 |
| BAC4_G22 | 147 | 17p11.2 | 21004734 | 21099992 | 95258 |
| BAC118_H17 | 148 | 17p11.2 | 21017214 | 21121333 | 104119 |
| BAC40_J07 | 149 | 17p11.2 | 21028143 | 21121354 | 93211 |
| BAC239_L05 | 150 | 17p11.2 | 21036358 | 21175468 | 139110 |
| BAC32_C06 | 151 | 17p11.2 | 21134931 | 21313722 | 178791 |
| BAC248_A23 | 152 | 17p11.2 | 21214133 | 21296062 | 81929 |
| BAC142_A03 | 153 | 17p11.2 | 21214197 | 21315277 | 101080 |
| BAC61_I13 | 154 | 17p11.2 | 21277948 | 21385816 | 107868 |
| BAC236_B19 | 155 | 17p11.2 | 21315182 | 21425247 | 110065 |
| BAC100_J04 | 156 | 17p11.2 | 21393550 | 21547843 | 154293 |
| BAC139_B06 | 157 | 17p11.2- | 21411493 | 25423821 | 4012328 |
| | | 17q11.1 | | | |
| BAC130_E22 | 158 | 17p11.2 | 21425175 | 21493695 | 68520 |
| BAC68_G08 | 159 | 17p11.2 | 21458460 | 21552363 | 93903 |
| BAC26_L13 | 160 | 17p11.2 | 21843173 | 21970661 | 127488 |
| BAC118_E14 | 161 | 17p11.2 | 21953893 | 22050818 | 96925 |
| BAC236_B22 | 162 | 17p11.2 | 22031416 | 22050802 | 19386 |
| BAC81_F21 | 163 | 17p11.2 | 22050715 | 22087819 | 37104 |
| BAC168_D15 | 164 | 17p11.2 | 22050731 | 22088509 | 37778 |
| BAC199_H24 | 165 | 17p11.2 | 22090493 | 22173463 | 82970 |

(15) Williams Syndrome

Williams syndrome can be diagnosed for Karyotype 7q11.2. A DNA chip for detecting Williams syndrome includes a microarray containing at least one immobilized genomic DNA fragment selected from the group consisting of genomic DNA fragment 1 to 282 of Table 17, at least one oligomer of 20 to 100 bp consecutive nucleotide within the fragment, or at least one cDNA of 100 bp to 10 kbp derived from the fragment. When at least two kinds of probes are mixed and used, the probes can be mixed in equal proportions and immobilized in a single spot.

TABLE 17

| | | Chromo- | | | |
|---|---|---|---|---|---|
| | | somal | Chromosomal | | |
| | | location | location in Human | | |
| | | identified | genome | | |
| BAC DNA | No. | by FISH | Start point | End point | size |
| BAC31_J17 | 1 | 7p11.21 | 61898867 | 62012925 | 114058 |
| BAC230_H11 | 2 | 7q11.21 | 61906746 | 62006391 | 99645 |
| BAC219_N06 | 3 | 7q11.21 | 62003557 | 62086976 | 83419 |
| BAC125_M01 | 4 | 7q11.21 | 62006308 | 62089408 | 83100 |
| BAC79_J10 | 5 | 7q11.21 | 62006309 | 62089407 | 83098 |
| BAC235_C03 | 6 | 7q11.21 | 62487299 | 62647481 | 160182 |
| BAC161_E01 | 7 | 7q11.21 | 62511674 | 62617299 | 105625 |
| BAC146_C03 | 8 | 7q11.21 | 62558809 | 62692316 | 133507 |
| BAC197_C09 | 9 | 7q11.21 | 62617209 | 62709653 | 92444 |
| BAC177_O21 | 10 | 7q11.21 | 62667885 | 62779586 | 111701 |
| BAC46_C20 | 11 | 7q11.21 | 62692225 | 62710922 | 18697 |
| BAC119_P24 | 12 | 7q11.21 | 62735353 | 62826753 | 91400 |
| BAC82_I06 | 13 | 7q11.21 | 63089309 | 63177668 | 88359 |
| BAC46_M19 | 14 | 7q11.21 | 63214056 | 63300526 | 86470 |
| BAC69_D08 | 15 | 7q11.21 | 63342168 | 63460174 | 118006 |
| BAC198_P18 | 16 | 7q11.21 | 63397374 | 63490335 | 92961 |
| BAC51_G10 | 17 | 7q11.21 | 63470602 | 63580492 | 109890 |
| BAC238_P05 | 18 | 7q11.21 | 63561375 | 63639888 | 78513 |
| BAC53_G18 | 19 | 7q11.21 | 63661454 | 63757857 | 96403 |
| BAC123_F15 | 20 | 7q11.21 | 63720133 | 63817862 | 97729 |
| BAC237_F05 | 21 | 7q11.21 | 63811783 | 63907144 | 95361 |
| BAC191_G16 | 22 | 7q11.21 | 63865238 | 63955867 | 90629 |
| BAC81_I18 | 23 | 7q11.21 | 63972748 | 64116723 | 143975 |
| BAC84_I14 | 24 | 7q11.21 | 63983037 | 64116724 | 133687 |
| BAC148_E09 | 25 | 7q11.21 | 64116617 | 64231528 | 114911 |
| BAC232_H16 | 26 | 7q11.21 | 64133870 | 64212364 | 78494 |
| BAC238_K05 | 27 | 7q11.21 | 64139129 | 64236855 | 97726 |
| BAC46_K09 | 28 | 7q11.21 | 64139131 | 64267840 | 128709 |
| BAC187_J19 | 29 | 7q11.21 | 64142063 | 64231511 | 89448 |
| BAC242_C06 | 30 | 7q11.21 | 64386614 | 64461703 | 75089 |
| BAC125_G24 | 31 | 7q11.21 | 64612386 | 64616485 | 4099 |
| BAC126_I06 | 32 | 7q11.21 | 64691631 | 64776208 | 84577 |
| BAC146_M01 | 33 | 7q11.21 | 64832971 | 64931155 | 98184 |
| BAC46_D05 | 34 | 7q11.21 | 64845556 | 64929573 | 84017 |
| BAC36_G09 | 35 | 7q11.21 | 64859927 | 64958456 | 98529 |
| BAC76_D12 | 36 | 7q11.21 | 64859943 | 64984074 | 124131 |
| BAC36_D03 | 37 | 7q11.21 | 64947598 | 65051538 | 103940 |
| BAC97_F16 | 38 | 7q11.21 | 65047197 | 65140959 | 93762 |
| BAC161_J18 | 39 | 7q11.21 | 65123430 | 65219021 | 95591 |
| BAC208_N14 | 40 | 7q11.21 | 65314771 | 65415296 | 100525 |
| BAC165_N08 | 41 | 7q11.21 | 65321478 | 65424083 | 102605 |
| BAC117_G15 | 42 | 7q11.21 | 65327111 | 65424151 | 97040 |
| BAC61_A07 | 43 | 7q11.21 | 65335669 | 65430762 | 95093 |
| BAC217_M14 | 44 | 7q11.21 | 65339164 | 65430744 | 91580 |
| BAC148_M14 | 45 | 7q11.21 | 65424001 | 65526472 | 102471 |
| BAC83_P10 | 46 | 7q11.21 | 65483170 | 65601991 | 118821 |
| BAC69_M13 | 47 | 7q11.21 | 65497300 | 65581852 | 84552 |
| BAC40_L06 | 48 | 7q11.21 | 65661295 | 65743736 | 82441 |
| BAC51_E12 | 49 | 7q11.21 | 65665572 | 65766437 | 100865 |
| BAC227_L03 | 50 | 7q11.21 | 65682942 | 65766456 | 83514 |
| BAC160_B04 | 51 | 7q11.21 | 65687456 | 65783744 | 96288 |
| BAC51_F07 | 52 | 7q11.21 | 65693948 | 65761645 | 67697 |
| BAC117_P20 | 53 | 7q11.21 | 65783640 | 65902101 | 118461 |
| BAC33_O12 | 54 | 7q11.21 | 65973887 | 66086068 | 112181 |
| BAC76_F19 | 55 | 7q11.21 | 66011777 | 66105693 | 93916 |
| BAC215_P15 | 56 | 7q11.21 | 66011784 | 66105710 | 93926 |
| BAC120_H09 | 57 | 7q11.21 | 66011790 | 66111430 | 99640 |
| BAC222_C15 | 58 | 7q11.21 | 66058776 | 66191269 | 132493 |
| BAC57_L05 | 59 | 7q11.21 | 66072861 | 66163678 | 90817 |
| BAC228_B08 | 60 | 7q11.21 | 66105630 | 66191263 | 85633 |
| BAC188_G06 | 61 | 7q11.21 | 66184302 | 66265387 | 81085 |
| BAC8_H16 | 62 | 7q11.21 | 66220391 | 66345347 | 124956 |
| BAC78_E05 | 63 | 7q11.21- | 66381145 | 66474772 | 93627 |
| | | 7q11.22 | | | |
| BAC128_D08 | 64 | 7q11.21- | 66385404 | 66498401 | 112997 |
| | | 7q11.22 | | | |
| BAC139_E14 | 65 | 7q11.22 | 66419377 | 66519730 | 100353 |
| BAC107_N11 | 66 | 7q11.22 | 66425765 | 66519770 | 94005 |
| BAC161_L18 | 67 | 7q11.22 | 66428705 | 66547804 | 119099 |
| BAC128_J19 | 68 | 7q11.22 | 66482696 | 66519724 | 37028 |
| BAC82_O01 | 69 | 7q11.22 | 66538181 | 66649838 | 111657 |
| BAC133_O21 | 70 | 7q11.22 | 66547636 | 66649838 | 102202 |
| BAC169_F07 | 71 | 7q11.22 | 66567777 | 66653434 | 85657 |
| BAC158_L15 | 72 | 7q11.22 | 66567794 | 66652656 | 84862 |
| BAC218_O12 | 73 | 7q11.22 | 66571433 | 66675671 | 104238 |
| BAC90_G14 | 74 | 7q11.22 | 66571507 | 66671877 | 100370 |
| BAC236_L05 | 75 | 7q11.22 | 66571508 | 66675690 | 104182 |
| BAC20_H23 | 76 | 7q11.22 | 66649755 | 66774265 | 124510 |
| BAC137_C21 | 77 | 7q11.22 | 66675592 | 66783002 | 107410 |
| BAC8_M13 | 78 | 7q11.22 | 66675861 | 66754868 | 79007 |
| BAC218_J15 | 79 | 7q11.22 | 66679852 | 66781819 | 101967 |
| BAC143_J14 | 80 | 7q11.22 | 66679870 | 66741320 | 61450 |
| BAC46_C16 | 81 | 7q11.22 | 66681514 | 66782959 | 101445 |
| BAC6_P01 | 82 | 7q11.22 | 66779170 | 66870305 | 91135 |
| BAC125_N03 | 83 | 7q11.22 | 66781730 | 66964103 | 182373 |
| BAC65_P16 | 84 | 7q11.22 | 66786033 | 66902487 | 116454 |
| BAC196_F09 | 85 | 7q11.22 | 66806226 | 66913966 | 107740 |
| BAC179_A04 | 86 | 7q11.22 | 66852513 | 66961324 | 108811 |
| BAC106_D06 | 87 | 7q11.22 | 66909209 | 67024230 | 115021 |
| BAC102_M12 | 88 | 7q11.22 | 66996088 | 67082986 | 86898 |
| BAC183_D14 | 89 | 7q11.22 | 67028243 | 67136491 | 108248 |
| BAC5_H21 | 90 | 7q11.22 | 67096745 | 67191848 | 95103 |
| BAC8_O09 | 91 | 7q11.22 | 67096797 | 67187070 | 90273 |
| BAC10_G24 | 92 | 7q11.22 | 67096813 | 67188483 | 91670 |
| BAC142_I04 | 93 | 7q11.22 | 67096814 | 67215599 | 118785 |
| BAC187_G12 | 94 | 7q11.22 | 67096815 | 67182447 | 85632 |
| BAC150_J10 | 95 | 7q11.22 | 67108468 | 67215062 | 106594 |
| BAC105_G18 | 96 | 7q11.22 | 67176631 | 67268601 | 91970 |
| BAC11_N14 | 97 | 7q11.22 | 67188385 | 67287651 | 99266 |
| BAC15_K14 | 98 | 7q11.22 | 67196557 | 67284608 | 88051 |
| BAC163_N06 | 99 | 7q11.22 | 67239919 | 67324569 | 84650 |
| BAC112_A03 | 100 | 7q11.22 | 67324470 | 67414505 | 90035 |
| BAC145_I01 | 101 | 7q11.22 | 67359985 | 67452933 | 92948 |
| BAC59_I15 | 102 | 7q11.22 | 67359992 | 67501976 | 141984 |
| BAC241_N17 | 103 | 7q11.22 | 67360068 | 67460666 | 100598 |
| BAC155_E02 | 104 | 7q11.22 | 67397364 | 67479156 | 81792 |
| BAC60_C01 | 105 | 7q11.22 | 67419827 | 67554428 | 134601 |
| BAC181_C20 | 106 | 7q11.22 | 67450534 | 67554416 | 103882 |
| BAC91_G02 | 107 | 7q11.22 | 67586010 | 67715678 | 129668 |
| BAC97_O08 | 108 | 7q11.22 | 67614688 | 67732468 | 117780 |
| BAC109_F03 | 109 | 7q11.22 | 67618846 | 67700997 | 82151 |
| BAC168_J05 | 110 | 7q11.22 | 67651787 | 67740246 | 88459 |
| BAC220_B16 | 111 | 7q11.22 | 67651806 | 67750446 | 98640 |
| BAC176_G04 | 112 | 7q11.22 | 67704736 | 67786763 | 82027 |
| BAC145_L15 | 113 | 7q11.22 | 67764969 | 67850122 | 85153 |
| BAC179_H12 | 114 | 7q11.22 | 67843432 | 67934824 | 91392 |
| BAC55_N01 | 115 | 7q11.22 | 67881652 | 68016895 | 135243 |
| BAC231_H05 | 116 | 7q11.22 | 67883341 | 67998165 | 114824 |
| BAC229_E01 | 117 | 7q11.22 | 68030299 | 68108688 | 78389 |
| BAC75_E21 | 118 | 7q11.22 | 68067926 | 68157791 | 89865 |
| BAC150_O19 | 119 | 7q11.22 | 68075993 | 68192194 | 116201 |
| BAC149_J07 | 120 | 7q11.22 | 68186683 | 68277910 | 91227 |
| BAC142_F05 | 121 | 7q11.22 | 68192613 | 68321544 | 128931 |
| BAC141_C14 | 122 | 7q11.22 | 68245389 | 68351355 | 105966 |
| BAC26_B01 | 123 | 7q11.22 | 68261556 | 68402043 | 140487 |
| BAC248_N10 | 124 | 7q11.22 | 68261573 | 68367428 | 105855 |
| BAC27_K23 | 125 | 7q11.22 | 68395665 | 68486457 | 90792 |
| BAC204_C09 | 126 | 7q11.22 | 68410976 | 68524825 | 113849 |
| BAC229_J11 | 127 | 7q11.22 | 68583965 | 68671049 | 87084 |
| BAC151_F05 | 128 | 7q11.22 | 68616553 | 68703953 | 87400 |
| BAC190_H05 | 129 | 7q11.22 | 68630518 | 68715303 | 84785 |
| BAC250_I08 | 130 | 7q11.22 | 68754331 | 68837945 | 83614 |
| BAC186_P01 | 131 | 7q11.22 | 68761724 | 68846953 | 85229 |
| BAC237_B12 | 132 | 7q11.22 | 68875698 | 68954649 | 78951 |
| BAC57_M19 | 133 | 7q11.22 | 68887977 | 68972036 | 84059 |
| BAC26_L02 | 134 | 7q11.22 | 68891457 | 68991327 | 99870 |
| BAC228_C20 | 135 | 7q11.22 | 68903883 | 69005715 | 101832 |
| BAC8_K08 | 136 | 7q11.22 | 68949318 | 69049224 | 99906 |
| BAC134_D18 | 137 | 7q11.22 | 69005625 | 69083078 | 77453 |
| BAC247_N11 | 138 | 7q11.22 | 69065619 | 69168844 | 103225 |
| BAC124_P19 | 139 | 7q11.22 | 69189537 | 69310307 | 120770 |
| BAC149_C07 | 140 | 7q11.22 | 69222112 | 69329243 | 107131 |
| BAC119_K16 | 141 | 7q11.22 | 69255997 | 69368282 | 112285 |
| BAC5_I07 | 142 | 7q11.22 | 69258730 | 69354974 | 96244 |
| BAC144_M09 | 143 | 7q11.22 | 69265620 | 69363324 | 97704 |
| BAC36_K10 | 144 | 7q11.22 | 69281030 | 69371351 | 90321 |
| BAC152_A19 | 145 | 7q11.22 | 69354771 | 69426779 | 72008 |
| BAC127_B02 | 146 | 7q11.22 | 69367957 | 69460310 | 92353 |
| BAC161_C16 | 147 | 7q11.22 | 69456694 | 69536435 | 79741 |
| BAC121_P11 | 148 | 7q11.22 | 69484783 | 69601538 | 116755 |
| BAC146_N11 | 149 | 7q11.22 | 69505354 | 69591913 | 86559 |
| BAC33_D12 | 150 | 7q11.22 | 69620195 | 69783847 | 163652 |
| BAC197_O10 | 151 | 7q11.22 | 69661519 | 69770910 | 109391 |
| BAC219_J03 | 152 | 7q11.22 | 69696624 | 69800842 | 104218 |
| BAC88_O22 | 153 | 7q11.22 | 69730954 | 69811102 | 80148 |
| BAC80_D02 | 154 | 7q11.22 | 69758496 | 69863737 | 105241 |
| BAC24_C16 | 155 | 7q11.22 | 69770808 | 69877770 | 106962 |
| BAC13_P23 | 156 | 7q11.22 | 69978188 | 70092291 | 114103 |
| BAC45_A14 | 157 | 7q11.22 | 69978242 | 70073209 | 94967 |
| BAC241_B22 | 158 | 7q11.22 | 69984695 | 70170914 | 186219 |
| BAC73_I10 | 159 | 7q11.22 | 70036420 | 70170938 | 134518 |
| BAC67_M22 | 160 | 7q11.22 | 70047697 | 70183032 | 135335 |
| BAC116_F24 | 161 | 7q11.22 | 70073050 | 70172097 | 99047 |
| BAC118_I21 | 162 | 7q11.22 | 70073115 | 70160635 | 87520 |
| BAC36_C22 | 163 | 7q11.22 | 70123932 | 70312645 | 188713 |
| BAC38_P24 | 164 | 7q11.22 | 70181209 | 70286515 | 105306 |
| BAC110_N02 | 165 | 7q11.22 | 70181223 | 70280310 | 99087 |
| BAC220_A08 | 166 | 7q11.22 | 70303416 | 70392561 | 89145 |
| BAC13_H20 | 167 | 7q11.22 | 70319739 | 70415660 | 95921 |
| BAC175_B13 | 168 | 7q11.22 | 70356455 | 70471013 | 114558 |
| BAC88_E11 | 169 | 7q11.22 | 70356468 | 70434511 | 78043 |
| BAC140_A08 | 170 | 7q11.22 | 70384910 | 70471030 | 86120 |
| BAC127_L05 | 171 | 7q11.22 | 70444120 | 70506602 | 62482 |
| BAC150_H21 | 172 | 7q11.22 | 70527282 | 70612550 | 85268 |
| BAC195_E21 | 173 | 7q11.22 | 70549601 | 70659056 | 109455 |
| BAC167_H24 | 174 | 7q11.22 | 70559455 | 70645623 | 86168 |
| BAC76_D15 | 175 | 7q11.22 | 70559526 | 70658999 | 99473 |
| BAC192_B09 | 176 | 7q11.22 | 70559541 | 70674033 | 114492 |
| BAC35_K16 | 177 | 7q11.22 | 70645272 | 70780823 | 135551 |
| BAC106_C16 | 178 | 7q11.22 | 70645272 | 70774345 | 129073 |
| BAC100_L06 | 179 | 7q11.22 | 70663949 | 70763379 | 99430 |
| BAC125_P12 | 180 | 7q11.22 | 70673930 | 70775142 | 101212 |
| BAC40_E19 | 181 | 7q11.22 | 70797107 | 70910130 | 113023 |
| BAC248_E16 | 182 | 7q11.22 | 70803310 | 70876515 | 73205 |
| BAC41_A17 | 183 | 7q11.22 | 70810148 | 70934423 | 124275 |
| BAC84_O12 | 184 | 7q11.22 | 70859891 | 70980889 | 120998 |
| BAC57_K15 | 185 | 7q11.22 | 70909989 | 71015621 | 105632 |
| BAC26_B04 | 186 | 7q11.22 | 70955187 | 71066589 | 111402 |
| BAC168_L23 | 187 | 7q11.22 | 70959661 | 71043294 | 83633 |
| BAC33_H06 | 189 | 7q11.22 | 71104722 | 71198792 | 94070 |
| BAC53_K11 | 190 | 7q11.22 | 71241480 | 71435280 | 193800 |
| BAC104_O23 | 191 | 7q11.22 | 71266522 | 71380695 | 114173 |
| BAC173_B21 | 192 | 7q11.22 | 71380529 | 71472255 | 91726 |
| BAC73_E21 | 193 | 7q11.22 | 71410817 | 71548080 | 137263 |
| BAC38_A17 | 194 | 7q11.22 | 71481704 | 71582665 | 100961 |
| BAC82_A21 | 195 | 7q11.22- | 71554112 | 71664069 | 109957 |
| | | 7q11.23 | | | |
| BAC127_E09 | 196 | 7q11.23 | 71620043 | 71708632 | 88589 |
| BAC147_P19 | 197 | 7q11.23 | 71708488 | 71792707 | 84219 |
| BAC86_I11 | 198 | 7q11.23 | 71849362 | 76272417 | 4423055 |
| BAC191_H07 | 199 | 7q11.23 | 71863754 | 73792943 | 1929189 |
| BAC201_A03 | 200 | 7q11.23 | 72064130 | 72156285 | 92155 |
| BAC205_J04 | 201 | 7q11.23 | 72166745 | 72263182 | 96437 |
| BAC208_B07 | 202 | 7q11.23 | 72166760 | 72275964 | 109204 |
| BAC128_M22 | 203 | 7q11.23 | 72176334 | 72273688 | 97354 |
| BAC173_M16 | 204 | 7q11.23 | 72184676 | 72281076 | 96400 |
| BAC170_F12 | 205 | 7q11.23 | 72184724 | 72281015 | 96291 |
| BAC117_C10 | 206 | 7q11.23 | 72193701 | 72281002 | 87301 |
| BAC176_F03 | 207 | 7q11.23 | 72193724 | 72281020 | 87296 |
| BAC186_C15 | 208 | 7q11.23 | 72202285 | 72290021 | 87736 |
| BAC217_N14 | 209 | 7q11.23 | 72202285 | 72273685 | 71400 |
| BAC143_O02 | 210 | 7q11.23 | 72202289 | 72290099 | 87810 |
| BAC95_E13 | 211 | 7q11.23 | 72263098 | 72342773 | 79675 |
| BAC198_K24 | 212 | 7q11.23 | 72447636 | 72472477 | 24841 |
| BAC247_M06 | 213 | 7q11.23 | 72459549 | 72577616 | 118067 |
| BAC143_M09 | 214 | 7q11.23 | 72501967 | 72583330 | 81363 |
| BAC45_F05 | 215 | 7q11.23 | 72523890 | 72660399 | 136509 |
| BAC172_C11 | 216 | 7q11.23 | 72577525 | 72675932 | 98407 |
| BAC167_I04 | 217 | 7q11.23 | 72648003 | 72779145 | 131142 |
| BAC142_O19 | 218 | 7q11.23 | 72654551 | 72779156 | 124605 |
| BAC184_A15 | 219 | 7q11.23 | 72675777 | 72772799 | 97022 |
| BAC218_M18 | 220 | 7q11.23 | 72687307 | 72773571 | 86264 |
| BAC158_M12 | 221 | 7q11.23 | 72724729 | 72822397 | 97668 |
| BAC69_O08 | 223 | 7q11.23 | 72773474 | 72874048 | 100574 |
| BAC183_A12 | 224 | 7q11.23 | 73117350 | 73221401 | 104051 |
| BAC123_D05 | 225 | 7q11.23 | 73121131 | 73201329 | 80198 |
| BAC67_M07 | 226 | 7q11.23 | 73140091 | 73266423 | 126332 |
| BAC128_E24 | 227 | 7q11.23 | 73244451 | 73386632 | 142181 |
| BAC39_G01 | 228 | 7q11.23 | 73354500 | 73521308 | 166808 |
| BAC143_M17 | 229 | 7q11.23 | 73392466 | 73521327 | 128861 |
| BAC183_K06 | 230 | 7q11.23 | 73408419 | 73520437 | 112018 |
| BAC158_C18 | 231 | 7q11.23 | 73497892 | 73589952 | 92060 |
| BAC151_M20 | 232 | 7q11.23 | 73686211 | 73782545 | 96334 |
| BAC86_K17 | 233 | 7q11.23 | 73686227 | 73809984 | 123757 |
| BAC53_C18 | 234 | 7q11.23 | 73686284 | 74430267 | 743983 |
| BAC131_H10 | 235 | 7q11.23 | 73767131 | 73921866 | 154735 |
| BAC249_J16 | 236 | 7q11.23 | 73921730 | 74016279 | 94549 |
| BAC33_P15 | 237 | 7q11.23 | 73921785 | 74013189 | 91404 |
| BAC43_D03 | 238 | 7q11.23 | 73921800 | 74044547 | 122747 |
| BAC214_K20 | 239 | 7q11.23 | 74549957 | 74625358 | 75401 |
| BAC22_I04 | 240 | 7q11.23 | 74588061 | 74702637 | 114576 |
| BAC41_K08 | 241 | 7q11.23 | 74621581 | 74725185 | 103604 |
| BAC144_E01 | 242 | 7q11.23 | 74625275 | 74718398 | 93123 |
| BAC229_P01 | 243 | 7q11.23 | 74631979 | 74717975 | 85996 |
| BAC141_E12 | 245 | 7q11.23 | 74631981 | 74725132 | 93151 |
| BAC230_N13 | 246 | 7q11.23 | 74658700 | 74754101 | 95401 |
| BAC242_M21 | 247 | 7q11.23 | 74669114 | 74759508 | 90394 |
| BAC34_G23 | 248 | 7q11.23 | 74669157 | 74766600 | 97443 |
| BAC132_A16 | 249 | 7q11.23 | 74717922 | 74821295 | 103373 |
| BAC120_N24 | 250 | 7q11.23 | 74754214 | 74838860 | 84646 |
| BAC178_O12 | 251 | 7q11.23 | 74754219 | 74838868 | 84649 |
| BAC2_H21 | 252 | 7q11.23 | 74788470 | 74878771 | 90301 |
| BAC243_K17 | 253 | 7q11.23 | 74788477 | 74878516 | 90039 |
| BAC61_P24 | 254 | 7q11.23 | 74838773 | 74946373 | 107600 |
| BAC221_N02 | 255 | 7q11.23 | 74867469 | 74952024 | 84555 |
| BAC180_G20 | 256 | 7q11.23 | 74878685 | 74997131 | 118446 |
| BAC59_L19 | 257 | 7q11.23 | 74910875 | 74997142 | 86267 |
| BAC65_N09 | 258 | 7q11.23 | 74914078 | 74997130 | 83052 |
| BAC48_L04 | 259 | 7q11.23 | 75071319 | 75290438 | 219119 |
| BAC171_K05 | 260 | 7q11.23 | 75089619 | 75189211 | 99592 |
| BAC78_I13 | 261 | 7q11.23 | 75147619 | 75245296 | 97677 |
| BAC182_I23 | 262 | 7q11.23 | 75191944 | 75330285 | 138341 |
| BAC58_P18 | 263 | 7q11.23 | 75309825 | 75391206 | 81381 |
| BAC234_B11 | 264 | 7q11.23 | 75337652 | 75447403 | 109751 |
| BAC235_N07 | 265 | 7q11.23 | 75379051 | 75464758 | 85707 |
| BAC175_J10 | 266 | 7q11.23 | 75482826 | 75594646 | 111820 |
| BAC31_M21 | 267 | 7q11.23 | 75524436 | 75680271 | 155835 |
| BAC238_M17 | 268 | 7q11.23 | 75556202 | 75662768 | 106566 |
| BAC181_C12 | 269 | 7q11.23 | 75580395 | 75662759 | 82364 |
| BAC179_H21 | 270 | 7q11.23 | 75580402 | 75680271 | 99869 |
| BAC241_O09 | 271 | 7q11.23 | 75604228 | 75716244 | 112016 |
| BAC89_L08 | 272 | 7q11.23 | 75680181 | 75826330 | 146149 |
| BAC106_B03 | 273 | 7q11.23 | 75798372 | 75914006 | 115634 |
| BAC123_J16 | 274 | 7q11.23 | 76032346 | 76121091 | 88745 |
| BAC78_E10 | 275 | 7q11.23 | 76126457 | 76235798 | 109341 |
| BAC72_E18 | 276 | 7q11.23 | 76167483 | 76272486 | 105003 |
| BAC155_M16 | 278 | 7q11.23 | 76235460 | 76388642 | 153182 |
| BAC182_E12 | 279 | 7q11.23 | 76235639 | 76332386 | 96747 |
| BAC224_M13 | 280 | 7q11.23 | 76332297 | 76418638 | 86341 |
| BAC81_F13 | 281 | 7q11.23 | 76332927 | 76515224 | 182297 |
| BAC53_O03 | 282 | 7q11.23 | 76499901 | 76599150 | 99249 |

(16) Wolf-Hirschhorn Syndrome

Wolf-hirschhorn syndrome can be diagnosed for Karyotype deletion 4p16.3. A DNA chip for detecting Wolf-hirschhorn syndrome includes a microarray containing at least one immobilized genomic DNA fragment selected from the group consisting of genomic DNA fragment 1 to 72 of Table 18, at least one oligomer of 20 to 100 bp consecutive nucleotide within the fragment, or at least one cDNA of 100 bp to 10 kbp derived from the fragment. When at least two kinds of probes are mixed and used, the probes can be mixed in equal proportions and immobilized in a single spot.

TABLE 18

| | | Chromosomal | Chromosomal | | |
|---|---|---|---|---|---|
| | | location | location in Human | | |
| | | identified | genome | | |
| BAC DNA | No. | by FISH | Start point | End point | size |
| BAC236_H02 | 1 | 4p16.3 | 237802 | 326439 | 88637 |
| BAC69_O06 | 2 | 4p16.3 | 326590 | 459634 | 133044 |
| BAC221_P22 | 3 | 4p16.3 | 357208 | 438876 | 81668 |
| BAC8_J14 | 4 | 4p16.3 | 438738 | 523419 | 84681 |
| BAC34_F03 | 5 | 4p16.3 | 660907 | 773269 | 112362 |
| BAC240_P22 | 6 | 4p16.3 | 773182 | 881269 | 108087 |
| BAC8_K18 | 7 | 4p16.3 | 778791 | 908839 | 130048 |
| BAC201_E04 | 8 | 4p16.3 | 975976 | 1075769 | 99793 |
| BAC246_J21 | 9 | 4p16.3 | 1036292 | 1137719 | 101427 |
| BAC120_B11 | 10 | 4p16.3 | 1099898 | 1204523 | 104625 |
| BAC186_J20 | 11 | 4p16.3 | 1294037 | 1401323 | 107286 |
| BAC139_D23 | 12 | 4p16.3 | 1310487 | 1392382 | 81895 |
| BAC34_I14 | 13 | 4p16.3 | 1310582 | 1401343 | 90761 |
| BAC249_I06 | 14 | 4p16.3 | 1502051 | 1616984 | 114933 |
| BAC59_G04 | 15 | 4p16.3 | 1502108 | 1616993 | 114885 |
| BAC144_P04 | 16 | 4p16.3 | 1527393 | 1617675 | 90282 |
| BAC51_J17 | 17 | 4p16.3 | 1538512 | 1662717 | 124205 |
| BAC57_K06 | 18 | 4p16.3 | 1538513 | 1616980 | 78467 |
| BAC161_D24 | 19 | 4p16.3 | 1580196 | 1666779 | 86583 |
| BAC120_F12 | 20 | 4p16.3 | 1603988 | 1703810 | 99822 |
| BAC54_K17 | 21 | 4p16.3 | 1609860 | 1700697 | 90837 |
| BAC246_B02 | 22 | 4p16.3 | 1666660 | 1749494 | 82834 |
| BAC95_G12 | 23 | 4p16.3 | 1670782 | 1749551 | 78769 |
| BAC50_H08 | 24 | 4p16.3 | 1776527 | 1908757 | 132230 |
| BAC67_I12 | 25 | 4p16.3 | 1864852 | 1972573 | 107721 |
| BAC100_E03 | 26 | 4p16.3 | 1870452 | 1987961 | 117509 |
| BAC1_F06 | 27 | 4p16.3 | 1882624 | 1976908 | 94284 |
| BAC135_O20 | 28 | 4p16.3 | 1924077 | 2012501 | 88424 |
| BAC153_J14 | 29 | 4p16.3 | 2040880 | 2121244 | 80364 |
| BAC88_O15 | 30 | 4p16.3 | 2052609 | 2150615 | 98006 |
| BAC115_O12 | 31 | 4p16.3 | 2070582 | 2181127 | 110545 |
| BAC180_N19 | 32 | 4p16.3 | 2083590 | 2181158 | 97568 |
| BAC104_E06 | 33 | 4p16.3 | 2083592 | 2175704 | 92112 |
| BAC106_G23 | 34 | 4p16.3 | 2098695 | 2175715 | 77020 |
| BAC205_J02 | 35 | 4p16.3 | 2121159 | 2268093 | 146934 |
| BAC67_A15 | 36 | 4p16.3 | 2121176 | 2204189 | 83013 |
| BAC84_I10 | 37 | 4p16.3 | 2150502 | 2270631 | 120129 |
| BAC248_G23 | 38 | 4p16.3 | 2204031 | 2284420 | 80389 |
| BAC10_B17 | 39 | 4p16.3 | 2267979 | 2363306 | 95327 |
| BAC243_G05 | 40 | 4p16.3 | 2270548 | 2354350 | 83802 |
| BAC44_F18 | 41 | 4p16.3 | 2270551 | 2348220 | 77669 |
| BAC70_K18 | 42 | 4p16.3 | 2308475 | 2449077 | 140602 |
| BAC28_C01 | 43 | 4p16.3 | 2347639 | 2459649 | 112010 |
| BAC197_P06 | 44 | 4p16.3 | 2365995 | 2449102 | 83107 |
| BAC178_E16 | 45 | 4p16.3 | 2386696 | 2453233 | 66537 |
| BAC168_P10 | 46 | 4p16.3 | 2665843 | 2791008 | 125165 |
| BAC158_J15 | 47 | 4p16.3 | 2691131 | 2780498 | 89367 |
| BAC94_N09 | 48 | 4p16.3 | 2783578 | 2909639 | 126061 |
| BAC163_J20 | 49 | 4p16.3 | 2790797 | 2871745 | 80948 |
| BAC65_E06 | 50 | 4p16.3 | 2790853 | 2917387 | 126534 |
| BAC69_H12 | 51 | 4p16.3 | 2833237 | 2927038 | 93801 |
| BAC57_G03 | 52 | 4p16.3 | 2874989 | 2972737 | 97748 |
| BAC223_D21 | 53 | 4p16.3 | 2934228 | 3014295 | 80067 |
| BAC69_H12 | 51 | 4p16.3 | 2833237 | 2927038 | 93801 |
| BAC2_A11 | 54 | 4p16.3 | 2968579 | 3093901 | 125322 |
| BAC104_D06 | 55 | 4p16.3 | 2994610 | 3071589 | 76979 |
| BAC14_E23 | 56 | 4p16.3 | 2994665 | 3093876 | 99211 |
| BAC75_B05 | 57 | 4p16.3 | 3026122 | 3133836 | 107714 |
| BAC1_K04 | 58 | 4p16.3 | 3027988 | 3133840 | 105852 |
| BAC45_D08 | 59 | 4p16.3 | 3050938 | 3190266 | 139328 |
| BAC22_I03 | 60 | 4p16.3 | 3077018 | 3158980 | 81962 |
| BAC150_H14 | 61 | 4p16.3 | 3158882 | 3244513 | 85631 |
| BAC236_H20 | 62 | 4p16.3 | 3169307 | 3274343 | 105036 |
| BAC194_P13 | 63 | 4p16.3 | 3174446 | 3258519 | 84073 |
| BAC120_J05 | 64 | 4p16.3 | 3274662 | 3384835 | 110173 |
| BAC180_H03 | 65 | 4p16.3 | 3320490 | 3397871 | 77381 |
| BAC45_D09 | 66 | 4p16.3 | 3339121 | 3473278 | 134157 |
| BAC103_L05 | 67 | 4p16.3 | 3384745 | 3388167 | 3422 |
| BAC186_K08 | 68 | 4p16.3 | 3384789 | 3485826 | 101037 |
| BAC157_K14 | 69 | 4p16.3 | 3396916 | 3505239 | 108323 |
| BAC217_L15 | 70 | 4p16.3 | 3505011 | 3605672 | 100661 |
| BAC250_L02 | 71 | 4p16.3 | 3525104 | 3654803 | 129699 |
| BAC183_F04 | 72 | 4p16.3 | 3693162 | 3792741 | 99579 |

In another embodiment, the present invention provides a DNA microarray for detecting constitutional diseases checked pre- or post-natally. The DNA microarray is used for diagnosing target disease in before/after childbirth diagnosis, or for detecting the mutations on whole chromosome. The probe can be the genomic DNA for detecting a disease-specific region of the chromosome, or oligmer, cDNA, or RNA derived from genomic DNA. The DNA microarray includes one or several kinds of the probes for detecting the chromosomal mutation specific to disease immobilized thereon. The target chromosome region used for diagnosing the constitutional diseases can be largely selected from chromosome 13, 18, 21, X and Y. The examples of the constitutional diseases include Patau syndrome, Edwards syndrome, Turner syndrome, and Klinefelter syndrome, but do not limited thereto.

The genomic DNA microarray of the present invention can detect at least 250 bp of chromosome abnormality with a high resolution, and is very convenient compared to the conventional karyotype analysis and FISH.

The genomic DNA microarray of the present invention has advantages that the large quantity of desired genomic DNA fragments can be easily obtained according to the usage of the genomic DNA chip from the clones which have the identified chromosomal location and nucleotide sequence of inserted genomic DNA fragment. In addition, it is possible to easily detect chromosome abnormality on genome of the organism, to detect chromosome abnormality in intron region other than exon region, to suggest the relationship between specific disease and the detected chromosome, to observe the genomic mutation aspect caused by endogeneous or exogeneous factor, to provide a chip for diagnosing specific disease, and to help the research on polymorphism specific to race or human.

The genomic DNA microarray of the present invention is useful for diagnosing the disease caused by chromosomal mutation, and particularly for a disease caused by abnormality of the number of the chromosome and cancer.

The Microarray Immobilized by at Least Two Kinds of Probes in a Spot

The present invention provides a microarray immobilized by at least two kinds of probes in a spot. The mixture ratio of probe at one spot can be the same for the same kind of probe. The probes can be selected from RNA fragment, cDNA fragment, oligomer and genomic DNA fragment. At least two kinds of probes immobilized in a spot includes a nucleotide selected from the group consisting of whole chromosome, parts of chromosome, and a complementary DNA thereof showing the same phenotype in an abnormality of chromosome number, and the phenotype is a gene amplification, or a gene deletion. The genomic DNA is selected from the group consisting of the DNA 1 to 25851 as shown in Table 1, and can be RNA fragment, cDNA fragment or oligomer derived from the genomic DNA.

The Detection Method of Chromosome Abnormality with Microarray

The method of detecting chromosome abnormality according to the present invention contains the steps of:

(a) preparing a microarray for detecting chromosome abnormality;

(b) reacting sample genomic DNA in a sample which is labeled by first detectable labeling material, and the reference DNA labeled by second detectable labeling material in the same mixture ratio, with the probe immobilized on the microarray, and measuring hybridization ratio of the target genomic DNA or the reference DNA with the probe respectively; and

(c) analyzing chromosome abnormality of the target genomic DNA on the basis of the hybridization ratio.

In step (c), the chromosome abnormality is analyzed on the basis of:

No chromosome abnormality, if the hybridization ratio of the target genomic DNA is the same as that of the reference DNA is decided to produce no chromosome abnormality, Chromosome amplification, if the hybridization ratio of the target genomic DNA is higher than that of the reference DNA.

Chromosome deletion, if the hybridization ratio of the target genomic DNA is lower than that of the reference DNA.

The probe is genomic DNA fragment, oligomer, cDNA or RNA, and the examples of the genomic DNA are shown in Table 1.

The samples, tissue, cell, or body fluids which are selected from the group consisting of saliva, blood, urine, semen, and amniotic fluid, and can be isolated or separated from the sample according to the conventional method.

The first detectable labeling material and the second detectable labeling material are the same or different, for examples independently are radioactive isotope, fluorescent material, chemical luminescent, and enzyme. For examples, Cy3, Cy5, Alexa Fluor 350, Alexa Fluor 430, Alexa Fluor 488, Alexa Fluor 532, Alexa Fluor 546, Alexa Fluor 594, Alexa Fluor 658, Cyanine-3, Cyanine-5, fluorescein, [symbol] (bodipy), Texas red, FITC (Fluorescein Isothiocyanate), rhodamine, d-NTP (including d-UTP), reactive dye having amino-allyl modified dNTPs, horseradish peroxidase, biotin and etc. The method is explained in more detail as below.

Step 1: Isolation of Sample Genomic DNA

The genomic DNA is isolated from a sample according to the conventional method or known kit. The target genomic DNA is prepared at an amount of 100 ng to 1 ug.

Step 2: Labeling

The target genomic DNA and reference DNA are labeled by random priming with different labeling material. For example, the target genomic DNA is labeled with Cy3 (blue color), and the reference genomic DNA is labeled with Cy5 (red color).

Step 3: Reaction with Microarray Chip

The labeled target genomic DNA and reference genomic DNA are mixed in a weight ratio of 1:1, then reacted with microarray, and washed.

The amount of DNA mixture reacted with microarray is 200 to 2 ug. The DNA mixture can be suspended in hybridization buffer solution which includes known solution. The BAC chip can be treated with the material preventing the nonspecific hybridization, or example DNA of salmon sperm before treating with the DNA mixture.

Step 4: Analysis

The hybridization of the reacted microarray is analyzed by inserting the microarray into the scanner which can detect the labeling materials, and using the program which probe information and spot location input.

If the sample chromosome is normal, the ratio of the target DNA hybridized with the probe and reference genomic DNA is 1:1, and thus spot represents yellow color. If the sample chromosome includes chromosome amplification, the hybridization ratio of the target DNA is higher than that of the reference DNA, and thus spot represents blue color. If the sample chromosome includes chromosome deletion, the hybridization ratio of the target DNA is lower than that of the reference DNA, and thus spot represents red color. Accordingly, specific DNA amplification and deletion of the sample can be detecting by measuring the fluorescence of each spot.

Detection of the Chromosome Abnormality Specific to Disease

The present invention provides a method of detecting a disease-specific chromosome abnormality comprises the steps of:

(a) identifying a genomic DNA fragment inserted to each clone which is obtained from organism's genomic library;

(b) immobilizing the entire or a part of genomic DNA fragment identified in step (a) on a microarray;

(b) reacting the target genomic DNA of patient which is labeled by a first detectable labeling material, and a reference DNA labeled by a second detectable labeling material in the same mixture ratio, with the probe immobilized on the microarray, and measuring hybridization ratio of the target genomic DNA or the reference DNA with the probe respectively; and

(c) detecting a region and type of a chromosome abnormality of the patient by analyzing the hybridization ratio.

When the chromosome abnormality specific to a disease, and abnormal region are not identified, the method provides it to detect them easily.

In an embodiment, BAC chip can be prepared by using the BAC clone which is obtained from organism's genomic library. In this case, the BAC DNA present in the BAC chip may correspond to the whole human genome, a specific chromosome or a specific gene.

The BAC chip containing the BAC DNA corresponding to the whole human genome can be used for the following purposes.

(1) The BAC chip can be used for diagnosing the health condition and a disease associated with the chromosome abnormality by detecting chromosome abnormality in sample whole genome, when a disease is known to associate with the chromosome abnormality.

(2) The chromosome abnormality is analyzed by using DNA of patient with a specific disease as a sample DNA, in order to identify the chromosome abnormality associated with a specific disease. Thus, the target DNA can be used for diagnosing the other patient with the same disease as a probe.

(3) The BAC chip can provides a tool for analyzing chromosome abnormality caused by specific materials, for examples drugs or compounds.

(4) The BAC chip can be used for analyzing genome polymorphism by individuals or human race.

The BAC chip for a specific chromosome or gene may be used to more closely detect the chromosome abnormality at the target region.

The present invention is further explained in more detail with reference to the following examples. These examples, however, should not be interpreted as limiting the scope of the present invention in any manner.

### EXAMPLE 1

### Preparation of BAC Clone

1-1. Preparation of Genome Library

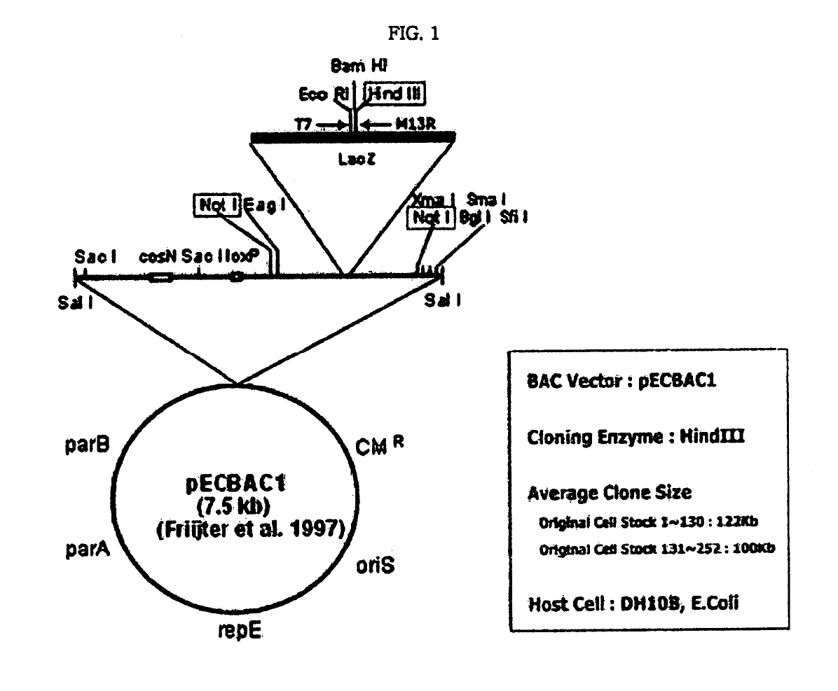

A genome was isolated from a Korean man, treated with HindIII enzyme and subjected to PFGE, to obtain a DNA fragment of about 100 Kb. The DNA fragment of the average size of 100 Kb was ligased to a BAC vector, and then, transformed into a host cell, E. coli. Herein, the E. coli cell line containing each DNA fragment was called as a clone, and 96,768 clones in total were obtained through the preparation of genome library as above (FIG. 1).

1-1-1. Isolation of Genome

20 Ml of the entire genomic DNA was isolated from semen of a Korean man, and subjected to a qualitative analysis and a quantitative analysis through agarose gel electrophoresis.

1-1-2. Fragmentation and Purification of Genome

The isolated genomic DNA was cleaved with BamHI, and subjected to PEG gel electrophoresis. The portion which was developed at the position of 100 Kb was collected, to isolate DNA fragments.

1-1-3. Transformation

The DNA fragments were inserted into BAC vectors (pECBAC1) at BamHI site, and transformed into host cells (Competent cell, E. coli DH10B BAC). Then, the host cells were cultivated in a solid culture media, to obtain clone of each cell. The clones were inoculated on 96-well format cell culture blot, and cultivated in a rotating incubator of 300 rpm at 370 for 18 hours.

1-1-4. Preparation of Library Cell Stock Solution

25 μl of 65% glycerol was put into 384-well plate, and 25 μl of cells cultivated in 96-well format cell culture blot was added thereto (cells cultivated in four 96-well format cell culture blots were collected and stored in one 384-well plate). The top of the 384-well was sealed and stored at −700 □.

1-2. End-Sequencing

500 bp of both ends of the 100 Kb DNA fragment were analyzed with a BAC vector specific primer, and the information for the inserted genomic DNA of each clone was confirmed and recorded.

1-2-1. Isolation of BAC DNA (Mini-Prep.)

1×LB 1 Ml was put into 96-well culture blot, and library cell stock solution 1 Ml was inoculated thereto. The cells stored in one 348-well plate were divisionally cultivated in four 96-well culture blots. The cells was cultivated in a rotating incubator of 300 rpm at 37 □ for 18 hours and, to prepare a sample stock solution in the amount of 25 Ml per a well in five times. The remnant was centrifuged at 1000 rpm for 15 minutes, to obtain cells. DNA was collected by using a kit, Montage mini-prep.

1-2-2. Sequencing

Both ends of the DNA fragment inserted into each clone was treated with T7 and M13R primers, and the base sequences thereof were analyzed by using an Automatic DNA Sequencer, ABL 3700.

1-3. Bioinformatics

The analyzed base sequences were subjected to the sequence identity analysis compared with the base sequence of the entire human genome analyzed through the human genome project using a bioinformatics technology. Through the above processes, the position of the BAC DNA in genome of each clone in which the BAC DNA is inserted and the entire base sequence thereof were determined.

1-3-1. Blast Search

A blast search was performed by using the analyzed end sequence, and its position in the genome of each clone was determined by using the sequence identity analysis.

1-3-2. Selection of Gene/Marker

Among the genomic DNA fragments of each clone obtained from genome library, the clone comprising a cancer-associated gene/STS marker was selected.

1-4. BAC Clone Mapping by FISH

In order to more exactly determine the position of each BAC clone on the genome, FISH was conducted by using the genomic DNA fragment contained in each BAC clone as a probe. In the FISH technique employing the DNA complementary binding principle, a probe which specifically binds to a specific region of a chromosome is directly bound to the chromosome fixed on a glass slide, to determine the position of the probe in the chromosome.

The position of the genomic DNA fragment of each determined BAC clone in the chromosome is shown in Table 1.

### EXAMPLE 2

### Fabrication of BAC Chip

2-1. Selection of Clone to be Used in Fabricating BAC Chip

1440 clones according to each chromosomal number were selected from the BAC library for the purpose of diagnosing a specific genetic disease associated with an abnormality in the chromosomal number. 1440 clones are shown in Table 19 below.

TABLE 19

| | | | marker | |
|---|---|---|---|---|
| | | | No. of | No. of |
| chromosome | | No. of | cancer/ | STS/ |
| number | BAC DNA | clone | gene | Terminal |
| 1 | BAC176_F17, BAC38_N15, BAC101_I16, BAC230_P11, BAC37_K05, BAC171_P09, BAC90_A08, BAC180_C01, BAC91_A07, BAC80_C03, | 103 | 20 | 83 |
| | BAC145_B18, BAC133_M03, BAC68_M07, BAC13_F18, BAC236_J05, BAC46_E05, BAC120_F15, BAC141_N19, BAC63_I09, BAC146_G09, | | | |
| | BAC124_B01, BAC20_I15, BAC96_E21, BAC92_N23, BAC33_L19, BAC162_J22, BAC215_J18, BAC108_F03, BAC108_H02, | | | |
| | BAC96_M17, BAC24_E20, BAC141_H16, BAC29_J22, BAC142_G14, BAC193_L08, BAC22_P12, BAC71_J20, BAC81_F02, BAC15_M17, | | | |
| | BAC104_G18, BAC77_K04, BAC189_F17, BAC78_B10, BAC74_F02, BAC118_I08, BAC59_G14, BAC58_B15, BAC53_D06, BAC153_B07, | | | |
| | BAC65_M16, BAC20_G18, BAC114_F11, BAC22_M23, BAC51_G07, BAC127_H20, BAC91_J05, BAC132_I13, BAC157_J21, | | | |
| | BAC227_N14, BAC113_H08, BAC59_B21, BAC79_K19, BAC60_F24, BAC29_M19, BAC123_I23, BAC78_H12, BAC1_O10, BAC85_N11, | | | |
| | BAC215_N06, BAC235_K22, BAC183_F06, BAC249_I07, BAC96_I22, BAC157_C20, BAC177_L21, BAC84_E09, BAC72_N18, BAC115_J12, | | | |
| | BAC81_C10, BAC165_M06, BAC109_M23, BAC82_F03, BAC25_H07, BAC59_M15, BAC80_G04, BAC118_I22, BAC84_O24, | | | |
| | BAC170_F20, BAC42_F06, BAC169_C15, BAC139_G10, BAC50_H09, BAC128_O05, BAC147_E05, BAC118_K14, BAC40_F20, BAC97_D21, | | | |
| | BAC132_D14, BAC6_N24, BAC37_P24, BAC168_C19, BAC92_E04, BAC134_B22 | | | |
| 2 | BAC153_L04, BAC19_D07, BAC34_P10, BAC165_O21, BAC29_C11, BAC76_D18, BAC247_B10, BAC99_G21, BAC126_O17, BAC239_O18, | 109 | 17 | 92 |
| | BAC167_M04, BAC64_M02, BAC146_F07, BAC37_E18, BAC176_M18, BAC97_N06, BAC252_B21, BAC137_N18, BAC175_J02, BAC90_P18, | | | |
| | BAC166_F20, BAC145_H13, BAC32_A06, BAC143_E05, BAC143_E05, BAC52_F13, BAC118_O22, BAC120_P03, BAC43_G23, | | | |
| | BAC65_K04, BAC142_G22, BAC122_G07, BAC209_G19, BAC15_M23, BAC12_G01, BAC140_N16, BAC10_H13, BAC145_L06, BAC83_P03, | | | |
| | BAC165_F20, BAC92_C01, BAC23_H04, BAC144_A07, BAC69_J21, BAC125_H18, BAC185_D04, BAC120_P19, BAC103_I04, | | | |
| | BAC96_P09, BAC24_D14, BAC65_B13, BAC82_P19, BAC20_N21, BAC83_K08, BAC167_P18, BAC92_C18, BAC169_P06, BAC63_N08, | | | |
| | BAC219_N21, BAC50_E06, BAC64_P09, BAC232_L16, BAC39_N20, BAC106_B08, BAC203_A08, BAC143_C23, BAC80_L11, BAC27_B01, | | | |
| | BAC1_H07, BAC61_N08, BAC183_G19, BAC2_G24, BAC80_A06, BAC143_A10, BAC16_C12, BAC84_K05, BAC13_D10, BAC12_F20, | | | |
| | BAC68_H23, BAC157_F03, BAC108_L17, BAC96_G06, BAC69_D21, BAC131_M09, BAC80_E09, BAC73_P04, BAC169_F10, | | | |
| | BAC11_G01, BAC106_A14, BAC84_P12, BAC51_G24, BAC52_M13, BAC53_J20, BAC142_B20, BAC189_L05, BAC57_M03, BAC232_M13, | | | |
| | BAC175_D13, BAC116_P06, BAC31_A16, BAC26_H23, BAC92_I07, BAC123_I06, BAC75_N17, BAC168_I09, BAC72_M15, BAC40_D08, | | | |
| | BAC15_M19, BAC131_H16 | | | |
| 3 | BAC45_F02, BAC179_L14, BAC83_J18, BAC51_F01, BAC154_G23, BAC169_O13, BAC241_P15, BAC165_K17, BAC142_N07, BAC156_D05, | 92 | 25 | 67 |
| | BAC12_O03, BAC195_G14, BAC60_B02, BAC144_C11, BAC175_I18, BAC85_G09, BAC202_D18, BAC1_J07, BAC57_M07, BAC230_M11, | | | |
| | BAC97_N23, BAC206_J21, BAC218_K17, BAC232_N06, BAC80_H11, BAC233_H20, BAC201_F16, BAC241_O06, BAC220_H15, | | | |
| | BAC82_N23, BAC209_K05, BAC117_E10, BAC167_G07, BAC237_F09, BAC24_K03 | | | |
| | BAC107_H04, BAC8_G11, BAC73_M11, BAC109_M19, BAC162_A19, BAC26_E03, BAC37_G21, BAC170_J09, BAC30_D18, BAC40_O18, | | | |
| | BAC189_J06, BAC131_I23, BAC150_J01, BAC71_P12, BAC71_C14, BAC152_C16, BAC22_O17, BAC77_F10, BAC13_P22, BAC89_H07, | | | |
| | BAC106_M21, BAC125_G19, BAC82_M18, BAC247_C06, BAC199_J09, BAC156_P13, BAC26_P08, BAC58_N16, BAC128_C03, BAC177_G04, | | | |
| | BAC155_H02, BAC129_C01, BAC23_N18, BAC96_G23, BAC133_H24, BAC25_B02, BAC173_F07, BAC22_K22, BAC27_D22, | | | |
| | BAC78_P09, BAC215_M07, BAC28_H10, BAC97_L20, BAC143_K08, BAC87_K21, BAC46_F17, BAC178_H09, BAC144_B04, BAC97_O04, | | | |
| | BAC195_F06, BAC213_I18, BAC144_N20, BAC74_E06, BAC119_D15, BAC173_O07, BAC61_O10, BAC243_N21 | | | |
| 4 | BAC120_B11, BAC192_N22, BAC50_H08, BAC67_I12, BAC163_J20, BAC139_E10, BAC100_B07, BAC75_N19, BAC193_F03, BAC21_I15, | 63 | 14 | 49 |
| | BAC12_K01, BAC41_K05, BAC84_I22, BAC168_J10, BAC33_M04, BAC42_P11, BAC165_H02, BAC75_G11, BAC214_I01, BAC100_O09, | | | |
| | BAC78_K15, BAC179_A18, BAC139_N16, BAC1_C16, BAC102_F24, BAC157_D13, BAC193_O16, BAC54_J04, BAC1_I15, BAC78_N01, | | | |
| | BAC30_I03, BAC10_A09, BAC27_J08, BAC95_P20, BAC229_P17, BAC34_N19, BAC83_F06, BAC221_K14, BAC247_H12, BAC161_L08, | | | |
| | BAC232_P08, BAC63_P04, BAC121_P19, BAC67_B03, BAC168_J03, BAC69_L20, BAC69_L20, BAC144_H01, BAC5_D24, | | | |
| | BAC1_P14, BAC140_G19, BAC233_I20, BAC186_J15, BAC89_D11, BAC169_F02, BAC162_P10, BAC92_B05, BAC152_C21, BAC83_J21, | | | |
| | BAC143_I10, BAC84_P22, BAC56_F05, BAC158_A23 | | | |
| 5 | BAC63_F14, BAC143_B15, BAC104_G21, BAC179_F12, BAC84_C03, BAC135_L06, BAC116_I11, BAC237_J04, BAC41_O16, BAC152_H04, | 73 | 17 | 56 |
| | BAC100_E07, BAC209_I22, BAC109_H04, BAC83_M16, BAC161_K04, BAC241_H11, BAC16_L22, BAC230_O24, BAC69_A02, BAC185_O16, | | | |
| | BAC171_A01, BAC135_P04, BAC144_P08, BAC140_C04, BAC8_P07, BAC166_A05, BAC154_I09, BAC119_I19, BAC96_B13, | | | |
| | BAC12_E06, BAC12_E06, BAC25_M19, BAC12_H03, BAC237_N24, BAC13_G12, BAC147_D19, BAC83_I19, BAC178_H02, BAC104_D13, | | | |
| | BAC152_J19, BAC45_L07, BAC126_A01, BAC179_F17, BAC168_M02, BAC161_C19, BAC5_I04, BAC53_A02, BAC222_P07, BAC141_I18, | | | |
| | BAC211_D16, BAC140_H19, BAC161_A19, BAC89_H06, BAC59_N13, BAC116_I13, BAC167_K06, BAC175_L19, BAC143_A21, | | | |
| | BAC200_J07, BAC83_D21, BAC123_K15, BAC176_G19, BAC57_G23, BAC196_J01, BAC111_D04, BAC176_E04, BAC89_H17, BAC131_C11, | | | |
| | BAC88_D19, BAC153_I06, BAC68_H03, BAC76_C20, BAC53_P23 | | | |
| 6 | BAC178_M23, BAC80_J07, BAC14_E06, BAC86_N04, BAC86_G12, BAC132_J18, BAC183_J04, BAC155_N23, BAC2_J04, BAC247_M13, | 81 | 21 | 60 |
| | BAC97_H14, BAC33_F13, BAC113_K09, BAC147_A07, BAC144_D21, BAC40_N06, BAC131_E23, BAC249_P17, BAC56_M01, BAC157_G20, | | | |
| | BAC219_C01, BAC104_F07, BAC100_M24, BAC97_I22, BAC97_H10, BAC134_F04, BAC90_M07, BAC116_P17, BAC92_I06, BAC171_B22, | | | |
| | BAC52_H13, BAC57_A01, BAC92_M03, BAC135_A14, BAC236_A07, BAC183_P10, BAC189_K20, BAC174_C09, BAC139_M20, | | | |
| | BAC162_J19, BAC162_J19, BAC71_N14, BAC81_H13, BAC133_M12, BAC120_A16, BAC76_A23, BAC162_D08, BAC81_G12, BAC86_L20, | | | |
| | BAC115_D12, BAC143_P17, BAC31_M14, BAC167_F05, BAC97_H04, BAC154_I05, BAC246_A19, BAC39_M24, BAC103_P05, | | | |
| | BAC80_O04, BAC22_E09, BAC228_A01, BAC83_D24, BAC1_F23, BAC129_J17, BAC90_H10, BAC236_B24, BAC113_O10, BAC68_C07, | | | |
| | BAC39_N24, BAC160_I11, BAC129_A04, BAC14_M20, BAC92_E13, BAC53_E01, BAC52_C20, BAC152_N22, BAC200_M04, BAC147_C16, | | | |
| | BAC19_O23, BAC121_P18, BAC12_F16, | | | |
| 7 | BAC39_K09, BAC161_M22, BAC100_K13, BAC139_D05, BAC127_G14, BAC65_K10, BAC62_J14, BAC62_N06, BAC112_K01, BAC199_B07, | 83 | 14 | 69 |
| | BAC131_G08, BAC31_C02, BAC218_N01, BAC139_K10, BAC100_E09, BAC161_H20, BAC161_H20, BAC75_J03, BAC89_K07, BAC24_A20, | | | |
| | BAC122_F23, BAC5_A09, BAC71_J10, BAC151_A17, BAC34_E12, BAC46_P02, BAC34_L05, BAC221_F17, BAC193_I24, BAC25_N13, | | | |
| | BAC80_I11, BAC15_B08, BAC1_I06, BAC85_D22, BAC217_M14, BAC61_A07, BAC57_L05, BAC145_L15, BAC100_L06, BAC78_I13, | | | |
| | BAC226_J16, BAC1_P18, BAC186_B10, BAC29_K23, BAC105_F06, BAC3_J18, BAC169_J07, BAC185_E15, BAC26_C02, | | | |
| | BAC42_D19, BAC144_G04, BAC109_M06, BAC96_K03, BAC76_I16, BAC144_C20, BAC100_N11, BAC232_P13, BAC1_B12, BAC202_P14, | | | |
| | BAC83_N01, BAC147_C10, BAC85_H06, BAC127_C14, BAC237_O10, BAC195_F16, BAC30_F19, BAC67_N20, BAC142_H10, BAC238_G23, | | | |
| | BAC32_O17, BAC79_G17, BAC125_H09, BAC221_K10, BAC33_O07, BAC120_E18, BAC175_L18, BAC80_H22, BAC124_O22, | | | |
| | BAC140_I02, BAC15_O23, BAC171_D10, BAC96_C15, BAC82_L19 | | | |
| 8 | BAC218_F21, BAC78_A19, BAC46_E24, BAC1_J24, BAC90_M06, BAC145_N24, BAC144_F08, BAC27_O21, BAC31_D08, BAC176_L07, | 53 | 14 | 39 |
| | BAC51_G11, BAC97_F20, BAC117_I18, BAC74_F23, BAC11_C18, BAC15_F07, BAC76_E16, BAC168_N13, BAC247_M21, BAC155_D08, | | | |
| | BAC19_P21, BAC145_H17, BAC78_O16, BAC106_L05, BAC152_G16, BAC50_F01, BAC36_H08, BAC177_N09, BAC217_O23, BAC178_P02, | | | |
| | BAC57_O15, BAC37_H09, BAC90_P15, BAC92_K10, BAC157_F12, BAC55_M03, BAC169_B05, BAC20_K18, BAC176_H16, BAC199_P22, | | | |
| | BAC200_E17, BAC248_F14, BAC192_N08, BAC53_L17, BAC195_N23, BAC100_A11, BAC113_P08, BAC8_N14, BAC139_M03, | | | |
| | BAC90_C04, BAC76_N02, BAC145_J12, BAC193_J11 | | | |
| 9 | BAC22_E06, BAC44_B22, BAC75_D17, BAC133_K20, BAC71_J11, BAC139_H09, BAC59_H01, BAC49_B18, BAC160_C10, BAC88_P21, | 56 | 15 | 41 |
| | BAC27_C20, BAC86_G04, BAC20_M12, BAC131_N18, BAC125_M17, BAC135_G04, BAC185_C04, BAC33_J02, BAC70_O18, BAC84_K23, | | | |
| | BAC104_M09, BAC40_J09, BAC84_F11, BAC42_K01, BAC91_J21, BAC6_B03, BAC84_E19, BAC165_B09, BAC90_K21, BAC37_F17, | | | |
| | BAC26_C01, BAC10_P08, BAC57_M21, BAC221_I19, BAC100_N15, BAC97_K18, BAC165_P10, BAC90_A22, BAC106_J20, BAC20_C01, | | | |
| | BAC96_N01, BAC157_D19, BAC130_H19, BAC63_L05, BAC193_F12, BAC65_E14, BAC118_A06, BAC142_B18, BAC125_M06, BAC108_J10, | | | |
| | BAC142_E13, BAC233_P12, BAC110_G13, BAC4_J24, BAC224_C15, BAC176_M06 | | | |
| 10 | BAC70_M18, BAC176_J17, BAC143_B19, BAC143_G07, BAC198_H13, BAC56_M02, BAC243_L13, BAC145_N12, BAC82_K06, BAC145_P22, | 69 | 21 | 47 |
| | BAC250_J11, BAC25_H08, BAC15_O16, BAC142_K07, BAC142_J05, BAC144_N08, BAC175_I06, BAC90_D13, BAC21_L23, BAC116_L03, | | | |
| | BAC155_H03, BAC81_M04, BAC51_G06, BAC140_E20, BAC193_I07, BAC193_A18, BAC4_P01, BAC169_F05, BAC83_F04, | | | |
| | BAC144_M14, BAC24_E16, BAC178_D22, BAC90_G04, BAC117_P03, BAC64_K18, BAC110_F03, BAC145_F01, BAC142_J19, BAC84_A21, | | | |
| | BAC106_B16, BAC41_E05, BAC56_I15, BAC86_I06, BAC15_P02, BAC222_E18, BAC244_H22, BAC152_K02, BAC155_M23, BAC62_C09, | | | |
| | BAC82_E10, BAC81_O02, BAC186_N19, BAC165_N05, BAC252_B16, BAC81_O19, BAC218_E11, BAC154_E03, BAC4_G24, | | | |
| | BAC82_K12, BAC245_F15, BAC223_O05, BAC48_I12, BAC100_I08, BAC97_D05, BAC167_G16, BAC9_B16, BAC92_D03, BAC42_H14 | | | |
| 11 | BAC165_D01, BAC121_J14, BAC252_K02, BAC60_J23, BAC193_P15, BAC121_J11, BAC91_J13, BAC219_F22, BAC96_J11, BAC185_G10, | 73 | 31 | 42 |
| | BAC32_K13, BAC195_A16, BAC252_G23, BAC88_B15, BAC82_C10, BAC82_C10, BAC35_I01, BAC90_G07, BAC117_D15, BAC1_K02, BAC163_D07, | | | |
| | BAC65_I22, BAC112_A18, BAC161_E20, BAC12_F24, BAC222_A06, BAC19_L09, BAC97_I05, BAC248_P18, BAC12_H05, BAC167_H05, | | | |
| | BAC33_M02, BAC59_H09, BAC50_K14, BAC101_B24, BAC197_D09, BAC195_K21, BAC228_L09, BAC185_L04, BAC122_I18, | | | |
| | BAC192_C22, BAC128_J04, BAC149_E07, BAC241_H14, BAC220_G02, BAC39_F05, BAC75_E17, BAC241_C13, BAC96_G07, BAC116_D07, | | | |
| | BAC222_F09, BAC144_K18, BAC38_N11, BAC184_B04, BAC153_K19, BAC100_H09, BAC12_L24, BAC44_C18, BAC120_O18, BAC33_D18, | | | |
| | BAC185_E02, BAC75_I11, BAC183_F14, BAC145_H08, BAC64_I15, BAC158_L17, BAC141_J03, BAC91_K14, BAC72_F10, BAC250_C10, | | | |
| | BAC121_O09, BAC21_O18, BAC173_G12 | | | |
| 12 | BAC155_L08, BAC252_M17, BAC73_O21, BAC36_D21, BAC115_B18, BAC13_A13, BAC90_L22, BAC188_D23, BAC34_J07, BAC157_O02, | 57 | 19 | 38 |
| | BAC25_K04, BAC174_C05, BAC88_B12, BAC140_E24, BAC107_B02, BAC168_P16, BAC69_O13, BAC121_J23, BAC247_C10, BAC42_L03, | | | |
| | BAC143_H17, BAC76_B24, BAC1_B14, BAC54_H10, BAC217_M07, BAC127_P09, BAC41_I18, BAC115_I13, BAC196_D16, BAC209_C17, | | | |
| | BAC108_G22, BAC46_N17, BAC10_M07, BAC65_E02, BAC232_J22, BAC234_L21, BAC154_D12, BAC97_B21, BAC59_C14, BAC234_E19, | | | |
| | BAC221_N20, BAC84_C20, BAC13_H06, BAC1_M18, BAC164_J21, BAC140_P13, BAC22_C06, BAC176_A04, BAC28_J11, BAC82_A13, BAC168_A15, | | | |
| | BAC8_N02, BAC162_L24, BAC142_E09, BAC71_K16, BAC22_N22, BAC136_E22 | | | |
| 13 | BAC199_L02, BAC77_M02, BAC171_B19, BAC91_O18, BAC158_N18, BAC142_B19, BAC157_J18, BAC43_L23, BAC29_D09, BAC88_K22, | 42 | 11 | 31 |
| | BAC147_I15, BAC80_F19, BAC101_E02, BAC122_P16, BAC181_L23, BAC235_D20, BAC66_B10, BAC92_J05, BAC27_O11, BAC29_C16, | | | |
| | BAC175_N19, BAC63_I22, BAC51_L06, BAC168_M20, BAC117_L08, BAC96_N17, BAC154_E21, BAC60_F12, BAC145_C09, BAC97_A19, | | | |
| | BAC29_K13, BAC166_A03, BAC84_E11, BAC143_B05, BAC73_O17, BAC155_B10, BAC233_L22, BAC193_H21, BAC41_E02, BAC123_J22, | | | |
| | BAC96_A02, BAC232_G07, | | | |
| 14 | BAC12_N17, BAC239_I12, BAC72_L13, BAC59_O07, BAC1_O24, BAC222_N17, BAC157_P13, BAC162_N24, BAC96_D15, BAC90_M15, BAC237_C10, | 44 | 12 | 32 |
| | BAC143_A23, BAC72_P15, BAC25_M16, BAC80_J19, BAC96_P02, BAC58_J08, BAC100_O07, BAC33_I21, BAC119_F08, BAC57_M11, | | | |
| | BAC97_I21, BAC102_L12, BAC65_N03, BAC145_C03, BAC248_M08, BAC233_P09, BAC175_D05, BAC11_N10, BAC22_P14, BAC81_E17, | | | |
| | BAC100_B22, BAC215_G20, BAC97_N03, BAC36_J22, BAC115_N23, BAC88_C21, BAC26_I17, BAC142_N17, BAC166_E01, BAC27_E18, | | | |
| | BAC232_G16, BAC69_K17, BAC13_B06 | | | |
| 15 | BAC220_P13, BAC88_I16, BAC91_O02, BAC182_D23, BAC59_F17, BAC19_L10, BAC242_O02, BAC127_N19, BAC170_H09, BAC197_N21, | 40 | 12 | 28 |
| | BAC182_L10, BAC83_E21, BAC23_H19, BAC49_P16, BAC161_M10, BAC91_O04, BAC100_H20, BAC66_J01, BAC40_C05, BAC68_C12, BAC187_A04, | | | |
| | BAC222_P21, BAC236_E23, BAC142_K19, BAC57_A19, BAC176_G02, BAC37_B13, BAC84_I01, BAC106_B15, BAC147_N01, | | | |
| | BAC138_F17, BAC197_J09, BAC81_K22, BAC10_E08, BAC128_O24, BAC102_M10, BAC231_E18, BAC11_J18, BAC120_J06, BAC8_I18 | | | |
| 16 | BAC184_K14, BAC142_G12, BAC252_I22, BAC59_B08, BAC60_M03, BAC49_J01, BAC100_H02, BAC20_K17, BAC68_B16, BAC97_C21, | 36 | 8 | 28 |
| | BAC152_G18, BAC31_G15, BAC246_C01, BAC51_A24, BAC191_F16, BAC62_C07, BAC59_L05, BAC171_P05, BAC153_M14, BAC36_L03, | | | |
| | BAC145_A03, BAC153_E19, BAC96_G02, BAC137_J20, BAC144_N12, BAC61_H22, BAC53_H20, BAC61_N20, BAC120_I17, BAC84_E07, | | | |
| | BAC200_K17, BAC170_D16, BAC96_I21, BAC54_O09, BAC101_G17, BAC72_M12 | | | |
| 17 | BAC132_D18, BAC49_N19, BAC122_B18, BAC73_C22, BAC155_O16, BAC106_L03, BAC195_B24, BAC88_C08, BAC1_L24, BAC224_E11, | 58 | 22 | 36 |
| | BAC59_B19, BAC140_N23, BAC36_P09, BAC132_L20, BAC90_H16, BAC10_I20, BAC132_O07, BAC244_N11, BAC97_C05, BAC57_D07, | | | |
| | BAC137_H11, BAC221_C05, BAC107_E15, BAC57_O05, BAC8_J15, BAC57_B10, BAC91_E18, BAC157_E20, BAC106_G05, BAC217_J07, | | | |
| | BAC68_N12, BAC84_G20, BAC195_G10, BAC65_F13, BAC128_M18, BAC130_L05, BAC211_I10, BAC163_I15, BAC193_O02, BAC84_D23, | | | |
| | BAC144_M06, BAC81_K18, BAC40_M12, BAC42_E17, BAC131_P23, BAC48_F01, BAC133_G08, BAC66_I09, BAC88_D15, BAC153_M16, | | | |
| | BAC56_P20, BAC38_O07, BAC60_H19, BAC48_H06, BAC59_E12, BAC171_D05, BAC36_L05, BAC82_B17 | | | |
| 18 | BAC43_M02, BAC72_K17, BAC191_N20, BAC106_H08, BAC34_E05, BAC30_E07, BAC167_F11, BAC86_A18, BAC210_D05, BAC127_L04, | 33 | 11 | 22 |
| | BAC131_F03, BAC86_N24, BAC19_B03, BAC171_I13, BAC111_I02, BAC241_H18, BAC1_E24, BAC144_H12, BAC120_C22, BAC251_G19, | | | |
| | BAC248_E22, BAC71_L11, BAC1_O22, BAC78_M19, BAC63_E12, BAC93_C13, BAC190_E09, BAC39_C18, BAC96_D20, BAC196_P15, | | | |
| | BAC97_B02, BAC51_E18, BAC92_P18 | | | |
| 19 | BAC75_B14, BAC37_C21, BAC65_N13, BAC33_L13, BAC15_C01, BAC110_G12, BAC57_D10, BAC78_A24, BAC24_P10, BAC242_P14, | 38 | 17 | 21 |
| | BAC193_N03, BAC96_L08, BAC50_B05, BAC131_D05, BAC232_P18, BAC63_L12, BAC246_J06, BAC192_L06, BAC195_K16, BAC144_L05, | | | |
| | BAC90_H02, BAC162_E14, BAC162_K04, BAC146_F01, BAC162_J04, BAC249_F13, BAC239_N12, BAC183_D06, BAC78_D10, BAC233_C05, | | | |
| | BAC30_K23, BAC168_H24, BAC130_J22, BAC165_O12, BAC53_O15, BAC168_E17, BAC223_A15, BAC48_A12 | | | |
| 20 | BAC40_E05, BAC109_E03, BAC127_K22, BAC85_D15, BAC155_K05, BAC143_E21, BAC86_O12, BAC247_K09, BAC165_M10, BAC86_O11, | 39 | 6 | 33 |
| | BAC81_M07, BAC81_K08, BAC29_A11, BAC132_A05, BAC96_K10, BAC44_G02, BAC75_H16, BAC63_I12, BAC110_E03, BAC36_M13, | | | |
| | BAC204_A05, BAC183_P06, BAC140_D09, BAC43_F22, BAC239_E02, BAC97_K09, BAC21_G16, BAC52_H17, BAC177_F04, BAC75_I24, | | | |
| | BAC12_K18, BAC193_H13, BAC72_N10, BAC51_F06, BAC97_O05, BAC63_G09, BAC142_K22, BAC42_C03, BAC90_N02 | | | |
| 21 | BAC29_L21, BAC190_J11, BAC183_C22, BAC100_H22, BAC65_O07, BAC249_C23, BAC36_D16, BAC84_O06, BAC11_L10, BAC63_J12, | 40 | 6 | 34 |
| | BAC155_L05, BAC203_B21, BAC182_I05, BAC56_C18, BAC82_F19, BAC65_N02, BAC117_H13, BAC166_I08, BAC203_M13, BAC165_I10, | | | |
| | BAC8_G15, BAC213_C09, BAC141_F01, BAC42_O16, BAC88_N19, BAC88_N19, BAC109_E20, BAC37_M17, BAC145_K01, BAC46_A15, | | | |
| | BAC8_N04, BAC96_C04, BAC1_C02, BAC155_O07, BAC80_E04, BAC136_D13, BAC209_K24, BAC207_B17, BAC88_N11, BAC147_A13 | | | |
| 22 | BAC185_A10, BAC173_K08, BAC82_N03, BAC73_G13, BAC45_L11, BAC135_D20, BAC235_E11, BAC122_J17, BAC209_P14, | 50 | 14 | 35 |
| | BAC170_F13, BAC193_N18, BAC96_M16, BAC199_I14, BAC40_G01, BAC12_F19, BAC161_N14, BAC143_A19, BAC229_K08, | | | |
| | BAC1_K13, BAC191_G18, BAC117_O16, BAC37_J03, BAC219_K02, BAC103_G23, BAC140_B15, BAC196_J04, BAC90_G19, | | | |
| | BAC19_A04, BAC19_A04, BAC131_H09, BAC240_E16, BAC25_B14, BAC15_I17, BAC190_O21, BAC161_F18, BAC200_M11, | | | |
| | BAC33_J12, BAC154_K23, BAC180_G12, BAC143_E09, BAC59_J18, BAC145_D16, BAC238_J21, BAC122_O04, BAC189_F08, | | | |
| | BAC78_L06, BAC131_L06, BAC73_C03, BAC33_J04, BAC248_E20 | | | |
| X | BAC32_O13, BAC153_J20, BAC143_P20, BAC166_K19, BAC72_G18, BAC161_F20, BAC122_M06, BAC16_E18, BAC180_G08, | 92 | 38 | 54 |
| | BAC43_L14, BAC230_I10, BAC96_B05, BAC89_J08, BAC62_H18, BAC46_K11, BAC120_N07, BAC63_K16, BAC118_L05, BAC63_O15, | | | |
| | BAC46_M08, BAC117_O07, BAC131_J03, BAC139_J06, BAC26_B20, BAC7_O11, BAC176_A16, BAC128_N06, BAC39_M13, | | | |
| | BAC128_N09, BAC122_O17, BAC31_P15, BAC39_H16, BAC247_M15, BAC24_I13, BAC141_P21, BAC59_K08, BAC103_A22, | | | |
| | BAC62_N08, BAC178_B15, BAC23_O07, BAC142_O01, BAC182_P03, BAC110_I17, BAC179_M02, BAC170_L05, BAC20_K10, | | | |
| | BAC76_H11, BAC211_I16, BAC6_B17, BAC57_E05, BAC209_I17, BAC43_D09, BAC141_P03, BAC56_M03, BAC200_I13, | | | |
| | BAC55_G15, BAC58_E08, BAC41_C23, BAC165_P14, BAC51_H24, BAC85_A21, BAC176_E20, BAC247_N09, BAC69_N21, | | | |
| | BAC31_O21, BAC247_B11, BAC247_B11, BAC21_J11, BAC122_G10, BAC170_L14, BAC214_M20, BAC36_B11, BAC41_L18, | | | |
| | BAC14_M14, BAC233_N08, BAC77_O23, BAC29_D20, BAC61_I07, BAC166_J20, BAC139_D06, BAC55_F19, BAC139_P13, | | | |
| | BAC139_P13, BAC43_D10, BAC90_L23, BAC154_L24, BAC31_I13, BAC27_B24, BAC123_P03, BAC170_P07, BAC171_F11, | | | |
| | BAC154_P04 | | | |
| Y | BAC202_C09, BAC52_L18, BAC158_N08, BAC10_I08, BAC58_H14, BAC94_F15, BAC36_J10, BAC126_D02, BAC220_P01, BAC80_J09, | 17 | 7 | 10 |
| | BAC60_N12, BAC147_N13, BAC37_G04, BAC132_A12, BAC170_J12, BAC41_I24, BAC194_K10 | | | |
| total | | 1440 | 392 | 1048 |
| No. | | | | |

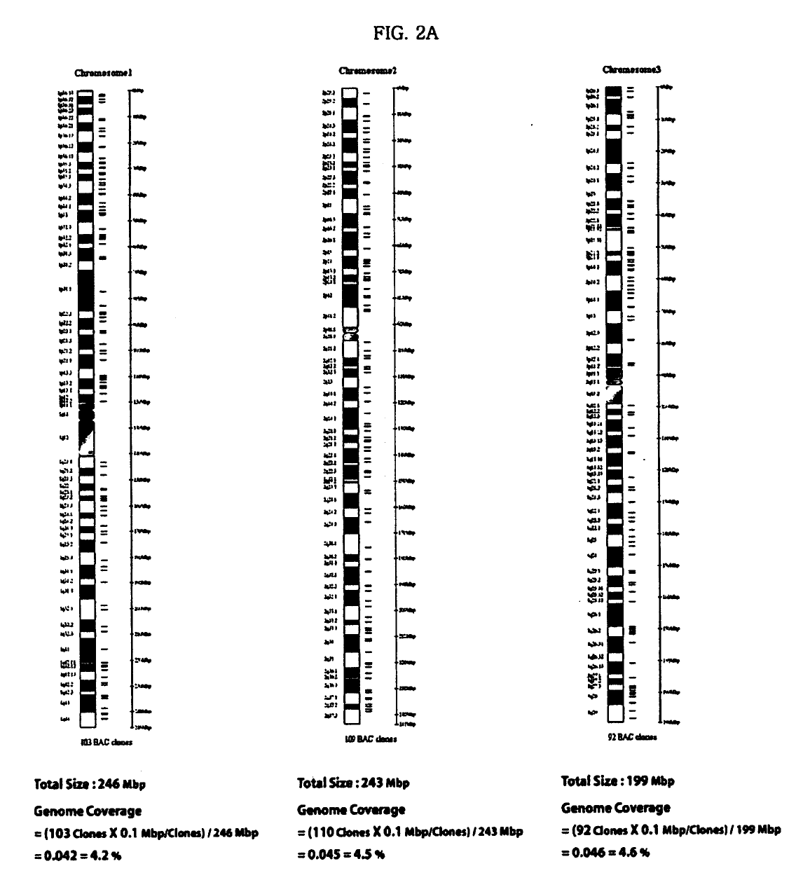

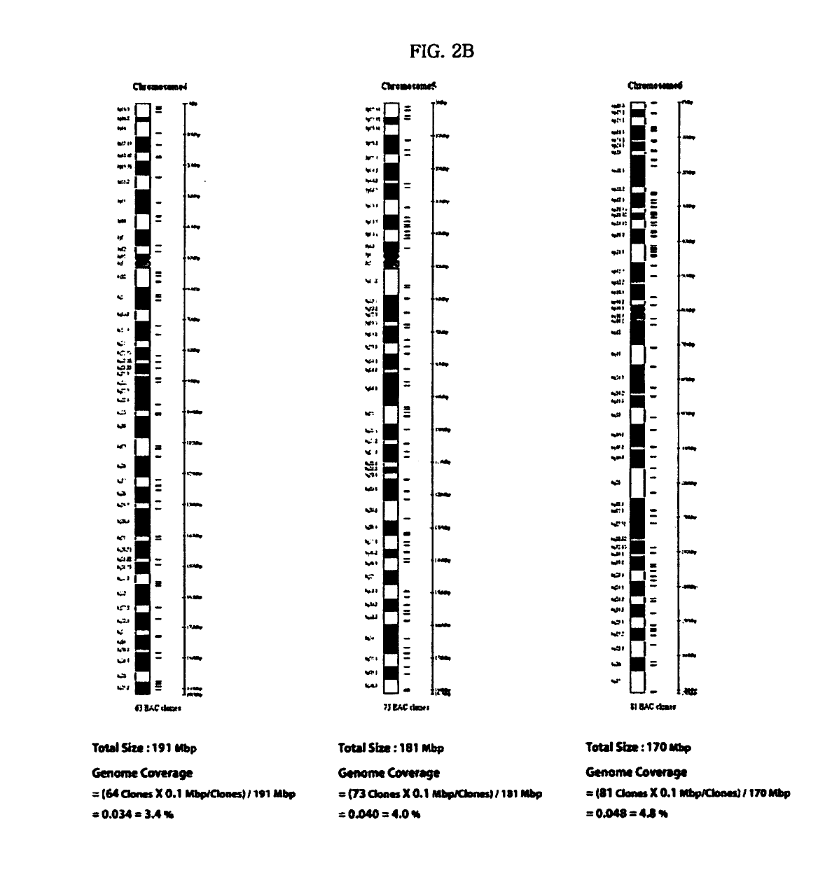

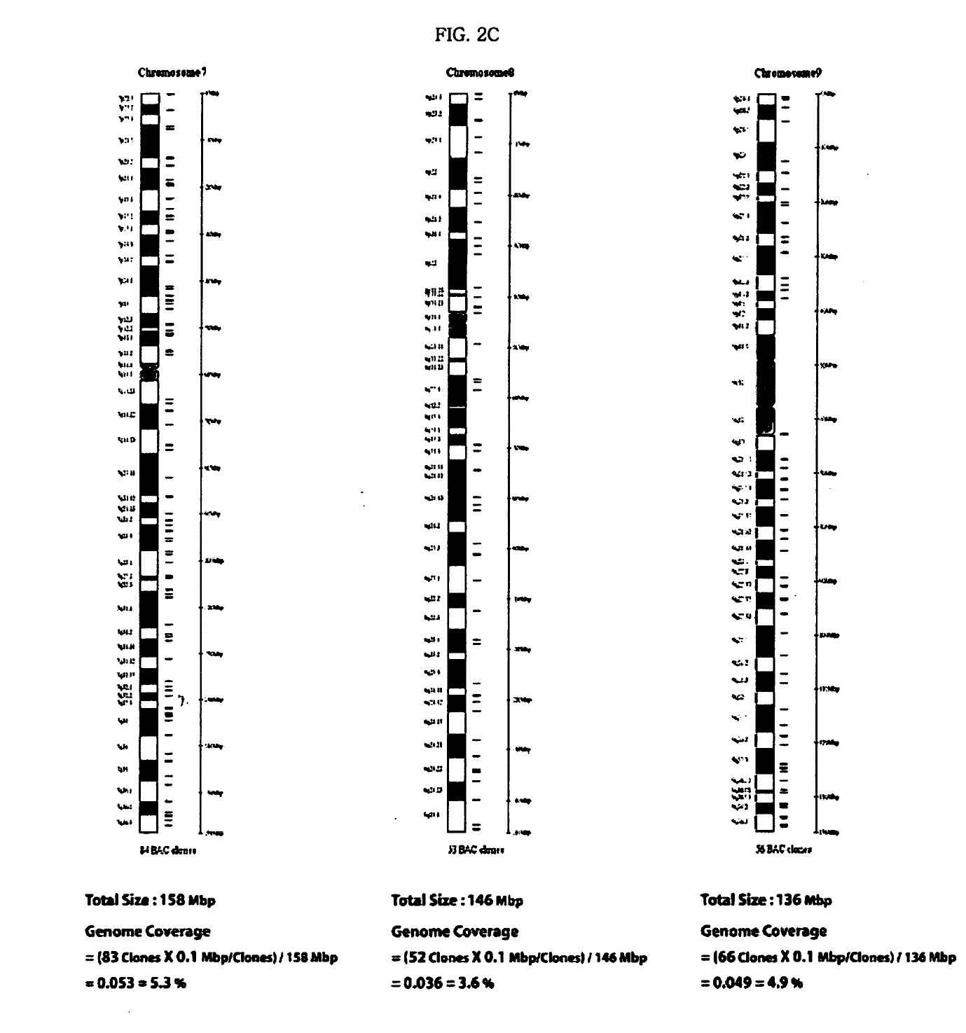

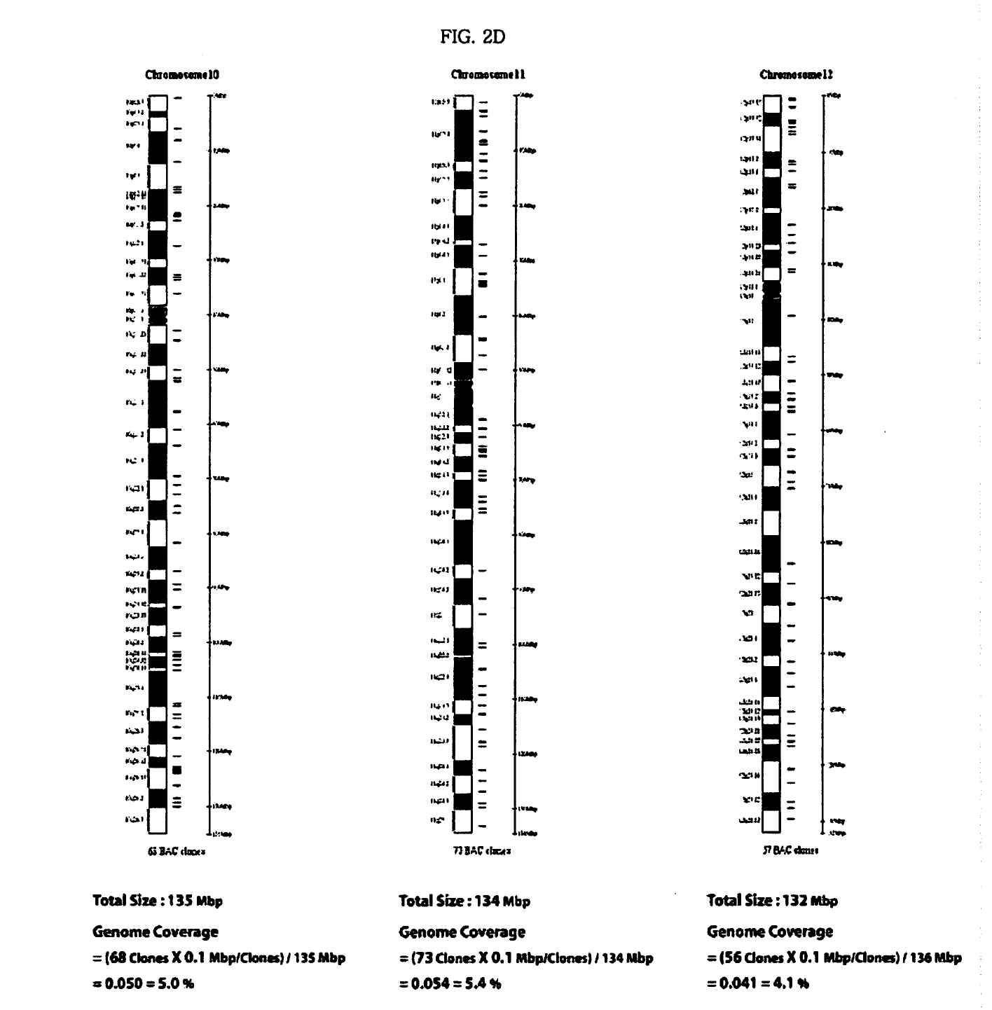

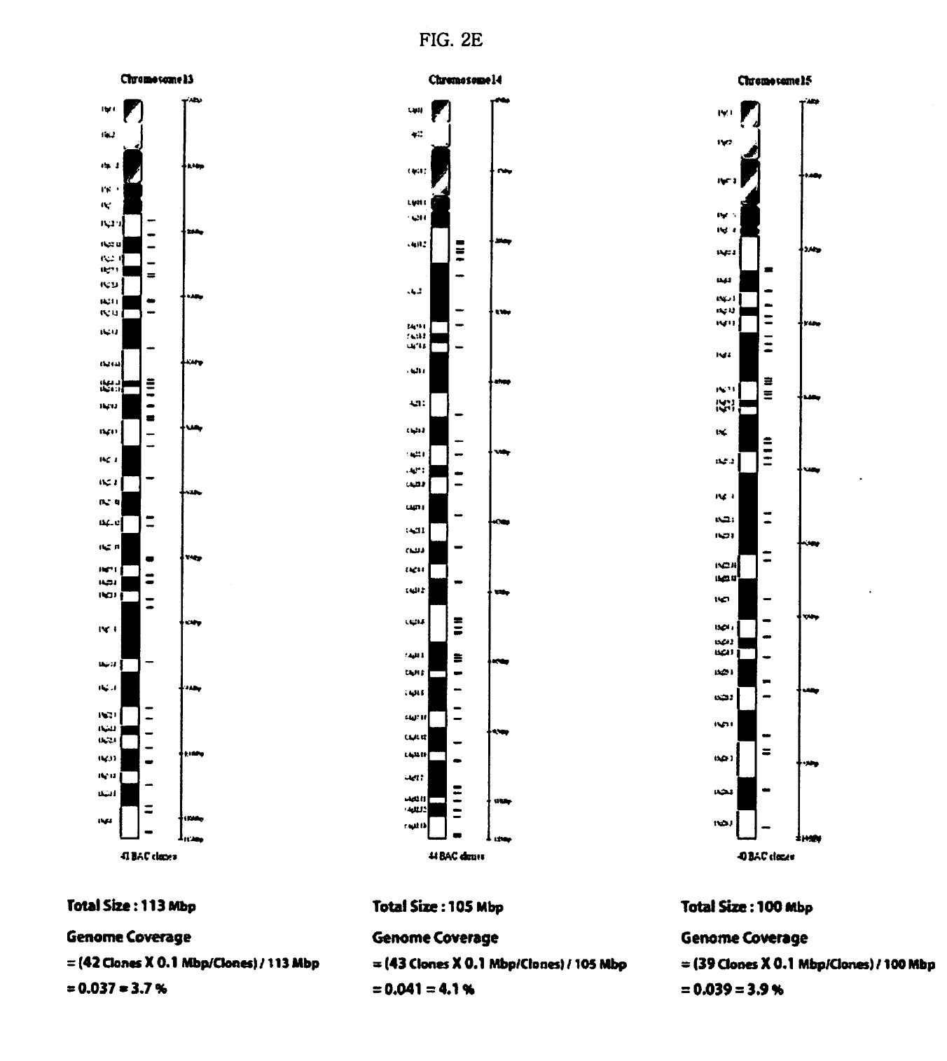

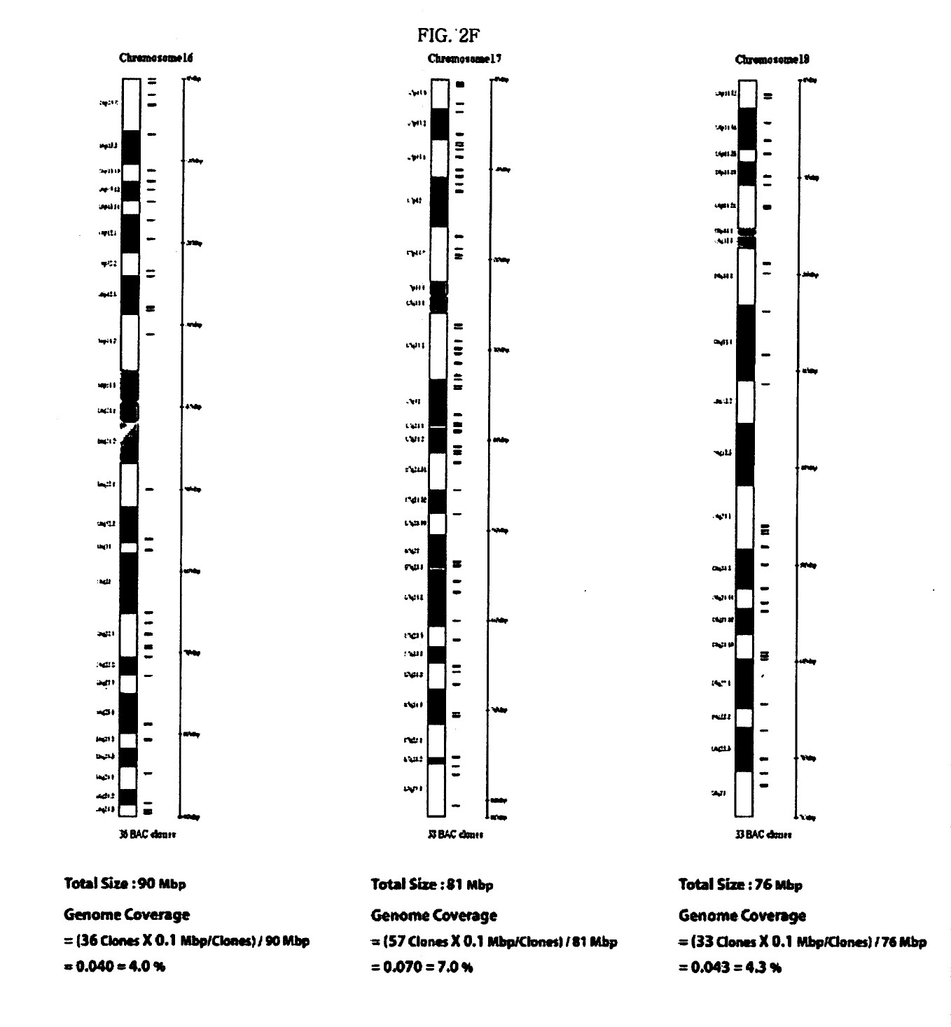

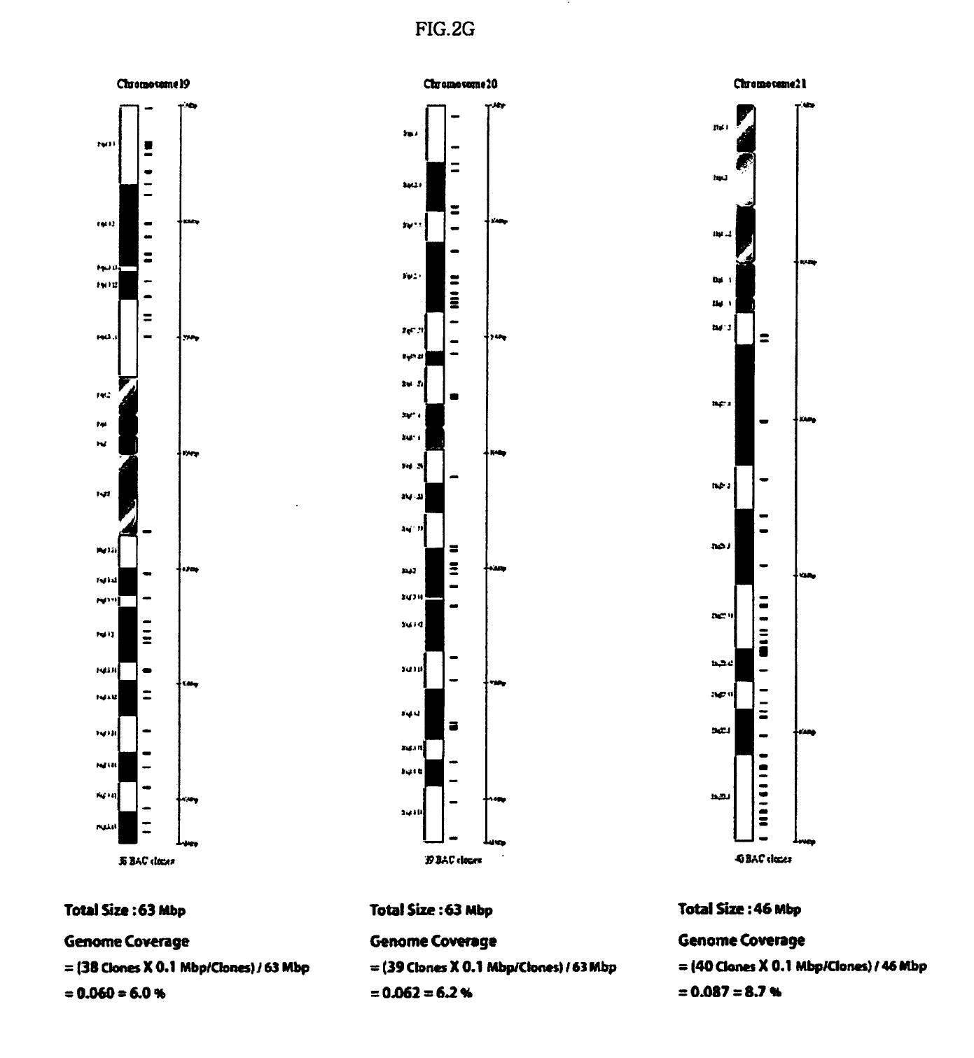

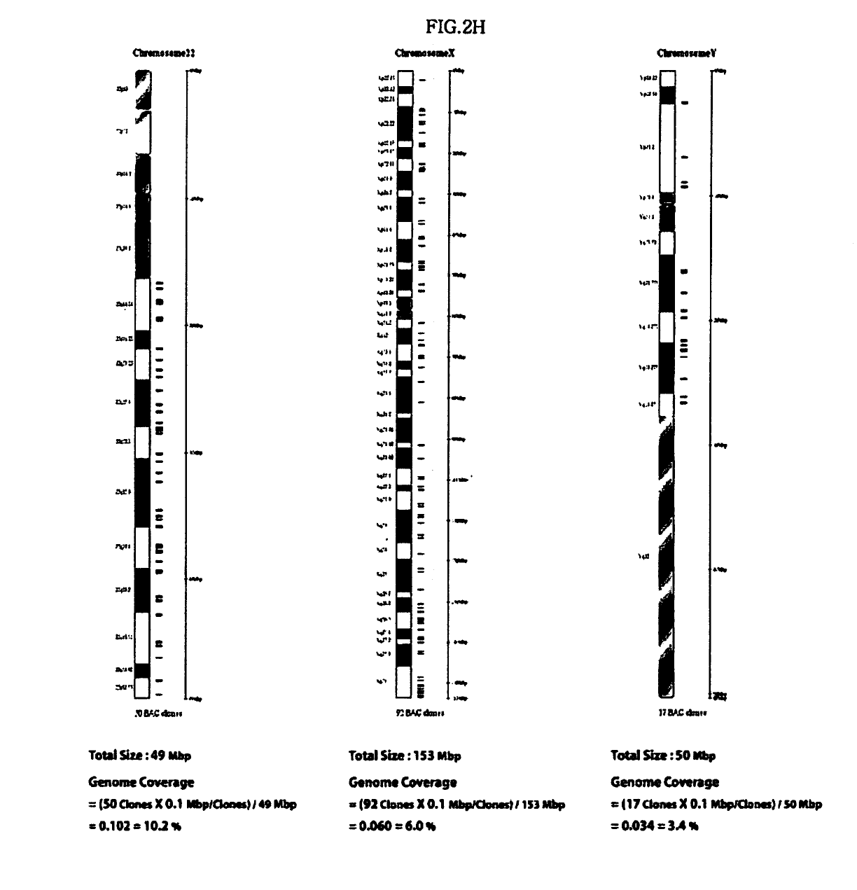

The position of each of 1440 genomic DNA fragments in chromosome and the size thereof are shown in Table 20 below and FIG. 2a-h.

TABLE 20

| | | No. of genomic DNA frament | Size of genomic DNA | Region corresponding |
|---|---|---|---|---|
| No. of chromosome | size (Mbp) | used in BAC chip | fragment (Mbp) | to the chromosome (%) |
| 1 | 246 | 103 | 0.1 | 4.2 |
| 2 | 243 | 110 | 0.1 | 4.5 |
| 3 | 199 | 92 | 0.1 | 4.6 |
| 4 | 191 | 64 | 0.1 | 3.4 |
| 5 | 181 | 73 | 0.1 | 4.0 |
| 6 | 170 | 81 | 0.1 | 4.8 |
| 7 | 158 | 83 | 0.1 | 5.3 |
| 8 | 146 | 52 | 0.1 | 3.6 |
| 9 | 136 | 66 | 0.1 | 4.9 |
| 10 | 135 | 68 | 0.1 | 5.0 |
| 11 | 134 | 73 | 0.1 | 5.4 |
| 12 | 132 | 56 | 0.1 | 4.1 |
| 13 | 113 | 42 | 0.1 | 3.7 |
| 14 | 105 | 43 | 0.1 | 4.1 |
| 15 | 100 | 39 | 0.1 | 3.9 |
| 16 | 90 | 36 | 0.1 | 4.0 |
| 17 | 81 | 57 | 0.1 | 7.0 |
| 18 | 76 | 33 | 0.1 | 4.3 |
| 19 | 63 | 38 | 0.1 | 6.0 |
| 20 | 63 | 39 | 0.1 | 6.2 |
| 21 | 46 | 40 | 0.1 | 8.7 |
| 22 | 49 | 50 | 0.1 | 10.2 |
| X | 153 | 92 | 0.1 | 6.0 |
| Y | 50 | 17 | 0.1 | 3.4 |
| total | 3060 | 1440 | 0.1 (mean) | 4.7 (mean) |

2-2. Isolation of Genomic DNA Fragment from Clone

2 μl of each BAC clone stock solution was inoculated in 85 Ml of 1×LB (DIFCO, Cat. No 244620) containing 400 μl of chloramphenicol solution (100% alcohol 100 Ml+chloramphenicol 340 mg). The reaction product is subjected to a rotating culture at 250 rpm under 37 □ for 15 hours, and a centrifugation at 6,000 rpm under 4 □ for 5 minutes, to obtain pellet. Then, a DNA was isolated by using QUIAGEN-tip 100 (QIAGEN plasmid midi kit).

Among 1 Ml of the BAC DNA, 850 μl was subjected to a ultrasonic fragmentation to be used as a spotting DNA, 3 μl was used for agarose gel electrophoresis. 20 μl of a part of randomly selected BAC DNAs was used for PFG (Pulse field gel electrophoresis) test.

2-2-1. Agarose Gel Electrophoresis

In order to confirm the isolated BAC DNA, 3 μl of BAC DNA dissolved in distilled water was treated and reacted with Not I restriction enzyme, and developed through 0.85% agarose gel. The developing profile was determined, to isolate a BAC DNA containing the genomic DNA fragment.

2-2-2. PFG Electrophoresis (CHEFF 162-00133, BIO-RAD)

Among 48 BAC clones which are a single isolation unit of a BAC DNA, 6 BAC clones were randomly selected, to be subjected to PFG electrophoresis. Even if among the 6 BAC clones, only one is poor, a new single isolation unit of 48 BAC clones was isolated again.

2-3. Spotting

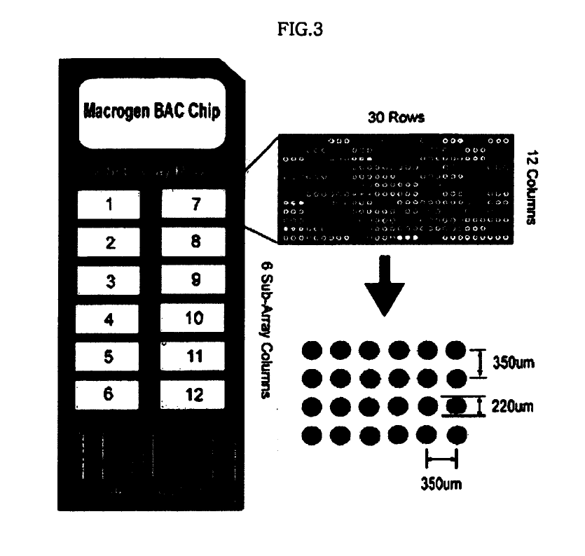

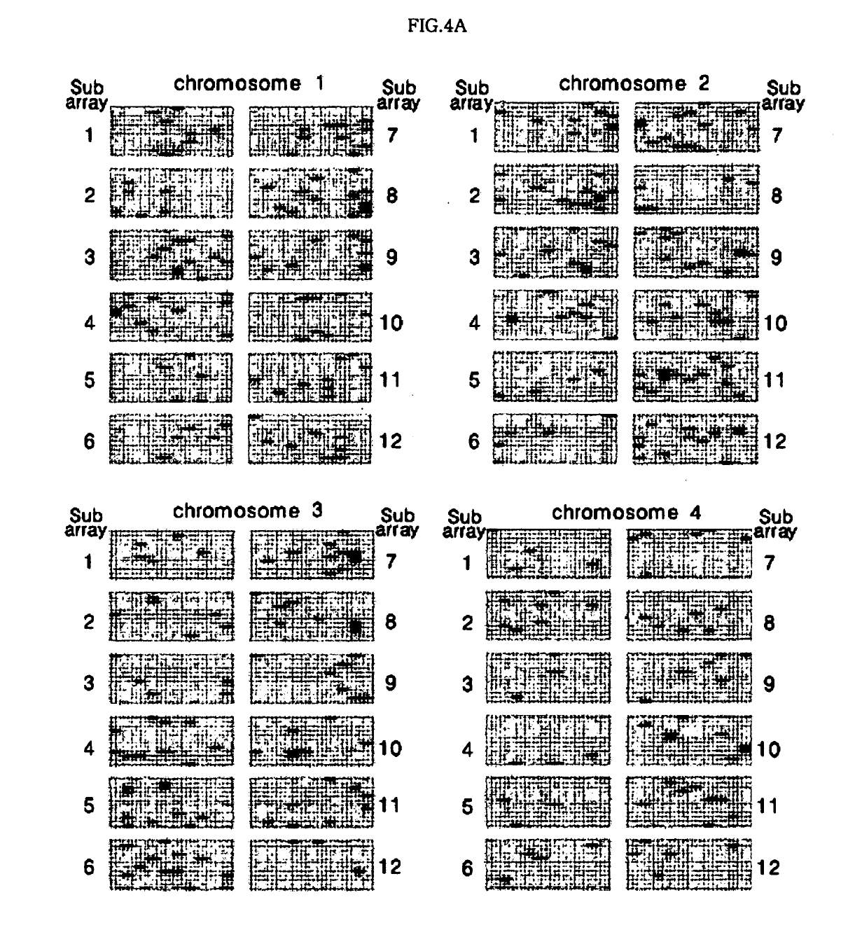

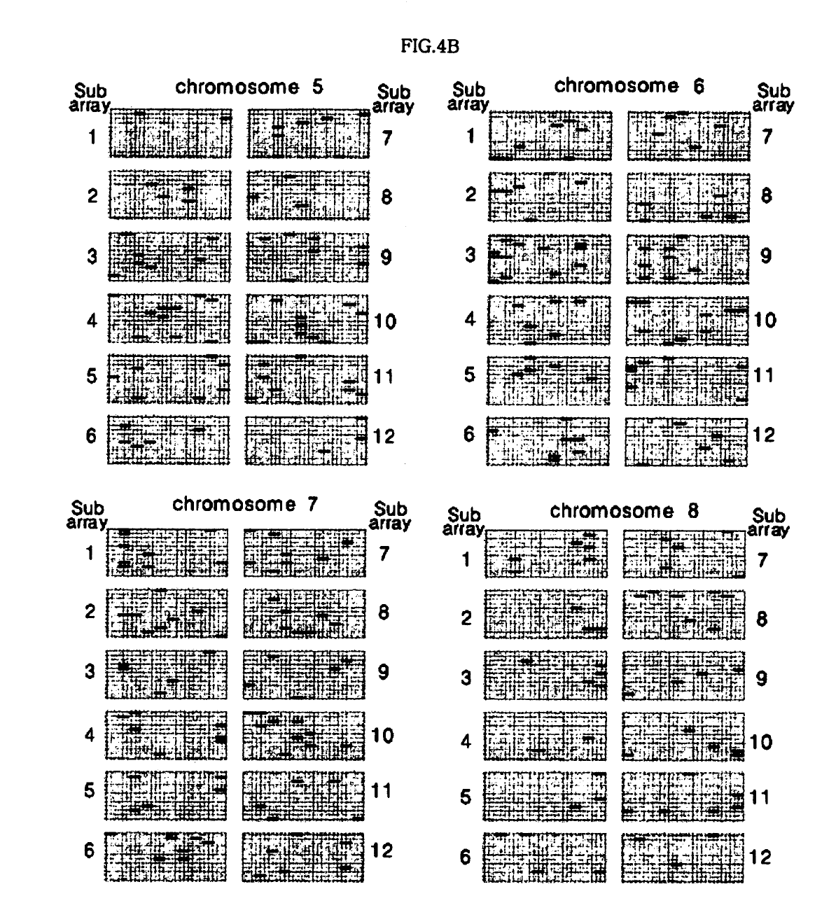

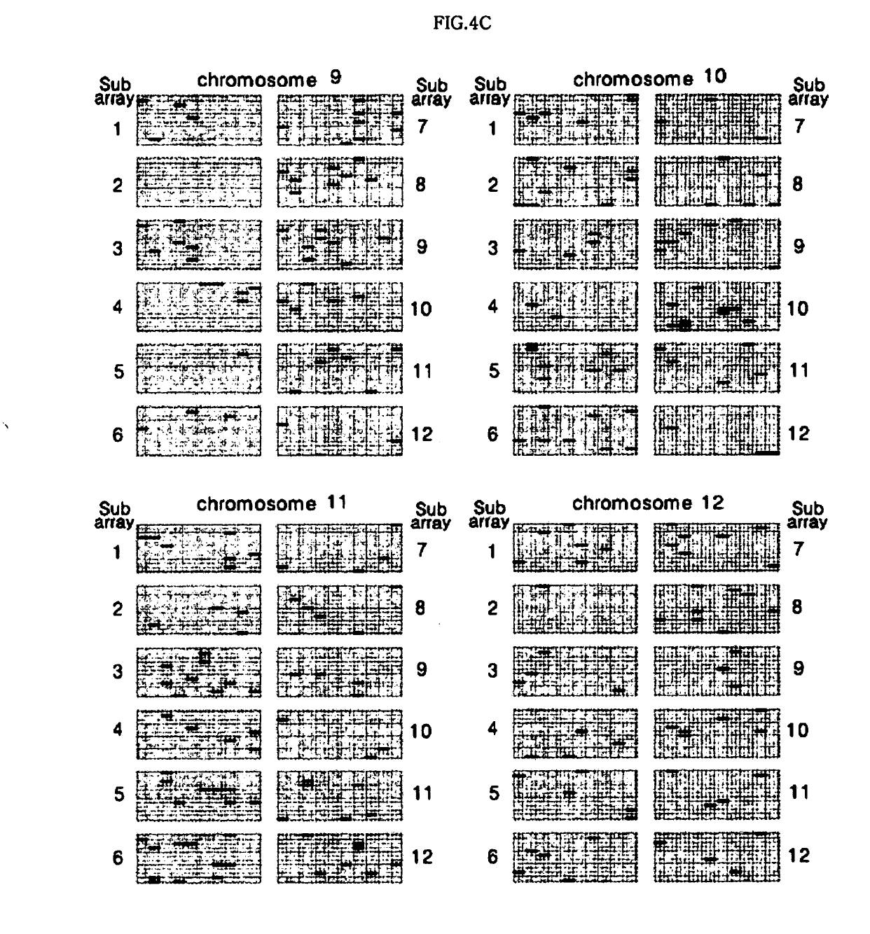

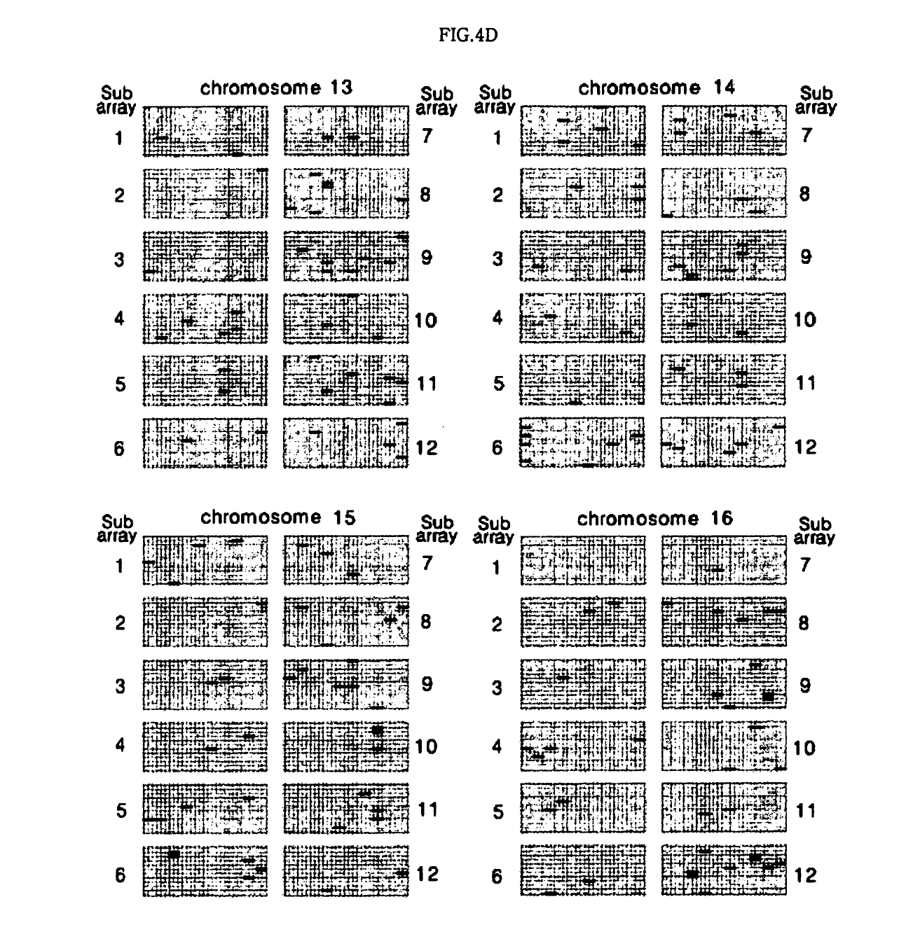

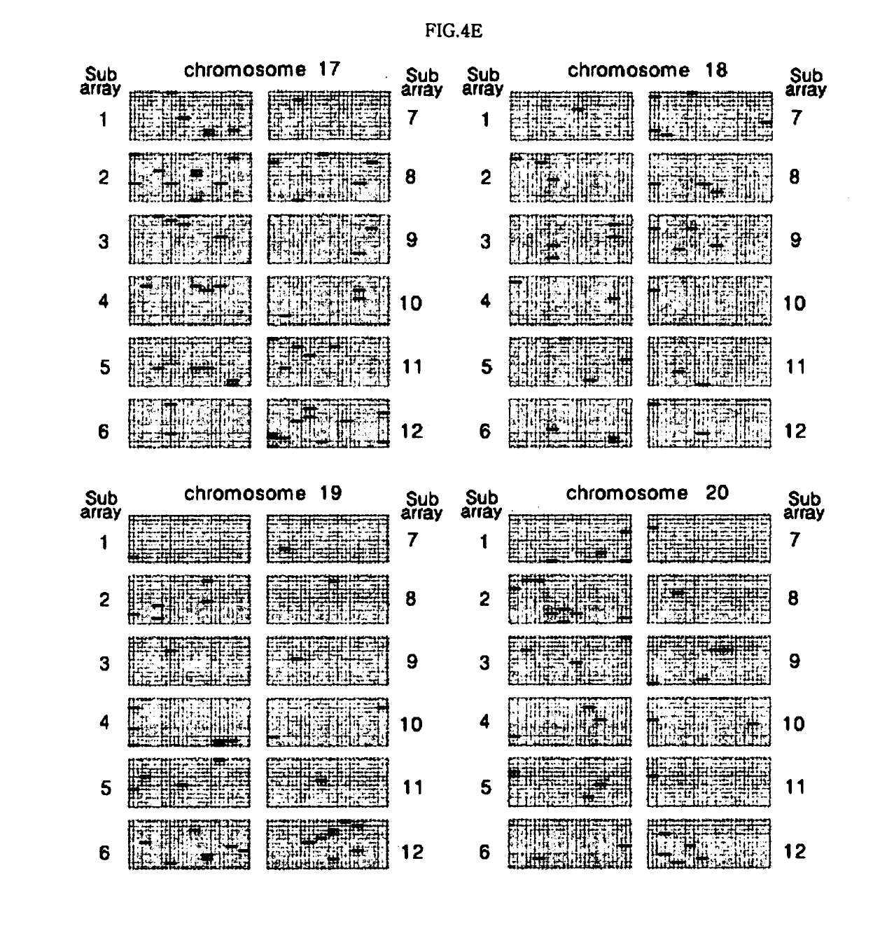

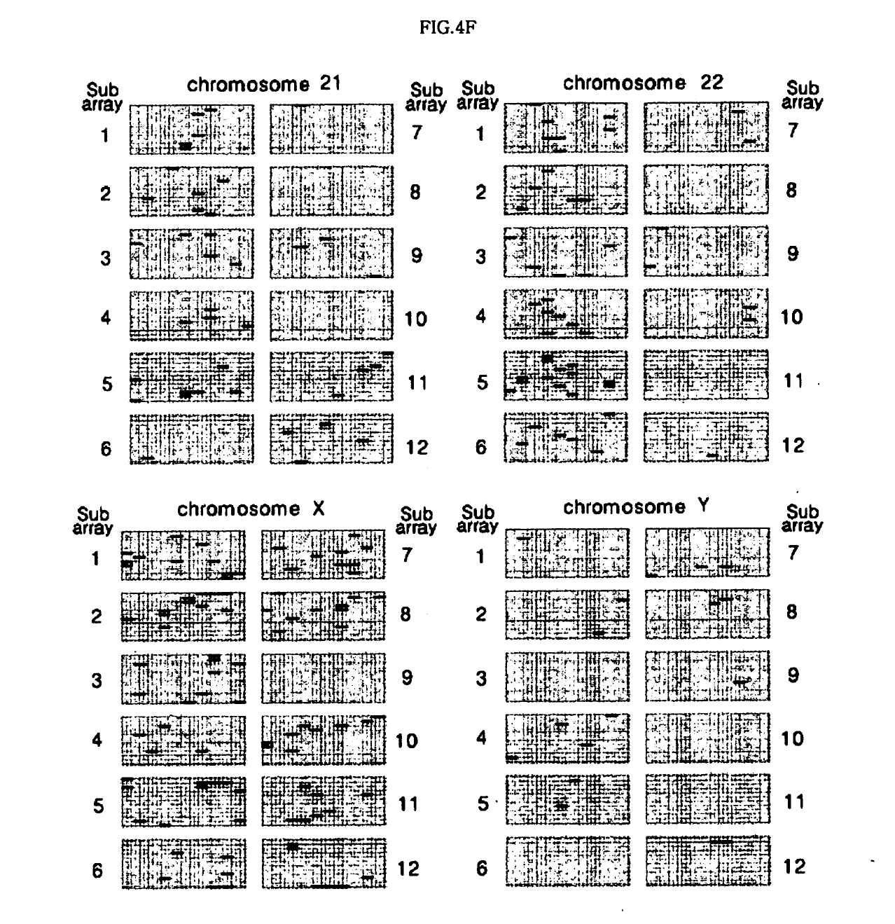

The genomic DNA fragments obtained from 1440 clones were spotted on a microarray. In the present example, 12 subarrays as shown in FIG. 3 were employed, wherein each subarray includes 360 (30×12) spots. The same genomic DNA fragment was spotted three times in horizontal direction. That is, 120 kinds of the different genomic DNA fragments are spotted in one subarray, and thus, 1440 kinds of different genomic DNA fragments are present in 12 subarrays. The number of the genomic DNA fragment spotted on each subarray and the target chromosome numbers are shown in Table 21 below and FIG. 4a-f.

TABLE 21

| No. of | No of subarray | | | | | | | | | | | | |
|---|---|---|---|---|---|---|---|---|---|---|---|---|---|
| chromosome | 1 | 2 | 3 | 4 | 5 | 6 | 7 | 8 | 9 | 10 | 11 | 12 | sum |
| 1 | 9 | 6 | 11 | 11 | 5 | 5 | 10 | 13 | 9 | 7 | 9 | 8 | 103 |
| 2 | 9 | 11 | 9 | 8 | 4 | 5 | 13 | 5 | 11 | 9 | 13 | 12 | 109 |
| 3 | 5 | 6 | 5 | 10 | 10 | 12 | 11 | 8 | 7 | 7 | 8 | 3 | 92 |
| 4 | 4 | 7 | 3 | 3 | 3 | 4 | 6 | 6 | 6 | 8 | 8 | 5 | 63 |
| 5 | 2 | 4 | 7 | 10 | 7 | 5 | 7 | 3 | 7 | 10 | 8 | 3 | 73 |
| 6 | 7 | 5 | 12 | 6 | 5 | 7 | 5 | 5 | 8 | 9 | 7 | 5 | 81 |
| 7 | 9 | 8 | 5 | 7 | 5 | 8 | 8 | 7 | 5 | 10 | 5 | 6 | 84 |
| 8 | 6 | 3 | 5 | 2 | 3 | 4 | 5 | 7 | 4 | 5 | 6 | 3 | 53 |
| 9 | 4 | 0 | 6 | 5 | 1 | 3 | 8 | 8 | 8 | 5 | 6 | 2 | 56 |
| 10 | 6 | 8 | 4 | 2 | 7 | 8 | 3 | 5 | 7 | 10 | 5 | 3 | 68 |
| 11 | 7 | 4 | 9 | 5 | 8 | 11 | 4 | 5 | 4 | 3 | 6 | 7 | 72 |
| 12 | 6 | 1 | 4 | 4 | 5 | 5 | 7 | 8 | 3 | 6 | 4 | 4 | 57 |
| 13 | 2 | 1 | 2 | 5 | 2 | 2 | 2 | 6 | 7 | 3 | 6 | 4 | 42 |
| 14 | 5 | 3 | 2 | 2 | 1 | 7 | 4 | 3 | 6 | 3 | 3 | 5 | 44 |
| 15 | 4 | 1 | 2 | 2 | 4 | 5 | 3 | 4 | 6 | 3 | 4 | 2 | 40 |
| 16 | 0 | 2 | 1 | 4 | 2 | 2 | 1 | 5 | 5 | 3 | 2 | 9 | 36 |
| 17 | 5 | 9 | 5 | 4 | 5 | 2 | 1 | 5 | 2 | 3 | 5 | 11 | 57 |
| 18 | 1 | 3 | 4 | 2 | 3 | 3 | 5 | 3 | 4 | 1 | 2 | 2 | 33 |
| 19 | 1 | 5 | 1 | 6 | 4 | 7 | 1 | 1 | 1 | 2 | 1 | 8 | 38 |
| 20 | 4 | 9 | 3 | 3 | 4 | 2 | 1 | 1 | 4 | 2 | 1 | 5 | 39 |
| 21 | 5 | 6 | 5 | 4 | 7 | 1 | 1 | 0 | 3 | 0 | 4 | 4 | 40 |
| 22 | 7 | 5 | 5 | 7 | 13 | 6 | 2 | 0 | 2 | 2 | 0 | 1 | 50 |
| X | 7 | 8 | 4 | 3 | 7 | 3 | 6 | 6 | 0 | 5 | 5 | 4 | 58 |
| Y | 1 | 2 | 0 | 4 | 2 | 0 | 3 | 2 | 1 | 0 | 0 | 2 | 17 |
| sum | 120 | 120 | 120 | 120 | 120 | 120 | 120 | 120 | 120 | 120 | 120 | 120 | 1440 |

2-3-1. Preparation of Sample

The BAC DNA solution was centrifuged at 12,000 rpm for 10 minutes, and then, the supernatant 850 μl was collected, and performed by sonication under the following conditions:

Conditions for the Sonication

Time: 5 sec., Temperature: 25 □, Pulse: 0.1/0.1

1440 BAC DNAs performed by sonication were subjected to 1% agarose gel electrophoresis, to confirm that the ultrasonic lysis has been properly accomplished. The ultrasonically lysed DNA is placed at the position ranged from 0.5 Kb to 10 Kb. The BAC DNA solution was concentrated/dried in SpeedVAC (SAVANT, AES2010-220), and dissolved in distilled water 45 μl, to determine the viscosity. The ultrasonic lysis was repeatedly performed for the samples having the high viscosity. Then, a dropping solution (100% DMSO, 1.2 ug/μl nitrocellulose) 45 μl was added thereto and transferred to 384 plate (30 μl per a well). Blank wells were filled with 50% DMSO 30 μl.

The prepared sample may be stored at −70 □ when the storage period is one month or longer. Otherwise, when the storage period is shorter than one month, the sample may be stored at −20 □. The stored sample is defrosted and subjected to spindown at 2000 rpm before being used.

2-3-2. Preparation of Substrate

A glass substrate in the size of 25 mm×75 mm×1 mm was prepared. The glass substrate was treated with amino-silane.

2-3-3. Spotting

5-10 pg was spotted per one spot on the substrate using GeneMachine MicroGrid 100. The value of the nitrogen cylinder was 103.4 kPa to 137.9 kPa, the pressure was maintained between 74.5 kPa and 101.6 kPa by using a vacuum pump.

### EXPERIMENTAL EXAMPLE 1

### Analysis of Abnormality of Chromosome Number

Specimen cells were collected from the patients suffering with Down's syndrome, Edwards syndrome, Patau syndrome, Turner syndrome and Klinefelter syndrome, and reacted on BAC chip of EXAMPLE 2.

[Procedure]

1. Isolation of Genomic DNA

500 ng of sample DNA was obtained from blood using commercially available Puregene DNA isolation Kit (Gentra, Cat. No D5500A). A reference sample was obtained by performing the same method for reference cells.

2. Probe Labeling

Each of the specimen and the reference samples was labeled with Cy3 (Cy3-dCTP) [symbol] Cy5 (Cy5-dCTP), respectively, using random priming, and purified by using Purification Kit (QIAGEN).

1) Labeling

(1) DNA (specimen or reference sample) 21 ul (500 ng) was added into a tube, and 2.5× random reaction buffer 20 ul was further added thereto.

(2) The tube was heated on a heating block at 100 □ for 5 minutes, and left on ice for 5 minutes immediately after heating.

(3) After spindown, 2 mM dNTP solution 5 ul was added into each tube [preparation of 2 mM dNTP solution: sufficiently mixing 100 mM dATP (20 ul), dGTP (20 ul), dTTP (20 ul) and dCTP (10 ul) with TE 30 ul].

(4) Cy3 3 ul was added to the specimen sample, and Cy5 3 ul was added to the reference sample.

(5) Klenow fragment 1.2 ul was added to each sample.

(6) The samples was pipetted, equally mixed, spindowned, and then, reacted in an incubator at 37 □.

2) Purification Using Spin Column (Qiagen, Qiaquick PCR Purification Kit)

(1) 250 ul of PB (binding buffer) was added to 50 ul of the reaction solution obtained, and then, dropped into the prepared spin column.

(2) The spin column was centrifuged at 13,200 rpm for 90 seconds.

(3) 750 ul of PE (washing buffer) was added into the spin column.

(4) The spin column was centrifuged at 13,200 rpm for 90 seconds.

(5) The solution collected in 2 ml tube was poured out.

(6) Further centrifugation was conducted.

(7) 50 ul of EB (eluding buffer) was dropped into the spin column, and left at the room temperature for 3 minutes.

(8) The spin column was centrifuged at 13,200 rpm for 90 seconds.

(9) 50 ul of EB was dropped into the column, and left at the room temperature for 3 minutes.

(10) The spin column was centrifuged at 13,200 rpm for 90 seconds.

3) Ethanol Precipitation

(1) Sample DNA (Cy3 labeling) 80 ul, reference DNA (Cy5 labeling) 80 ul, Cot I DNA 30 ul, 20 ul of 3M sodium citrate and cold 100% ethanol 500 ul were mixed.

(2) The admixture was left at −20 □ for 60 minutes.

(3) The reaction product was centrifuged at 13,000 rpm under 4 □ for 20 minutes.

(4) Violet precipitate was obtained after the centrifugation. The supernatant was poured out.

(5) Cold 70% ethanol 500 ul was mixed with the remaining precipitate, and then, the admixture was centrifuged at 13,000 rpm under 4 □ for 10 minutes.

(6) The supernatant was poured out, and a spindown was performed. Then, the remaining ethanol was removed by using pipette.

(7) The product obtained was left in a darkroom for 10 minutes.

(8) To the precipitate obtained, an 44 ul of array hybridizing solution and 4 ul of yeast tRNA solution were added. When the solution was left in a darkroom for 30 minutes or longer, the precipitate was dissolved and the solution became violet-colored. The solution was used for examining a probe.

4. Hybridization Reaction

BAC chip Preprocessing

(1) The upside and the downside of the BAC chip were marked with a diamond pen.

(2) Salmon sperm DNA 10 uL was added to a hybridizing solution 30 uL.

(3) The admixture was denatured on a heat block at 70 □ for 10 minutes, and then, put in ice for 5 minutes.

(4) The admixture 40 uL was loaded on the BAC chip. The chip was put in a wet slide box and left for 30 minutes.

(5) The BAC chip was immersed in triple-distilled water and washed twice.

(6) The BAC chip was washed with isopropanol, centrifuged at 550 rpm for 5 minutes, and the slide was dried.

Hybridization

(1) The violet-colored probe test solution 44 ul was denatured on the heat block at 70 □ for 15 minutes, and then, reacted on the BAC chip.

(2) The chip was left in the incubator at 37 □ for 1 hour.

(3) A hybridizing solution was loaded thereon.

(4) The chip was covered with a cover glass in the size of 20×40 mm.

(5) The chip was put in the slide box, and the slide box was put on a shker of about 5 rpm. The hybridization was conducted in the incubator wherein the constant conditions of the temperature of 37 □ and the humidity of 100% for 48-72 hours.

Washing

(1) The BAC chip was washed with the solution (pH7.3) of 46 □ containing for 15 minutes.

(2) The chip was washed with the solution of 46 □ containing 2×SSC and 0.1% SDS for 30 minutes.

(3) Another washing step was performed by using PN buffer [100 mL of mixed solution (pH 8.0) containing 0.2M Na₂HPO₄ (pH 9.0) and 0.2M NaH₂PO₄ (pH 4.3), 100 mL of distilled water and 200 uL NP40 at the room temperature for 15 minutes.

(4) The chip was washed with 2×SSC at the room temperature for 5 minutes.

(5) The chip was washed with 70%, 85% and 100% ethanol, successively, at the room temperature for 1 minute.

(6) A centrifugation was conducted at 550 rpm for 5 minutes, and the slide was dried.

4. Scanning and Analysis

The BAC chip was inserted into a microarray chip analyzer (MAC VIEWER, 2 [symbol] , A661200) and scanned, to obtain each scanned image through Cy3 filter and Cy5 filter. The scanned image was analyzed by using a BAC chip analyzing program (Macrogen) wherein the information for the genomic DNA fragment potted on the BAC chip had been inputted.

1) Scanning

a. Preparatory Matters Before Using Microarray Chip Analyzer

The microarray chip analyzer was connected to a PC with a built-in PCI. The recommendations for the PC are as follows:
IBM Pentium III or higher grade
Windows NT 4.0 or Windows 2000
768 MB RAM or higher
Hard disk of 30 GB or more
Writable/re-writable CD-ROM
1280×1024 display system (16M colors)

When scanning, the channel was selected at cy3 and cy5, and the exposure time was 1 sec. After adjusting the focus, scanning was conducted, and the scanned image was designated as a file name and stored.

2) Analysis

The scan file was operated with BAC Chip Analysis Program (Macrogen) which is an analyzing software containing the information for the BAC chip, to measure Log Ratio Chart.

The final experimental results obtained from the BAC chip show the log2 base T/R values for 1440 BAC clones equally containing all the autosomal chromosomes (1 to 22) and sex chromosome (X and Y) according to the chromosomal order. The T/R value is calculated by dividing the fluorescent intensity (CY3) of the sample by the fluorescent intensity (CY5) of the reference, wherein if the intensities of the fluorescent dyes are the same, the value is 0; if the intensity of the sample is stronger than that of the reference, the value is 0>0.2; and if the intensity of the reference is stronger than that of the sample, the value is 0<0.2. From the chromo-mean of the log2 base T/R values of each chromosome and the chromo-mean graph, the change in the number of chromosomes according to addition or deletion of each chromosome was determined.

In the case of the abnormal chromosome, the chromosome numbers of the sample and the reference were the same as 2 and the fluorescent intensities of the sample and the reference were also the same, and therefore, the chromo-mean of each chromosome became converged to 0. In this case, when a chromosome has an additional mutation, the chromo-mean of the chromosome increased to at least 0.2. In the case of the sex chromosome, according to the number of the chromosomes, the profile was the same with that of the autosomal chromosome, however determined by considering sex of the subjects. Since the reference was made by using male DNA, when the subject is male, the chromo-mean was determined by the same method as used for the autosomal chromosome. However, when the subject is female, the chromo-mean for Y chromosome was lower than −0.4, and the chromo-mean for X chromosome increased to at least 0.2. The abnormality in the number of each chromosome was determined according to the following criterions as shown in Table 22.

TABLE 22

| | | Chromosome |
|---|---|---|
| chromosome | kayotype | log2T/R mean (M) |
| Chromosome | Diploid | −0.2 < M < 0.2 |
| number 1 to 22 | deletion (Monosomy) | M < −0.2 |
| | Amplification (Trisomy) | M > 0.2 |
| X chromosome | Male (XY) | −0.2 < M < 0.2 |
| | Male, Duploid (XXY) | M > 0.2 |
| | Female (XX) | M > 0.2 |
| | Female, | −0.2 < M < 0.2 |
| | Deletion(Monosomy, XO) | |
| Y chromosome | Male (XY) | −0.4 < M < 0.4 |
| | Male, Duploid (XXY) | M > 0.4 |
| | Female (XX) | M < −0.4 |

[Result]

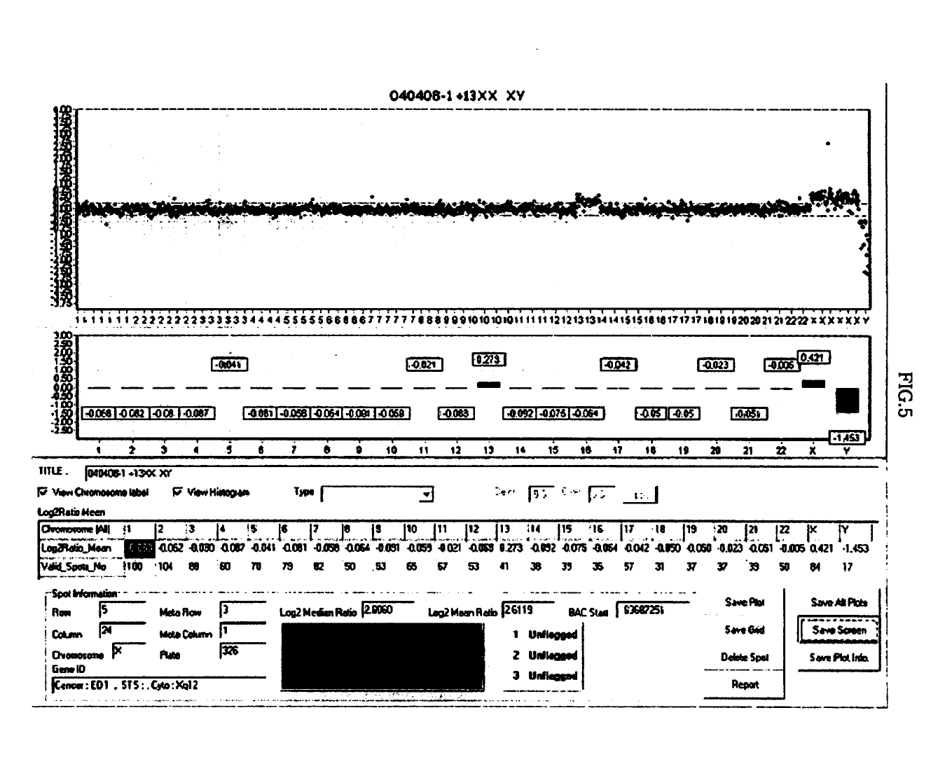

FIGS. 5 to 14 show the results from diagnosis for the abnormality in the number of the chromosomes in the sample DNA. In FIG. 5, it can be found that the log2 T/R value of X chromosome was 0.421, being higher than 0.2, from which sex of the subject was confirmed as female; the log2 T/R values of the autosomal chromosomes except Chromosome 13 ranged from −0.2 to 0.2, which showed that the autosomal chromosomes except Chromosome 13 are normal; however the log2 T/R value of Chromosome 13 was 0.273, which showed that Chromosome 13 was amplified. That is, the result of FIG. 5 shows that the subject is a female with Patau syndrome.

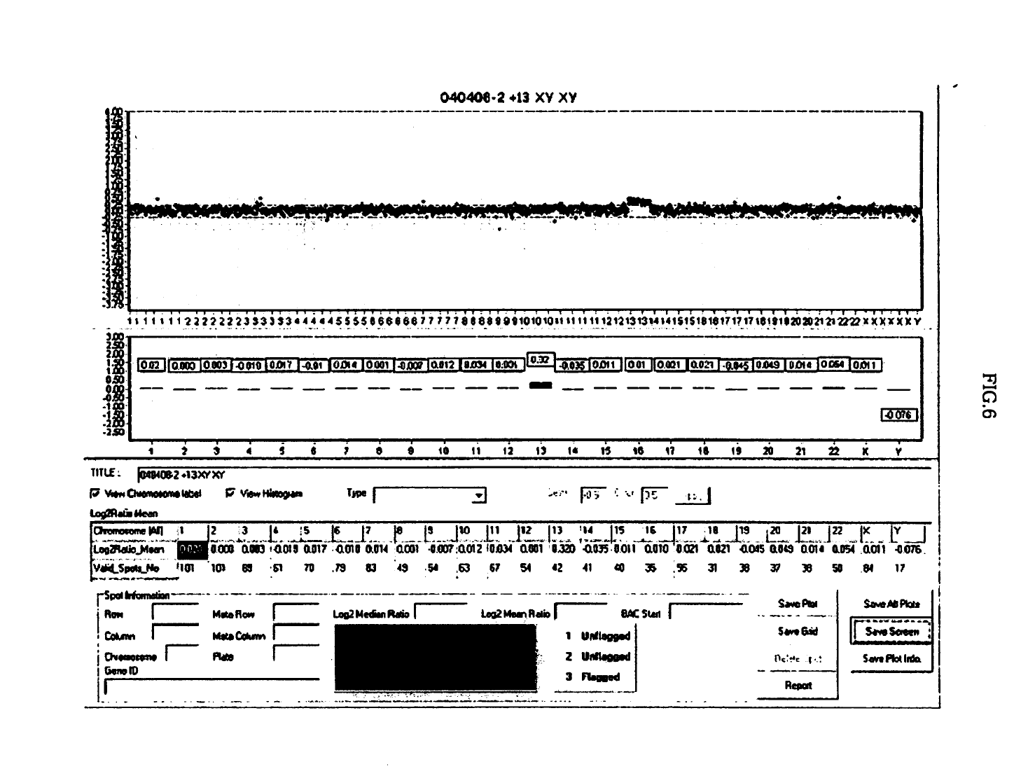

In FIG. 6, the log2 T/R value of Chromosome 13 was 0.32, the log2 T/R value of X chromosome was 0.011 and the log2 T/R value of Y chromosome was −0.076. From the result, the subject was diagnosed as a male with Patau syndrome.

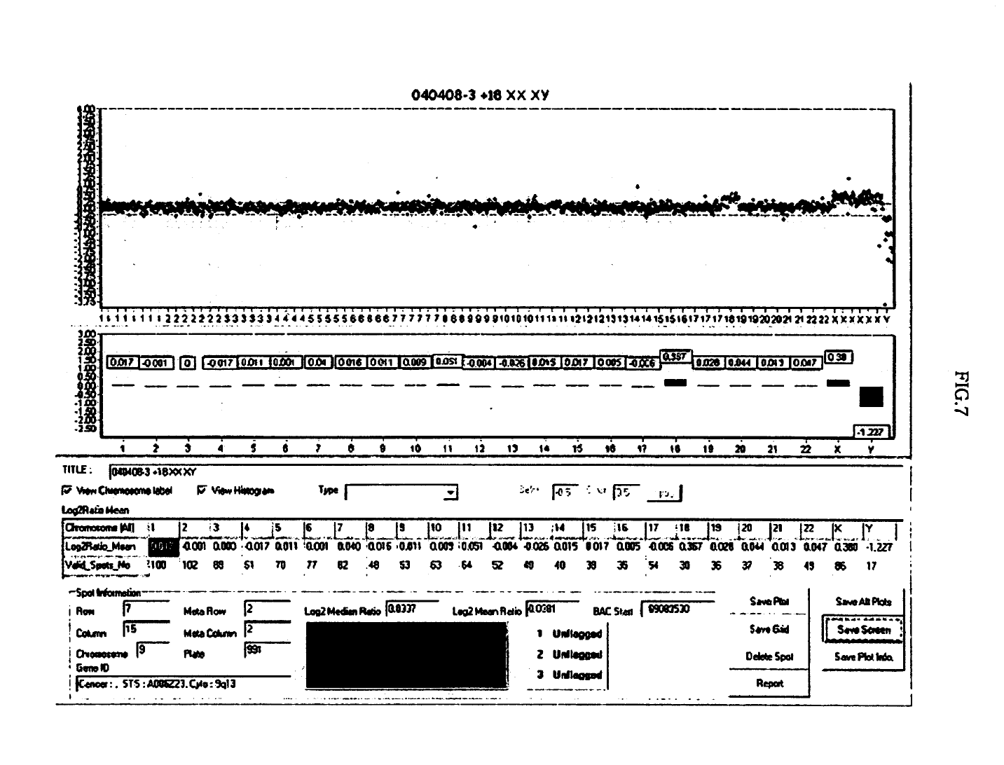

In FIG. 7, the log2 T/R value of Chromosome 18 was 0.390, the log2 T/R value of X chromosome was −0.004 and the log2 T/R value of Y chromosome was −0.118. From the result, the subject was diagnosed as a male with Edwards syndrome.

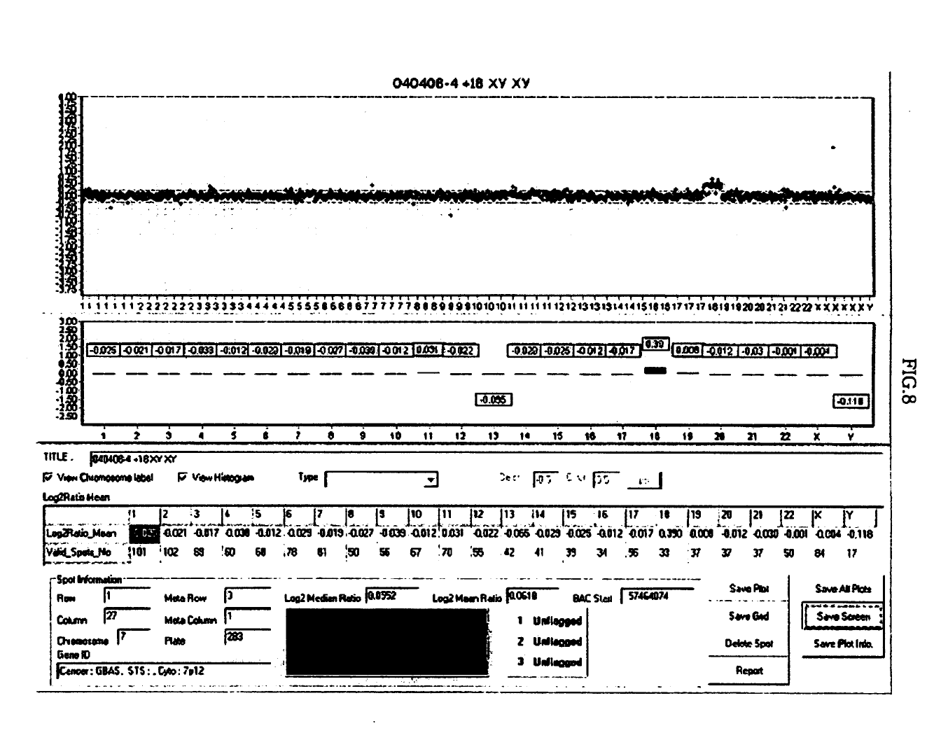

In FIG. 8, the log2 T/R value of Chromosome 18 was 0.367, the log2 T/R value of X chromosome was 0.380 and the log2 T/R value of Y chromosome was −1.227. From the result, the subject was diagnosed as a female with Edwards syndrome.

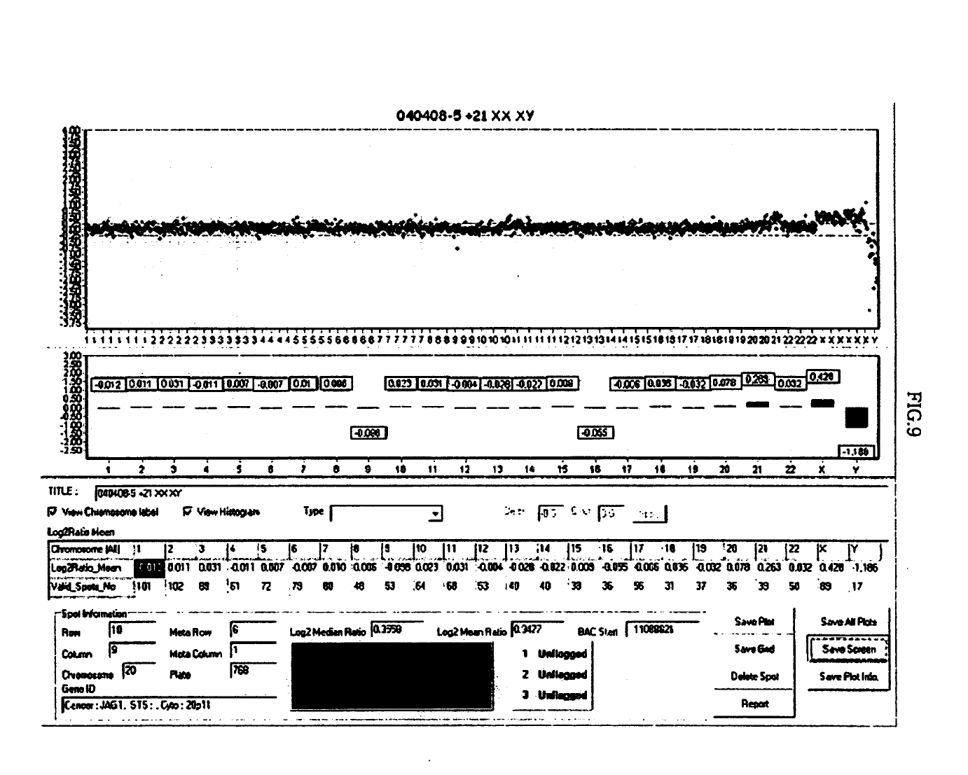

In FIG. 9, the log2 T/R value of Chromosome 21 was 0.263, the log2 T/R value of X chromosome was 0.428 and the log2 T/R value of Y chromosome was −1.186. From the result, the subject was diagnosed as a female with Down's syndrome.

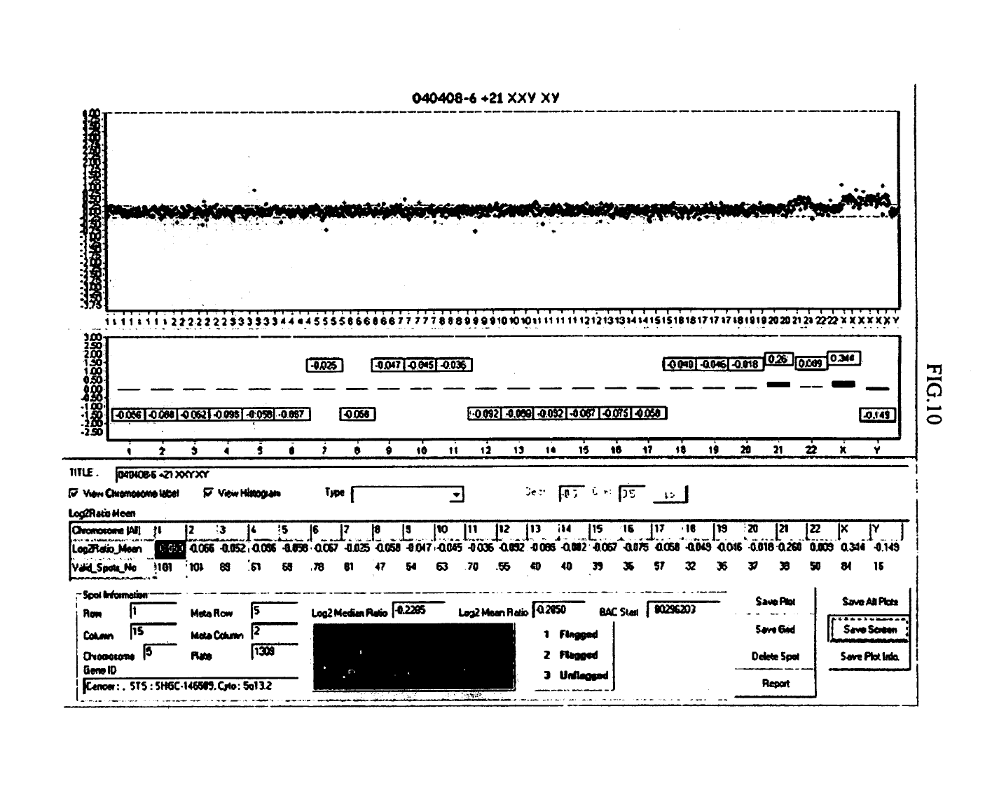

In FIG. 10, the log2 T/R value of Chromosome 21 was 0.260, the log2 T/R value of X chromosome was 0.344 and the log2 T/R value of Y chromosome was −0.149. From the result, it could be known that Chromosome 21 of the subject was amplified and X chromosome was duplicated, resulting in the karyotype of XXY.

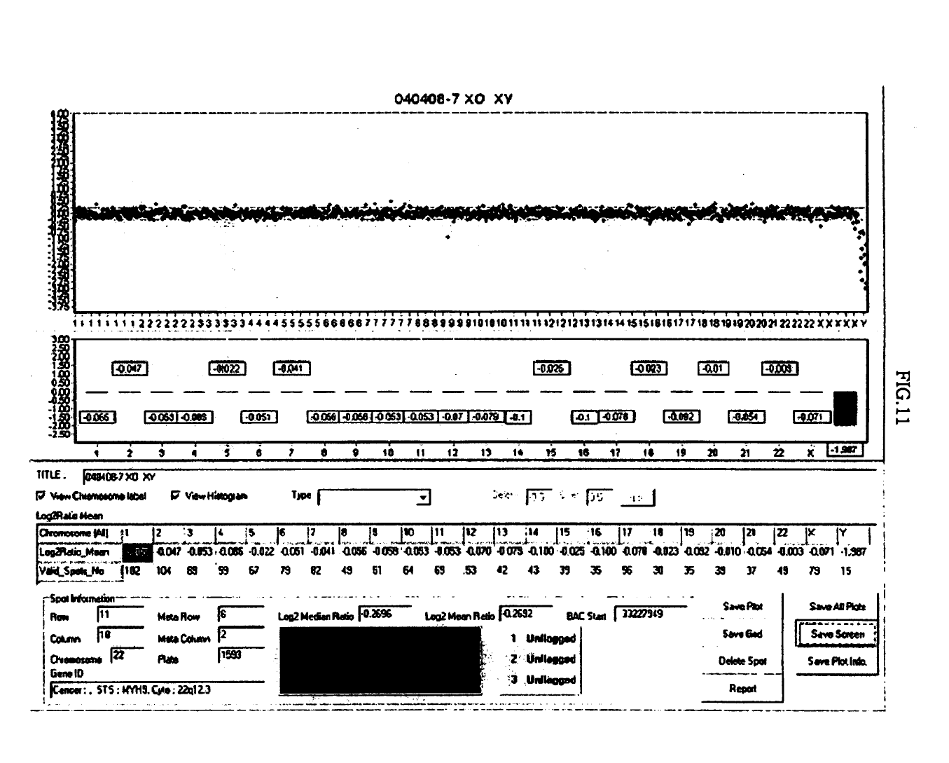

In FIG. 11, the log2 T/R value of X chromosome was −0.071 and the log2 T/R value of Y chromosome was −1.987. From the result, the subject was considered to have Turner syndrome with the karyotype of XO.

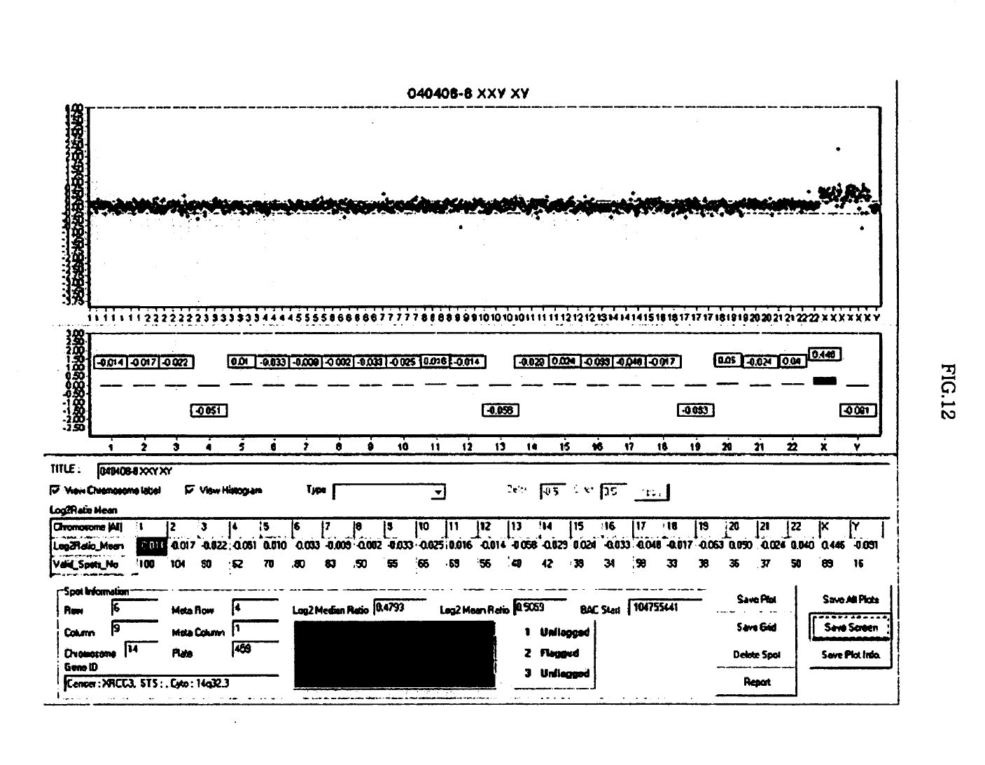

In FIG. 12, the log2 T/R value of X chromosome was 0.446 and the log2 T/R value of Y chromosome was −0.0091. From the result, the subject was considered to have Klinefelter syndrome with the karyotype of XXY.

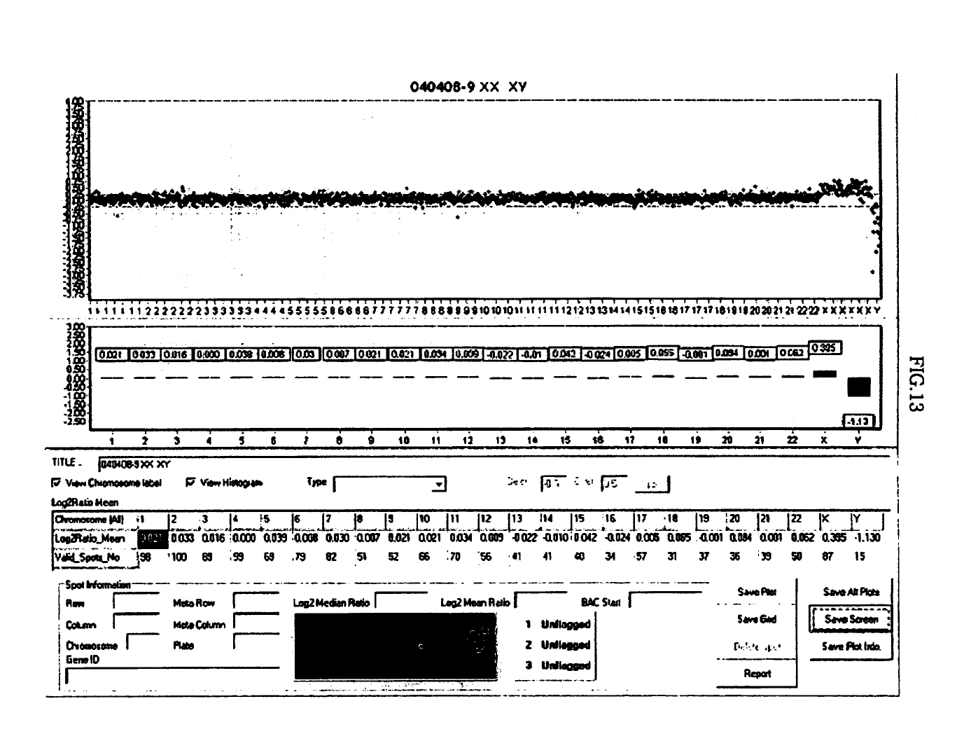

In FIG. 13, the log2 T/R value of X chromosome was 0.395 and the log2 T/R value of Y chromosome was −1.131. From the result, the subject was diagnosed as a normal female.

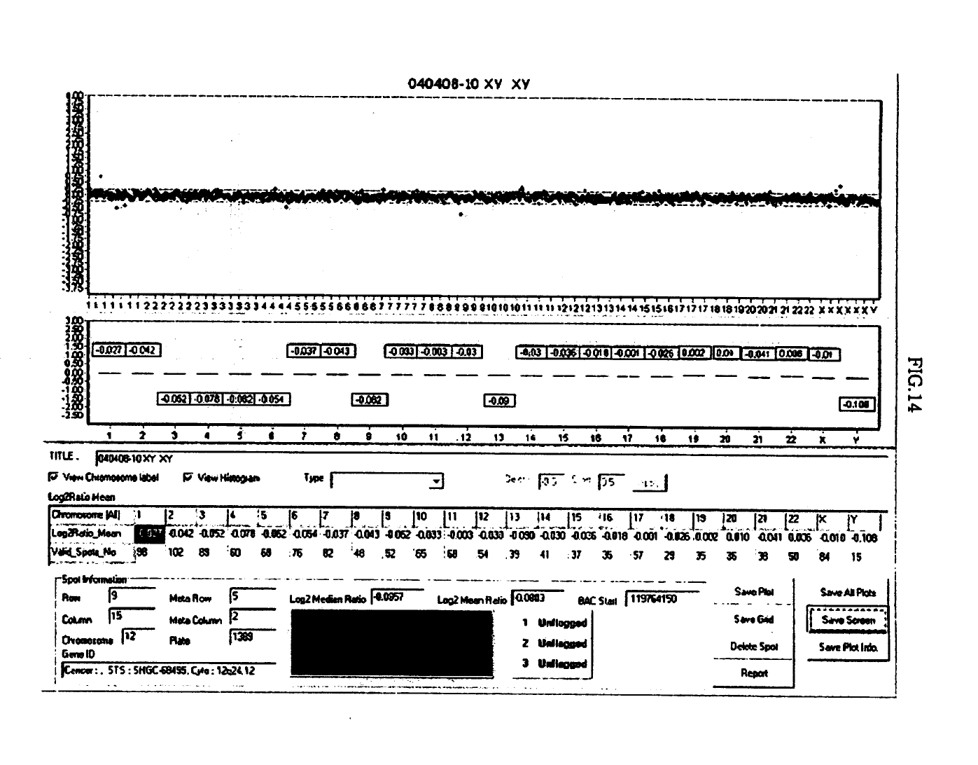

In FIG. 14, the log2 T/R value of X chromosome was −0.010 and the log2 T/R value of Y chromosome was −0.108. From the result, the subject was diagnosed as a normal male.

### EXPERIMENTAL EXAMPLE 2

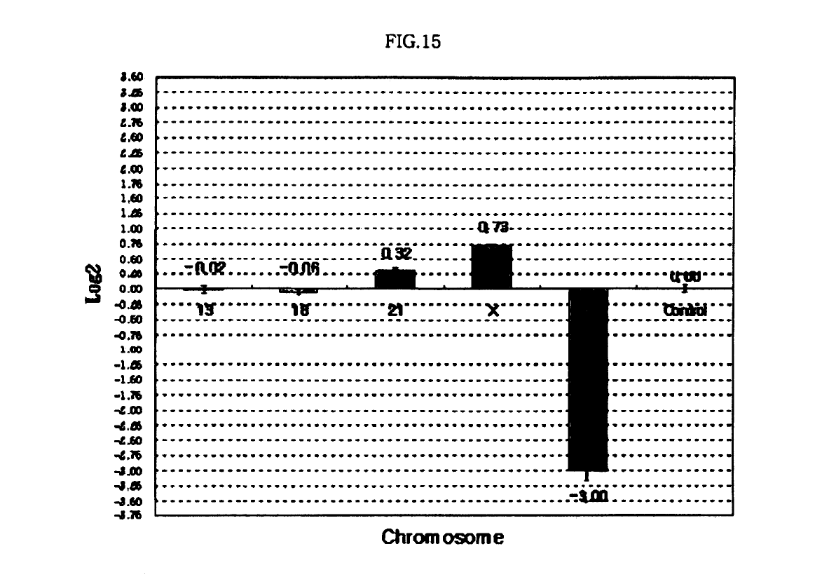

This experimental example was performed by the same method of Experimental Example 1, except that both of the sample and the reference are all labeled with Cye3. When scanning analyzing, the log2 T/R values of spot signals were used, and the analysis was conducted according to the criterions shown in Table 22. FIG. 15 shows the result of the BAC chip analysis from the subject with Down syndrome, wherein the signal showing that Chromosome 21 is duplicated was detected. $$$

While the present invention has been described in detail with reference to the preferred embodiments, those skilled in the art will appreciate that various modifications and substitutions can be made thereto without departing from the spirit and scope of the present invention as set forth in the appended claims.

LENGTHY TABLE REFERENCED HERE
US20070048742A1-20070301-T00001
Please refer to the end of the specification for access instructions.

LENGTHY TABLE
The patent application contains a lengthy table section. A copy of the table is available in electronic form from the USPTO web site (http://seqdata.uspto.gov/?pageRequest=docDetail&DocID=US20070048742A1). An electronic copy of the table will also be available from the USPTO upon request and payment of the fee set forth in 37 CFR 1.19(b)(3).

What is claimed is:

1. A method of preparation for genomic DNA microarray useful for detecting a chromosome abnormality comprising the steps of:
(a) identifying a genomic DNA fragment inserted to each clone which is obtained from an organism's genomic library;
(b) selecting a target chromosomal region to be detected detect for chromosome abnormality;
(c) selecting from the genomic DNA fragments identified in step (a), at least one genomic DNA fragment as a probe for detecting the target chromosomal region,
wherein the probe has a single chromosomal locus, includes repetitive sequence in the amount of at most 85% of the probe nucleotide, and its nucleotide sequence constitute whole or a part of the target chromosomal region, or complementary sequence thereof; and
(d) immobilizing the probe in a microarray.

2. The method of claim 1, wherein the genomic library is a vector selected from the group consisting of Bacterial Artificial Chromosome (BAC), Human Artificial Chromosome (HAC), Transformation competent Artificial Chromosome (TAC), Yeast Artificial Chromosome (YAC), P1-derived Artificial Chromosome (PAC), P1 (phage), and cosmid.

3. The method of claim 1, wherein the identifying step (a) comprises identification of information selected from the group consisting of nucleotide sequence, chromosomal location, function, and disease-association of the genomic DNA fragment.

4. The method of claim 1, wherein the genomic DNA in step (a) is at least one selected from the group consisting of table 1-1 to 1-338 as described in the detailed description of the present application.

5. The method of claim 1, wherein the genomic DNA in step (a) is at least one selected from the group consisting of table 2 as described in the detailed description of the present application.

6. The method of claim 1, wherein the target chromosomal region in step (b) includes a disease-specific mutation.

7. The method of claim 1, wherein the probe in step (c) is 10 to 300 kbp of nucleotide, and is immobilized at a concentration of 2 to 100 pg on a spot of the microarray.

8. The method of claim 1, wherein the microarray includes one or at least two probes immobilized in a spot.

9. The method of claim 1, wherein the chromosome abnormality is selected from the group consisting of mutation of nucleotide sequence, amplification in the number of chromosomes, deletion in the number of chromosomes, chromosome inversion, and chromosome translocation.

10. The method of claim 1, wherein the chromosome abnormality is an abnormality of chromosome number, and the abnormality of chromosome number is selected from the group consisting of Down's syndrome, Patau syndrome, Edwards syndrome, Turner syndrome, Klinefelter syndrome, ATR-16 (alpha-thalassemia retardation-16), CMT1A (Charcot-Marie-Tooth neuropathy 1A), Cri-du-chat syndrome, Digeorge syndrome, HNPP disease (Hereditary Neuropathy with Liability to Pressure palsies), Kallmann syndrome, Miller-Dieker syndrome, Prader-willi syndrome, Rubinstein-taybi syndrome, Steroid-sulfatase gene syndrome, Smith-magenes syndrome, Williams syndrome and Wolf-hirschhorn syndrome.

11. The method of claim 10, wherein the probe for detecting Down's syndrome is at least one genomic DNA fragment selected from the group consisting of DNA fragment 1 to 34 of Table 3 as described in the detailed description of the present application,
the probe for detecting Patau syndrome is at least one genomic DNA fragment selected from the group consisting of DNA fragment 1 to 34 of Table 4 as described in the detailed description of the present application,
the probe for detecting Edwards syndrome is at least one genomic DNA fragment selected from the group consisting of DNA fragment 1 to 28 of Table 5 as described in the detailed description of the present application,
the probe for detecting Turner syndrome is at least one genomic DNA fragment selected from the group consisting of DNA fragment 1 to 75 of Table 6 as described in the detailed description of the present application,
the probe for detecting Klinefelter syndrome is at least one genomic DNA fragment selected from the group consisting of DNA fragment 1 to 10 of Table 7 as described in the detailed description of the present application,
the probe for detecting ATR-16 (alpha-thalassemia retardation-16) syndrome is at least one genomic DNA fragment selected from the group consisting of DNA fragment 1 to 141 of Table 8 as described in the detailed description of the present application,
the probe for detecting CMT1A (Charcot-Marie-Tooth neuropathy 1A) syndrome is at least one genomic DNA fragment selected from the group consisting of DNA fragment 1 to 235 of Table 9 as described in the detailed description of the present application,
the probe for detecting Cri-du-chat syndrome is at least one genomic DNA fragment selected from the group consisting of DNA fragment 1 to 299 of Table 10 as described in the detailed description of the present application,
the probe for detecting Digeorge syndrome is at least one genomic DNA fragment selected from the group consisting of DNA fragment 1 to 231 of Table 11 as described in the detailed description of the present application,
the probe for detecting HNPP (Hereditary Neuropathy with Liability to Pressure palsies) disease is at least one genomic DNA fragment selected from the group consisting of DNA fragment 1 to 165 of Table 12 as described in the detailed description of the present application,
the probe for detecting Miller-Dieker syndrome is at least one genomic DNA fragment selected from the group consisting of DNA fragment 1 to 137 of Table 13 as described in the detailed description of the present application,
the probe for detecting Prader-willi syndrome is at least one genomic DNA fragment selected from the group consisting of DNA fragment 1 to 321 of Table 14 as described in the detailed description of the present application,
the probe for detecting Rubinstein-taybi syndrome is at least one genomic DNA fragment selected from the group consisting of DNA fragment 1 to 141 of Table 15 as described in the detailed description of the present application,
the probe for detecting Smith-magenes syndrome is at least one genomic DNA fragment selected from the group consisting of DNA fragment 1 to 165 of Table 16 as described in the detailed description of the present application,
the probe for detecting Williams syndrome or is at least one genomic DNA fragment selected from the group consisting of DNA fragment 1 to 282 of Table 17 as described in the detailed description of the present application, or
the probe for detecting Wolf-hirschhorn syndrome is at least one genomic DNA fragment selected from the group consisting of DNA fragment 1 to 72 of Table 18 as described in the detailed description of the present application.

12. A method of preparation for a microarray comprising a step of immobilizing at least one probe thereon selected from the group consisting of RNA fragment, cDNA fragment, oligomer and genomic DNA fragment,
wherein the microarray includes at least two kinds of probes immobilized in one spot, and the immobilized probes includes nucleotide selected from the group consisting of a whole chromosome, a part of a chromosome, and complementary DNA thereof showing the same phenotype in an abnormality of chromosome number, and the phenotype is a gene amplification, or a gene deletion.

13. The method of claim 12, wherein the genomic DNA fragment is selected from the group consisting of BAC DNA 1 to 25851 of Table 1 and BAC DNA 1 to 1440 of Table 2 as described in the detailed description of the present application.

14. A microarray for detecting chromosome abnormality, wherein the microarray is prepared according to claim 1 or 12.

15. A DNA chip for detecting chromosome abnormality in the whole human genomic DNA, the DNA chip comprising a microarray containing at least one immobilized genomic DNA fragment selected from the group consisting of DNA fragments 1 to 25851 of Table 1 as described in the detailed description of the present application, at least one oligomer including 20 to 100 bp of consecutive nucleotide sequence of the genomic DNA fragment, or at least one 100 bp to 10 kbp cDNA derived from the genomic DNA fragment.

16. A DNA chip for detecting chromosome abnormality in the whole human genomic DNA, the DNA chip comprising a microarray containing at least one immobilized genomic DNA fragment selected from the group consisting of DNA fragments 1 to 1440 of Table 2 as described in the detailed description of the present application, at least one oligomer including 20 to 100 bp of consecutive nucleotide sequence of the genomic DNA fragment, or at least one 100 bp to 10 kbp cDNA derived from the genomic DNA fragment.

17. A DNA chip for detecting Down's syndrome, the DNA chip comprising a microarray containing at least one immobilized genomic DNA fragment selected from the group consisting of DNA fragments 1 to 34 of Table 3 as described in the detailed description of the present application, at least one oligomer including 20 to 100 bp of consecutive nucleotide sequence of the genomic DNA fragment, or at least one 100 bp to 10 kbp cDNA derived from the genomic DNA fragment.

18. A DNA chip for detecting Patau syndrome, the DNA chip comprising a microarray containing at least one immobilized genomic DNA fragment selected from the group consisting of DNA fragments 1 to 34 of Table 4 as described in the detailed description of the present application, at least one oligomer including 20 to 100 bp of consecutive nucleotide sequence of the genomic DNA fragment, or at least one 100 bp to 10 kbp cDNA derived from the genomic DNA fragment.

19. A DNA chip for detecting Edwards syndrome, the DNA chip comprising a microarray containing at least one immobilized genomic DNA fragment selected from the group consisting of DNA fragments 1 to 28 of Table 5 as described in the detailed description of the present application, at least one oligomer including 20 to 100 bp of consecutive nucleotide sequence of the genomic DNA fragment, or at least one 100 bp to 10 kbp cDNA derived from the genomic DNA fragment.

20. A DNA chip for detecting Turner syndrome, the DNA chip comprising a microarray containing at least one immobilized genomic DNA fragment selected from the group consisting of DNA fragments 1 to 75 of Table 6 as described in the detailed description of the present application, at least one oligomer including 20 to 100 bp of consecutive nucleotide sequence of the genomic DNA fragment, or at least one 100 bp to 10 kbp cDNA derived from the genomic DNA fragment.

21. A DNA chip for detecting Klinefelter syndrome, the DNA chip comprising a microarray containing at least one immobilized genomic DNA fragment selected from the group consisting of DNA fragments 1 to 10 of Table 7 as described in the detailed description of the present application, at least one oligomer including 20 to 100 bp of consecutive nucleotide sequence of the genomic DNA fragment, or at least one 100 bp to 10 kbp cDNA derived from the genomic DNA fragment.

22. A DNA chip for detecting ATR-16 syndrome, the DNA chip comprising a microarray containing at least one immobilized genomic DNA fragment selected from the group consisting of DNA fragments 1 to 141 of Table 8 as described in the detailed description of the present application, at least one oligomer including 20 to 100 bp of consecutive nucleotide sequence of the genomic DNA fragment, or at least one 100 bp to 10 kbp cDNA derived from the genomic DNA fragment.

23. A DNA chip for detecting CMT1A (Charcot-Marie-Tooth neuropathy 1A), the DNA chip comprising a microarray containing at least one immobilized genomic DNA fragment selected from the group consisting of DNA fragments 1 to 235 of Table 9 as described in the detailed description of the present application, at least one oligomer including 20 to 100 bp of consecutive nucleotide sequence of the genomic DNA fragment, or at least one 100 bp to 10 kbp cDNA derived from the genomic DNA fragment.

24. A DNA chip for detecting Cri-du-chat syndrome, the DNA chip comprising a microarray containing at least one immobilized genomic DNA fragment selected from the group consisting of DNA fragments 1 to 299 of Table 10 as described in the detailed description of the present application, at least one oligomer including 20 to 100 bp of consecutive nucleotide sequence of the genomic DNA fragment, or at least one 100 bp to 10 kbp cDNA derived from the genomic DNA fragment.

25. A DNA chip for detecting Digeorge syndrome, the DNA chip comprising a microarray containing at least one immobilized genomic DNA fragment selected from the group consisting of DNA fragments 1 to 231 of Table 11 as described in the detailed description of the present application, at least one oligomer including 20 to 100 bp of consecutive nucleotide sequence of the genomic DNA fragment, or at least one 100 bp to 10 kbp cDNA derived from the genomic DNA fragment.

26. A DNA chip for detecting HNPP (Hereditary Neuropathy with Liability to Pressure palsies), the DNA chip comprising a microarray containing at least one immobilized genomic DNA fragment selected from the group consisting of DNA fragments 1 to 165 of Table 12 as described in the detailed description of the present application, at least one oligomer including 20 to 100 bp of consecutive nucleotide sequence of the genomic DNA fragment, or at least one 100 bp to 10 kbp cDNA derived from the genomic DNA fragment.

27. A DNA chip for detecting Miller-Dieker syndrome, the DNA chip comprising a microarray containing at least one immobilized genomic DNA fragment selected from the group consisting of DNA fragments 1 to 137 of Table 13 as described in the detailed description of the present application, at least one oligomer including 20 to 100 bp of consecutive nucleotide sequence of the genomic DNA fragment, or at least one 100 bp to 10 kbp cDNA derived from the genomic DNA fragment.

28. A DNA chip for detecting Prader-willi syndrome, the DNA chip comprising a microarray containing at least one immobilized genomic DNA fragment selected from the group consisting of DNA fragments 1 to 321 of Table 14 as described in the detailed description of the present application, at least one oligomer including 20 to 100 bp of consecutive nucleotide sequence of the genomic DNA fragment, or at least one 100 bp to 10 kbp cDNA derived from the genomic DNA fragment.

29. A DNA chip for detecting Rubinstein-taybi syndrome, the DNA chip comprising a microarray containing at least one immobilized genomic DNA fragment selected from the group consisting of DNA fragments 1 to 141 of Table 15 as described in the detailed description of the present application, at least one oligomer including 20 to 100 bp of consecutive nucleotide sequence of the genomic DNA fragment, or at least one 100 bp to 10 kbp cDNA derived from the genomic DNA fragment.

30. A DNA chip for detecting Smith-magenes syndrome, the DNA chip comprising a microarray containing at least one immobilized genomic DNA fragment selected from the group consisting of DNA fragments 1 to 165 of Table 16 as described in the detailed description of the present application, at least one oligomer including 20 to 100 bp of consecutive nucleotide sequence of the genomic DNA fragment, or at least one 100 bp to 10 kbp cDNA derived from the genomic DNA fragment.

31. A DNA chip for detecting Williams syndrome, the DNA chip comprising a microarray containing at least one immobilized genomic DNA fragment selected from the group consisting of DNA fragments 1 to 282 of Table 17 as described in the detailed description of the present application, at least one oligomer including 20 to 100 bp of consecutive nucleotide sequence of the genomic DNA fragment, or at least one 100 bp to 10 kbp cDNA derived from the genomic DNA fragment.

32. A DNA chip for detecting Wolf-hirschhorn syndrome, the DNA chip comprising a microarray containing at least one immobilized genomic DNA fragment selected from the group consisting of DNA fragments 1 to 72 of Table 18 as described in the detailed description of the present application, at least one oligomer including 20 to 100 bp of consecutive nucleotide sequence of the genomic DNA fragment, or at least one 100 bp to 10 kbp cDNA derived from the genomic DNA fragment.

33. A DNA chip for detecting constitutional diseases checked pre- or post-natally, the DNA chip comprising a microarray containing at least one immobilized genomic DNA fragment selected from the group consisting of DNA fragments of Table 2 to Table 18 as described in the detailed description of the present application, at least one oligomer including 20 to 100 bp of consecutive nucleotide sequence of the genomic DNA fragment, or at least one 100 bp to 10 kbp cDNA derived from the genomic DNA fragment.

34. A method of detecting chromosome abnormality comprising the steps of:
(a) preparing a microarray for detecting chromosome abnormality according to the method of claim 1 or 12;
(b) reacting a sample genomic DNA which is labeled by a first detectable labeling material, and a reference DNA labeled by a second detectable labeling material mixed in 1:1 weight ratio, with the probe immobilized on the microarray, and measuring hybridization ratio of the target genomic DNA or the reference DNA with the probe respectively; and
(c) analyzing chromosome abnormality of the target genomic DNA on the basis of the hybridization ratio.

35. The method according to claim 34, wherein the first detectable labeling material and the second detectable labeling material are the same or different.

36. The method according to claim 34, wherein the labeling material is selected from the group consisting of radioactive isotope, fluorescent material, chemical luminescent, and enzyme.

37. The method according to claim 34, wherein the sample is tissue, cell, or body fluids which are selected from the group consisting of saliva, blood, urine, semen, and amniotic fluid.

38. The method according to claim 34, wherein in the step (c), the chromosome abnormality is analyzed as follows:
No chromosome abnormality, if the hybridization ratio of the target genomic DNA is the same as that of the reference DNA,
Chromosome amplification, if the hybridization ratio of the target genomic DNA is higher than that of the reference DNA.
Chromosome deletion, if the hybridization ratio of the target genomic DNA is lower than that of the reference DNA.

39. A method of detecting chromosome abnormality comprising the steps of:
(a) selecting a probe including consecutive 20 to 100 bp of genomic DNA fragment, and immobilizing the probe on a microarray, wherein the genomic DNA fragment is BAC DNA in Table 1 or BAC DNA in Table 2 as described in the detailed description of the present application, and the probe has a single chromosomal locus, and includes repetitive sequence in the amount of at most 85% of the probe nucleotide;
(b) reacting the sample genomic DNA in a sample which is labeled by first detectable labeling material, and the reference DNA labeled by second detectable labeling material in the same mixture ratio, with the probe immobilized on the microarray, and measuring hybridization ratio of the sample genomic DNA or the reference DNA with the probe respectively; and
(c) deciding chromosome abnormality of the sample genomic DNA on the basis of:
No chromosome abnormality, if the hybridization ratio of the sample genomic DNA is the same as that of the reference DNA is decided to produce no chromosome abnormality,
Chromosome amplification, if the hybridization ratio of the sample genomic DNA is higher than that of the reference DNA.
Chromosome deletion, if the hybridization ratio of the sample genomic DNA is lower than that of the reference DNA.

40. The method according to claim 39, wherein the microarray includes one or at least two kinds of probes in a spot.

41. The method according to claim 39, wherein the chromosome abnormality is abnormality of chromosome number selected from the group consisting of Down's syndrome, Patau syndrome, Edwards syndrome, Turner syndrome, Klinefelter syndrome, ATR-16 (alpha-thalassemia retardation-16), syndrome CMT1A (Charcot-Marie-Tooth neuropathy 1A), Cri-du-chat syndrome, Digeorge syndrome, HNPP (Hereditary Neuropathy with Liability to Pressure palsies), Miller-Dieker syndrome, Prader-willi syndrome, Rubinstein-taybi syndrome, Smith-magenes syndrome, Williams syndrome and Wolf-hirschhorn syndrome.

42. The method according to claim 39, wherein the probe for detecting Down's syndrome is at least one genomic DNA fragment selected from the group consisting of DNA fragments 1 to 34 of Table 3 as described in the detailed description of the present application,
the probe for detecting Patau syndrome is at least one genomic DNA fragment selected from the group consisting of DNA fragments 1 to 34 of Table 4 as described in the detailed description of the present application,
the probe for detecting Edwards syndrome is at least one genomic DNA fragment selected from the group consisting of DNA fragments 1 to 28 of Table 5 as described in the detailed description of the present application,
the probe for detecting Turner syndrome is at least one genomic DNA fragment selected from the group consisting of DNA fragments 1 to 75 of Table 6 as described in the detailed description of the present application,
the probe for detecting Klinefelter syndrome is at least one genomic DNA fragment selected from the group consisting of DNA fragments 1 to 10 of Table 7 as described in the detailed description of the present application,
the probe for detecting ATR-16 (alpha-thalassemia retardation-16) syndrome is at least one genomic DNA fragment selected from the group consisting of DNA fragments 1 to 141 of Table 8 as described in the detailed description of the present application,
the probe for detecting CMT1A (Charcot-Marie-Tooth neuropathy 1A) syndrome is at least one genomic DNA fragment selected from the group consisting of DNA fragments 1 to 235 of Table 9 as described in the detailed description of the present application,
the probe for detecting Cri-du-chat syndrome is at least one genomic DNA fragment selected from the group consisting of DNA fragments 1 to 299 of Table 10 as described in the detailed description of the present application,
the probe for detecting Digeorge syndrome is at least one genomic DNA fragment selected from the group consisting of DNA fragments 1 to 231 of Table 11 as described in the detailed description of the present application,
the probe for detecting HNPP (Hereditary Neuropathy with Liability to Pressure palsies) disease is at least one genomic DNA fragment selected from the group consisting of DNA fragments 1 to 165 of Table 12 as described in the detailed description of the present application,
the probe for detecting Miller-Dieker syndrome is at least one genomic DNA fragment selected from the group consisting of DNA fragments 1 to 137 of Table 13 as described in the detailed description of the present application,
the probe for detecting Prader-willi syndrome is at least one genomic DNA fragment selected from the group consisting of DNA fragments 1 to 321 of Table 14 as described in the detailed description of the present application,
the probe for detecting Rubinstein-taybi syndrome is at least one genomic DNA fragment selected from the group consisting of DNA fragments 1 to 141 of Table 15 as described in the detailed description of the present application,
the probe for detecting Smith-magenes syndrome is at least one genomic DNA fragment selected from the group consisting of DNA fragments 1 to 165 of Table 16 as described in the detailed description of the present application,
the probe for detecting Williams syndrome is at least one genomic DNA fragment selected from the group consisting of DNA fragments 1 to 282 of Table 17 as described in the detailed description of the present application, or
the probe for detecting Wolf-hirschhorn syndrome is at least one genomic DNA fragment selected from the group consisting of DNA fragments 1 to 72 of Table 18 as described in the detailed description of the present application.

43. A method of detecting abnormality of chromosome number comprising the steps of:
(a) immobilizing at least one probe thereon selected from the group consisting of RNA fragment, cDNA fragment, oligomer and genomic DNA fragment,
wherein the microarray includes at least two kinds of probes immobilized in a spot, and the probes immobilized in a spot includes a nucleotide selected from the group consisting of a whole chromosome, a part of a chromosome, and complementary DNA thereof showing the same phenotype in an abnormality of chromosome number, and the phenotype is a gene amplification, or a gene deletion.
(b) reacting sample genomic DNA in a sample which is labeled by first detectable labeling material, and the reference DNA labeled by second detectable labeling material in the same mixture ratio, with the probe immobilized on the microarray, and measuring hybridization ratio of the sample genomic DNA or the reference DNA with the probe respectively; and
(c) deciding chromosome abnormality of the sample genomic DNA on the basis of:
No chromosome abnormality, if the hybridization ratio of the sample genomic DNA is the same as that of the reference DNA is decided to produce no chromosome abnormality,
Chromosome amplification, if the hybridization ratio of the sample genomic DNA is higher than that of the reference DNA.
Chromosome deletion, if the hybridization ratio of the sample genomic DNA is lower than that of the reference DNA.

44. The method according to claim 43, wherein the probe for detecting Down's syndrome is at least one genomic DNA fragment selected from the group consisting of DNA fragments 1 to 34 of Table 3 as described in the detailed description of the present application, or is at least one selected from the group consisting of DNA oligomers having 20 bp to 100 bp consecutive nucleotide of the genomic DNA fragments,
the probe for detecting Patau syndrome is at least one genomic DNA fragment selected from the group consisting of DNA fragments 1 to 34 of Table 4 as described in the detailed description of the present application, or is at least one selected from the group consisting of DNA oligomers having 20 bp to 100 bp consecutive nucleotide of the genomic DNA fragments,
the probe for detecting Edwards syndrome is at least one genomic DNA fragment selected from the group consisting of DNA fragments 1 to 28 of Table 5 as described in the detailed description of the present application or is at least one selected from the group consisting of DNA oligomers having 20 bp to 100 bp consecutive nucleotide of the genomic DNA fragments,
the probe for detecting Turner syndrome is at least one genomic DNA fragment selected from the group consisting of DNA fragments 1 to 75 of Table 6 as described in the detailed description of the present application, or is at least one selected from the group consisting of DNA oligomers having 20 bp to 100 bp consecutive nucleotide of the genomic DNA fragments,
the probe for detecting Klinefelter syndrome is at least one genomic DNA fragment selected from the group consisting of DNA fragments 1 to 10 of Table 7 as described in the detailed description of the present application, or is at least one selected from the group consisting of DNA oligomers having 20 bp to 100 bp consecutive nucleotide of the genomic DNA fragments,
the probe for detecting ATR-16 (alpha-thalassemia retardation-16) syndrome is at least one genomic DNA fragment selected from the group consisting of DNA fragments 1 to 141 of Table 8 as described in the detailed description of the present application, or is at least one selected from the group consisting of DNA oligomers having 20 bp to 100 bp consecutive nucleotide of the genomic DNA fragments,
the probe for detecting CMT1A (Charcot-Marie-Tooth neuropathy 1A) syndrome is at least one genomic DNA fragment selected from the group consisting of DNA fragments 1 to 235 of Table 9 as described in the detailed description of the present application, or is at least one selected from the group consisting of DNA oligomers having 20 bp to 100 bp consecutive nucleotide of the genomic DNA fragments,
the probe for detecting Cri-du-chat syndrome is at least one genomic DNA fragment selected from the group consisting of DNA fragments 1 to 299 of Table 10 as described in the detailed description of the present application, or is at least one selected from the group consisting of DNA oligomers having 20 bp to 100 bp consecutive of the genomic DNA fragments,
the probe for detecting Digeorge syndrome is at least one genomic DNA fragment selected from the group consisting of DNA fragments 1 to 231 of Table 11 as described in the detailed description of the present application, or is at least one selected from the group consisting of DNA oligomers having 20 bp to 100 bp consecutive of the genomic DNA fragments,
the probe for detecting HNPP (Hereditary Neuropathy with Liability to Pressure palsies) disease is at least one genomic DNA fragment selected from the group consisting of DNA fragments 1 to 165 of Table 12 as described in the detailed description of the present application, or is at least one selected from the group consisting of DNA oligomers having 20 bp to 100 bp consecutive nucleotide of the genomic DNA fragments,
the probe for detecting Miller-Dieker syndrome is at least one genomic DNA fragment selected from the group consisting of DNA fragments 1 to 137 of Table 13 as described in the detailed description of the present application, or is at least one selected from the group consisting of DNA oligomers having 20 bp to 100 bp consecutive nucleotide of the genomic DNA fragments,
the probe for detecting Prader-willi syndrome is at least one genomic DNA fragment selected from the group consisting of DNA fragments 1 to 321 of Table 14 as described in the detailed description of the present application, or is at least one selected from the group consisting of DNA oligomers having 20 bp to 100 bp consecutive nucleotide of the genomic DNA fragments,
the probe for detecting Rubinstein-taybi syndrome is at least one genomic DNA fragment selected from the group consisting of DNA fragments 1 to 141 of Table 15 as described in the detailed description of the present application, or is at least one selected from the group consisting of DNA oligomers having 20 bp to 100 bp consecutive nucleotide of the genomic DNA fragments,
the probe for detecting Smith-magenes syndrome is at least one genomic DNA fragment selected from the group consisting of DNA fragment 1 to 165 of Table 16 as described in the detailed description of the present application, or is at least one selected from the group consisting of DNA oligomers having 20 bp to 100 bp consecutive nucleotide of the genomic DNA fragments,
the probe for detecting Williams syndrome is at least one genomic DNA fragment selected from the group consisting of DNA fragments 1 to 282 of Table 17 as described in the detailed description of the present application, or is at least one selected from the group consisting of DNA oligomers having 20 bp to 100 bp consecutive nucleotide of the genomic DNA fragments, or
the probe for detecting Wolf-hirschhorn syndrome is at least one genomic DNA fragment selected from the group consisting of DNA fragments 1 to 72 of Table 18 as described in the detailed description of the present application, or is at least one selected from the group consisting of DNA oligomers having 20 bp to 100 bp consecutive nucleotide of the genomic DNA fragments

45. A method of detecting a disease-specific chromosome abnormality comprising the steps of:
(a) identifying a genomic DNA fragment inserted into each clone which is obtained from an organism's genomic library;
(b) immobilizing the whole or a part of genomic DNA fragment identified in step (a) on a microarray;
(b) reacting the sample genomic DNA of a patient which is labeled by a first detectable labeling material, and a reference DNA labeled by a second detectable labeling material in the same mixture ratio, with the probe immobilized on the microarray, and measuring hybridization ratio of the sample genomic DNA or the reference DNA with the probe respectively; and
(c) detecting a region and type of a chromosome abnormality of the patient by analyzing the hybridization ratio.

46. The method according to claim 45, wherein the first detectable labeling material and the second detectable labeling material are the same or different.

47. The method according to claim 46, wherein the labeling material is selected from the group consisting of radioactive isotope, fluorescent material, chemical luminescent, and enzyme.
